(19) United States
(12) Patent Application Publication
(10) Pub. No.: US 2005/0043344 A1
(43) Pub. Date: Feb. 24, 2005

(54) Heteroarylalkanoic acids as integrin receptor antagonists derivatives

(75) Inventors: Mark L. Boys, Brighton, MI (US); Lori A. Schretzman, East Hanover, NJ (US); Michael B. Tollefson, Dardenne Prairie, MO (US); Nizal Samuel Chandrakumar, Grafton, MA (US); Ish Kumar Khanna, Libertyville, IL (US); Maria Nguyen, Ann Arbor, MI (US); Victoria L. Downs, Pinckney, MI (US); Scott B. Mohler, Chicago, IL (US); Glen J. Gesicki, Chicago, IL (US); Thomas D. Penning, Elmhurst, IL (US); Barbara B. Chen, Glenview, IL (US); Yaping Wang, Acton, MA (US); Albert Khilevich, Buffalo Grove, IL (US); Bipinchandra N. Desai, Vernon Hills, IL (US); Yi Yu, Glenview, IL (US); John A. Wendt, South Lyon, MI (US); Heather Stenmark, Chicago, IL (US); Hongwei Wu, Buffalo Grove, IL (US); Renee M. Huff, Park Ridge, IL (US); Srinivasan Raj Nagarajan, Chesterfield, MO (US); Balekudru Devadas, Chesterfield, MO (US); Hwang-Fun Lu, Ballwin, MO (US); Mark Russell, Gurnee, IL (US); Dale P. Spangler, San Diego, CA (US); Mihir D. Parikh, Chesterfield, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/743,354

(22) Filed: Dec. 22, 2003

Related U.S. Application Data
(60) Provisional application No. 60/435,467, filed on Dec. 20, 2002.

(51) Int. Cl. A61K 31/4745

(52) U.S. Cl. 514/300

(57) ABSTRACT

The present invention relates to pharmaceutical compositions comprising compounds of the Formula I, or a pharmaceutically acceptable salt thereof, and methods of selectively inhibiting or antagonizing the α_{V}β₃ and/or the α_{V}β₅ integrin without significantly inhibiting the α_{V}β₆ integrin.

### CROSS-REFERENCE RELATED TO APPLICATION

This application claims priority from Provisional Application Ser. No. 60/435,467 filed Dec. 20, 2002, which is hereby incorporated by reference in its entirety.

### FIELD OF INVENTION

The present invention relates to pharmaceutical agents (compounds) which are α_{V}β₃ and/or α_{V}β₅ integrin antagonists and as such are useful in pharmaceutical compositions and in methods for treating conditions mediated by α_{V}β₃ and/or α_{V}β₅ integrins.

### BACKGROUND OF THE INVENTION

The integrin α_{V}β₃ (also known as vitronectin receptor), is a member of the integrin family of heterodimeric transmembrane glycoprotein complexes that mediate cellular adhesion events and signal transduction processes. Integrin α_{V}β₃ is expressed in number of cell types and has been shown to mediate several biologically relevant processes, including adhesion of osteoclasts to the bone matrix, vascular smooth muscle cell migration and angiogenesis.

The integrin avb3 has been shown to play a role in various conditions or disease states including tumor metastasis, solid tumor growth (neoplasia), osteoporosis, Paget's disease, humoral hypercalcemia of malignancy, osteopenia, angiogenesis, including tumor angiogenesis, retinopathy including macular degeneration, arthritis, including rheumatoid arthritis, periodontal disease, psoriasis and smooth muscle cell migration (e.g. restenosis artherosclerosis). The compounds of the present invention are α_{V}β₃ antagonists and can be used, alone or in combination with other therapeutic agents, in the treatment or modulation of various conditions or disease states described above. Additionally, it has been found that such agents would be useful as antivirals, antifungals and antimicrobials

The integrin α_{V}β₅ plays a role in neovascularization. Therefore the compounds of this invention which act as antagonists of the α_{V}β₅ integrin will inhibit neovascularization and will be useful for treating and preventing angiogenesis metastasis, tumor growth, macular degeneration and diabetic retinopathy.

Antagonists of α_{V}β₃ or dual α_{V}β₃/α_{V}β₅ antagonists can be useful therapeutic agents for treating many pathological conditions, including the treatment or prevention of osteopenia or osteoporosis, or other bone disorders, such as Paget's disease or humoral hypercalcemia of malignancy; neointimal hyperplasia, which can cause artherosclerosis or restenosis after vascular procedures; periodontal disease; treatment and prevention of viral infections or other pathogens; the treatment of neoplasia; pathological angiogenesis or neovascularization such as tumor metastasis, diabetic retinopathy, macular degeneration, rheumatoid arthritis, or osteoarthritis.

Compounds that antagonize the α_{V}β₅ and/or the α_{V}β₃ receptor have been reprinted in the literature. For example, WO 01/96334 provides heteroarylalkanoic acid compounds useful as α_{V}β₃ and/or α_{V}β₅ inhibitors.

### SUMMARY OF THE INVENTION

As evidenced by the continuing research in integrin antagonists and by the shortcomings of the compounds and methods of the art, there still remains a need for small-molecule, non-peptidic selective α_{V}β₃ and/or α_{V}β₅ antagonist that displays decreased side-effects, and improved potency, pharmacodynamic, and pharmacokinetic properties, such as oral bioavailability and duration of action, over already described compounds. Such compounds would prove to be useful for the treatment, prevention, or suppression of various pathologies enumerated above that are mediated by α_{V}β₃ and/or α_{V}β₅ receptor binding and cell adhesion and activation.

The compounds of this invention include 1) α_{V}β₃ integrin antagonists; or 2) α_{V}β₅ integrin antagonists; or 3) mixed or dual α_{V}β₃/α_{V}β₅ antagonists. The present invention includes compounds which inhibit the respective integrins and also includes pharmaceutical compositions comprising such compounds. The present invention further provides for methods for treating or preventing conditions mediated by the α_{V}β₃ and/or α_{V}β₅ receptors in a mammal in need of such treatment comprising administering a therapeutically effective amount of the compounds of the present invention and pharmaceutical compositions of the present invention. Administration of such compounds and compositions of the present invention inhibits angiogenesis, tumor metastasis, tumor growth, skeletal malignancy of breast cancer, osteoporosis, Paget's disease, humoral hypercalcemia of malignancy, retinopathy, macular degeneration, arthritis including rheumatoid, periodontal disease, smooth muscle cell migration, including restenosis and artherosclerosis, and microbial or viral diseases.

The compounds of the present invention can be used, alone or in combination with other therapeutic agents, in the treatment or modulation of various conditions or disease states described above.

In order to prevent bleeding side effects associated with the inhibition of α_{V}β₃, it would be beneficial to have a high selectivity ratio of α_{V}β₃ and α_{V}β₅ over α_{V}β₃. The compounds of the present invention include selective antagonists of α_{V}β₃ over α_{V}β₃.

The present invention relates to a class of compounds represented by Formula I 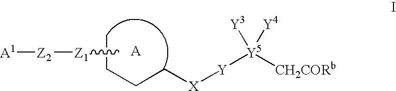
or a pharmaceutically acceptable salt thereof, wherein 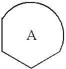
is a 4-8 membered monocyclic or a 7-12 membered bicyclic ring, containing 1 to 5 heteroatoms, selected from the group consisting of O, N or S; optionally saturated or unsaturated, optionally substituted with one or more substituents selected from the group consisting of alkyl, haloalkyl, aryl, heteroaryl, halogen, alkoxyalkyl, aminoalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, and —(CH₂)ₘCOR wherein m is 0-2 and R is hydroxy, alkoxy, alkyl or amino; with the proviso that when Y⁴ in formula I is H, the ring A may not be an oxazole, with X-Y containing side-chain connected at the carbon-2 as in 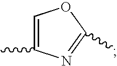
The ring A may further contain a carboxamide, sulfone, sulfonamide or an acyl group.

A¹ is a 5-9 membered monocyclic or 8-14 membered poly-cyclic heterocycle of the formula 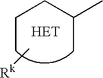
containing at least one nitrogen atom and optionally 1 to 4 heteroatoms or groups, selected from O, N, S, SO₂ or CO; optionally saturated or unsaturated; optionally substituted by one or more R^{k} selected from the group consisting of hydroxy, alkyl, alkoxy, alkoxyalkyl, thioalkyl, haloalkyl, cyano, amino, alkylamino, halogen, acylamino, sulfonamide and —COR wherein R is hydroxy, alkoxy, alkyl or amino; or A¹ is 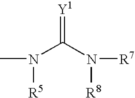
wherein Y¹ is selected from the group consisting of N—R², O, and S;
R² is selected from the group consisting of H; alkyl; aryl; hydroxy; alkoxy; cyano; amido; alkylcarbonyl; arylcarbonyl; alkoxycarbonyl; aryloxycarbonyl; haloalkylcarbonyl; haloalkoxycarbonyl; alkylthiocarbonyl; arylthiocarbonyl; acyloxymethoxycarbonyl;
R² taken together with R⁷ forms a 4-12 membered dinitrogen containing heterocycle optionally substituted with one or more substituent selected from the group consisting of lower alkyl, thioalkyl, alkylamino, hydroxy, keto, alkoxy, halo, phenyl, amino, carboxyl or carboxyl ester; or
R² taken together with R⁷ forms a 4-12 membered heterocycle containing one or more heteroatom selected from O, N and S optionally unsaturated; or
R² taken together with R⁷ forms a 5 membered heteroaromatic ring fused with an aryl or heteroaryl ring;
R⁷ (when not taken together with R²) and R⁸ are independently selected from the group consisting of H; alkyl; aralkyl; amino; alkylamino; hydroxy; alkoxy; arylamino; amido, alkylcarbonyl, arylcarbonyl; alkoxycarbonyl; aryloxy; aryloxycarbonyl; haloalkylcarbonyl; haloalkoxycarbonyl; alkylthiocarbonyl; arylthiocarbonyl; acyloxymethoxycarbonyl; cycloalkyl; bicycloalkyl; aryl; acyl; benzoyl; or
NR⁷ and R⁸ taken together form a 4-12 membered mononitrogen containing monocyclic or bicyclic ring optionally substituted with one or more substituent selected from lower alkyl, carboxyl derivatives, aryl or hydroxy and wherein said ring optionally contains a heteroatom selected from the group consisting of O, N and S;
R⁵ is selected from the group consisting of H and alkyl; or
   A¹ is 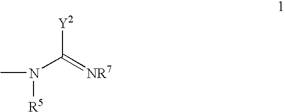
   wherein Y² is selected from the group consisting of alkyl; cycloalkyl; bicycloalkyl; aryl; monocyclic heterocycles;
Z₁ is selected from the group consisting of CH₂, CH₂O, O, NH, CO, S, SO, CH(OH) and SO₂;
Z₂ is a 1-5 carbon linker optionally containing one or more heteroatom selected from the group consisting of O, S and N;
alternatively Z₁-Z₂ may further contain a carboxamide, sulfone, sulfonamide, alkenyl, alkynyl, or acyl group;
   wherein the carbon and nitrogen atoms of Z₁-Z₂ are optionally substituted by alkyl, alkoxy, thioalkyl, alkylsulfone, aryl, alkoxyalkyl, hydroxy, alkylamino, heteroaryl, alkenyl, alkynyl, carboxyalkyl, halogen, haloalkyl or acylamino;
Additionally, Z₁-Z₂ may contain a 5- or 6-membered aryl or heteroaryl ring optionally substituted with R^{c}, wherein the heteroaryl ring may contain 1-3 heteroatoms selected from the group consisting of O, N and S; R^{c} is selected from the group consisting of H, alkyl, haloalkyl, aryl, heteroaryl, halogen, alkoxyalkyl, aminoalkyl, hydroxy, alkoxy, carboxamide, or cyano.

X is selected from the group consisting of —CHR^{e}—, —NR^{f}—, —O—, —S—, —SO₂—, and —CO— wherein R^{e} is H, lower alkyl, alkoxy, cycloalkyl, alkoxyalkyl, hydroxy, alkynyl, alkenyl, haloalkyl, thioalkyl or aryl; wherein when R^{e} is hydroxy, the hydroxy group can optionally form a lactone with the carboxylic acid function of the chain; wherein R^{f} is selected from the group consisting of H, alkyl, aryl, aralkyl, and haloalkyl;
Y is selected from the group consisting of (CH₂)ₚ, —CHR^{g}—, —NR^{g}—, CO and SO₂, wherein R^{g} is selected from the group consisting of H, alkyl, haloalkyl, alkoxyalkyl, alkynyl, aryl, heteroaryl, aralkyl, hydroxy, alkoxy, and carboxyalkyl; wherein p is 0 or 1.

Optionally the group X-Y can contain a moiety selected from the group consisting of acyl, alkyl, sulfonyl, amino, ether, thioether, carboxamido, sulfonamido, aminosulfonyl and olefins;
Y³ and Y⁴ are independently selected from the group consisting of H, alkyl, haloalkyl, halogen, aryl, aralkyl, heteroaralkyl, heteroaryl, hydroxyalkyl, alkenes, and alkyne; wherein the alkyl chain may be straight or branched and optionally containing one or more heteroatoms selected from the group consisting of N, O, and S, and may further contain a sulfone, sulfonamide, nitrile, carboxamide, carboalkoxy or carboxyl group; wherein aryl and heteroaryl rings may be monocyclic or bicyclic optionally containing 1-5 heteroatoms and wherein said ring may be saturated or unsaturated, and such rings may optionally be substituted by one or more substituent selected from the group consisting of alkyl, haloalkyl, aryl, heteroaryl, halogen, alkoxyalkyl, aminoalkyl, hydroxy, nitro, alkoxy, hydroxyalkyl, thioalkyl, amino, alkylamino, arylamino, alkylsulfonamide, acyl, acylamino, alkylsulfone, sulfonamide, allyl, alkenyl, methylenedioxy, ethylenedioxy, alkynyl, carboxamide, cyano, and —(CH₂)ₘCOR wherein m is 0-2 and R is hydroxy, alkoxy, alkyl or amino; with the proviso that when Y³ or Y⁴ is H, Y⁵ may be C or N, otherwise Y⁵ is C; or
Y³ taken together with Y⁴ forms a 3-8 membered monocyclic or a 7-11 membered bicyclic ring B, 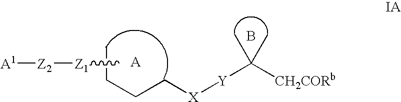
optionally containing one or more double bonds, optionally containing one or more heteroatom or functional group selected from O, NR^{g}, S, CO or SO₂, optionally substituted with one or more substituent selected from the group consisting of alkyl, hydroxy, halogen, haloalkyl, alkoxy, alkyne, cyano, alkylsulfone, sulfonamide, carboalkoxy and carboxyalkyl; or
X taken together with Y³ forms a 3-7 membered monocyclic ring C, 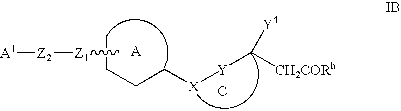
optionally containing one or more double bonds, optionally containing one or more heteroatom or functional group selected from O, NR^{g}, S, CO or SO₂, optionally substituted with one or more substituent selected from the group consisting of alkyl, halogen, alkoxy, haloalkyl, hydroxyalkyl, or alkoxyalkyl; and
R^{b} is X₂—R^{h} wherein X₂ is selected from the group consisting of O, S and NR^{j} wherein R^{h} and Rⁱ are independently selected from the group consisting of H, alkyl, aryl, aralkyl, acyl and alkoxyalkyl.

The compounds of the present invention comprise novel heteroarylalkanoic integrin antagonists.

The present invention relates to the following compounds:
3-(3,5-ditert-butylphenyl)-4-{3-[3-(5,6,7,8-tetrahydro-1,8-naphthyridin-2-yl)propyl]-1,2,4-oxadiazol-5-yl}butanoic acid (TFA salt);
3-(3-tert-butyl-5-iodophenyl)-4-{3-[3-(5,6,7,8-tetrahydro-1,8-naphthyridin-2-yl)propyl]-1,2,4-oxadiazol-5-yl}butanoic acid;
3-(3-tert-butyl-5-bromophenyl)-4-{3-[3-(5,6,7,8-tetrahydro-1,8-naphthyridin-2-yl)propyl]-1,2,4-oxadiazol-5-yl}butanoic acid;
3-(5-tert-Butyl-2-hydroxyphenyl)-4-{3-[3-(5,6,7,8-tetrahydro-1,8-naphthyridin-2-yl)propyl]-1,2,4-oxadiazol-5-yl}butanoic acid;
3-[3,5-Ditert-butyl-2-(carboxymethoxy)phenyl]-4-{3-[3-(5,6,7,8-tetrahydro-1,8-naphthyridin-2-yl)propyl]-1,2,4-oxadiazol-5-yl}butanoic acid;
3-(5-tert-Butyl-2-methoxyphenyl)-4-{3-[3-(5,6,7,8-tetrahydro-1,8-naphthyridin-2-yl)propyl]-1,2,4-oxadiazol-5-yl}butanoic acid;
3-(3,5-Ditert-butyl-4-methoxyphenyl)-4-{3-[3-(5,6,7,8-tetrahydro-1,8-naphthyridin-2-yl)propyl]-1,2,4-oxadiazol-5-yl}butanoic acid;
3-{3-tert-Butyl-5-[2,2,2-trifluoro-1-hydroxy-1-(trifluoromethyl)ethyl]-phenyl}-4-{3-[3-(5,6,7,8-tetrahydro-1,8-naphthyridin-2-yl)propyl]-1,2,4-oxadiazol-5-yl}butanoic acid;
3-(3,4-Dichlorophenyl)-4-{3-[3-(5,6,7,8-tetrahydro-1,8-naphthyridin-2-yl)propyl]-1,2,4-oxadiazol-5-yl}butanoic acid trifluoroacetate;
3-(3-Fluoro-4-methylphenyl)-4-{3-[3-(5,6,7,8-tetrahydro-1,8-naphthyridin-2-yl)propyl]-1,2,4-oxadiazol-5-yl}butanoic acid hydrochloride;
3-(4-Phenoxyphenyl)-4-{3-[3-(5,6,7,8-tetrahydro-1,8-naphthyridin-2-yl)propyl]-1,2,4-oxadiazol-5-yl}butanoic acid trifluoroacetate;
3-(1-Benzofuran-2-yl)-4-{3-[3-(5,6,7,8-tetrahydro-1,8-naphthyridin-2-yl)propyl]-1,2,4-oxadiazol-5-yl}butanoic acid trifluoroacetate;
3-[4-(Benzyloxy)phenyl]4-{3-[3-(5,6,7,8-tetrahydro-1,8-naphthyridin-2-yl)propyl]-1,2,4-oxadiazol-5-yl}butanoic acid trifluoroacetate;
3-[4-(Methylsulfonyl)phenyl]-4-{3-[3-(5,6,7,8-tetrahydro-1,8-naphthyridin-2-yl)propyl]-1,2,4-oxadiazol-5-yl}butanoic acid trifluoroacetate;
4-{3-[3-(5,6,7,8-Tetrahydro-1,8-naphthyridin-2-yl)propyl]-1,2,4-oxadiazol-5-yl}-3-[4-(trifluoromethoxy)phenyl]butanoic acid trifluoroacetate;
3-(3-Furyl)-4-{3-[3-(5,6,7,8-tetrahydro-1,8-naphthyridin-2-yl)propyl]-1,2,4-oxadiazol-5-yl}butanoic acid trifluoroacetate;
4-{3-[3-(5,6,7,8-Tetrahydro-1,8-naphthyridin-2-yl)propyl]-1,2,4-oxadiazol-5-yl}-3-thien-3-ylbutanoic acid trifluoroacetate;
3-(2,3-Dihydro-1,4-benzodioxin-6-yl)-4-{3-[3-(5,6,7,8-tetrahydro-1,8-naphthyridin-2-yl)propyl]-1,2,4-oxadiazol-5-yl}butanoic acid hydrochloride;
4-{3-[3-(5,6,7,8-Tetrahydro-1,8-naphthyridin-2-yl)propyl]-1,2,4-oxadiazol-5-yl}-3-[3-(trifluoromethoxy)phenyl]butanoic acid hydrochloride;
4-{3-[3-(5,6,7,8-Tetrahydro-1,8-naphthyridin-2-yl)propyl]-1,2,4-oxadiazol-5-yl}-3-(3,4,5-trifluorophenyl)butanoic acid hydrochloride;
3-(2,2-Difluoro-1,3-benzodioxol-5-yl)-4-{3-[3-(5,6,7,8-tetrahydro-1,8-naphthyridin-2-yl)propyl]-1,2,4-oxadiazol-5-yl}butanoic acid hydrochloride;
3-[3-Fluoro-5-(trifluoromethyl)phenyl]4-{3-[3-(5,6,7,8-tetrahydro-1,8-naphthyridin-2-yl)propyl]-1,2,4-oxadiazol-5-yl}butanoic acid hydrochloride;
3-(6-Methoxy-2-naphthyl)-4-{3-[3-(5,6,7,8-tetrahydro-1,8-naphthyridin-2-yl)propyl]-1,2,4-oxadiazol-5-yl}butanoic acid hydrochloride;
3-(6-Methoxypyridin-3-yl)-4-{3-[3-(5,6,7,8-tetrahydro-1,8-naphthyridin-2-yl)propyl]-1,2,4-oxadiazol-5-yl}butanoic acid;
3-(4-Cyanophenyl)-4-{3-[3-(5,6,7,8-tetrahydro-1,8-naphthyridin-2-yl)propyl]-1,2,4-oxadiazol-5-yl}butanoic acid;
3-(3-Cyanophenyl)-4-{3-[3-(5,6,7,8-tetrahydro-1,8-naphthyridin-2-yl)propyl]-1,2,4-oxadiazol-5-yl}butanoic acid;
3-benzyl-4-{3-[3-(5,6,7,8-tetrahydro-1,8-naphthyridin-2-yl)propyl]-1,2,4-oxadiazol-5-yl}butanoic acid trifluoroacetate;
3-(4-fluoro-3-methoxyphenyl)-4-{3-[3-(5,6,7,8-tetrahydro-1,8-naphthyridin-2-yl)propyl]-1,2,4-oxadiazol-5-yl}butanoic acid trifluoroacetate;
3-(3-Fluoro-5-methoxyphenyl)-4-{3-[3-(5,6,7,8-tetrahydro-1,8-naphthyridin-2-yl)propyl]-1,2,4-oxadiazol-5-yl}butanoic acid hydrochloride;
3-(2-Methyl-1,3-benzothiazol-5-yl)-4-{3-[3-(5,6,7,8-tetrahydro-1,8-naphthyridin-2-yl)propyl]-1,2,4-oxadiazol-5-yl}butanoic acid hydrochloride;
3-[2-(4-Chlorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride;
3-[2-(4-Methoxyphenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride;
3-(2-Methyl-1,3-benzothiazol-5-yl)-4-{3-[3-(5,6,7,8-tetrahydro-1,8-naphthyridin-2-yl)propyl]-1,2,4-oxadiazol-5-yl}butanoic acid hydrochloride;
3-[2-(4-Fluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride;
3-[2-(3,5-Difluorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride;
3-[2-(3,4-Difluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride;
3-[2-(2-Furyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride;
3-(3,4-Dimethoxyphenyl)-4-{3-[3-(5,6,7,8-tetrahydro-1,8-naphthyridin-2-yl)propyl] 1,2,4-oxadiazol-5-yl}butanoic acid trifluoroacetate;
3-(3,5-Dimethoxyphenyl)-4-{3-[3-(5,6,7,8-tetrahydro-1,8-naphthyridin-2-yl)propyl]1,2,4-oxadiazol-5-yl}butanoic acid trifluoroacetate;
3-(3,5-Dichlorophenyl)-4-{3-[3-(5,6,7,8-tetrahydro-1,8-naphthyridin-2-yl)propyl]-1,2,4-oxadiazol-5-yl}butanoic acid trifluoroacetate;
3-(3,5-Difluorophenyl)-4-{3-[3-(5,6,7,8-tetrahydro-1,8-naphthyridin-2-yl)propyl]-1,2,4-oxadiazol-5-yl}butanoic acid trifluoroacetate;
3-(3-Fluoro-4-methoxyphenyl)-4-{3-[3-(5,6,7,8-tetrahydro-1,8-naphthyridin-2-yl)propyl]-1,2,4-oxadiazol-5-yl}butanoic acid trifluoroacetate;
4-{3-[3-(5,6,7,8-Tetrahydro-1,8-naphthyridin-2-yl)propyl]-1,2,4-oxadiazol-5-yl}-3-[4-(trifluoromethyl)phenyl]butanoic acid trifluoroacetate;
3-(2-Methyl-1,3-thiazol-5-yl)-4-{3-[3-(5,6,7,8-tetrahydro-1,8-naphthyridin-2-yl)propyl]-1,2,4-oxadiazol-5-yl}butanoic acid trifluoroacetate;
3-(1-Phenyl-1H-pyrazol-4-yl)-4-{3-[3-(5,6,7,8-tetrahydro-1,8-naphthyridin-2-yl)propyl]-1,2,4-oxadiazol-5-yl}butanoic acid trifluoroacetate;
3-(1-Benzofuran-6-yl)-4-{3-[3-(5,6,7,8-tetrahydro-1,8-naphthyridin-2-yl)propyl]-1,2,4-oxadiazol-5-yl}butanoic acid hydrochloride;
3-(2,3-dihydro-1-benzofuran-6-yl)-4-{3-[3-(5,6,7,8-tetrahydro-1,8-naphthyridin-2-yl)propyl]-1,2,4-oxadiazol-5-yl}butanoic acid hydrochloride;
3-(1,3-Benzodioxol-5-yl)-4-(3-{3-[(pyridin-2-ylamino)methyl]phenyl}-1,2,4-oxadiazol-5-yl)butanoic acid hydrochloride;
3-(7-Fluoro-1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate;
3-(1,3-Benzoxazol-6-yl)-4-{3-[3-(5,6,7,8-tetrahydro-1,8-naphthyridin-2-yl)propyl]-1,2,4-oxadiazol-5-yl}butanoic acid hydrochloride;
3-(3-Methyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate;
3-(3-Ethyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate;
3-(3-Phenyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate;
[1-Benzoyl-4-({3-[3-(5,6,7,8-tetrahydro-1,8-naphthyridin-2-yl)propyl]-1,2,4-oxadiazol-5-yl}methyl)piperidin-4-yl]acetic acid trifluoroacetate;
[1-Benzoyl-4-({3-[4-(pyridin-2-ylamino)butyl]-1,2,4-oxadiazol-5-yl}methyl)piperidin-4-yl]acetic acid trifluoroacetate;
[1-(tert-Butoxycarbonyl)-4-({3-[3-(5,6,7,8-tetrahydro-1,8-naphthyridin-2-yl)propyl]-1,2,4-oxadiazol-5-yl}methyl)piperidin-4-yl]acetic acid trifluoroacetate;
[1-(tert-Butoxycarbonyl)-4-({3-[4-(pyridin-2-ylamino)butyl]-1,2,4-oxadiazol-5-yl}methyl)piperidin-4-yl]acetic acid trifluoroacetate;
3-(4-Methylphenyl)-4-{3-[3-(5,6,7,8-tetrahydro-1,8-naphthyridin-2-yl)propyl]-1,2,4-oxadiazol-5-yl}butanoic acid trifluoroacetate;
3-(3-Chlorophenyl)-4-{3-[3-(5,6,7,8-tetrahydro-1,8-naphthyridin-2-yl)propyl]-1,2,4-oxadiazol-5-yl}butanoic acid hydrochloride;
3-(4-Methoxy-3-methylphenyl)-4-{3-[3-(5,6,7,8-tetrahydro-1,8-naphthyridin-2-yl)propyl]-1,2,4-oxadiazol-5-yl}butanoic acid hydrochloride;
3-[4-(Methylthio)phenyl]-4-{3-[3-(5,6,7,8-tetrahydro-1,8-naphthyridin-2-yl)propyl]-1,2,4-oxadiazol-5-yl}butanoic acid trifluoroacetate;
3-(1-Methyl-1H-indol-3-yl)-4-{3-[3-(5,6,7,8-tetrahydro-1,8-naphthyridin-2-yl)propyl]-1,2,4-oxadiazol-5-yl}butanoic acid trifluoroacetate;
3-(1,1′-Biphenyl-4-yl)-4-{3-[3-(5,6,7,8-tetrahydro-1,8-naphthyridin-2-yl)propyl]-1,2,4-oxadiazol-5-yl}butanoic acid hydrochloride;
3-(3-Bromophenyl)-4-{3-[3-(5,6,7,8-tetrahydro-1,8-naphthyridin-2-yl)propyl]-1,2,4-oxadiazol-5-yl}butanoic acid trifluoroacetate;
3-(4-Bromophenyl)-4-{3-[3-(5,6,7,8-tetrahydro-1,8-naphthyridin-2-yl)propyl]-1,2,4-oxadiazol-5-yl}butanoic acid hydrochloride;
3-(3-Phenoxyphenyl)-4-{3-[3-(5,6,7,8-tetrahydro-1,8-naphthyridin-2-yl)propyl]-1,2,4-oxadiazol-5-yl}butanoic acid trifluoroacetate;
3-[3-(Benzyloxy)phenyl]4{3-[3-(5,6,7,8-tetrahydro-1,8-naphthyridin-2-yl)propyl]-1,2,4-oxadiazol-5-yl}butanoic acid hydrochloride;
3-(3-Bromo-4-methoxyphenyl)-4-{3-[3-(5,6,7,8-tetrahydro-1,8-naphthyridin-2-yl)propyl]-1,2,4-oxadiazol-5-yl}butanoic acid trifluoroacetate;
4-{3-[3-(5,6,7,8-Tetrahydro-1,8-naphthyridin-2-yl)propyl]-1,2,4-oxadiazol-5-yl}-3-(3,4,5-trimethoxyphenyl)butanoic acid trifluoroacetate;
3-(2-Naphthyl)-4-{3-[3-(5,6,7,8-tetrahydro-1,8-naphthyridin-2-yl)propyl]-1,2,4-oxadiazol-5-yl}butanoic acid hydrochloride;
3-(3-Nitrophenyl)-4-{3-[3-(5,6,7,8-tetrahydro-1,8-naphthyridin-2-yl)propyl]-1,2,4-oxadiazol-5-yl}butanoic acid hydrochloride;
3-(3-Methylphenyl)-4-{3-[3-(5,6,7,8-tetrahydro-1,8-naphthyridin-2-yl)propyl]-1,2,4-oxadiazol-5-yl}butanoic acid hydrochloride;
3-(2-Fu ryl)-4-{3-[3-(5,6,7,8-tetrahydro-1,8-naphthyridin-2-yl)propyl]-1,2,4-oxadiazol-5-yl}butanoic acid hydrochloride;
3-(2-Methylphenyl)-4-{3-[3-(5,6,7,8-tetrahydro-1,8-naphthyridin-2-yl)propyl]-1,2,4-oxadiazol-5-yl}butanoic acid hydrochloride;
3-(1,3-benzodioxol-5-yl)-4-{3-[3-(3,4-dihydro-2H-pyrido[3,2-b][1,4]oxazin-6-yl)propyl]-1,2,4-oxadiazol-5-yl}butanoic acid, TFA;
4-{3-[3-(3,4-dihydro-2H-pyrido[3,2-b][1,4]oxazin-6-yl)propyl]-1,2,4-oxadiazol-5-yl}-3-(3,5-dimethoxyphenyl)butanoic acid, TFA;
3-Benzo[1,3]dioxol-5-yl-4-{3-[3-(6,7,8,9-tetrahydro-5-oxa-1,9-diaza-benzocyclohepten-2-yl)-propyl]-[1,2,4]oxadiazol-5-yl}-butyric acid;
3-(3-Fluoro-4-methoxyphenyl)-4-{3-[3-(6,7,8,9-tetrahydro-5-oxa-1,9-diaza-benzocyclohepten-2-yl)-propyl]-[1,2,4]oxadiazol-5-yl}-butyric acid;
3-(3,5-Difluorophenyl)-4-{3-[3-(6,7,8,9-tetrahydro-5-oxa-1,9-diaza-benzocyclohepten-2-yl)-propyl]-[1,2,4]oxadiazol-5-yl}-butyric acid;
3-(3,5-Dimethoxyphenyl)-4-{3-[3-(6,7,8,9-tetrahydro-5-oxa-1,9-diaza-benzocyclohepten-2-yl)-propyl]-[1,2,4]oxadiazol-5-yl}-butyric acid;
3-(2-Methylbenzothiazol-5-yl)-4-{3-[3-(6,7,8,9-tetrahydro-5-oxa-1,9-diaza-benzocyclohepten-2-yl)-propyl]-[1,2,4]oxadiazol-5-yl}-butyric acid;
3-(1,3-benzodioxol-5-yl)-4-{3-[3-(1,2,3,5-tetrahydropyrido[2,3-e][1,4]oxazepin-8-yl)propyl]-1,2,4-oxadiazol-5-yl}butanoic acid, TFA;
3-(3,5-dimethoxyphenyl)-4-{3-[3-(1,2,3,5-tetrahydropyrido[2,3-e][1,4]oxazepin-8-yl)propyl]-1,2,4-oxadiazol-5-yl}butanoic acid, TFA;
3-(1,3-Benzodioxol-5-yl)-4-(3-{3-[6-(methylamino)pyridin-2-yl]propyl}-1,2,4-oxadiazol-5-yl)butanoic acid hydrochloride;
3-(3-Fluorophenyl)-4-(3-{3-[6-(methylamino)pyridin-2-yl]propyl}-1,2,4-oxadiazol-5-yl)butanoic acid trifluoroacetate;
3-(1,3-benzodioxol-5-yl)-4-(3-3-[6-(ethylamino)pyridin-2-yl]propyl-1,2,4-oxadiazol-5-yl)butanoic acid trifluoroacetate;
3-(3-Fuorophenyl)-4-(3-{3-[6-(methylamino) pyridin-2-yl]propyl-1,2,4-oxadiazol-5-yl)butanoic acid trifluoroacetate;
3-(1,3-Benzodioxol-5-yl)-4-(3-{4-[(4-methylpyridin-2-yl)amino]butyl}-1,2,4-oxadiazol-5-yl)butanoic acid;
3-(1,3-benzodioxol-5-yl)-4-(3-{4-[(6-methylpyridin-2-yl)amino]butyl}-1,2,4-oxadiazol-5-yl)butanoic acid;
(2-{6-[2-(5,6,7,8-Tetrahydro-1,8-naphthyridin-2-yl)ethoxy]pyridin-3-yl}cyclopropyl)acetic acid;
3-Methyl-4-{6-[2-(5,6,7,8-tetrahydro-1,8-naphthyridin-2-yl)ethoxy]pyridin-3-yl}butanoic acid;
3-(1,3-benzodioxol-5-yl)-4-{5-[3-(5,6,7,8-tetrahydro-1,8-naphthyridin-2-yl)propyl]-1,3,4-oxadiazol-2-yl}butanoic acid trifluoroacetate;
3-(3-fluorophenyl)-4-{5-[3-(5,6,7,8-tetrahydro-1,8-naphthyridin-2-yl)propyl]-1,3,4-oxadiazol-2-yl}butanoic acid trifluoroacetate;
3-(3-Fluoro-4-methoxyphenyl)-4-{5-[3-(5,6,7,8-tetrahydro-1,8-naphthyridin-2-yl)propyl]-1,3,4-oxadiazol-2-yl}butanoic acid trifluoroacetate;
3-(3,5-Dimethoxyphenyl)-4-{5-[3-(5,6,7,8-tetrahydro-1,8-naphthyridin-2-yl)propyl]-1,3,4-oxadiazol-2-yl}butanoic acid trifluoroacetate;
3-(2-Methyl-1,3-thiazol-5-yl)-4-{5-[3-(5,6,7,8-tetrahydro-1,8-naphthyridin-2-yl)propyl]-1,3,4-oxadiazol-2-yl}butanoic acid trifluoroacetate;
3-(4-Fluorophenyl)-4-{5-[3-(5,6,7,8-tetrahydro-1,8-naphthyridin-2-yl)propyl]-1,3,4-oxadiazol-2-yl}butanoic acid trifluoroacetate;
3-(3,5-Difluorophenyl)-4-{5-[3-(5,6,7,8-tetrahydro-1,8-naphthyridin-2-yl)propyl]-1,3,4-oxadiazol-2-yl}butanoic acid trifluoroacetate;
3-(3,5-Difluorophenyl)-4-{5-[3-(5,6,7,8-tetrahydro-1,8-naphthyridin-2-yl)propyl]-1,3,4-thiadiazol-2-yl}butanoic acid trifluoroacetate;
3-(4-Fluorophenyl)-4{5-[3-(5,6,7,8-tetrahydro-1,8-naphthyridin-2-yl)propyl]-1,3,4-thiadiazol-2-yl}butanoic acid trifluoroacetate;
3-(2-Methyl-1,3-thiazol-5-yl)-4-{5-[3-(5,6,7,8-tetrahydro-1,8-naphthyridin-2-yl)propyl]-1,3,4-thiadiazol-2-yl}butanoic acid trifluoroacetate;
3-(1,3-Benzodioxol-5-yl)-4-{5-[3-(5,6,7,8-tetrahydro-1,8-naphthyridin-2-yl)propyl]-1,3,4-thiadiazol-2-yl}butanoic acid trifluoroacetate;
3-(1,3-benzodioxol-5-yl)-4-{3-[2-(5,6,7,8-tetrahydro-1,8-naphthyridin-2-yl)ethoxy]isoxazol-5-yl}butanoic acid;
3-(1,3-benzodioxol-5-yl)-4-{5-[3-(5,6,7,8-tetrahydro-1,8-naphthyridin-2-yl)propyl]-2H-tetraazol-2-yl}butanoic acid;
3-(1,3-benzodioxol-5-yl)-4-{5-[3-(5,6,7,8-tetrahydro-1,8-naphthyridin-2-yl)propyl]-1H-tetraazol-1-yl}butanoic acid;
3-(1,3-benzodioxol-5-yl)-4-{3-[2-(5,6,7,8-tetrahydro-1,8-naphthyridin-2-yl)ethoxy]-1H-pyrazol-5-yl}butanoic acid;
3-(1,3-benzodioxol-5-yl)-4-{3-[3-(4,5-dihydro-1H-imidazol-2-ylamino)propoxy]isoxazol-5-yl}butanoic acid;
3-[2-(4-chlorophenyl)-1,3-thiazol-5-yl]4-{3-[2-(5,6,7,8-tetrahydro-1,8-naphthyridin-2-yl)ethoxy]isoxazol-5-yl}butanoic acid;
3-Benzo[1,3]dioxol-5-yl-4-{3-[2-(6,7,8,9-tetrahydro-5-oxa-1,9-diaza-benzocyclohepten-2-yl)-ethoxy]-isoxazol-5-yl}-butyric acid;
3-Benzo[1,3]dioxol-5-yl-4-{3-oxo-2-[2-(6,7,8,9-tetrahydro-5-oxa-1,9-diaza-benzocyclohepten-2-yl)-ethyl]-2,3-dihydro-isoxazol-5-yl}-butyric acid;
3-(1,3-benzodioxol-5-yl)-4-{3-[2-(3,4-dihydro-2H-pyrido[3,2-b][1,4]oxazin-6-yl)ethoxy]isoxazol-5-yl}butanoic acid, TFA;
3-(1,3-benzodioxol-5-yl)-4-{2-[2-(3,4-dihydro-2H-pyrido[3,2-b][1,4]oxazin-6-yl)ethyl]-3-oxo-2,3-dihydroisoxazol-5-yl}butanoic acid, TFA;
3-(1,3-benzodioxol-5-yl)-4-{3-[2-(1,2,3,5-tetrahydropyrido[2,3-e][1,4]oxazepin-8-yl)ethoxy]isoxazol-5-yl}butanoic acid, TFA;
3-(1,3-benzodioxol-5-yl)-4-{3-oxo-2-[2-(1,2,3,5-tetrahydropyrido[2,3-e][1,4]oxazepin-8-yl)ethyl]-2,3-dihydroisoxazol-5-yl}butanoic acid, TFA;
3-(1,3-benzodioxol-5-yl)-4-(3-{2-[5-(methoxymethyl)-6-(methylamino)pyridin-2-yl]ethoxy}isoxazol-5-yl)butanoic acid, TFA;
3-(1,3-Benzodioxol-5-yl)-4-{3-[3-(5,5-dimethyl-5,6,7,8-tetrahydro-1,8-naphthyridin-2-yl)propyl]-1,2,4-oxadiazol-5-yl}butanoic acid;
3-(1,3-benzodioxol-5-yl)-4-{3-[3-(1-methyl-1,2,3,4-tetrahydropyrido[2,3-b]pyrazin-6-yl)propyl]-1,2,4-oxadiazol-5-yl}butanoic acid;
3-(2-methyl-1,3-benzothiazol-5-yl)-4-{3-[3-(1-methyl-1,2,3,4-tetrahydropyrido[2,3-b]pyrazin-6-yl)propyl]-1,2,4-oxadiazol-5-yl}butanoic acid;
3-(3-fluoro-4-methoxyphenyl)-4-{3-[3-(1-methyl-1,2,3,4-tetrahydropyrido[2,3-b]pyrazin-6-yl)propyl]-1,2,4-oxadiazol-5-yl}butanoic acid;
3-(6-methoxypyridin-3-yl)-4-{3-[3-(1-methyl-1,2,3,4-tetrahydropyrido[2,3-b]pyrazin-6-yl)propyl]-1,2,4-oxadiazol-5-yl}butanoic acid;
3-(1,3-benzodioxol-5-yl)-4-(3-{[2-(5,6,7,8-tetrahydro-1,8-naphthyridin-2-yl)ethyl]thio}-1H-1,2,4-triazol-5-yl)butanoic acid;
3-(1,3-benzodioxol-5-yl)-4-(1-methyl-5-{[2-(5,6,7,8-tetrahydro-1,8-naphthyridin-2-yl)ethyl]thio}-1H-1,2,4-triazol-3-yl)butanoic acid;
3-(1,3-benzodioxol-5-yl)-4-(4-methyl-5-{[2-(5,6,7,8-tetrahydro-1,8-naphthyridin-2-yl)ethyl]thio}4H-1,2,4-triazol-3-yl)butanoic acid;
3-(1,3-benzodioxol-5-yl)-4-{3-[2-(1-methyl-1,2,3,4-tetrahydropyrido[2,3-b]pyrazin-6-yl)ethoxy]isoxazol-5-yl}butanoic acid;
3-(1,3-benzodioxol-5-yl)-4-(3-{2-[6-(methylamino)pyridin-2-yl]ethoxy}isoxazol-5-yl)butanoic acid; and
3-(6-methoxypyridin-3-yl)-4-{3-[2-(5,6,7,8-tetrahydro-1,8-naphthyridin-2-yl)ethoxy]isoxazol-5-yl}butanoic acid.

In another embodiment, the present invention may also include the following compounds:
3-methyl-4-(3-{3-[(pyridin-2-ylamino)methyl]phenyl}-1,2,4-oxadiazol-5-yl)butanoic acid;
3-methyl-4-(3-{4-[(pyridin-2-ylamino)methyl]phenyl}-1,2,4-oxadiazol-5-yl)butanoic acid;
3,3-dimethyl-4-{4-[4-(pyridin-2-ylamino)butyl]-1,3-thiazol-2-yl}butanoic acid;
[1-({3-[4-(pyridin-2-ylamino)butyl]-1,2,4-oxadiazol-5-yl}methyl)cyclo-pentyl]-acetic acid;
4-phenyl-4-{3-[4-(pyridin-2-ylamino)butyl]-1,2,4-oxadiazol-5-yl}-butanoic acid;
2-phenyl-4-{3-[4-(pyridin-2-ylamino)butyl]-1,2,4-oxadiazol-5-yl}-butanoic acid;
3,3-dimethyl-4-{3-[2-(2-methyl-5,6,7,8-tetrahydro-1,8-naphthyridin-3-yl)ethyl]-1,2,4-oxadiazol-5-yl}butanoic acid;
[1-({3-[2-(2-methyl-5,6,7,8-tetrahydro-1,8-naphthyridin-3-yl)ethyl]-1,2,4-oxadiazol-5-yl}methyl)cyclopentyl]acetic acid;
4-{3-[2-(2-methyl-5,6,7,8-tetrahydro-1,8-naphthyridin-3-yl)ethyl]-1,2,4-oxadiazol-5-yl}4-phenylbutanoic acid;
4-{3-[2-(2-methyl-5,6,7,8-tetrahydro-1,8-naphthyridin-3-yl)ethyl]-1,2,4-oxadiazol-5-yl}-2-phenylbutanoic acid;
4-{3-[2-(2-methyl-5,6,7,8-tetrahydro-1,8-naphthyridin-3-yl)ethyl]-1,2,4-oxadiazol-5-yl}-2-phenylbutanoic acid;
3,3-dimethyl-4-{3-[3-(5,6,7,8-tetrahydro-1,8-naphthyridin-2-yl)propyl]-1,2,4-oxadiazol-5-yl}butanoic acid;
[1-({3-[3-(5,6,7,8-tetrahydro-1,8-naphthyridin-2-yl)propyl]-1,2,4-oxadiazol-5-yl}methyl)cyclopentyl]acetic acid;
4-phenyl-4-{3-[3-(5,6,7,8-tetrahydro-1,8-naphthyridin-2-yl)propyl]-1,2,4-oxadiazol-5-yl}butanoic acid;
2-phenyl-4-{3-[3-(5,6,7,8-tetrahydro-1,8-naphthyridin-2-yl)propyl]-1,2,4-oxadiazol-5-yl}butanoic acid;
3-(1,3-benzodioxol-5-yl)-4-{3-[3-(5,6,7,8-tetrahydro-1,8-naphthyridin-2-yl)propyl]-1,2,4-oxadiazol-5-yl}butanoic acid;
3-(1,3-benzodioxol-5-yl)-4-{3-[4-(pyridin-2-ylamino)butyl]-1,2,4-oxadiazol-5-yl}butanoic acid;
3-quinolin-3-yl-4-{3-[3-(5,6,7,8-tetrahydro-1,8-naphthyridin-2-yl)-propyl]-1,2,4-oxadiazol-5-yl}butanoic acid;
3-quinolin-3-yl 4-{3-[4-(pyridin-2-ylamino)butyl]-1,2,4-oxadiazol-5-yl}-butanoic acid;
3-(3-methoxyphenyl)-4-{3-[3-(5,6,7,8-tetrahydro-1,8-naphthyridin-2-yl)propyl]-1,2,4-oxadiazol-5-yl}butanoic acid;
3-(3-methoxyphenyl)-4-{3-[4-(pyridin-2-ylamino)butyl]-1,2,4-oxadiazol-5-yl}butanoic acid;
3-(4-methoxyphenyl)-4-{3-[3-(5,6,7,8-tetrahydro-1,8-naphthyridin-2-yl)propyl]-1,2,4-oxadiazol-5-yl}butanoic acid;
3-(4-methoxyphenyl)-4-{3-[4-(pyridin-2-ylamino)butyl]-1,2,4-oxadiazol-5-yl}butanoic acid;
3-(3-fluorophenyl)-4-{3-[3-(5,6,7,8-tetrahydro-1,8-naphthyridin-2-yl)propyl]-1,2,4-oxadiazol-5-yl}butanoic acid;
3-(3-fluorophenyl)-4-{3-[4-(pyridin-2-ylamino)butyl]-1,2,4-oxadiazol-5-yl}butanoic acid;
3-(4-fluorophenyl)-4-{3-[3-(5,6,7,8-tetrahydro-1,8-naphthyridin-2-yl)-propyl]-1,2,4-oxadiazol-5-yl}butanoic acid;
3-(4-fluorophenyl)-4-{3-[4-(pyridin-2-ylamino)butyl]-1,2,4-oxadiazol-5-yl}butanoic acid;
4-{3-[3-(5,6,7,8-tetrahydro-1,8-naphthyridin-2-yl)propyl]-1,2,4-oxadiazol-5-yl}-3-[3-(trifluoromethyl)phenyl]butanoic acid;
4-{3-[4-(pyridin-2-ylamino)butyl]-1,2,4-oxadiazol-5-yl}-3-[3-(trifluoro-methyl)-phenyl]butanoic acid;
3-(3-hydroxyphenyl)-4-{3-[3-(5,6,7,8-tetrahydro-1,8-naphthyridin-2-yl)propyl]-1,2,4-oxadiazol-5-yl}butanoic acid;
3-(3-hydroxyphenyl)-4-{3-[4-(pyridin-2-ylamino)butyl]-1,2,4-oxadiazol-5-yl}butanoic acid;
3-pyridin-3-yl-4-{3-[3-(5,6,7,8-tetrahydro-1,8-naphthyridin-2-yl)-propyl]-1,2,4-oxadiazol-5-yl}butanoic acid;
3-pyridin-3-yl-4-{3-[4-(pyridin-2-ylamino)butyl]-1,2,4-oxadiazol-5-yl}-butanoic acid;
3-phenyl-4-{3-[3-(5,6,7,8-tetrahydro-1,8-naphthyridin-2-yl)propyl]-1,2,4-oxadiazol-5-yl}butanoic acid;
3-phenyl-4-{3-[4-(pyridin-2-ylamino)butyl]-1,2,4-oxadiazol-5-yl}butanoic acid;
3-methyl-3-({3-[4-(pyridin-2-ylamino)butyl]-1,2,4-oxadiazol-5-yl}-methyl)pentanoic acid;
[1-({3-[4-(pyridin-2-ylamino)butyl]-1,2,4-oxadiazol-5-yl}methyl)-cyclohexyl]acetic acid;
3-methyl-3-({3-[4-(pyridin-2-ylamino)butyl]-1,2,4-oxadiazol-5-yl}-methyl)-hexanoic acid;
3,4-dimethyl-3-({3-[4-(pyridin-2-ylamino)butyl]-1,2,4-oxadiazol-5-yl}methyl)-pentanoic acid;
3-ethyl-3-({3-[4-(pyridin-2-ylamino)butyl]-1,2,4-oxadiazol-5-yl}methyl)-pentanoic acid;
4-{3-[4-(pyridin-2-ylamino)butyl]-1,2,4-oxadiazol-5-yl}butanoic acid;
3-methyl-3-phenyl-4-{3-[4-(pyridin-2-ylamino)butyl]-1,2,4-oxadiazol-5-yl}butanoic acid;
3-Methyl-3-{3-[3-(5,6,7,8-tetrahydro-[1,8]naphthyridin-2-yl)-propyl]-[1,2,4]oxadiazol-5-ylmethyl}-pentanoic acid;
3-Methyl-3-{3-[3-(5,6,7,8-tetrahydro-[1,8]naphthyridin-2-yl)-propyl]-[1,2,4]oxadiazol-5-ylmethyl}-hexanoic acid;
3,4-Dimethyl-3-{3-[3-(5,6,7,8-tetrahydro-[1,8]naphthyridin-2-yl)-propyl]-[1,2,4]oxadiazol-5-ylmethyl}-pentanoic acid;
3-Ethyl-3-{3-[3-(5,6,7,8-tetrahydro-[1,8]naphthyridin-2-yl)-propyl]-[1,2,4]oxadiazol-5-ylmethyl}-pentanoic acid;
3-Methyl-3-phenyl-4-{3-[3-(5,6,7,8-tetrahydro-[1,8]naphthyridin-2-yl)-propyl]-[1,2,4]oxadiazol-5-yl}-butyric acid;
3-Phenyl-3-{3-[3-(5,6,7,8-tetrahydro-[1,8]naphthyridin-2-yl)-propyl]-[1,2,4]oxadiazol-5-ylmethyl}-pentanoic acid;
3-Phenyl-3-{3-[3-(5,6,7,8-tetrahydro-[1,8]naphthyridin-2-yl)-propyl]-[1,2,4]oxadiazol-5-ylmethyl}-hexanoic acid;
4-{3-[3-(5,6,7,8-Tetrahydro-[1,8]naphthyridin-2-yl)-propyl]-[1,2,4]oxa-diazol-5-yl}-butyric acid;
3-Methyl-3-pyridin-3-yl-4-{3-[3-(5,6,7,8-tetrahydro-[1,8]naphthyridin-2-yl)-propyl]-[1,2,4]oxadiazol-5-yl}-butyric acid;
(1-Acetyl-4-{3-[3-(5,6,7,8-tetrahydro-[1,8]naphthyridin-2-yl)-propyl]-[1,2,4]oxadiazol-5-ylmethyl}-piperidin-4-yl)-acetic acid;
(1-{3-[3-(5,6,7,8-Tetrahydro-[1,8]naphthyridin-2-yl)propyl]-[1,2,4]oxadiazol-5-ylmethyl}-cyclohexyl)-acetic acid;
3-Methyl-3-pyridin-3-yl-4-{3-[4-(pyridin-2-ylamino)butyl]-[1,2,4]oxadiazol-5-yl}-butyric acid;
4-(benzyloxy)-3-({3-[4-(pyridin-2-ylamino)butyl]-1,2,4-oxadiazol-5-yl}methyl)-butanoic acid;
4-[4-(N-pyridin-2-yl-beta-alanyl)piperazin-1-yl]butanoic acid;
4-{4-[3-(pyridin-2-ylamino)propyl]piperazin-1-yl}butanoic acid;
2-methyl-6-[3(2-pyridylamino)propoxy)-3-pyridinebutanoic acid;
β,β-dimethyl-3-[5-(2-pyridinylamino)pentyl]-1,2,4-oxadiazole-5-butanoic acid;
β,β-dimethyl-3-[4-(2-pyridinylamino)butyl]-1,2,4-oxadiazole-5-butanoic acid;
β,β-dimethyl-3-[[[2-(2-pyridinylamino)ethyl]thio]methyl]-1,2,4-oxadiazole-5-butanoic acid;
4-Carboxymethyl-4-{3-[3-(5,6,7,8-tetrahydro-[1,8]naphthyridin-2-yl)-propyl]-[1,2,4]oxadiazol-5-ylmethyl}-piperidine-1-carboxylic acid tert-butyl ester;
(1-Benzoyl-4-{3-[3-(5,6,7,8-tetrahydro-[1,8]naphthyridin-2-yl)-propyl]-[1,2,4]oxadiazol-5-ylmethyl}-piperidin-4-yl)-acetic acid;
[4-{3-[3-(5,6,7,8-Tetrahydro-[1,8]naphthyridin-2-yl)-propyl]-[1,2,4]oxadiazol-5-ylmethyl}-1-(2,2,2-trifluoroacetyl)-piperidin-4-yl]-acetic acid;
4-(phenylthio)-3-({3-[4-(pyridin-2-ylamino)butyl]-1,2,4-oxadiazol-5-yl}methyl)butanoic acid;
4-(phenylthio)-3-({3-[3-(5,6,7,8-tetrahydro-1,8-naphthyridin-2-yl)propyl]-1,2,4-oxadiazol-5-yl}methyl)butanoic acid;
3-methyl-4-{3-[4-(pyridin-2-ylamino)butyl]-1,2,4-oxadiazol-5-yl}butanoic acid hydrochloride;
3-methyl-4-{3-[3-(5,6,7,8-tetrahydro-1,8-naphthyridin-2-yl)propyl]-1,2,4-oxadiazol-5-yl}butanoic acid;
((1S,2R)-2-{3-[3-(5,6,7,8-tetrahydro-1,8-naphthyridin-2-yl)propyl]-1,2,4-oxadiazol-5-yl}cyclopropyl)acetic acid;
((1S,2S)-2-{3-[3-(5,6,7,8-tetrahydro-1,8-naphthyridin-2-yl)propyl]-1,2,4-oxadiazol-5-yl}cyclopropyl)acetic acid;
3-Pyridin-3-yl-4-{5-[3-(5,6,7,8-tetrahydro-[1,8]naphthyridin-2-yl)-propyl]4H-[1,2,4]triazol-3-yl}-butyric acid;
3-Benzo[1,3]dioxol-5-yl-4-{5-[3-(5,6,7,8-tetrahydro-[1,8]naphthyridin-2-yl)-propyl]-tetrazol-2-yl}-butyric acid;
(2-{5-[3-(5,6,7,8-Tetrahydro-[1,8]naphthyridin-2-yl)-propyl]-[1,3,4]oxa-diazol-2-yl}-cyclopropyl)-acetic acid;
3-Phenyl-4-{5-[3-(5,6,7,8-tetrahydro-[1,8]naphthyridin-2-yl)-propyl]-[1,3,4]oxadiazol-2-yl}-butyric acid;
3-(2,3-Dihydro-benzofuran-6-yl)-4-{5-[3-(5,6,7,8-tetrahydro-[1,8]naphthyridin-2-yl)-propyl]-[1,3,4]oxadiazol-2-yl}-butyric acid;
3-(3-Fluoro-phenyl)-4-{5-[3-(5,6,7,8-tetrahydro-[1,8]naphthyridin-2-yl)-propyl]-[1,3,4]oxadiazol-2-yl}-butyric acid;
3-Benzo[1,3]dioxol-5-yl-4-{5-[3-(5,6,7,8-tetrahydro-[1,8]naphthyridin-2-yl)-propyl]-[1,3,4]oxadiazol-2-yl}-butyric acid;
(2-{2-[3-(5,6,7,8-Tetrahydro-[1,8]naphthyridin-2-yl)-propyl]-2H-tetrazol-5-yl}-cyclopropyl)-acetic acid;
3-Phenyl-4-{2-[3-(5,6,7,8-tetrahydro-[1,8]naphthyridin-2-yl)-propyl]-2H-tetrazol-5-yl}-butyric acid;
3-(2,3-Dihydro-benzofuran-6-yl)-4-{2-[3-(5,6,7,8-tetrahydro-[1,8]naphthyridin-2-yl)-propyl]-2H-tetrazol-5-yl}-butyric acid;
3-(2,3-Dihydro-benzofuran-6-yl)-4-{2-[3-(5,6,7,8-tetrahydro-[1,8]naphthyridin-2-yl)-propyl]-2H-tetrazol-5-yl}-butyric acid;
3-(3-Fluoro-phenyl)-4-{2-[3-(5,6,7,8-tetrahydro-[1,8]naphthyridin-2-yl)-propyl]-2H-tetrazol-5-yl}-butyric acid;
3-Pyridin-3-yl-4-{2-[3-(5,6,7,8-tetrahydro-[1,8]naphthyridin-2-yl)-propyl]-2H-tetrazol-5-yl}-butyric acid;
3-Benzo[1,3]dioxol-5-yl-4-{2-[3-(5,6,7,8-tetrahydro-[1,8]naphthyridin-2-yl)-propyl]-2H-tetrazol-5-yl}-butyric acid;
(2-{5-[3-(5,6,7,8-Tetrahydro-[1,8]naphthyridin-2-yl)-propyl]-isoxazol-3-yl}-cyclopropyl)-acetic acid;
3-Phenyl-4-{5-[3-(5,6,7,8-tetrahydro-[1,8]naphthyridin-2-yl)-propyl]-isoxazol-3-yl}-butyric acid;
3-(2,3-Dihydro-benzofuran-6-yl)-4-{5-[3-(5,6,7,8-tetrahydro-[1,8]naphthyridin-2-yl)-propyl]-isoxazol-3-yl}-butyric acid;
3-(3-Fluoro-phenyl)-4-{5-[3-(5,6,7,8-tetrahydro-[1,8]naphthyridin-2-yl)-propyl]-isoxazol-3-yl}-butyric acid;
3-Pyridin-3-yl-4-{5-[3-(5,6,7,8-tetrahydro-[1,8]naphthyridin-2-yl)-propyl]-isoxazol-3-yl}-butyric acid;
3-Benzo[1,3]dioxol-5-yl-4-{5-[3-(5,6,7,8-tetrahydro-[1,8]naphthyridin-2-yl)-propyl]-isoxazol-3-yl}-butyric acid;
3-(2,3-Dihydro-benzofuran-6-yl)-4-{3-[3-(5,6,7,8-tetrahydro-[1,8]naphthyridin-2-yl)-propyl]-isoxazol-5-yl}-butyric acid;
3-(3-Fluoro-phenyl)-4-{3-[3-(5,6,7,8-tetrahydro-[1,8]naphthyridin-2-yl)-propyl]-isoxazol-5-yl}-butyric acid;
3-Pyridin-3-yl-4-{3-[3-(5,6,7,8-tetrahydro-[1,8]naphthyridin-2-yl)-propyl]-isoxazol-5-yl}-butyric acid;
3-Benzo[1,3]dioxol-5-yl-4-{3-[3-(5,6,7,8-tetrahydro-[1,8]naphthyridin-2-yl)-propyl]-isoxazol-5-yl}-butyric acid;
3-Phenyl-4-{5-[3-(5,6,7,8-tetrahydro-[1,8]naphthyridin-2-yl)-propyl]-1H-pyrazol-3-yl}-butyric acid;
3-(2,3-Dihydro-benzofuran-6-yl)-4-{5-[3-(5,6,7,8-tetrahydro-[1,8]naphthyridin-2-yl)-propyl]-1H-pyrazol-3-yl-butyric acid;
3-(3-Fluoro-phenyl)-4-{5-[3-(5,6,7,8-tetrahydro-[1,8]naphthyridin-2-yl propyl]-1H-pyrazol-3-yl}-butyric acid;
3-Pyridin-3-yl-4-{5-[3-(5,6,7,8-tetrahydro-[1,8]naphthyridin-2-yl)-propyl]-1H-pyrazol-3-yl}-butyric acid;
3-Benzo[1,3]dioxol-5-yl-4-{5-[3-(5,6,7,8-tetrahydro-[1,8]naphthyridin-2-yl)-propyl]-1H-pyrazol-3-yl}-butyric acid;
(2-{3-[3-(5,6,7,8-Tetrahydro-[1,8]naphthyridin-2-yl)-propyl]-isoxazol-5-yl}-cyclopropyl)-acetic acid;
(2-{5-[3-(5,6,7,8-Tetrahydro-[1,8]naphthyridin-2-yl)-propyl]-1H-pyrazol-3-yl}-cyclopropyl)-acetic acid;
(2-{4-[3-(5,6,7,8-Tetrahydro-[1,8]naphthyridin-2-yl)-propyl]-thiazol-2-yl}-cyclopropyl)-acetic acid; 3-Phenyl-4-{4-[3-(5,6,7,8-tetrahydro-[1,8]naphthyridin-2-yl)-propyl]-thiazol-2-yl}-butyric acid;
3-(2,3-Dihydro-benzofuran-6-yl)-4-{4-[3-(5,6,7,8-tetrahydro-[1,8]naphthyridin-2-yl)-propyl]-thiazol-2-yl}-butyric acid;
3-(3-Fluoro-phenyl)-4-{4-[3-(5,6,7,8-tetrahydro-[1,8]naphthyridin-2-yl)-propyl]-thiazol-2-yl}-butyric acid;
3-Pyridin-3-yl-4-{4-[3-(5,6,7,8-tetrahydro-[1,8]naphthyridin-2-yl)-propyl]-thiazol-2-yl}-butyric acid;
3-Benzo[1,3]dioxol-5-yl-4-{4-[3-(5,6,7,8-tetrahydro-[1,8]naphthyridin-2-yl)-propyl]-thiazol-2-yl}-butyric acid;
3-Phenyl-4-{3-[3-(5,6,7,8-tetrahydro-[1,8]naphthyridin-2-yl)-propyl]-pyrazol-1-yl}-butyric acid;
3-(2,3-Dihydro-benzofuran-6-yl)-4-{3-[3-(5,6,7,8-tetrahydro-[1,8]naphthyridin-2-yl)-propyl]-pyrazol-1-yl}-butyric acid;
3-(3-Fluoro-phenyl)-4-{3-[3-(5,6,7,8-tetrahydro-[1,8]naphthyridin-2-yl)-propyl]-pyrazol-1-yl}-butyric acid;
3-Pyridin-3-yl-4-{3-[3-(5,6,7,8-tetrahydro-[1,8]naphthyridin-2-yl)-propyl]-pyrazol-1-yl}-butyric acid;
3-Pyridin-3-yl-4-{3-[3-(5,6,7,8-tetrahydro-[1,8]naphthyridin-2-yl)-propyl]-pyrazol-1-yl}-butyric acid;
3-Benzo[1,3]dioxol-5-yl-4-{3-[3-(5,6,7,8-tetrahydro-[1,8]naphthyridin-2-yl)-propyl]-pyrazol-1-yl}-butyric acid;
3-Phenyl-4-{4-[3-(5,6,7,8-tetrahydro-[1,8]naphthyridin-2-yl)-propyl]-imidazol-1-yl}-butyric acid;
3-(2,3-Dihydro-benzofuran-6-yl)-4-{4-[3-(5,6,7,8-tetrahydro-[1,8]naphthyridin-2-yl)-propyl]-imidazol-1-yl}-butyric acid;
3-(3-Fluoro-phenyl)-4-{4-[3-(5,6,7,8-tetrahydro-[1,8]naphthyridin-2-yl)-propyl]-imidazol-1-yl}-butyric acid;
3-Pyridin-3-yl-4-{4-[3-(5,6,7,8-tetrahydro-[1,8]naphthyridin-2-yl)-propyl]-imidazol-1-yl}-butyric acid;
3-Benzo[1,3]dioxol-5-yl-4-{4-[3-(5,6,7,8-tetrahydro-[1,8]naphthyridin-2-yl)-propyl]-imidazol-1-yl}-butyric acid3-Phenyl-4-{3-[2-(5,6,7,8-tetrahydro-[1,8]naphthyridin-2-yl)-ethoxy]-isoxazol-5-yl}-butyric acid;
3-(2,3-Dihydro-benzofuran-6-yl)-4-{3-[2-(5,6,7,8-tetrahydro-[1,8]naphthyridin-2-yl)-ethoxy]-isoxazol-5-yl}-butyric acid;
3-(3-Fluoro-phenyl)-4-{3-[2-(5,6,7,8-tetrahydro-[1,8]naphthyridin-2-yl)-ethoxy]-isoxazol-5-yl}-butyric acid;
3-Pyridin-3-yl-4-{3-[2-(5,6,7,8-tetrahydro-[1,8]naphthyridin-2-yl)-ethoxy]-isoxazol-5-yl}-butyric acid;
3-Benzo[1,3]dioxol-5-yl-4-{3-[2-(5,6,7,8-tetrahydro-[1,8]naphthyridin-2-yl)-ethoxy]-isoxazol-5-yl}-butyric acid;
3-(3-Fluoro-phenyl)-4-{5-[2-(5,6,7,8-tetrahydro-[1,8]naphthyridin-2-yl)-ethoxy]-2H-pyrazol-3-yl}-butyric acid;
3-(2,3-Dihydro-benzofuran-6-yl)-4-{5-[2-(5,6,7,8-tetrahydro-[1,8]naphthyridin-2-yl)-ethoxy]-2H-pyrazol-3-yl}-butyric acid;
3-Phenyl-4-{5-[2-(5,6,7,8-tetrahydro-[1,8]naphthyridin-2-yl)-ethoxy]-2H-pyrazol-3-yl}-butyric acid;
3-Pyridin-3-yl-4-{5-[2-(5,6,7,8-tetrahydro-[1,8]naphthyridin-2-yl)-ethoxy]-2H-pyrazol-3-yl}-butyric acid;
3-Benzo[1,3]dioxol-5-yl-4-{5-[2-(5,6,7,8-tetrahydro-[1,8]naphthyridin-2-yl)-ethoxy]-2H-pyrazol-3-yl}-butyric acid;
3-Phenyl-4-[4-(3-5,6,7,8-tetrahydro-[1,8]naphthyridin-2-yl-propionyl)-imidazol-1-yl]-butyric acid;
3-(2,3-Dihydro-benzofuran-6-yl)-4-[4-(3-5,6,7,8-tetrahydro-[1,8]naphthyridin-2-yl-propionyl imidazol-1-yl]-butyric acid;
3-(3-Fluoro-phenyl)-4-[4-(3-5,6,7,8-tetrahydro-[1,8]naphthyridin-2-yl-propionyl)-imidazol-1-yl]-butyric acid;
3-Pyridin-3-yl-4-[4-(3-5,6,7,8-tetrahydro-[1,8]naphthyridin-2-yl-propionyl)-imidazol-1-yl]-butyric acid;
3-Benzo[1,3]dioxol-5-yl-4-[4-(3-5,6,7,8-tetrahydro-[1,8]naphthyridin-2-yl-propionyl)-imidazol-1-yl]-butyric acid;
4-{4-[1-Hydroxy-3-(5,6,7,8-tetrahydro-[1,8]naphthyridin-2-yl)-propyl]-imidazol-1-yl}-3-phenyl-butyric acid;
3-(2,3-Dihydro-benzofuran-6-yl)-4-{4-[1-hydroxy-3-(5,6,7,8-tetrahydro-[1,8]naphthyridin-2-yl)-propyl]-imidazol-1-yl}-butyric acid;
3-(3-Fluoro-phenyl)-4-{4-[1-hydroxy-3-(5,6,7,8-tetrahydro-[1,8]naphthyridin-2-yl)-propyl]-imidazol-1-yl}-butyric acid;
4-{4-[1-Hydroxy-3-(5,6,7,8-tetrahydro-[1,8]naphthyridin-2-yl)-propyl]-imidazol-1-yl}-3-pyridin-3-yl-butyric acid;
4-{4-[1-Hydroxy-3-(5,6,7,8-tetrahydro-[1,8]naphthyridin-2-yl)-propyl]-imidazol-1-yl}-3-pyridin-3-yl-butyric acid;
4-{4-[3-Hydroxy-3-(5,6,7,8-tetrahydro-[1,8]naphthyridin-2-yl)-prop-1-ynyl]-imidazol-1-yl}-3-phenyl-butyric acid;
3-(2,3-Dihydro-benzofuran-6-yl)-4-{4-[3-hydroxy-3-(5,6,7,8-tetrahydro-[1,8]naphthyridin-2-yl)-prop-1-ynyl]-imidazol-1-yl}-butyric acid;
3-(3-Fluoro-phenyl)-4-{4-[3-hydroxy-3-(5,6,7,8-tetrahydro-[1,8]naphthyridin-2-yl)-prop-1-ynyl]-imidazol-1-yl}-butyric acid;
4-{4-[3-Hydroxy-3-(5,6,7,8-tetrahydro-[1,8]naphthyridin-2-yl)-prop-1-ynyl]-imidazol-1-yl}-3-pyridin-3-yl-butyric acid;
3-(2,3-Dihydro-benzofuran-6-yl)-4-{2-[3-(5,6,7,8-tetrahydro-[1,8]naphthyridin-2-yl)-propyl]-2H-tetrazol-5-yl}-butyric acid;
3-(3-Fluoro-phenyl)-4-{2-[3-(5,6,7,8-tetrahydro-[1,8]naphthyridin-2-yl)-propyl]-2H-tetrazol-5-yl}-butyric acid;
3-Pyridin-3-yl-4-{2-[3-(5,6,7,8-tetrahydro-[1,8]naphthyridin-2-yl)-propyl]-2H-tetrazol-5-yl}-butyric acid;
3-Benzo[1,3]dioxol-5-yl-4-{2-[3-(5,6,7,8-tetrahydro-[1,8]naphthyridin-2-yl)-propyl]-2H-tetrazol-5-yl}-butyric acid;
(2-{5-[3-(5,6,7,8-Tetrahydro-[1,8]naphthyridin-2-yl)-propyl]-isoxazol-3-yl}-cyclopropyl)-acetic acid;
3-Phenyl-4-{5-[3-(5,6,7,8-tetrahydro-[1,8]naphthyridin-2-yl)-propyl]-isoxazol-3-yl}-butyric acid;
3-(2,3-Dihydro-benzofuran-6-yl)-4-{5-[3-(5,6,7,8-tetrahydro-[1,8]naphthyridin-2-yl)-propyl]-isoxazol-3-yl}-butyric acid;
3-(3-Fluoro-phenyl)-4-{5-[3-(5,6,7,8-tetrahydro-[1,8]naphthyridin-2-yl)-propyl]-isoxazol-3-yl}-butyric acid;
3-Pyridin-3-yl-4-{5-[3-(5,6,7,8-tetrahydro-[1,8]naphthyridin-2-yl)-propyl]-isoxazol-3-yl}-butyric acid;
3-Benzo[1,3]dioxol-5-yl-4-{5-[3-(5,6,7,8-tetrahydro-[1,8]naphthyridin-2-yl)-propyl]-isoxazol-3-yl}-butyric acid;
3-(2,3-Dihydro-benzofuran-6-yl)-4-{3-[3-(5,6,7,8-tetrahydro-[1,8]naphthyridin-2-yl)-propyl]-isoxazol-5-yl}-butyric acid;
3-(3-Fluoro-phenyl)-4-{3-[3-(5,6,7,8-tetrahydro-[1,8]naphthyridin-2-yl)-propyl]-isoxazol-5-yl}-butyric acid;
3-Pyridin-3-yl-4-{3-[3-(5,6,7,8-tetrahydro-[1,8]naphthyridin-2-yl)-propyl]-isoxazol-5-yl}-butyric acid;
3-Benzo[1,3]dioxol-5-yl-4-{3-[3-(5,6,7,8-tetrahydro-[1,8]naphthyridin-2-yl)-propyl]-isoxazol-5-yl}-butyric acid;
3-Phenyl-4-{5-[3-(5,6,7,8-tetrahydro-[1,8]naphthyridin-2-yl)-propyl]-1H-pyrazol-3-yl}-butyric acid;
3-(2,3-Dihydro-benzofuran-6-yl)-4-{5-[3-(5,6,7,8-tetrahydro-[1,8]naphthyridin-2-yl)-propyl]-1H-pyrazol-3-yl}-butyric acid;
3-(3-Fluoro-phenyl)-4-{5-[3-(5,6,7,8-tetrahydro-[1,8]naphthyridin-2-yl)-propyl]-1H-pyrazol-3-yl}-butyric acid;
3-Pyridin-3-yl-4-{5-[3-(5,6,7,8-tetrahydro-[1,8]naphthyridin-2-yl)-propyl]-1H-pyrazol-3-yl}-butyric acid;
3-Benzo[1,3]dioxol-5-yl-4-{5-[3-(5,6,7,8-tetrahydro-[1,8]naphthyridin-2-yl)-propyl]-1H-pyrazol-3-yl}-butyric acid;
(2-{3-[3-(5,6,7,8-Tetrahydro-[1,8]naphthyridin-2-yl)-propyl]-isoxazol-5-yl}-cyclopropyl)-acetic acid;
(2-{5-[3-(5,6,7,8-Tetrahydro-[1,8]naphthyridin-2-yl)-propyl]-1H-pyrazol-3-yl}-cyclopropyl)-acetic acid;
(2-{4-[3-(5,6,7,8-Tetrahydro-[1,8]naphthyridin-2-yl)-propyl]-thiazol-2-yl}-cyclopropyl)-acetic acid;
3-Phenyl-4-{-[3-(5,6,7,8-tetrahydro-[1,8]naphthyridin-2-yl)-propyl]-thiazol-2-yl}butyric acid;
3-(2,3-Dihydro-benzofuran-6-yl)-4-{4-[3-(5,6,7,8-tetrahydro-[1,8]naphthyridin-2-yl)-propyl]-thiazol-2-yl}-butyric acid;
3-(3-Fluoro-phenyl)-4-{4-[3-(5,6,7,8-tetrahydro-[1,8]naphthyridin-2-yl)-propyl]-thiazol-2-yl}-butyric acid;
3-Pyridin-3-yl-4-{4-[3-(5,6,7,8-tetrahydro-[1,8]naphthyridin-2-yl)-propyl]-thiazol-2-yl}-butyric acid;
3-Benzo[1,3]dioxol-5-yl-4-{4-[3-(5,6,7,8-tetrahydro-[1,8]naphthyridin-2-yl)-propyl]-thiazol-2-yl}-butyric acid;
3-Phenyl-4-{3-[3-(5,6,7,8-tetrahydro-[1,8]naphthyridin-2-yl)-propyl]-pyrazol-1-yl}-butyric acid;
3-(2,3-Dihydro-benzofuran-6-yl)-4-{3-[3-(5,6,7,8-tetrahydro-[1,8]naphthyridin-2-yl)-propyl]-pyrazol-1-yl}-butyric acid;
3-(3-Fluoro-phenyl)-4-{3-[3-(5,6,7,8-tetrahydro-[1,8]naphthyridin-2-yl)-propyl]-pyrazol-1-yl}-butyric acid;
3-Pyridin-3-yl-4-{3-[3-(5,6,7,8-tetrahydro-[1,8]naphthyridin-2-yl)-propyl]-pyrazol-1-yl}-butyric acid;
3-Pyridin-3-yl-4-{3-[3-(5,6,7,8-tetrahydro-[1,8]naphthyridin-2-yl)-propyl]-pyrazol-1-yl}-butyric acid;
3-Benzo[1,3]dioxol-5-yl-4-{3-[3-(5,6,7,8-tetrahydro-[1,8]naphthyridin-2-yl)-propyl]-pyrazol-1-yl}-butyric acid;
3-Phenyl-4-{4-[3-(5,6,7,8-tetrahydro-[1,8]naphthyridin-2-yl)-propyl]-imidazol-1-yl}-butyric acid;
3-(2,3-Dihydro-benzofuran-6-yl)-4-{4-[3-(5,6,7,8-tetrahydro-[1,8]naphthyridin-2-yl)-propyl]-imidazol-1-yl}-butyric acid;
3-(3-Fluoro-phenyl)-4-{4-[3-(5,6,7,8-tetrahydro-[1,8]naphthyridin-2-yl)-propyl]-imidazol-1-yl}-butyric acid;
3-Pyridin-3-yl-4-{4-[3-(5,6,7,8-tetrahydro-[1,8]naphthyridin-2-yl)-propyl]-imidazol-1-yl}-butyric acid;
3-Benzo[1,3]dioxol-5-yl-4-{4-[3-(5,6,7,8-tetrahydro-[1,8]naphthyridin-2-yl)-propyl]-imidazol-1-yl}-butyric acid3-Phenyl-4-{3-[2-(5,6,7,8-tetrahydro-[1,8]naphthyridin-2-yl)-ethoxy]-isoxazol-5-yl}-butyric acid;
3-(2,3-Dihydro-benzofuran-6-yl)-4-{3-[2-(5,6,7,8-tetrahydro-[1,8]naphthyridin-2-yl)-ethoxy]-isoxazol-5-yl}-butyric acid;
3-(3-Fluoro-phenyl)-4-{3-[2-(5,6,7,8-tetrahydro-[1,8]naphthyridin-2-yl)-ethoxy]-isoxazol-5-yl}-butyric acid;
3-Pyridin-3-yl-4-{3-[2-(5,6,7,8-tetrahydro-[1,8]naphthyridin-2-yl)-ethoxy]-isoxazol-5-yl}-butyric acid;
3-Benzo[1,3]dioxol-5-yl-4-{3-[2-(5,6,7,8-tetrahydro-[1,8]naphthyridin-2-yl)-ethoxy]-isoxazol-5-yl}-butyric acid;
3-(3-Fluoro-phenyl)-4-{5-[2-(5,6,7,8-tetrahydro-[1,8]naphthyridin-2-yl)-ethoxy]-2H-pyrazol-3-yl}-butyric acid;
3-(2,3-Dihydro-benzofuran-6-yl)-4-{5-[2-(5,6,7,8-tetrahydro-[1,8]naphthyridin-2-yl)-ethoxy]-2H-pyrazol-3-yl}-butyric acid;
3-Phenyl-4-{5-[2-(5,6,7,8-tetrahydro-[1,8]naphthyridin-2-yl)-ethoxy]-2H-pyrazol-3-yl}-butyric acid;
3-Pyridin-3-yl-4-{5-[2-(5,6,7,8-tetrahydro-[1,8]naphthyridin-2-yl)-ethoxy]-2H-pyrazol-3-yl}-butyric acid;
3-Benzo[1,3]dioxol-5-yl-4-{5-[2-(5,6,7,8-tetrahydro-[1,8]naphthyridin-2-yl)-ethoxy]-2H-pyrazol-3-yl}-butyric acid;
3-Phenyl-4-[4-(3-5,6,7,8-tetrahydro-[1,8]naphthyridin-2-yl-propionyl)-imidazol-1-yl]-butyric acid;
3-(2,3-Dihydro-benzofuran-6-yl)-4-[4-(3-5,6,7,8-tetrahydro-[1,8]naphthyridin-2-yl-propionyl)-imidazol-1-yl]-butyric acid;
3-(3-Fluoro-phenyl)-4-[4-(3-5,6,7,8-tetrahydro-[1,8]naphthyridin-2-yl-propionyl)-imidazol-1-yl]-butyric acid;
3-Pyridin-3-yl-4-[4-(3-5,6,7,8-tetrahydro-[1,8]naphthyridin-2-yl-propionyl)-imidazol-1-yl]-butyric acid;
3-Benzo[1,3]dioxol-5-yl-4-[4-(3-5,6,7,8-tetrahydro-[1,8]naphthyridin-2-yl-propionyl)-imidazol-1-yl]-butyric acid;
4-{4-[1-Hydroxy-3-(5,6,7,8-tetrahydro-[1,8]naphthyridin-2-yl)-propyl]-imidazol-1-yl}-3-phenyl-butyric acid;
3-(2,3-Dihydro-benzofuran-6-yl)-4-{4-[1-hydroxy-3-(5,6,7,8-tetrahydro-[1,8]naphthyridin-2-yl)-propyl]-imidazol-1-yl}-butyric acid;
3-(3-Fluoro-phenyl)-4-{4-[1-hydroxy-3-(5,6,7,8-tetrahydro-[1,8]naphthyridin-2-yl)-propyl]-imidazol-1-yl}butyric acid;
4-{4-[1-Hydroxy-3-(5,6,7,8-tetrahydro-[1,8]naphthyridin-2-yl propyl]-imidazol-1-yl}-3-pyridin-3-yl-butyric acid;
4-{4-[1-Hydroxy-3-(5,6,7,8-tetrahydro-[1,8]naphthyridin-2-yl)-propyl]-imidazol-1-yl}-3-pyridin-3-yl-butyric acid;
4-{4-[3-Hydroxy-3-(5,6,7,8-tetrahydro-[1,8]naphthyridin-2-yl)-prop-1-ynyl]-imidazol-1-yl}-3-phenyl-butyric acid;
3-(2,3-Dihydro-benzofuran-6-yl)-4-{4-[3-hydroxy-3-(5,6,7,8-tetrahydro-[1,8]naphthyridin-2-yl)-prop-1-ynyl]-imidazol-1-yl}-butyric acid;
3-(3-Fluoro-phenyl)-4-{4-[3-hydroxy-3-(5,6,7,8-tetrahydro-[1,8]naphthyridin-2-yl)-prop-1-ynyl]-imidazol-1-yl}-butyric acid;
4-{4-[3-Hydroxy-3-(5,6,7,8-tetrahydro-[1,8]naphthyridin-2-yl)-prop-1-ynyl]-imidazol-1-yl}-3-pyridin-3-yl-butyric acid;
3-Benzo[1,3]dioxol-5-yl-4-{4-[3-hydroxy-3-(5,6,7,8-tetrahydro-[1,8]naphthyridin-2-yl)-prop-1-ynyl]-imidazol-1-yl}-butyric acid;
4-{4-[3-Hydroxy-3-(5,6,7,8-tetrahydro-[1,8]naphthyridin-2-yl)-prop-1-ynyl]-pyrazol-1-yl}-3-phenyl-butyric acid;
3-(2,3-Dihydro-benzofuran-6-yl)-4-{4-[3-hydroxy-3-(5,6,7,8-tetrahydro-[1,8]naphthyridin-2-yl)-prop-1-ynyl]-pyrazol-1-yl}-butyric acid;
3-(3-Fluoro-phenyl)-4-{-[3-hydroxy-3-(5,6,7,8-tetrahydro-[1,8]naphthyridin-2-yl)-prop-1-ynyl]-pyrazol-1-yl}-butyric acid;
4-{4-[3-Hydroxy-3-(5,6,7,8-tetrahydro-[1,8]naphthyridin-2-yl)-prop-1-ynyl]-pyrazol-1-yl}-3-pyridin-3-yl-butyric acid3-Benzo[1,3]dioxol-5-yl-4-{4-[3-hydroxy-3-(5,6,7,8-tetrahydro-[1,8]naphthyridin-2-yl)-prop-1-ynyl]-pyrazol-1-yl}-butyric acid4-{4-[3-Hydroxy-3-(5,6,7,8-tetrahydro-[1,8]naphthyridin-2-yl)-propenyl]-pyrazol-1-yl}-3-phenyl-butyric acid;
3-(2,3-Dihydro-benzofuran-6-yl)-4-{4-[3-hydroxy-3-(5,6,7,8-tetrahydro-[1,8]naphthyridin-2-yl)-propenyl]-pyrazol-1-yl}-butyric acid3-(3-Fluoro-phenyl)-4-{4-[3-hydroxy-3-(5,6,7,8-tetrahydro-[1,8]naphthyridin-2-yl)-propenyl]-pyrazol-1-yl}-butyric acid;
4-{4-[3-Hydroxy-3-(5,6,7,8-tetrahydro-[1,8]naphthyridin-2-yl)-propenyl]-pyrazol-1-yl}-3-pyridin-3-yl-butyric acid;
3-Benzo[1,3]dioxol-5-yl-4-{4-[3-hydroxy-3-(5,6,7,8-tetrahydro-[1,8]naphthyridin-2-yl)-propenyl]-pyrazol-1-yl}-butyric acid;
3-Benzo[1,3]dioxol-5-yl-4-{4-[3-hydroxy-3-(5,6,7,8-tetrahydro-[1,8]naphthyridin-2-yl)-propenyl]-pyrazol-1-yl}-butyric acid;
4-{4-[3-Hydroxy-3-(5,6,7,8-tetrahydro-[1,8]naphthyridin-2-yl)-propenyl]-imidazol-1-yl}-3-phenyl-butyric acid;
3-(2,3-Dihydro-benzofuran-6-yl)-4-{4-[3-hydroxy-3-(5,6,7,8-tetrahydro-[1,8]naphthyridin-2-yl)-propenyl]-imidazol-1-yl}-butyric acid;
3-(3-Fluoro-phenyl)-4-{4-[3-hydroxy-3-(5,6,7,8-tetrahydro-[1,8]naphthyridin-2-yl)-propenyl]-imidazol-1-yl}-butyric acid;
4-{4-[3-Hydroxy-3-(5,6,7,8-tetrahydro-[1,8]naphthyridin-2-yl)-propenyl]-imidazol-1-yl}-3-pyridin-3-yl-butyric acid;
3-Benzo[1,3]dioxol-5-yl-4-{4-[3-hydroxy-3-(5,6,7,8-tetrahydro-[1,8]naphthyridin-2-yl)-propenyl]-imidazol-1-yl}-butyric acid;
4-{4-[3-Hydroxy-3-(5,6,7,8-tetrahydro-[1,8]naphthyridin-2-yl)-propyl]-imidazol-1-yl}-3-phenyl-butyric acid;
3-(2,3-Dihydro-benzofuran-6-yl)-4-{4-[3-hydroxy-3-(5,6,7,8-tetrahydro-[1,8]naphthyridin-2-yl)-propyl]-imidazol-1-yl}-butyric acid;
3-(3-Fluoro-phenyl)-4-{4-[3-hydroxy-3-(5,6,7,8-tetrahydro-[1,8]naphthyridin-2-yl)-propyl]-imidazol-1-yl}-butyric acid;
4-{4-[3-Hydroxy-3-(5,6,7,8-tetrahydro-[1,8]naphthyridin-2-yl)-propyl]-imidazol-1-yl}-3-pyridin-3-yl-butyric acid;
3-Benzo[1,3]dioxol-5-yl]-{4-[3-hydroxy-3-(5,6,7,8-tetrahydro-[1,8]naphthyridin-2-yl)-propyl]-imidazol-1-yl}-butyric acid;
4-{4-[3-Hydroxy-3-(5,6,7,8-tetrahydro-[1,8]naphthyridin-2-yl)-propyl]-pyrazol-1-yl}-3-phenyl-butyric acid;
3-(2,3-Dihydro-benzofuran-6-yl)-4-(4-[3-hydroxy-3-(5,6,7,8-tetrahydro-[1,8]naphthyridin-2-yl)-propyl]-pyrazol-1-yl}-butyric acid;
3-(3-Fluoro-phenyl)-4-{4-[3-hydroxy-3-(5,6,7,8-tetrahydro-[1,8]naphthyridin-2-yl)-propyl]-pyrazol-1-yl}-butyric acid;
3-Benzo[1,3]dioxol-5-yl-4-{4-[3-hydroxy-3-(5,6,7,8-tetrahydro-[1,8]naphthyridin-2-yl)-propyl]-pyrazol-1-yl}-butyric acid;
4-{4-[3-Hydroxy-3-(5,6,7,8-tetrahydro-[1,8]naphthyridin-2-yl)-propyl]-pyrazol-1-yl}-3-pyridin-3-yl-butyric acid;
3-(3-Fluoro-phenyl)-4-{4-[3-(5,6,7,8-tetrahydro-[1,8]naphthyridin-2-yl)-propenyl]-imidazol-1-yl}-butyric acid;
3-Phenyl-4-{4-[3-(5,6,7,8-tetrahydro-[1,8]naphthyridin-2-yl)-propenyl]-imidazol-1-yl}-butyric acid;
3-(2,3-Dihydro-benzofuran-6-yl)-4-{4-[3-(5,6,7,8-tetrahydro-[1,8]naphthyridin-2-yl)-propenyl]-imidazol-1-yl}-butyric acid;
3-Pyridin-3-yl-4-{4-[3-(5,6,7,8-tetrahydro-[1,8]naphthyridin-2-yl)-propenyl]-imidazol-1-yl}-butyric acid;
3-Benzo[1,3]dioxol-5-yl-4-{4-[3-(5,6,7,8-tetrahydro-[1,8]naphthyridin-2-yl)-propenyl]-imidazol-1-yl}-butyric acid;
3-Benzo[1,3]dioxol-5-yl-4-{4-[3-(5,6,7,8-tetrahydro-[1,8]naphthyridin-2-yl)-propenyl]-pyrazol-1-yl}-butyric acid;
3-Pyridin-3-yl-4-{4-[3-(5,6,7,8-tetrahydro-[1,8]naphthyridin-2-yl)-propenyl]-pyrazol-1-yl}-butyric acid;
3-Phenyl-4-{4-[3-(5,6,7,8-tetrahydro-[1,8]naphthyridin-2-yl)-propenyl]-pyrazol-1-yl}-butyric acid;
3-hydroxy-4-{3-[4-(pyridin-2-ylamino)butyl]-1,2,4-oxadiazol-5-yl}butanoic acid;
3-hydroxy-4-{3-[3-(5,6,7,8-tetrahydro-1,8-naphthyridin-2-yl)propyl]-1,2,4-oxadiazol-5-yl}butanoic acid;
3-Benzo[1,3]dioxol-5-yl-4-{3-[4-(1H-imidazol-2-ylamino)-butyl]-[1,2,4]oxadiazol-5-yl}-butyric acid;
3-(3-Fluoro-phenyl)-4-{3-[4-(1H-imidazol-2-ylamino)-butyl]-[1,2,4]oxadiazol-5-yl}-butyric acid;
3-Benzo[1,3]dioxol-5-yl-4-{3-[4-(2H-pyrazol-3-ylamino)-butyl]-[1,2,4]oxadiazol-5-yl}-butyric acid;
3-(3-Fluoro-phenyl)-4-{3-[4-(2H-pyrazol-3-ylamino)-butyl]-[1,2,4]oxadiazol-5-yl}-butyric acid;
3-Benzo[1,3]dioxol-5-yl-4-{3-[4-(3H-imidazol-4-ylamino)-butyl]-[1,2,4]oxadiazol-5-yl}-butyric acid;
3-(3-Fluoro-phenyl)-4-{3-[4-(3H-imidazol-4-ylamino)-butyl]-[1,2,4]oxadiazol-5-yl}-butyric acid;
3-Benzo[1,3]dioxol-5-yl-4-{3-[3-(6-methylamino-pyridin-2-yl)-propyl]-[1,2,4]oxadiazol-5-yl}-butyric acid;
3-(3-Fluoro-phenyl)-4-{3-[3-(6-methylamino-pyridin-2-yl)-propyl]-[1,2,4]oxadiazol-5-yl}-butyric acid;
4-{3-[3-(6-Ethylamino-pyridin-2-yl)-propyl]-[1,2,4]oxadiazol-5-yl}-3-(3-fluoro-phenyl)-butyric acid;
3-(3-Fluoro-phenyl)-4-(3-{3-[6-(2-methoxy-ethylamino)-pyridin-2-yl]-propyl}-[1,2,4]oxadiazol-5-yl)-butyric acid;
3-(3-Fluoro-phenyl)-4-(3-{3-[6-(3-methoxy-propylamino)-pyridin-2-yl]-propyl}-[1,2,4]oxadiazol-5-yl)-butyric acid;
3-(3-Fluoro-phenyl)-4-(3-{3-[6-(2,2,2-trifluoro-ethylamino)-pyridin-2-yl]-propyl}-[1,2,4]oxadiazol-5-yl)-butyric acid;
3-(3-Fluoro-phenyl)-4-{3-[3-(5-oxo-5,6,7,8-tetrahydro-[1,8]naphthyridin-2-yl)-propyl]-[1,2,4]oxadiazol-5-yl}-butyric acid;
4-{3-[3-(5,5-Dimethyl-5,6,7,8-tetrahydro-[1,8]naphthyridin-2-yl)-propyl]-[1,2,4]oxadiazol-5-yl}-3-(3-fluoro-phenyl)-butyric acid;
4-{3-[3-(5,5-Difluoro-5,6,7,8-tetrahydro-[1,8]naphthyridin-2-yl)-propyl]-[1,2,4]oxadiazol-5-yl}-3-(3-fluoro-phenyl)-butyric acid; and
3-(1,3-benzodioxol-5-yl)-4-{3-[(5,6,7,8-tetrahydro-1,8-naphthyridin-2-ylmethoxy)methyl]-1,2,4-oxadiazol-5-yl}butanoic acid.

### DETAILED DESCRIPTION OF THE PREFERRED EMBODIMENTS

The present invention relates to a class of compounds represented by the Formula I, described above.

In another embodiment of the present invention 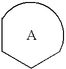
is a heteroaryl substituted by one or more substituents selected from lower alkyl, alkynyl, alkenyl, halogen, alkoxy, hydroxy, cyano, amino, alkylamino, dialkylamino or methylsulfonamide. More specifically, some examples of heteroaryl include oxadiazole, pyridine, pyrimidine, imidazole, thiadiazole, triazole, tetrazole, pyrazole, isoxazole, and thiazole.

Other embodiments of 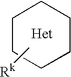
include the following heterocyclic ring systems containing at least one nitrogen atom: 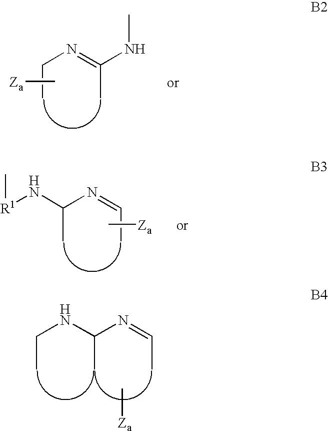
wherein Zₐ is H, alkyl, alkoxy, hydroxy, amine, alkylamine, dialkylamine, carboxyl, alkoxycarbonyl, hydroxyalkyl, halogen or haloalkyl and R¹ is H, alkyl, alkoxyalkyl, acyl, haloalkyl or alkoxycarbonyl. More specifically some examples include pyridylamino, imidazolylamino, morpholinopyridine, tetrahydronaphthyridine, oxazolylamino, thiazolylamino, pyrimidinylamino, quinoline, tetrahydroquinoline, imidazopyridine, benzimidazole, pyridone or quinolone.

The following heteroaryls include the ring systems described above.

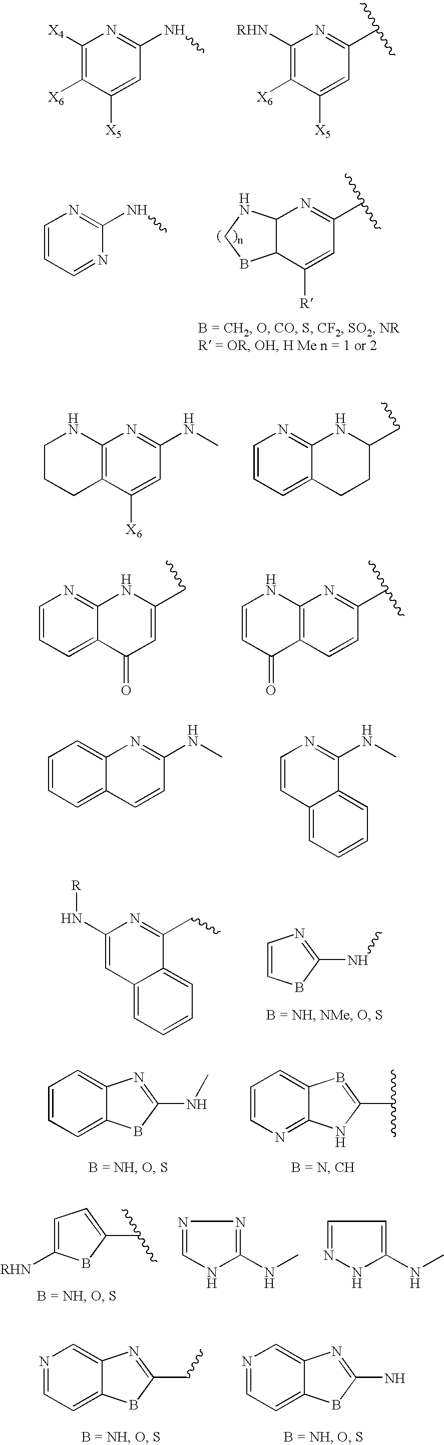

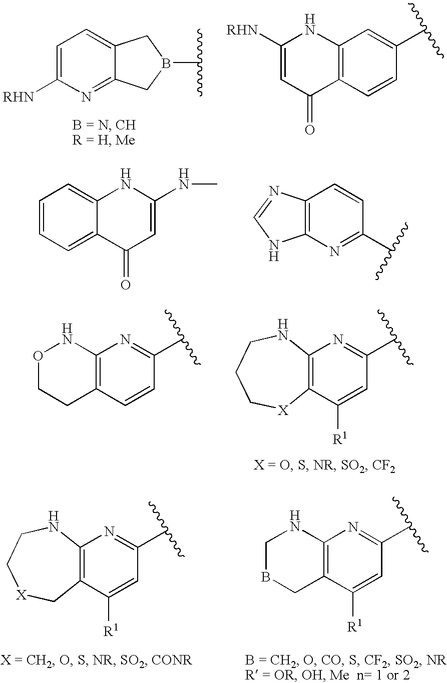

For the pyridyl derived heterocycle, the substituents X₄ and X₅ are selected from the group consisting of H, alkyl, branched alkyl, alkylamino, alkoxyalkylamino, haloalkyl, thioalkyl, halogen, amino, alkoxy, aryloxy, alkoxyalkyl, hydroxy, cyano or acylamino groups.

In another embodiment of the invention, the substituents X₄ and X₅ can be methyl, methoxy, amine, methylamine, trifluoromethyl, dimethylamine, hydroxy, chloro, bromo, fluoro and cyano. X₆ may preferentially be H, alkyl, hydroxy, halogen, alkoxy and haloalkyl. Alternately, the pyridyl ring can be fused with a 4-8 membered ring, optionally saturated or unsaturated. Some examples of these ring systems include tetrahydronaphthyridine, quinoline, tetrahydroquinoline, azaquinoline, morpholinopyridine, imidazopyridine and the like. The monocyclic ring systems such as imidazole, thiazole, oxazole, pyrazole, and the like, may contain an amino or alkylamino substituent at any position within the ring.

In another embodiment of the present invention, when Z₁ of Formula I is CO or SO₂, the linkage A¹-Z₂ of Formula I includes the heterocycle derived ring systems such as: pyridine, imidazole, thiazole, oxazole, benzimidazole, imidazopyridine and the like.

Other heterocycles for A¹-Z₂ of the present invention include 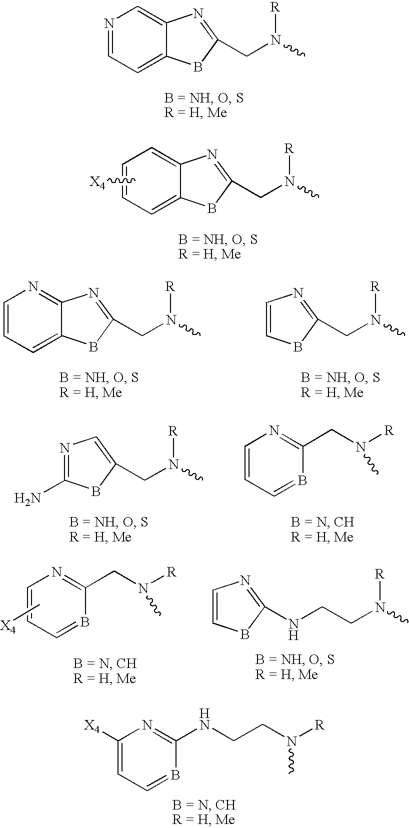
wherein X₄ is as defined above.

In another embodiment, Y³ or Y⁴ is an aryl or a heteroaryl group selected from phenyl, benzofuran, benzothiophene, indole, quinoline, isoquinoline, benzimidazole, benzoxazole, 1,3-benzodioxole, 1,4-benzodioxane, benzopyran, quinolone, imidazopyridine, tetrahydro-quinoline, benzotriazole, dihydroindole, dihydrobenzofuran, furan, thiophene, phenyl, oxazole, thiazole, isoxazole, pyrazole, imidazole, pyrrole, pyridine, pyrimidine, pyridone, triazole, thiadiazole and the like. The aryl system can be optionally substituted at one or more positions with alkyl, alkoxy, hydroxy, cyano, halogen or haloalkyl.

In another embodiment of the present invention, Y³ or Y⁴ may be an amine, alkylamine, acylamine, aminosulfone (NHSO₂R), arylamine, alkoxyalkylamine, aralkylamine, or heterocyclic amine.

In another embodiment of the present invention, Y³ taken together with Y⁴ forms a 3-8 membered monocyclic or a 7-11 membered bicyclic ring B, 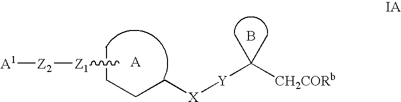
optionally containing one or more double bonds, optionally containing one or more heteroatoms or functional groups selected from O, NR^{g}, S, CO or SO₂, optionally substituted with one or more substituent selected from the group consisting of alkyl, haloalkyl, halogen, haloalkyl, alkoxy, alkyne, cyano, alkylsulfone, sulfonamide, carboalkoxy and carboxyalkyl; wherein R^{g} is selected from the group consisting of H, alkyl, haloalkyl, alkoxyalkyl, aryl, heteroaryl, aralkyl, and carboxyalkyl.

In another embodiment of the present invention, X taken together with Y³ forms a 3-7 membered monocyclic ring C, 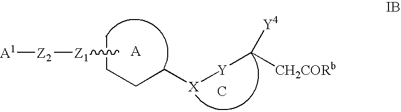
optionally containing one or more double bonds, optionally containing one or more heteroatom or functional group selected from O, NR^{g}, S, CO or SO₂, optionally substituted with one or more substituent selected from the group consisting of alkyl, halogen, alkoxy, haloalkyl, hydroxyalkyl, or alkoxyalkyl; wherein R^{g} is selected from the group consisting of H, alkyl, haloalkyl, alkoxyalkyl, aryl, heteroaryl, aralkyl, and carboxyalkyl.

The invention further relates to pharmaceutical compositions containing therapeutically effective amounts of the compounds of Formula I.

The invention also relates to a method of selectively inhibiting or antagonizing the α_{V} β₃ integrin and/or the α_{V} β₅ integrin and more specifically relates to a method of inhibiting bone resorption, periodontal disease, osteoporosis, humoral hypercalcemia of malignancy, Paget's disease, tumor metastasis, solid tumor growth (neoplasia), angiogenesis, including tumor angiogenesis, retinopathy including macular degeneration and diabetic retinopathy, arthritis, including rheumatoid arthritis, smooth muscle cell migration and restenosis by administering a therapeutically effective amount of a compound of the Formula I to achieve such inhibition together with a pharmaceutically acceptable carrier. More specifically it has been found that it is advantageous to administer compounds which are αᵥβ₃ and/or αᵥβ₅ selective and that such selectivity is beneficial in reducing unwanted side-effects.

The following is a list of definitions of various terms used herein:

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

As used herein, the terms “alkyl” or “lower alkyl” refer to a straight chain or branched chain hydrocarbon radicals having from about 1 to about 10 carbon atoms, and more preferably 1 to about 6 carbon atoms. Examples of such alkyl radicals are methyl, ethyl, n-propyl, isopropyl, n-butyl, isobutyl, sec-butyl, t-butyl, pentyl, neopentyl, hexyl, isohexyl, and the like.

As used herein the term “alkenyl” embraces linear or branched hydrocarbon radicals having at least one carbon-carbon double bond of two to about twenty carbon atoms or, preferably, two to about twelve carbon atoms. More preferred alkyl radicals are “lower alkenyl” radicals having two to about ten carbon atoms. In another embodiment, the alkenyl radicals are lower alkenyl radicals having two to about 6 carbon atoms. Examples of alkenyl radicals include ethenyl, propenyl, allyl, propenyl, butenyl and 4-methylbutenyl. The terms “alkenyl”, “lower alkenyl”, embrace radicals having “cis” and “trans” orientations, or alternatively, “E” and “Z” orientations.

As used herein the term “alkynyl” denotes linear or branched carbon or hydrocarbon radicals having two to about twenty carbon atoms or, preferably, two to about twelve carbon atoms. More preferred alkynyl radicals are “lower alkynyl” radicals having two to about ten carbon atoms. In another embodiment, the alkynyl radicals are lower alkynyl radicals having two to about six carbon atoms. Examples of such radicals include propargyl, butynyl, and the like.

The term “cycloalkyl” as used herein means saturated or partially unsaturated cyclic carbon radicals containing 3 to about 8 carbon atoms and more preferably 4 to about 6 carbon atoms. Examples of such cycloalkyl radicals include cyclopropyl, cyclopropenyl, cyclobutyl, cyclopentyl, cyclohexyl, 2-cyclohexen-1-yl, and the like.

The term “aryl” as used herein denotes aromatic ring systems composed of one or more aromatic rings. Preferred aryl groups are those consisting of one, two or three aromatic rings. The term embraces aromatic radicals such as phenyl, pyridyl, naphthyl, thiophene, furan, biphenyl and the like.

The “substituted aryl” moieties described herein are aryl moieties which are substituted with at least one atom, including moieties in which a carbon chain atom is substituted with a hetero atom such as nitrogen, oxygen, silicon, phosphorous, boron, sulfur, or a halogen atom. These substituents include halogen, heterocyclo, hydrocarbyloxy such as alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

As used herein, the term “cyano” is represented by a radical of the formula

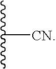

The terms “hydroxy” and “hydroxyl” as used herein are synonymous and are represented by a radical of the formula

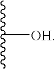

The term “lower alkylene” or “alkylene” as used herein refers to divalent linear or branched saturated hydrocarbon radicals of 1 to about 6 carbon atoms.

As used herein the term “alkoxy” refers to straight or branched chain oxy containing radicals of the formula —OR²⁰, wherein R²⁰ is an alkyl group as defined above. Examples of alkoxy groups encompassed include methoxy, ethoxy, n-propoxy, n-butoxy, isopropoxy, isobutoxy, sec-butoxy, t-butoxy and the like.

As used herein the terms “arylalkyl” or “aralkyl” refer to a radical of the formula 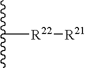
wherein R²¹ is aryl as defined above and R²² is an alkylene as defined above. Examples of aralkyl groups include benzyl, pyridylmethyl, naphthylpropyl, phenethyl and the like.

As used herein the term “nitro” is represented by a radical of the formula

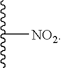

As used herein the term “halo” or “halogen” refers to bromo, chloro, fluoro or iodo.

As used herein the term “haloalkyl” refers to alkyl groups as defined above substituted with one or more of the same or different halo groups at one or more carbon atom. Examples of haloalkyl groups include trifluoromethyl, dichloroethyl, fluoropropyl and the like.

As used herein the term “carboxyl” or “carboxy” refers to a radical of the formula —COOH.

As used herein the term “carboxyl ester” refers to a radical of the formula —COOR²³ wherein R²³ is selected from the group consisting of H, alkyl, aralkyl or aryl as defined above.

As used herein the term “carboxyl derivative” refers to a radical of the formula 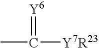
wherein Y⁶ and Y⁷ are independently selected from the group consisting of O, N or S and R²³ is selected from the group consisting of H, alkyl, aralkyl or aryl as defined above.

As used herein the term “amino” refers to the group —NT²T³, where each of T² and T³ is independently selected from the group consisting of hydrogen, hydrocarbyl, substituted hydrocarbyl, aryl, or heteroaryl. T² and T³ may also form a mono or polycyclic amino ring. The term “cyclicamino” embraces saturated heterocyclic radicals having three to eight atoms, at least one of which is nitrogen, but may also contain other heteroatoms such as oxygen, silicon, phosphorous, boron, sulfur, or a halogen.

As used herein the term “alkylsulfonyl” or “alkylsulfone” refers to a radical of the formula 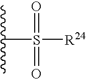
wherein R²⁴ is alkyl as defined above.

As used herein the term “alkylthio” refers to a radical of the formula —SR²⁴ wherein R²⁴ is alkyl as defined above.

As used herein the term “sulfonic acid” refers to a radical of the formula 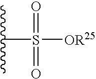
wherein R²⁵ is alkyl as defined above.

As used herein the term “sulfonamide” or “sulfonamido” refers to a radical of the formula 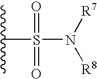
wherein R⁷ and R⁸ are as defined above.

As used herein the term “fused aryl” refers to an aromatic ring such as the aryl groups defined above fused to one or more phenyl rings. Embraced by the term “fused aryl” is the radical naphthyl and the like.

As used herein the terms “monocyclic heterocycle” or “monocyclic heterocyclic” refer to a monocyclic ring containing from 4 to about 12 atoms, and more preferably from 5 to about 10 atoms, wherein 1 to 3 of the atoms are heteroatoms selected from the group consisting of oxygen, nitrogen and sulfur with the understanding that if two or more different heteroatoms are present at least one of the heteroatoms must be nitrogen. Representative of such monocyclic heterocycles are imidazole, furan, pyridine, oxazole, pyran, triazole, thiophene, pyrazole, thiazole, thiadiazole, and the like.

As used herein the term “fused monocyclic heterocycle” refers to a monocyclic heterocycle as defined above with a benzene fused thereto. Examples of such fused monocyclic heterocycles include benzofuran, benzopyran, benzodioxole, benzothiazole, benzothiophene, benzimidazole and the like.

As used herein the term “methylenedioxy” refers to the radical 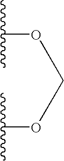
and the term “ethylenedioxy” refers to the radical

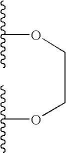

As used herein the term “4-12 membered dinitrogen containing heterocycle refers to a radical of the formula 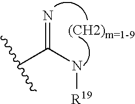
wherein m is 1 or 2 and R¹⁹ is H, alkyl, aryl, or aralkyl and more preferably refers to 4-9 membered ring and includes rings such as imidazoline.

As used herein the term “5-membered optionally substituted heteroaromatic ring” includes for example a radical of the formula 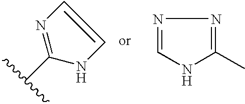
and “5-membered heteroaromatic ring fused with a phenyl” refers to such a “5-membered heteroaromatic ring” with a phenyl fused thereto. Representative of such 5-membered heteroaromatic rings fused with a phenyl is benzimidazole.

As used herein the term “bicycloalkyl” refers to a bicyclic hydrocarbon radical containing 6 to about 12 carbon atoms which is saturated or partially unsaturated.

As used herein the term “acyl” refers to a radical of the formula 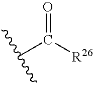
wherein R²⁶ is alkyl, alkenyl, alkynyl, aryl or aralkyl and optionally substituted thereon as defined above. Encompassed by such radical are the groups acetyl, benzoyl and the like.

As used herein the term “thio” refers to a radical of the formula

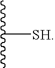

As used herein the term “sulfonyl” refers to a radical of the formula 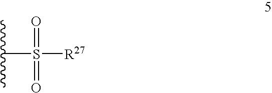
wherein R²⁷ is alkyl, aryl or aralkyl as defined above.

As used herein the term “haloalkylthio” refers to a radical of the formula —S—R²⁸ wherein R²⁸ is haloalkyl as defined above.

As used herein the term “aryloxy” refers to a radical of the formula 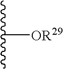
wherein R²⁹ is aryl as defined above.

As used herein the term “acylamino” refers to a radical of the formula 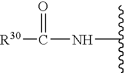
wherein R³⁰ is alkyl, aralkyl or aryl as defined above.

As used herein the term “amido” refers to a radical of the formula

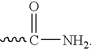

As used herein the term “alkylamino” refers to a radical of the formula —NHR³² wherein R³² is alkyl as defined above.

As used herein the term “dialkylamino” refers to a radical of the formula —NR³³R³⁴ wherein R³³ and R³⁴ are the same or different alkyl groups as defined above.

As used herein the term “trifluoromethyl” refers to a radical of the formula

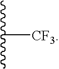

As used herein the term “trifluoroalkoxy” refers to a radical of the formula 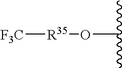
wherein R³⁵ is a bond or an alkylene as defined above.

As used herein the term “alkylaminosulfonyl” or “aminosulfonyl” refers to a radical of the formula 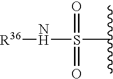
wherein R³⁶ is alkyl as defined above.

As used herein the term “alkylsulfonylamino” or “”alkylsulfonamide” refers to a radical of the formula 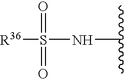
wherein R³⁶ is alkyl as defined above.

As used herein the term “trifluoromethylthio” refers to a radical of the formula

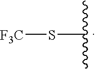

As used herein the term “trifluoromethylsulfonyl” refers to a radical of the formula

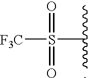

As used herein the term “4-12 membered mono-nitrogen containing monocyclic or bicyclic ring” refers to a saturated or partially unsaturated monocyclic or bicyclic ring of 4-12 atoms and more preferably a ring of 4-9 atoms wherein one atom is nitrogen. Such rings may optionally contain additional heteroatoms selected from nitrogen, oxygen or sulfur. Included within this group are morpholine, piperidine, piperazine, thiomorpholine, pyrrolidine, proline, azacycloheptene and the like.

As used herein the term “benzyl” refers to the radical

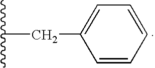

As used herein the term “phenethyl” refers to the radical

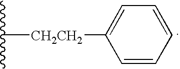

As used herein the term “4-12 membered mono-nitrogen containing monosulfur or monooxygen containing heterocyclic ring” refers to a ring consisting of 4 to 12 atoms and more preferably 4 to 9 atoms wherein at least one atom is a nitrogen and at least one atom is oxygen or sulfur. Encompassed within this definition are rings such as thiazoline and the like.

As used herein the term “arylsulfonyl” or “arylsulfone” refers to a radical of the formula 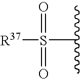
wherein R³⁷ is aryl as defined above.

As used herein the terms “alkylsulfoxide” or “arylsulfoxide” refer to radicals of the formula 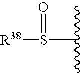
wherein R³⁸ is, respectively, alkyl or aryl as defined above.

As used herein the term “arylthio” refers to a radical of the formula 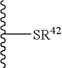
wherein R⁴² is aryl as defined above.

As used herein the term “monocyclic heterocycle thio” refers to a radical of the formula 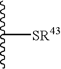
wherein R⁴³ is a monocyclic heterocycle radical as defined above.

As used herein the terms “monocyclic heterocycle sulfoxide” and “monocyclic heterocycle sulfone” refer, respectively, to radicals of the formula 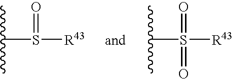
wherein R⁴³ is a monocyclic heterocycle radical as defined above.

As used herein the term “alkylcarbonyl” refers to a radical of the formula 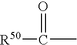
wherein R⁵⁰ is alkyl as defined above.

As used herein the term “arylcarbonyl” refers to a radical of the formula 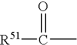
wherein R⁵¹ is aryl as defined above.

As used herein the term “alkoxycarbonyl” refers to a radical of the formula 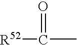
wherein R⁵² is alkoxy as defined above.

As used herein the term “aryloxycarbonyl” refers to a radical of the formula 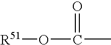
wherein R⁵¹ is aryl as defined above.

As used herein the term “haloalkylcarbonyl” refers to a radical of the formula 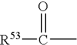
wherein R⁵³ is haloalkyl as defined above.

As used herein the term “haloalkoxycarbonyl” refers to a radical of the formula 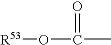
wherein R⁵³ is haloalkyl as defined above.

As used herein the term “alkylthiocarbonyl” refers to a radical of the formula 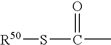
wherein R⁵⁰ is alkyl as defined above.

As used herein the term “arylthiocarbonyl” refers to a radical of the formula 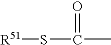
wherein R⁵′ is aryl as defined above.

As used herein the term “acyloxymethoxycarbonyl” refers to a radical of the formula 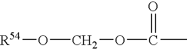
wherein R⁵⁴ is acyl as defined above.

As used herein the term “arylamino” refers to a radical of the formula R⁵¹—NH— wherein R⁵¹ is aryl as defined above.

As used herein the term “acyloxy” refers to a radical of the formula R⁵⁵—O— wherein R⁵⁵ is acyl as defined above.

As used herein the term “alkenylalkyl” refers to a radical of the formula R⁵⁰—R⁵⁷— wherein R⁵⁰ is an alkenyl as defined above and R⁵⁷ is alkylene as defined above.

As used herein the term “alkenylene” refers to a linear hydrocarbon radical of 1 to about 8 carbon atoms containing at least one double bond.

As used herein the term “alkoxyalkyl” refers to a radical of the formula R⁵⁶—R⁵⁷—wherein R⁵⁶ is alkoxy as defined above and R⁵⁷ is alkylene as defined above.

As used herein the term “alkynylalkyl” refers to a radical of the formula R⁵⁹—R⁶⁰—wherein R⁵⁹ is alkynyl as defined as above and R⁶⁰ is alkylene as defined as above.

As used herein the term “alkynylene” refers to divalent alkynyl radicals of 1 to about 6 carbon atoms.

As used herein the term “allyl” refers of a radical of the formula —CH₂CH═CH₂.

As used herein the term “aminoalkyl” refers to a radical of the formula H₂N—R wherein R⁶¹ is alkylene as defined above.

As used herein the term “benzoyl” refers to the aryl radical C₆H₅—CO—.

As used herein the term “carboxamide” or “carboxamido” refer to a radical of the formula —CO—NH₂.

As used herein the term “carboxyalkyl” refers to a radical HOOC—R⁶²—wherein R⁶² is alkylene as defined as above.

As used herein the term “carboxylic acid” refers to the radical —COOH.

As used herein the term “ether” refers to a radical of the formula R⁶³—O— wherein R⁶³ is selected from the group consisting of alkyl, aryl and heteroaryl.

The term “heteroatom” shall mean atoms other than carbon and hydrogen.

The term “heterocyclo” and “heterocyclic” embraces saturated, partially unsaturated and unsaturated heteroatom-containing ring-shaped radicals containing 3 to 10 members, including at least 1 carbon atom and up to 9 additional members independently selected from carbon, nitrogen, sulfur and oxygen. This includes, for example, the following structures: 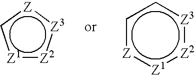
wherein Z, Z¹, Z² or Z³ is C, S, O, or N, with the proviso that one of Z, Z¹, Z² or Z³ is other than carbon, but is not O or S when attached to another Z atom by a double bond or when attached to another O or S atom. Furthermore, the optional substituents are understood to be attached to Z, Z¹, Z² or Z³ only when each is C.

Examples of saturated heterocyclyl radicals include saturated 3 to 8-membered heteromonocylic group containing 1 to 4 nitrogen atoms (e.g. pyrrolidinyl, imidazolidinyl, piperidino, piperazinyl, etc.); saturated 3 to 8-membered heteromonocyclic group containing 1 to 2 oxygen atoms and 1 to 3 nitrogen atoms (e.g. morpholinyl, etc.); saturated 3 to 8-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The “substituted heterocyclo” moieties described herein are heterocyclo moieties which are substituted with at least one atom, including moieties in which a carbon chain atom is substituted with a hetero atom such as nitrogen, oxygen, silicon, phosphorous, boron, sulfur, or a halogen atom. These substituents include halogen, heterocyclo, hydrocarbyloxy such as alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

As used herein the term “haloalkylsulfonyl” refers to a radical of the formula 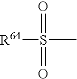
wherein the R⁶⁴ is haloalkyl as defined above.

As used herein the term “heteroaryl” refers to an aryl radical contain at least one heteroatom.

As used herein the term “hydroxyalkyl” refers to a radical of the formula HO—R⁶⁵— wherein R⁶⁵ is alkylene as defined above.

As used herein the term “keto” refers to a carbonyl group joined to 2 carbon atoms.

As used herein the term “lactone” refers to an anhydro cyclic ester produced by intramolecular condensation of a hydroxy acid with the elimination of water.

As used herein the term “olefin” refers to an unsaturated hydrocarbon radical of the type CₙH₂ₙ.

As used herein the term “sulfone” refers to a radical of the formula R⁶⁶—SO₂—.

As used herein the term “thioalkyl” refers to a radical of the formula R⁷⁷—S— wherein R⁷⁷ is alkyl as defined above.

As used herein the term “thioether” refers to a radical of the formula R⁷⁸—S— wherein R⁷⁸ is alkyl, aryl or heteroaryl.

As used herein the term “trifluoroalkyl” refers to an alkyl radical as defined above substituted with three halo radicals as defined above.

The term “composition” as used herein means a product that results from the mixing or combining of more than one element or ingredient.

The term “pharmaceutically acceptable carrier”, as used herein means a pharmaceutically acceptable material, composition or vehicle, such as a liquid or solid filler, diluent, excipient, solvent or encapsulating material, involved in carrying or transporting a chemical agent.

The term “therapeutically effective amount” shall mean that amount of drug or pharmaceutical agent that will elicit the biological or medical response of a tissue, system or animal that is being sought by a researcher or clinician.

As used herein, the term “treatment” is meant the medical management of a subject, e.g. an animal or human, with the intent that a prevention, cure, stabilization, or amelioration of the symptoms or condition will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder. “Treating” a condition with the compounds of the invention involves administering such a compound, alone or in combination and by any appropriate means, to an animal, cell, lysate or extract derived from a cell, or a molecule derived from a cell.

The following is a list of abbreviations and the corresponding meanings as used interchangeably herein:
¹H-NMR=proton nuclear magnetic resonance
AcOH=acetic acid
BOC=tert-butoxycarbonyl
BuLi=butyl lithium
Cat. =catalytic amount
CDI=Carbonyldiimidazole
CH₂Cl₂=dichloromethane
CH₃CN=acetonitrile
CH₃₁=iodomethane
CHN analysis=carbon/hydrogen/nitrogen elemental analysis
CHNCl analysis=carbon/hydrogen/nitrogen/chlorine elemental analysis
CHNS analysis=carbon/hydrogen/nitrogen/sulfur elemental analysis
DEAD=diethylazodicarboxylate
DIAD=diisopropylazodicarboxylate
DI water=deionized water
DMA=N,N-dimethylacetamide
DMAC=N,N-dimethylacetamide
DMF=N,N-dimethylformamide
EDC=1-(3-dimethylaminopropyl)-3-ethylcarbodiimide hydrochloride
Et=ethyl
Et₂O=diethyl ether
Et₃N=triethylamine
EtOAc=ethyl acetate
EtOH=ethanol
FAB MS=fast atom bombardment mass spectroscopy
g=gram(s)
HOBT=1-hydroxybenzotriazole hydrate
HPLC=high performance liquid chromatography
i-Pr=isopropyl
i-Prop=isopropyl
K₂CO₃=potassium carbonate
KMnO₄=potassium permanganate
KOH=potassium hydroxide
KSCN=potassium thiocyanate
L=Liter
LiOH=lithium hydroxide
Me=methyl
MeOH=methanol
mg=milligram
MgSO₄=magnesium sulfate
ml=milliliter
mL=milliliter
MS=mass spectroscopy
NaH—sodium hydride
NaHCO₃=sodium bicarbonate
NaOH=sodium hydroxide
NaOMe=sodium methoxide
NH₄⁺HCO₂⁻=ammonium formate
NMR=nuclear magnetic resonance
Pd=palladium
Pd/C=palladium on carbon
Ph=phenyl
Pt=platinum
Pt/C=platinum on carbon
RPHPLC=reverse phase high performance liquid chromatography
RT=room temperature
t-BOC=tert-butoxycarbonyl
TFA=trifluoroacetic acid
THF=tetrahydrofuran
TLC—thin layer chromatography
TMS=trimethylsilyl
Δ=heating the reaction mixture

The compounds as shown above can exist in various isomeric forms and all such isomeric forms are meant to be included. Tautomeric forms are also included as well as pharmaceutically acceptable salts of such isomers and tautomers.

In the structures and formulas herein, a bond drawn across a bond of a ring can be to any available atom on the ring.

The term “pharmaceutically acceptable salt” refers to a salt prepared by contacting a compound of Formula I with an acid whose anion is generally considered suitable for human consumption. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid. Representative salts include the following: benzenesulfonate, hydrobromide and hydrochloride. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. All of the pharmacologically acceptable salts may be prepared by conventional means. (See Berge et al., J. Pharm. Sci., 66(1), 1-19 (1977) for additional examples of pharmaceutically acceptable salts.)

The compounds of the present invention can have chiral centers and occur as racemates, racemic mixtures, diastereomeric mixtures, and as individual diastereomers or enantiomers, with all isomeric forms included in the present invention. Therefore, where a compound is chiral, the separate enantiomers or diastereomers, substantially free of the other, are included within the scope of the present invention; further included are all mixtures of the enantiomers or diastereomers. Also included within the scope of the invention are polymorphs, or hydrates or other modifiers of the compounds of invention.

The present invention includes within its scope prodrugs of the compounds of this invention. In general, such prodrugs will be functional derivatives of the compounds of this invention that are readily convertible in vivo into the required compound. For example, prodrugs of a carboxylic acid may include an ester, an amide, or an ortho-ester. Thus, in the methods of treatment of the present invention, the term “administering” shall encompass the treatment of the various conditions described with the compound specifically disclosed or with a compound which may not be specifically disclosed, but which converts to the compound of Formula I in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

For the selective inhibition or antagonism of αᵥβ₃ and/or α_{V}β₅ integrins, compounds of the present invention may be administered orally, parenterally, or by inhalation spray, or topically in unit dosage formulations containing conventional pharmaceutically acceptable carriers, adjuvants and vehicles. The term parenteral as used herein includes, for example, subcutaneous, intravenous, intramuscular, intrasternal, transmuscular infusion techniques or intraperitonally.

The compounds of the present invention are administered by any suitable route in the form of a pharmaceutical composition adapted to such a route, and in a dose effective for the treatment intended. Therapeutically effective doses of the compounds required to prevent or arrest the progress of or to treat the medical condition are readily ascertained by one of ordinary skill in the art using preclinical and clinical approaches familiar to the medicinal arts.

Accordingly, the present invention provides a method of treating conditions mediated by selectively inhibiting or antagonizing the α_{V}β₃ and/or α_{V} β₅ cell surface receptor which method comprises administering a therapeutically effective amount of a compound selected from the class of compounds depicted in the above formulas, wherein one or more compound is administered in association with one or more non-toxic, pharmaceutically acceptable carriers and/or diluents and/or adjuvants (collectively referred to herein as “carrier” materials) and if desired other active ingredients. More specifically, the present invention provides a method for selective antagonism of the α_{V}β₃ and/or α_{V}β₅ cell surface receptors over α_{IIb}β₃ or αᵥβ₆ integrin receptors. Most preferably the present invention provides a method for inhibiting bone resorption, treating osteoporosis, inhibiting humoral hypercalcemia of malignancy, treating Paget's disease, inhibiting tumor metastasis, inhibiting neoplasia (solid tumor growth), inhibiting angiogenesis including tumor angiogenesis, treating retinopathy including macular degeneration and diabetic retinopathy, inhibiting arthritis, psoriasis and periodontal disease, and inhibiting smooth muscle cell migration including restenosis.

Based upon standard laboratory experimental techniques and procedures well known and appreciated by those skilled in the art, as well as comparisons with compounds of known usefulness, the compounds of Formula I can be used in the treatment of patients suffering from the above pathological conditions. One skilled in the art will recognize that selection of the most appropriate compound of the invention is within the ability of one with ordinary skill in the art and will depend on a variety of factors including assessment of results obtained in standard assay and animal models.

Treatment of a patient afflicted with one of the pathological conditions comprises administering to such a patient an amount of compound of the Formula I which is therapeutically effective in controlling the condition or in prolonging the survivability of the patient beyond that expected in the absence of such treatment. As used herein, the term “inhibition” of the condition refers to slowing, interrupting, arresting or stopping the condition and does not necessarily indicate a total elimination of the condition. It is believed that prolonging the survivability of a patient, beyond being a significant advantageous effect in and of itself, also indicates that the condition is beneficially controlled to some extent.

As stated previously, the compounds of the invention can be used in a variety of biological, prophylactic or therapeutic areas. It is contemplated that these compounds are useful in prevention or treatment of any disease state or condition wherein the α_{V} β₃ and/or α_{V} β₅ integrin plays a role.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions.

Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 1.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, 200 or 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regiment.

For administration to a mammal in need of such treatment, the compounds in a therapeutically effective amount are ordinarily combined with one or more adjuvants appropriate to the indicated route of administration. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulphuric acids, gelatin, acacia, sodium alginate, polyvinylpyrrolidone, and/or polyvinyl alcohol, and tableted or encapsulated for convenient administration. Alternatively, the compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The pharmaceutical compositions useful in the present invention may be subjected to conventional pharmaceutical operations such as sterilization and/or may contain conventional pharmaceutical adjuvants such as preservatives, stabilizers, wetting agents, emulsifiers, buffers, etc.

The general synthetic sequences for preparing the compounds useful in the present invention are outlined in Schemes 1-42. Both an explanation of, and the actual procedures for, the various aspects of the present invention are described where appropriate. The following Schemes and Examples are intended to be merely illustrative of the present invention, and not limiting thereof in either scope or spirit. Those with skill in the art will readily understand that known variations of the conditions and processes described in the Schemes and Examples can be used to synthesize the compounds of the present invention.

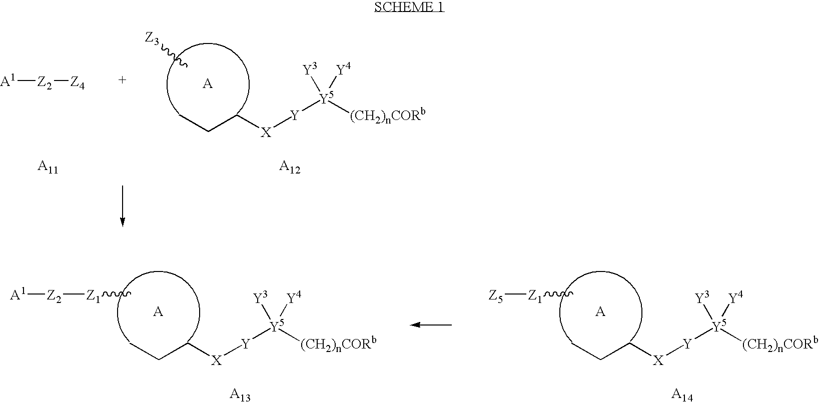

### Scheme 1

The compounds of formula A₁₃, wherein the ring A is preferentially a 6-member heteroaryl or a bicyclic heteroaryl, can be prepared by reacting an intermediate of formula A₁₁ with a compound of the formula A₁₂. For example, when Z₃ is OH, SH or NHR, A₁₂ may be alkylated with A₁₁ (Z₄=Br or OMs) using base such as (sodium hydride, potassium hydride) preferably in a solvent such as dimethylsulfoxide or DMF. These reactions may preferentially be carried at 0° C. to approx. 40° C. Alternately, when Z₃ and Z₄ are both OH, the ether formation to product A₁₃ may be accomplished by using Mitsunobu reaction. This reaction may preferentially be carried out using triarylphosphine (such as triphenylphoshine) and azodicarboxylate (such as diethyl azodicarboxylate, di-tert-butyl azodicarboxylate, diisopropyl azodicarboxylate) in solvents such as DMF, methylene chloride, THF and the like. When Z₃ carries a carboxylic acid and Z₄ is an amine, the standard coupling conditions may be used to synthesize the carboxamide (CONH) containing targets A₁₃

Alternately, the compounds of formula A₁₃ may be prepared by starting with compounds of general formula A₁₄. For example, when Z₅ in A₁₄ is NH₂, cyclic or acyclic guanidino containing compounds of formula A₁₃ may be synthesized by adopting the methodologies discussed, for example in U.S. Pat. Nos. 5,852,210 and 5,773,646. Similarly, compounds of formula A₁₄ (Z₅=CHO) may be treated with amino containing heteroaromatic system (such as 2-aminopyridine) to give the target compounds A₁₃. This reaction may preferentially be carried out by reductive amination procedures using reducing agents such as sodium triacetoxyborohydride, sodium cyanoborohydride or sodium borohydride.

### EXAMPLE 1

### 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid

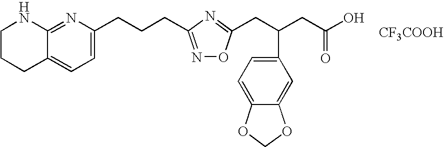 Step 1
Diethyl 2-(1,3-benzodioxol-5-yl)-4-hydroxy-4-methyl-6-oxocyclohexane-1,3-dicarboxylate:

Following the procedure of Brown, E.; Dhal, R.; Papin, N.; Tetrahedron, 1995, 51, 13061-13072: piperonal (22.5 g; 150 mmoles), ethyl acetoacetate (38.24 ml; 300 mmoles), and piperidine (1.5 ml) were combined in a 500 ml round bottom flask and stirred at room temperature. After 72 hours, the mixture solidified and was re-crystallized using ethanol to give 42.4 g. of product (72%). ¹H NMR (DMSO-d₆) δ 6.95 (m, 1H), 6.8 (m, J=7.5 Hz, 1H), 6.71 (m, J=7.5 Hz, 1H), 5.97 (s, 2H), 4.9 (s, 1H), 4.0-3.7 (m, 6H), 3.25 (d, J=11 Hz, 1H), 2.9 (d, J=14 Hz, 1H), 2.35 (d, J=14 Hz, 1H), 1.23 (s, 3H), 1.0 (t, 3H), 0.92 (t, 3H).

Step 2

3-(1,3-benzodioxol-5-yl)pentanedioic acid:

Diethyl 2-(1,3-benzodioxol-5-yl)-4-hydroxy-4-methyl-6-oxocyclohexane-1,3-dicarboxylate (19 g) was suspended in ethanol (140 ml) and an aqueous solution of NaOH (50%, 270 ml). The mixture was heated at reflux for one hour. After the mixture was cooled to room temperature, the ethanol was removed under reduced pressure. Then, concentrated HCl was added until pH 1 was achieved while maintaining the temperature below 50° C. The mixture was filtered. The solid was washed with ether. The two layers were separated. The aqueous layer was extracted with ether (3×). The ether layers were combined, dried, and concentrated to give product. ¹H NMR (DMSO-d₆) δ 12.06 (br s, 2H), 6.88-6.68 (m, 3H), 5.98 (s, 2H), 3.4-3.3 (m, 1H), 2.62-2.42 (m, 4H).

Step 3

4-(1,3-benzodioxol-5-yl)dihydro-2H-pyran-2,6(3H)-dione:

3-(1,3-benzodioxol-5-yl)pentanedioic acid (2 g, 7.9 mmoles) was suspended in acetic anhydride (50 ml) and refluxed for two hours. The reaction mixture was allowed to cool to room temperature and the solvent was removed under reduced pressure. The residue was triturated with ether to give the product (1.3 g, 70%). ¹H NMR (DMSO-d₆) δ 6.91(m, 1H), 6.89 (m, 1H), 6.72 (m, 1H), 6.01 (s, 2H), 3.53-3.41 (m, 1H), 3.07-2.89 (m, 4H).

Step 4

3-(1,3-benzodioxol-5-yl)-4-{3-[3-(5,6,7,8-tetrahydro-1,8-naphthyridin-2-yl)-propyl]-1,2,4-oxadiazol-5-yl}butanoic acid trifluoroacetate:

The title compound was prepared using the following general procedure:

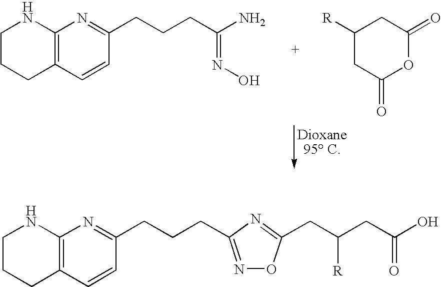

100 mg of the amide oxime (prepared according to the method as described in WO 99/30709) was added to an equivalent of the anhydride suspended in dioxane (5 ml). The reaction mixture was heated to 95° C. overnight, the solvent was removed and the residue purified on HPLC (Gilson) using acetonitrile gradient 10-50% in 12 minutes for all compounds except the pyridine and quinoline derivatives used a gradient 5-35% in 12 minutes. ¹H NMR (DMSO-d₆) δ 12.2 (br s, 1H), 8.13 (br s, 1H), 7.59 (d, J=7.5 Hz, 1H), 6.91-6.52 (m, 4H), 5.91 (s, 2H), 3.52-3.39 (m, 3H), 3.3-3.14 (m, 2H), 2.77-2.56 (m, 8H), 1.99-1.90 (m, 2H), 1.88-1.76 (m, 2H). Anal. Calcd. for C₂₄H₂₆N₄O₅ plus 1.2 CF₃CO₂H and 1.0H₂O: C, 52.38; H, 4.86; N, 9.26. Found: C, 52.47; H, 4.47; N, 9.27.

### EXAMPLE 2

3-(3,4-Dichlorophenyl)-4-{3-[3-(5,6,7,8-tetrahydro-1,8-naphthyridin-2-yl)propyl]-1,2,4-oxadiazol-5-yl}butanoic acid trifluoroacetate

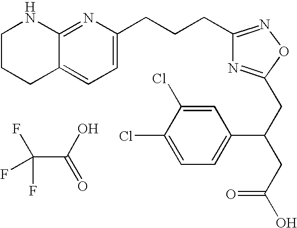

The title compound was prepared according to the method described for preparing the product of EXAMPLE 1 using 3,4-dichlorophenylbenzaldehyde: ¹H NMR (CD₃OD) δ 7.55 (d, 1H), 7.50-7.40 (m, 2H), 7.30-7.20 (m, 1H), 6.55 (d, 1H), 3.75-3.65 (m, 1H), 3.50-3.45 (m, 2H), 3.40-3.23 (m, 2H), 2.90-2.60 (m, 8H), 2.10-2.00 (m, 2H), 2.00-1.90 (m, 2H); MS (ESI+) for C₂₃H₂₄Cl₂N₄O₃ m/z 476.1 (M+H)⁺.

### EXAMPLE 3

3-(3-Fluoro-4-methylphenyl)-4-{3-[3-(5,6,7,8-tetrahydro-1,8-naphthyridin-2-yl)propyl]-1,2,4-oxadiazol-5-yl}butanoic acid hydrochloride.

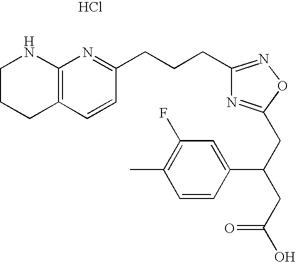

The title compound was prepared according to the method described for preparing the product of EXAMPLE 1 using 3-fluoro-4-methylbenzaldehyde: ¹H NMR (CDCl₃) δ 8.45 (broad s, 1H), 7.35 (d, 1H), 7.15-7.10 (m, 1H), 6.95-6.85 (m, 2H), 6.40 (d, 1H), 3.80-3.70 (m, 1H), 3.55-3.47 (m, 2H), 3.20-3.15 (m, 2H), 2.9-2.7 (m, 8H), 2.3-2.2 (m, 2H), 2.23 (s, 3H), 2.0-1.9 (m, 2H); MS (ESI+) for C₂₄H₂₇FN₄O₃ m/z 439.19 (M+H)⁺.

### EXAMPLE 4

3-(4-Phenoxyphenyl)-4-{3-[3-(5,6,7,8-tetrahydro-1,8-naphthyridin-2-yl)propyl]-1,2,4-oxadiazol-5-yl}butanoic acid trifluoroacetate

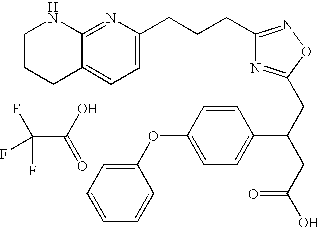

The title compound was prepared according to the method described for preparing the product of EXAMPLE 1 of using 4-phenoxybenzaldehyde: ¹H NMR (CD₃OD) δ 7.51 (d, 1H), 7.35-7.28 (m, 2H), 7.23 (d, 2H), 7.13-7.05 (m, 1H), 6.93-6.83 (m, 4H), 6.34 (d, 1H), 3.76-3.66 (m, 1H), 3.52-3.45 (m, 2H), 3.40-3.30 (m, 1H), 3.29-3.20 (m, 1H), 2.90-2.63 (m, 8H), 2.10-2.00 (m, 2H), 1.96-1.88 (m, 2H); MS (ESI+) for C₂₉H₃₀N₄O₄ m/z 499.23 (M+H)⁺.

### EXAMPLE 5

3-(1-Benzofuran-2-yl)-4-{3-[3-(5,6,7,8-tetrahydro-1,8-naphthyridin-2-yl)propyl]-1,2,4-oxadiazol-5-yl}butanoic acid trifluoroacetate

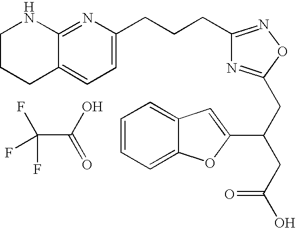

The title compound was prepared according to the method described for preparing the product of EXAMPLE 1 of using benzofuran-2-aldehyde: ¹H NMR (CD₃OD) δ 7.50-7.44 (m, 2H), 7.39 (d, 1H), 7.23-7.13 (m, 2H), 6.55 (s, 1H), 6.48 (d, 1H), 4.13-3.93 (m, 1H), 3.50-3.40 (m, 2H), 2.96-2.83 (m, 2H), 2.80-2.76 (m, 2H), 2.75-2.70 (m, 2H), 2.66-2.60 (m, 2H), 2.06-1.98 (m, 2H), 1.98-1.90 (m, 2H). MS (ESI+) for C₂₅H₂₆N₄O₄ m/z 447.14 (M+H)⁺.

### EXAMPLE 6

3-[4-(Benzyloxy)phenyl]4-{3-[3-(5,6,7,8-tetrahydro-1,8-naphthyridin-2-yl)propyl]-1,2,4-oxadiazol-5-yl}butanoic acid trifluoroacetate

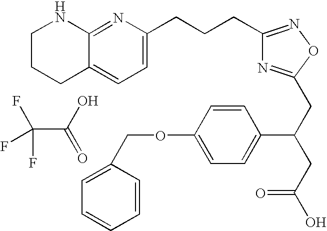

The title compound was prepared according to the method described for preparing the product of EXAMPLE 1 using 4-benzyloxybenzaldehyde: ¹H NMR (CD₃OD) δ 7.56-7.53 (d, 1H), 7.48-7.34 (m, 5H), 7.23 (d, 2H), 6.94 (d, 2H), 6.55 (d, 1H), 5.50-5.30 (s, 2H), 3.78-3.68 (m, 1H), 3.56-3.49 (m, 2H), 3.44-3.35 (m, 1H), 3.33-3.25 (m, 1H), 2.93-2.84 (m, 1H), 2.83-2.73 (m, 5H), 2.70-2.63 (m, 2H), 2.15-2.05 (m, 2H), 2.00-1.93 (m, 2H); MS (ESI+) for C₃₀H₃₂N₄O₄ m/z 513.88 (M+H)⁺.

### EXAMPLE 7

3-[4-(Methylsulfonyl)phenyl]-4-{3-[3-(5,6,7,8-tetrahydro-1,8-naphthyridin-2-yl)propyl]-1,2,4-oxadiazol-5-yl}butanoic acid trifluoroacetate

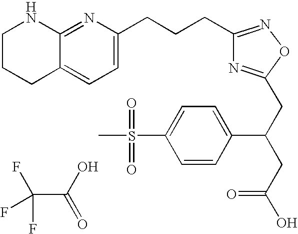

The title compound was prepared according to the method described for preparing the product of EXAMPLE 1 using 4-methylsufonylbenzaldehyde: ¹H NMR (CD₃OD) δ 7.85 (d, 2H), 7.56 (d, 3H), 6.55 (d, 1H), 3.90-3.80 (m, 1H), 3.54-3.50 (m, 2H), 3.46-3.30 (m, 2H), 3.09 (s, 3H), 2.80-2.90 (m, 1H), 2.86-2.80 (m, 3H), 2.75-2.69 (m, 2H), 2.69-2.63 (m, 2H), 2.06-1.93 (m, 4H); MS (ESI+) for C₂₄H₂₈N₄O₅S m/z 485.12 (M+H)⁺.

### EXAMPLE 8

4-{3-[3-(5,6,7,8-Tetrahydro-1,8-naphthyridin-2-yl)propyl]-1,2,4-oxadiazol-5-yl}-3-[4-(trifluoromethoxy)phenyl]butanoic acid trifluoroacetate

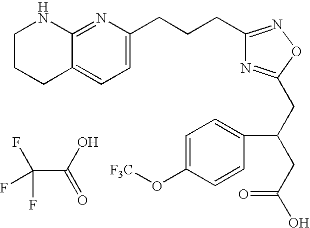

The title compound was prepared according to the method described for preparing the product of EXAMPLE 1 using 4-trifluoromethoxybenzaldehyde: ¹H NMR (DMSO-d6) δ) 8.05 (broad s, 1H), 7.59 (d, 1H), 7.40 (d, 2H), 7.25 (d, 2H), 6.55 (d, 1H), 3.65-3.55 (m, 1H), 3.39-3.21 (m, 4H), 2.85-2.60 (m, 8H), 1.95-1.86 (m, 2H), 1.85-1.78 (m, 2H); MS (ESI+) for C₂₄H₂₅F₃N₄O₄ m/z 491.12 (M+H)⁺.

### EXAMPLE 9

3-(3-Furyl)-4-{3-[3-(5,6,7,8-tetrahydro-1,8-naphthyridin-2-yl)propyl]-1,2,4-oxadiazol-5-yl}butanoic acid trifluoroacetate

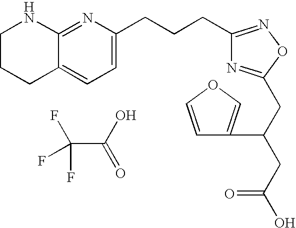

The title compound was prepared according to the method described for preparing the product of EXAMPLE 1 using 3-furaldehyde: ¹H NMR (DMSO-d₆) δ 8.00 (broad s, 1H), 7.56 (d, 1H), 7.52 (s, 1H), 7.41 (s, 1H), 6.56 (d, 1H), 6.43 (s, 1H), 3.55-3.45 (m, 1H), 3.29-3.11 (m, 4H), 2.75-2.53 (m, 8H), 2.01-1.91 (m, 2H), 1.85-1.75 (m, 2H); MS (ESI+) for C₂₁H₂₄N₄O₄ m/z 397.55 (M+H)⁺.

### EXAMPLE 10

4-{3-[3-(5,6,7,8-Tetrahydro-1,8-naphthyridin-2-yl)propyl]-1,2,4-oxadiazol-5-yl}-3-thien-3-ylbutanoic acid trifluoroacetate

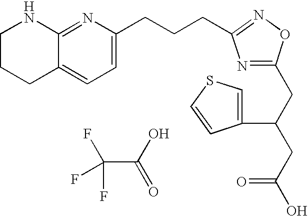

The title compound was prepared according to the method described for preparing the product of EXAMPLE 1 using 3-thiophenecarboxaldehyde: ¹H NMR (DMSO-d₆) δ 8.13 (broad s, 1H), 7.59 (d, 1H), 7.44-7.40 (m, 1H), 7.20 (m, 1H), 7.05 (m, 1H), 6.56 (d, 1H), 3.74-3.65 (m, 1H), 3.33-3.18 (m, 4H), 2.78-2.60 (m, 8H), 2.00-1.91 (m, 2H), 1.86-1.78 (m, 2H); MS (ESI+) for C₂₁H₂₄N₄O₃S m/z 413.10 (M+H)⁺.

### EXAMPLE 11

3-(2,3-Dihydro-1,4-benzodioxin-6-yl)-4-{3-[3-(5,6,7,8-tetrahydro-1,8-naphthyridin-2-yl)propyl]-1,2,4-oxadiazol-5-yl}butanoic acid hydrochloride

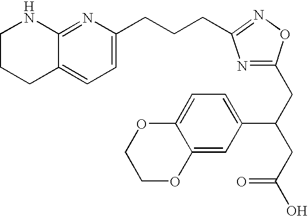

The title compound was prepared according to the method described for preparing the product of EXAMPLE 1 using 3,4-dihydrobenzodioxinaldehyde: ¹H NMR (CDCl₃) δ 7.25 (d, 1H), 6.78-6.63 (m, 3H), 6.31 (d, 1H), 4.27 (s, 4H), 3.65-3.58 (m, 1H), 3.42 (t, 2H), 3.15-3.02 (m, 2H), 2.80-2.60 (m, 8H), 2.21-2.12 (m, 2H), 1.90-1.83 (m, 2H); MS (ESI+) for C₂₅H₂₈N₄O₅ m/z 465.18 (M+H)⁺.

### EXAMPLE 12

4-{3-[3-(5,6,7,8-Tetrahydro-1,8-naphthyridin-2-yl)propyl]-1,2,4-oxadiazol-5-yl}-3-[3-(trifluoromethoxy)phenyl]butanoic acid hydrochloride

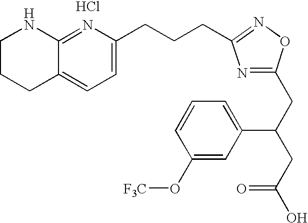

The title compound was prepared according to the method described for preparing the product of EXAMPLE 1 using 3-trifluoromethoxybenzaldehyde: ¹H NMR (CDCl₃) δ 7.34-7.24 (m, 2H), 7.20-7.00 (m, 3H), 6.33 (d, 1H), 3.81-3.71 (m, 1H), 3.46-3.39 (m, 2H), 3.21-3.10 (m, 2H), 2.89-2.60 (m, 8H), 2.21-2.10 (m, 2H), 1.92-1.83 (m, 2H); MS (ESI+) for C₂₄H₂₅F₃N₄O₄ m/z 491.15 (M+H)⁺.

### EXAMPLE 13

4-{3-[3-(5,6,7,8-Tetrahydro-1,8-naphthyridin-2-yl)propyl]-1,2,4-oxadiazol-5-yl}-3-(3,4,5-trifluorophenyl)butanoic acid hydrochloride

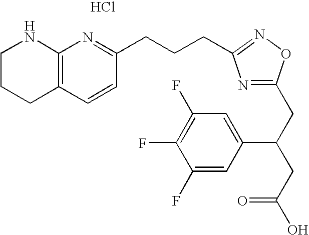

The title compound was prepared according to the method described for preparing the product of EXAMPLE 1 using 3,4,5-trifluorobenzaldehyde: ¹H NMR (CDCl₃) δ 8.38 (broad s, 1H), 7.36 (d, 1H), 6.93-6.88 (m, 2H), 6.40 (d, 1H), 3.80-3.71 (m, 1H), 3.53-3.46 (m, 2H), 3.20-3.15 (m, 2H), 2.95-2.71 (m, 8H), 2.29-2.20 (m, 2H), 2.00-1.90 (m, 2H); MS (ESI+) for C₂₃H₂₃F₃N₄O₃ m/z 461.15 (M+H)⁺.

### EXAMPLE 14

3-(2,2-Difluoro-1,3-benzodioxol-5-yl)-4-{3-[3-(5,6,7,8-tetrahydro-1,8-naphthyridin-2-yl)propyl]-1,2,4-oxadiazol-5-yl}butanoic acid hydrochloride

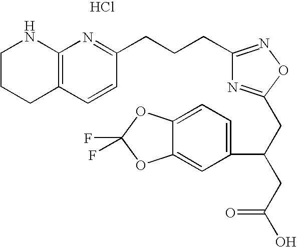

The title compound was prepared according to the method described for preparing the product of EXAMPLE 1 using 2,2-difluoro-1,3-benzodiol-5-aldehyde: ¹H NMR (CDCl₃) δ 8.49 (broad s, 1H), 7.40 (d, 1H), 7.08-7.00 (m, 3H), 6.45 (d, 1H), 3.90-3.81 (m, 1H), 3.58-3.51 (m, 2H), 3.28-3.21 (m, 2H), 3.00-2.78 (m, 8H), 2.33-2.25 (m, 2H), 2.04-1.96 (m, 2H); MS (ESI+) for C₂₄H₂₄F₂N₄O₅ m/z 486.90 (M+H)⁺.

### EXAMPLE 15

3-[3-Fluoro-5-(trifluoromethyl)phenyl]-4-{3-[3-(5,6,7,8-tetrahydro-1,8-naphthyridin-2-yl)propyl]-1,2,4-oxadiazol-5-yl}butanoic acid hydrochloride

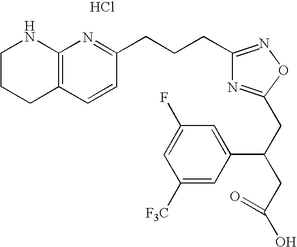

The title compound was prepared according to the method described for preparing the product of EXAMPLE 1 using 3-fluoro-5-trifluoromethyl-benzaldehyde: ¹H NMR (CDCl₃) δ 8.62 (broad s, 1H), 7.58 (d, 1H), 7.55 (s, 1H), 7.45-7.39 (m, 2H), 6.63 (d, 1H), 4.15-4.06 (m, 1H), 3.75-3.69 (m, 2H), 3.48-3.41 (m, 2H), 3.23-3.14 (m, 1H), 3.03-2.95 (m, 8H), 2.50-2.41 (m, 2H), 2.20-2.13 (m, 2H); MS (ESI+) for C₂₄H₂₄F₄N₄O₃ m/z 493.13 (M+H)⁺.

### EXAMPLE 16

3-(6-Methoxy-2-naphthyl)-4-{3-[3-(5,6,7,8-tetrahydro-1,8-naphthyridin-2-yl)propyl]-1,2,4-oxadiazol-5-yl}butanoic acid hydrochloride

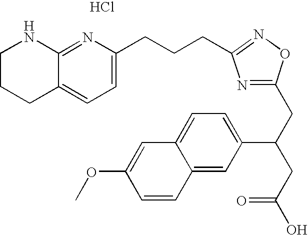

The title compound was prepared according to the method described for preparing the product of EXAMPLE 1 using 6-methoxy-2-naphthaldehyde: ¹H NMR (CDCl₃) δ 7.65-7.59 (m, 3H), 7.61 ((7.29-7.21 (M, 2H), 7.09-7.01 (M, 2H), 6.04 (d, 1H), 3.84 (s, 3H), 3.45-3.39 (m, 2H), 3.25-3.20 (d, 2H), 2.95-2.86 (m, 1H), 2.83-2.64 (m, 8H), 2.20-2.10 (m, 2H), 1.90-1.83 (m, 2H); MS (ESI+) for C₂₈H₃₀N₄O₄ m/z 487.30 (M+H)⁺.

### EXAMPLE 17

3-(6-Methoxypyridin-3-yl)-4-{3-[3-(5,6,7,8-tetrahydro-1,8-naphthyridin-2-yl)propyl]-1,2,4-oxadiazol-5-yl}butanoic acid 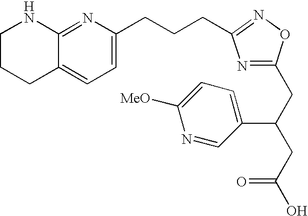 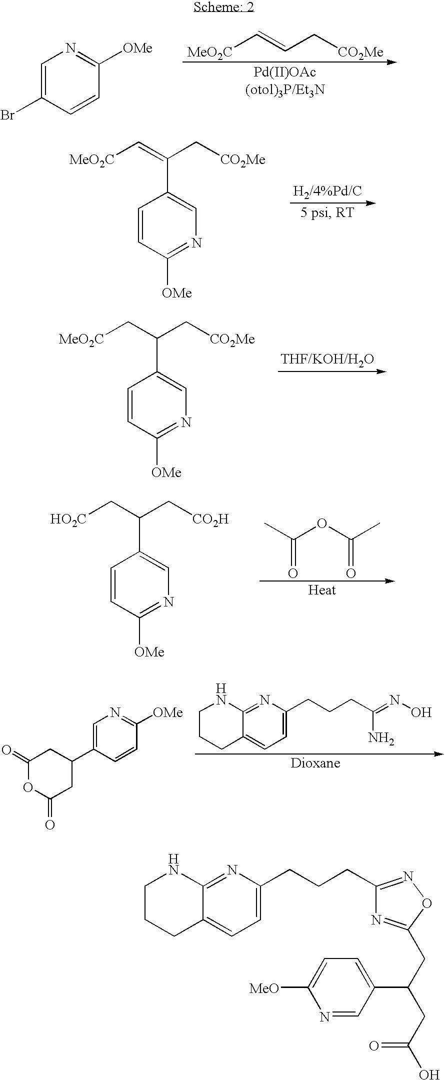
Step 1. Synthesis of dimethyl 3-(6-methoxypyridin-3-yl)pent-2-enedicarboxylate.

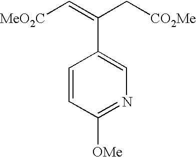

A mixture of dimethyl pent-2-enedicarboxylate (2.86 g, 18.09 mmol), Palladium (II) acetate (0.12 g, 0.53 mmol), tri-o-tolyphosphine (0.405 g, 1.33 mmol), and triethylamine (2.0 mL) in DMF (2.13 mL) was degassed and heated at 90 C. The 5-Bromo-2-methoxy pyridine (1) was added dropwise to the mixture and heated at 90 C overnight. The reaction mixture was cooled to rt and the solid was filtered. The filtrate was diluted with water and this mixture was extracted with ethyl acetate (3×100 mL). The organic layers were combined, washed with brine, dried over Na₂SO₄ and concentrated under reduced pressure. The resulting residue was purified by flash column chromatography using 5-25% EtOAc/Hexane to give dimethyl 3-(6-methoxypyridin-3-yl)pent-2-enedicarboxylate as light yellow oil (0.301 g, 21%). ¹H NMR (CD₃OD) δ 8.31 (d, 1H), 7.88-7.84 (m, 1H), 6.84 (d, 1H), 6.33 (s, 1H), 4.86 (s, 2H), 3.95 (s, 3H), 3.75 (s, 3H), 3.68 (s, 3H);
Step 2. Synthesis of dimethyl 3-(6-methoxypyridin-3-yl)pentanedicarboxylate.

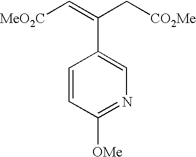

A standard par bottle was charged with dimethyl 3-(6-methoxypyridin-3-yl)pent-2-enedicarboxylate (0.301 g, 1.13 mmol) in MeOH and 4% Palladium on carbon. The hydrogenation was carried out at 5 psi at rt for two hours. MS (ESI+) for C₁₃H₁₇NO₅ m/z 268.40 (M+H)⁺.
Step 3. Synthesis of 3-(6-methoxypyridin-3-yl)pentanedioic acid.

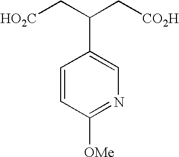

To dimethyl 3-(6-methoxypyridin-3-yl)pentanedicarboxylate (0.276 g, 1.034 mmol) in THF (17.20 mL) was added water (17.20 mL) and KOH (0.58 g). The reaction mixture was stirred at rt for overnight. Concentrated HCl was then added until the pH=2.0. During the addition, the temperature was kept below 50 C. The mixture was extracted with ethyl acetate (3×50 mL). The organic layers were combined, washed with brine, dried over Na₂SO₄ and concentrated to produce off white solid 3-(6-methoxypyridin-3-yl)pentanedioic acid (0.145 g, 59%). ¹H NMR (CD₃OD) δ 8.05 (d, 1H), 7.69-7.65 (m, 1H), 6.78 (d, 1H), 3.89 (s, 3H), 3.60-3.51 (m, 1H), 2.80-2.73 (m, 2H), 2.65-2.58 (m, 2H); MS (ESI+) for C₁₁H₁₃NO₅ m/z 240.30 (M+H)⁺.
Step 4. Synthesis of 4-(6-methoxypyridin-3-yl)dihydropyran-2,6(3H)-dione:

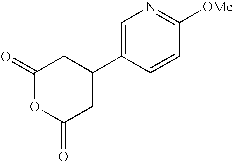

To 3-(6-methoxypyridin-3-yl)pentanedioic acid (0.276 g, 1.15 mmol) was added acetic anhydride (10.0 mL). The reaction mixture was stirred and heated at 100 C for 5 hours. The reaction mixture was cooled to rt. The solvent was removed under reduced pressure to give dark brown solid of 4-(6-methoxypyridin-3-yl)dihydropyran-2,6(3H)-dione (0.086 g, 34%). LCMS was done by diluting the sample with acetonitrile and adding 50 uL of Piperidine, LCMS indicated mass product 307.40 m/z (M+Piperidine).
Step 5. Synthesis of 3-(6-Methoxypyridin-3-yl)-4-{3-[3-(5,6,7,8-tetrahydro-1,8-naphthyridin-2-yl)propyl]-1,2,4-oxadiazol-5-yl}butanoic acid:

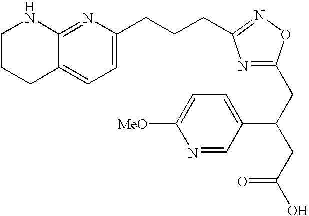

This compound was prepared as in EXAMPLE 1, step 4 starting from 4-(6-methoxypyridin-3-yl)dihydropyran-2,6(3H)-dione. Yield (0.038 g, 22%). ¹H NMR (CD₃OD) δ 8.09 (d, 1H), 7.04-7.69 (m, 1H), 7.45 (d, 1H), 6.80 (d, 1H), 6.55 (d, 1H), 3.89 (s, 3H), 3.79-3.70 (m, 1H), 3.46-3.41 (m, 2H), 3.21-3.18 (m, 2H), 2.71-2.55 (m, 8H), 2.19-2.05 (m, 2H), 1.98-1.89 (m, 2H). MS (ESI+) for C₂₃H₂₇N₅O₄ m/z 438.20 (M+H)⁺.

### EXAMPLE 18

3-(4-Cyanophenyl)-4-{3-[3-(5,6,7,8-tetrahydro-1,8-naphthyridin-2-yl)propyl]-1,2,4-oxadiazol-5-yl}butanoic acid 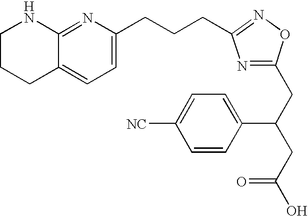
Step 1. Diethyl 3-(4-cyanophenyl)pentanedioate.

A sample of the diethyl 3-(4-bromophenyl)pentanedioate, prepared as in step 1, EXAMPLE 1, (1.0 g, 2.91 mmol) in degassed DMF was added water, zinc cyanide (0.212 g, 1.806 mmol), tris(dibenzylideacetone)dipalladium (0) (0.133 g, 0.145 mmol), and Bis(diphenylphosphino)ferrocene (0.333 g, 0.601 mmol). The reaction mixture was heated at 120 C for overnight. The reaction mixture was cooled to rt and filtered. The filtrate was diluted with water and extracted with ethyl acetate (3×50 mL). The organic layers were combined and washed with brine, dried over Na₂SO₄, and concentrated under reduced pressure. The residue was purified by flash master chromatography using 0-10% ethyl acetate/Hexane to obtain pale green oil (0.280 g, 33%). ¹H NMR (CD₃OD) δ 7.69 (d, 2H), 7.50 (d, 2H), 4.064.00 (m, 4H), 3.72-3.63 (m, 1H), 2.85-2.78 (m, 2H), 2.75-2.65 (m, 2H), 1.14 (t, 6H);

Step 2. 3-(4-cyanophenyl)-4-{3-[3-(5,6,7,8-tetrahydro-1,8-naphthyridin-2-yl)propyl]-1,2,4-oxadiazol-5-yl}butanoic acid.

This was prepared according to the method described for preparing the product in EXAMPLE 1, starting from diethyl 3-(4-cyanophenyl)pentanedioate: ¹H NMR (DMSO-d₆) δ 7.72 (d, 2H), 7.50 (d, 2H), 7.02 (d, 1H), 6.39 (broad s, 1H), 6.20 (d, 1H), 3.69-3.60 (m, 1H), 3.40-3.28 (m, 2H), 3.26-3.20 (m, 2H), 2.85-2.78 (m, 1H), 2.71-2.62 (m, 1H), 2.62-2.51 (m, 4H), 2.42-2.33 (m, 2H), 1.91-1.83 (m, 2H), 1.79-1.70 (m, 2H); MS (ESI+) for C₂₄H₂₅N₅O₃ m/z 432.60 (M+H)⁺.

### EXAMPLE 19

3-(3-Cyanophenyl)-4-{3-[3-(5,6,7,8-tetrahydro-1,8-naphthyridin-2-yl)propyl]-1,2,4-oxadiazol-5-yl}butanoic acid.

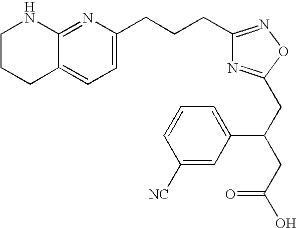

The title compound was prepared according to the method as described for preparing Example 18, by starting from the corresponding bromo compound. ¹H NMR (DMSO-d₆) δ 7.82 (s, 1H), 7.68-7.60 (m, 2H), 7.50-7.45 (m, 1H), 7.06 (d, 1H), 6.42 (broad s, 1H), 6.22 (d, 1H), 3.68-3.58 (m, 1H), 3.40-3.28 (m, 2H), 3.28-3.20 (m, 2H), 2.87-2.79 (m, 1H), 2.75-2.65 (m, 1H), 2.65-2.53 (m, 4H), 2.40 (t, 2H), 1.92-1.83 (m, 2H), 1.79-1.71 (m, 2H); MS (ESI+) for C₂₄H₂₅N₅O₃ m/z 432.20 (M+H)⁺.

### EXAMPLE 20

Preparation of 3-benzyl-4-{3-[3-(5,6,7,8-tetrahydro-1,8-naphthyridin-2-yl)propyl]-1,2,4-oxadiazol-5-yl}butanoic acid trifluoroacetate. 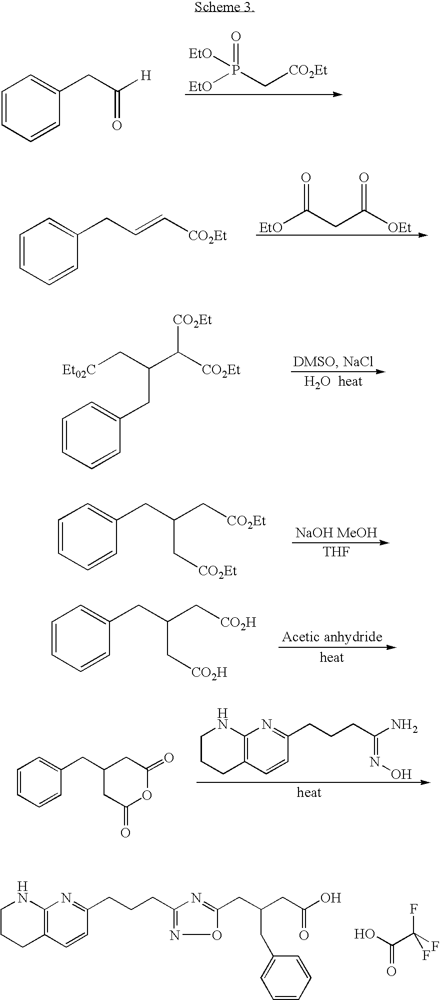
Step 1. 4-benzyldihydro-2H-pyran-2,6(3H)-dione:

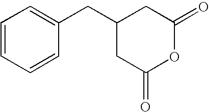

Ethyl 4-phenylbut-2-enoate prepared according to Legters, J.; Thijs, L.; Zwanenburg, B.; Recl. Trav. Chim. Pays-Bas 111; 1; 1992; 1-15 was used as the starting material to synthesize 4-benzyldihydro-2H-pyran-2,6(3H)-dione according to procedures outlined in Tokoroyama, Takashi; Kusaka, Hisashi; Can. J. Chem.; 74; 12; 1996; 2487-2502., and Victory, Pedro; Alvarez-Larena, Angel; Barbera, Eduardo; Batlori. Xavier; Borrell, Jose I.; Cordoba, Carlos; J. Chem. Res. Miniprint; 4; 1989; 0631-0674.
Step 2. 3-benzyl-4-{3-[3-(5,6,7,8-tetrahydro-1,8-naphthyridin-2-yl)propyl]-1,2,4-oxadiazol-5-yl}butanoic acid trifluoroacetate

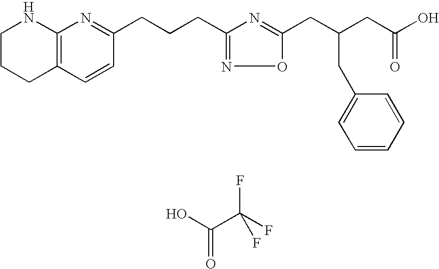

3-Benzyl-4-{3-[3-(5,6,7,8-tetrahydro-1,8-naphthyridin-2-yl)propyl]-1,2,4-oxadiazol-5-yl}butanoic acid trifluoroacetate was prepared according to the method described for preparing 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate (EXAMPLE 1, Step 4) using 4-benzyldihydro-2H-pyran-2,6-(3H)-dione as the anhydride (prepared according to the general procedure outlined for 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate (EXAMPLE 1, Steps 1-3)): ¹H NMR (400 MHz) DMSO-d₆ δ 8.04 (br s, 1H), 7.66 (d, 1H), 7.22 (m, 5H), 6.61 (d, 1H), 3.40 (m, 3H), 2.88 (m, 2H), 2.72-2.55 (m, 8H), 2.27 (d, 2H), 1.99 (m, 2H), 1.81 (m, 2H) Mass Spectrum: (MH⁺)=421.3.

### EXAMPLE 21

3-(4-fluoro-3-methoxyphenyl)-4-{3-[3-(5,6,7,8-tetrahydro-1,8-naphthyridin-2-yl)propyl]-1,2,4-oxadiazol-5-yl}butanoic acid trifluoroacetate.

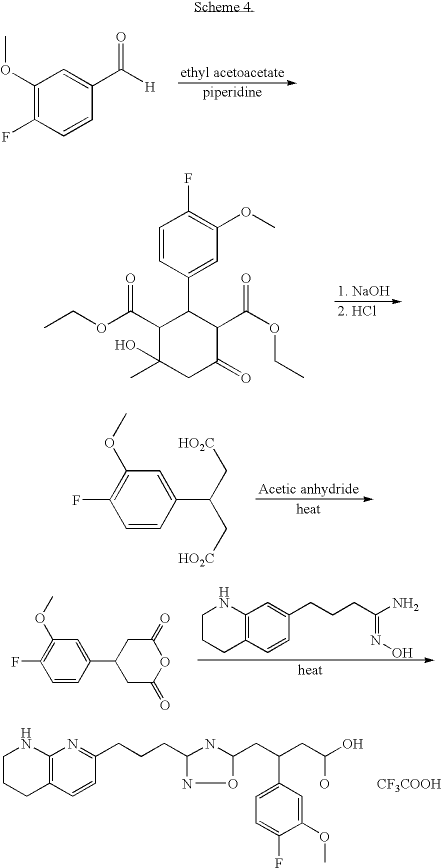

3-(4-fluoro-3-methoxyphenyl)-4-{3-[3-(5,6,7,8-tetrahydro-1,8-naphthyridin-2-yl)propyl]-1,2,4-oxadiazol-5-yl}butanoic acid trifluoroacetate was prepared according to the method as described for preparing 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate (Example 1, Step 4) using the appropriate anhydride (prepared according to the general procedure outlined for 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate (Example 1, Steps 1-3): ¹H NMR(400 MHZ) DMSO-d₆ δ 7.81 (br s, 1H), 7.59 (d, 1H), 7.08-7.02 (m, 2H), 6.82-6.75 (m, 1H), 6.55 (d, 1H), 3.78 (s, 3H), 3.6-3.2 (m, 5H), 2.8-2.6 (m, 8H), 1.99-1.91 (m, 2H), 1.89-1.79 (m, 2H) Mass Spectrum: (MH⁺)=455.2.

### EXAMPLE 22

3-(3-Fluoro-5-methoxyphenyl)-4-{3-[3-(5,6,7,8-tetrahydro-1,8-naphthyridin-2-yl)propyl]-1,2,4-oxadiazol-5-yl}butanoic acid hydrochloride. 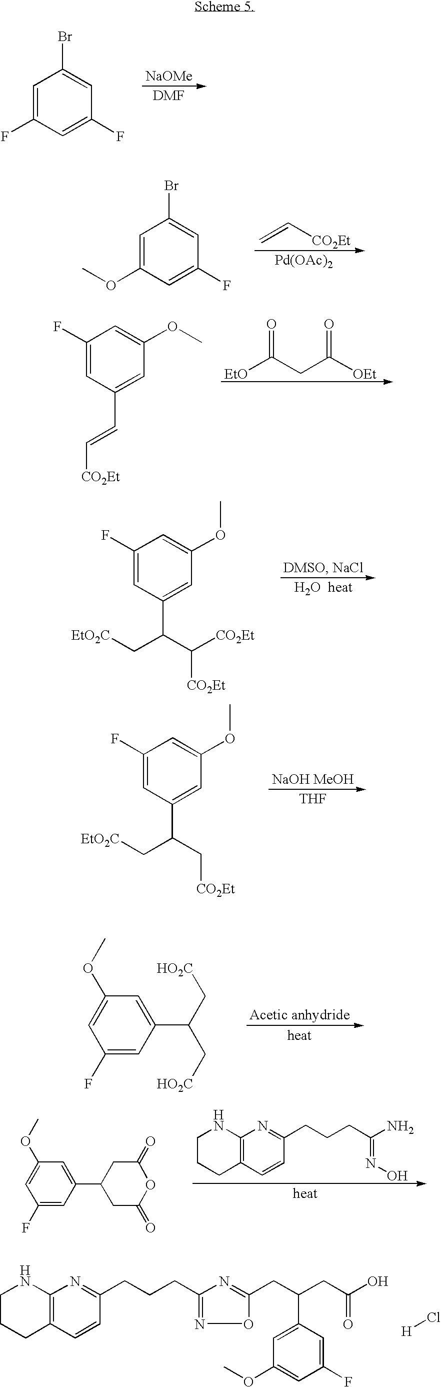
Step 1. 1-bromo-3-fluoro-5-methoxybenzene.

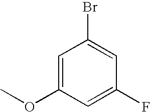

1-Bromo-3,5-difluorobenzene (20 g, 103.63 mmol) was dissolved in N,N-dimethylformamide (50 mL) under nitrogen and the solution was cooled to 0.5° C. Sodium methoxide was then added gradually to ensure that the temperature did not go above 5° C. The mixture was allowed to stir at room temperature for 60 hours. The reaction mixture was diluted using methylene chloride (50 mL). The solution was washed with water (2×50 mL), and then dried over magnesium sulfate and filtered. The solvent was removed under vacuum to give the product 1-bromo-3-fluoro-5-methoxybenzene (6.34 g; 30%). ¹H NMR (400 MHz) CDCl₃ δ 7.09-7.06 (m, 1H), 6.86-6.76 (m, 1H), 6.58-6.54 (m, 1H), 3.81 (trip, 3H).
Step 2. Ethyl(2E)-3-(3-fluoro-5-methoxyphenyl)prop-2-enoate.

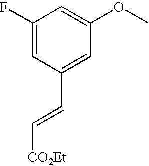

1-Bromo-3-fluoro-5-methoxybenzene (2 g, 9.75 mmol), ethyl acrylate (1.32 mL, 12.19 mmol), palladium acetate (0.109 g, 0.49 mmol), sodium acetate (4 g, 48.75 mmol), and N,N-dimethylformamide (10 mL) were added to a small vial and allowed to stir for 16 hours at 100° C. A silica column was then run using 50 g silica and a 9:1 hexane to ethyl acetate solution as the eluent. The product was still impure so another silica column was run using 50 g silica and a 9:1 methylene chloride to ethyl acetate as the eluent. The clean fractions were collected and condensed to give the product ethyl (2E)-3-(3-fluoro-5-methoxyphenyl)prop-2-enoate (2 g or 91%). ¹H NMR (400 MHz) CDCl₃ δ 7.59-7.51 (d, 1H), 6.85-6.82 (m, 2H), 6.66-6.61 (m, 1H), 6.42-6.38 (d, 1H), 4.30-4.23 (quar, 2H), 3.82 (s, 3H), 1.37-1.32 (trip, 3H).
Step 3. 4-(3-Fluoro-5-methoxyphenyl)dihydro-2H-pyran-2,6(3H)-dione.

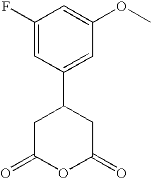

4-(3-Fluoro-5-methoxyphenyl)dihydro-2H-pyran-2,6(3H)-dione was synthesized from the product of Step 2 according to procedures outlined in Tokoroyama, Takashi; Kusaka, Hisashi; Can. J. Chem.; 74; 12; 1996; 2487-2502 and Victory, Pedro; Alvarez-Larena, Angel; Barbera, Eduardo; Batilori. Xavier; Borrell, Jose I.; Cordoba, Carlos; J. Chem. Res. Miniprint; 4; 1989; 0631-0674.
Step 4. 3-(3-Fluoro-5-methoxyphenyl)-4-{3-[3-(5,6,7,8-tetrahydro-1,8-naphthyridin-2-yl)propyl]-1,2,4-oxadiazol-5-yl}butanoic acid hydrochloride.

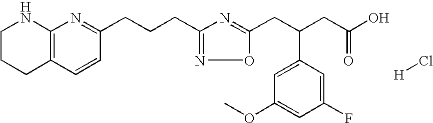

3-(3-Fluoro-5-methoxyphenyl)-4-{3-[3-(5,6,7,8-tetrahydro-1,8-naphthyridin-2-yl)propyl]-1,2,4-oxadiazol-5-yl}butanoic acid hydrochloride was prepared according to the method as described for preparing 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate (Example 1, Step 4). ¹H NMR (DMSO-d₆) [symbol] 7.75 (br s, 1H), 7.58 (d, 1H), 6.72-6.65 (m, 2H), 6.64-6.62 (m, 1H), 6.54 (d, 1H), 3.71 (s, 3H), 3.6-3.2 (m, 5H), 2.8-2.6 (m, 8H), 1.99-1.91 (m, 2H), 1.89-1.79 (m, 2H) Mass Spectrum: (MH⁺)=455.2.

### EXAMPLE 23

3-(2-Methyl-1,3-benzothiazol-5-yl)-4-{3-[3-(5,6,7,8-tetrahydro-1,8-naphthyridin-2-yl)propyl]-1,2,4-oxadiazol-5-yl}butanoic acid hydrochloride.

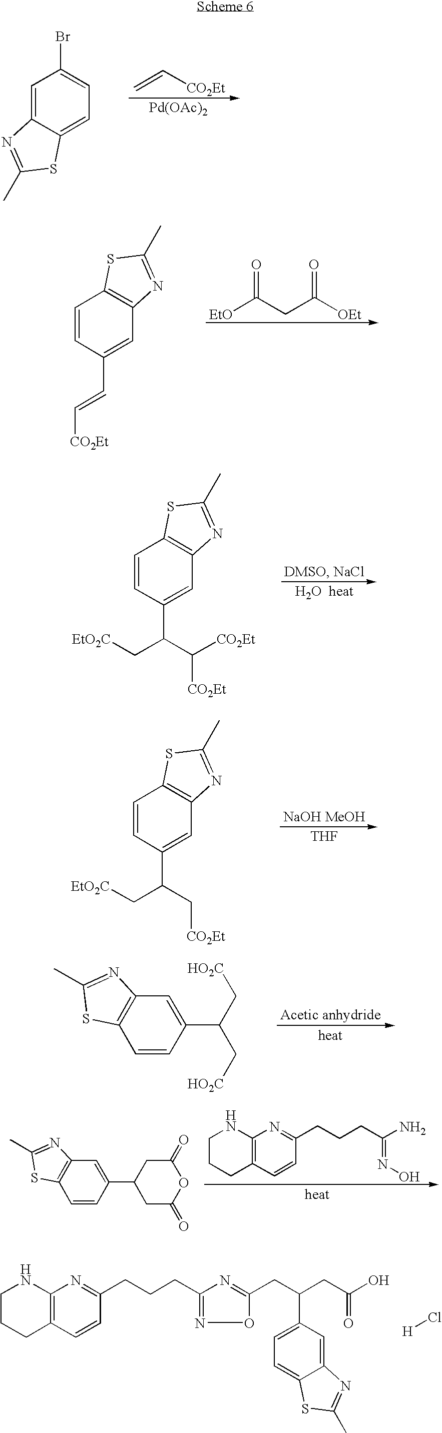

3-(2-Methyl-1,3-benzothiazol-5-yl)-4-{3-[3-(5,6,7,8-tetrahydro-1,8-naphthyridin-2-yl)propyl]-1,2,4-oxadiazol-5-yl}butanoic acid hydrochloride was made according to the method described for preparing 3-(3-fluoro-5-methoxyphenyl)-4-{3-[3-(5,6,7,8-tetrahydro-1,8-naphthyridin-2-yl)propyl]-1,2,4-oxadiazol-5-yl}butanoic acid hydrochloride; using commercially available 5-bromo-2-methylbenzothiazole: ¹H NMR (400 MHZ) DMSO-d₆ δ 7.89-7.87 (d, 1H), 7.78-7.73 (m, 2H), 7.60-7.58 (d, 1H), 7.31-7.29 (m, 1H), 6.52-6.50 (d, 1H), 3.73-3.66 (m, 1H), 3.41-3.30 (m, 4H), 2.89-2.57 (m, 11H), 1.93-1.79 (m, 4H) Mass Spectrum: (MH⁺)=520.

### EXAMPLE 24

3-[2-(4-Chlorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride. 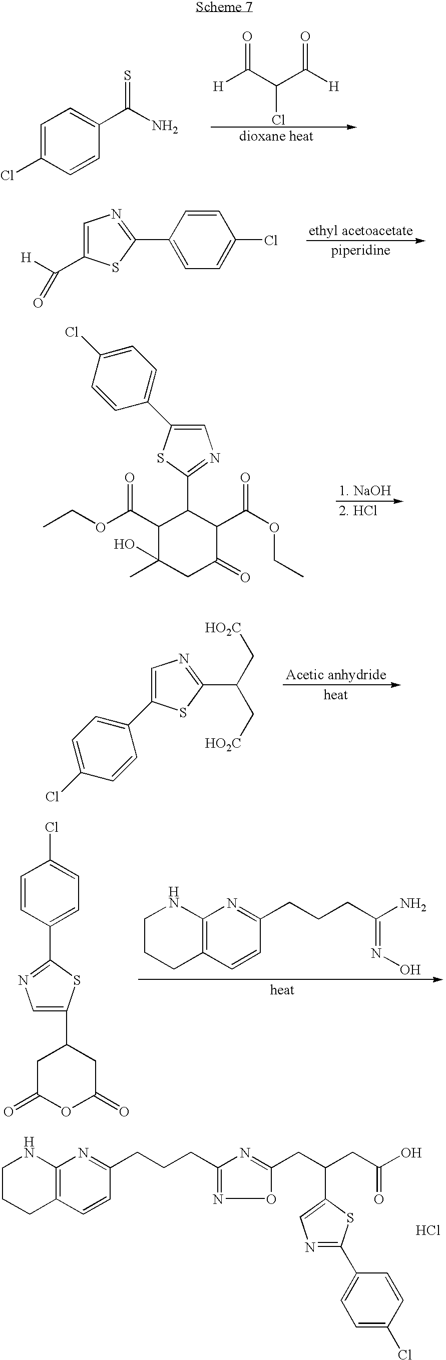
Step 1. 2-(4-Chlorophenyl)-1,3-thiazole-5-carbaldehyde.

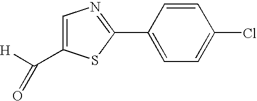

4-Chlorobenzene-1-carbothioamide (5 g, 29.1 mmol), magnesium carbonate hydroxide pentahydrate (7.06 g, 14.55 mmol), and 2-chloromalonaldehyde (4.65 g, 43.65 mmol) (Cornforth, Fawaz, Goldsworthy and Robinson; J. Chem. Soc. 1949, 1550) were added to a flask and allowed to stir under nitrogen at 60° C. for three hours. The reaction mixture was then passed through a plug of silica and washed with ethyl acetate. The solvent was removed under vacuum to give the product 2-(4-chlorophenyl)-1,3-thiazole-5-carbaldehyde (6 g, 92%) ¹H NMR (400 MHz) CDCl₃ δ 10.06 (s, 1H), 8.43 (s, 1H), 7.99-7.96 (m, 2H), 7.49-7.46 (2H).
Step 2. 4-[2-(4-Chlorophenyl)-1,3-thiazol-5-yl]dihydro-2H-pyran-2,6(3H)-dione 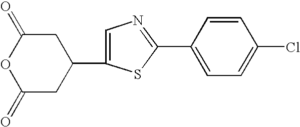
4-[2-(4-Chlorophenyl)-1,3-thiazol-5-yl]dihydro-2H-pyran-2,6(3H)-dione was prepared according to the general procedure outlined for 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate (Example 1, Steps 1-3)
Step 3. 3-[2-(4-Chlorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride

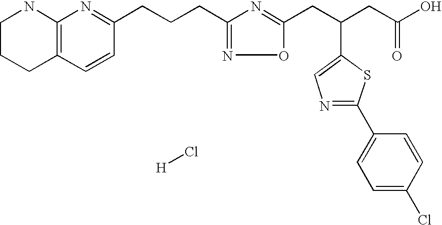

3-[2-(4-Chlorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride was prepared according to the method as described for preparing 3-(1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate (Example 1, Step 4). ¹H NMR (400 mHz) DMSO-d₆ [symbol] 7.85-7.82 (m, 2H), 7.78 (br s, 1H), 7.70 (s, 1H), 7.54-7.51 (m, 3H), 6.50 (d, 1H), 3.44-3.40 (m, 5H), 2.9-2.6 (m, 8H), 1.96-1.91 (m, 2H), 1.81 (m, 2H), Mass Spectrum: (MH⁺)=525.

### EXAMPLE 25

3-[2-(4-Methoxyphenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride.

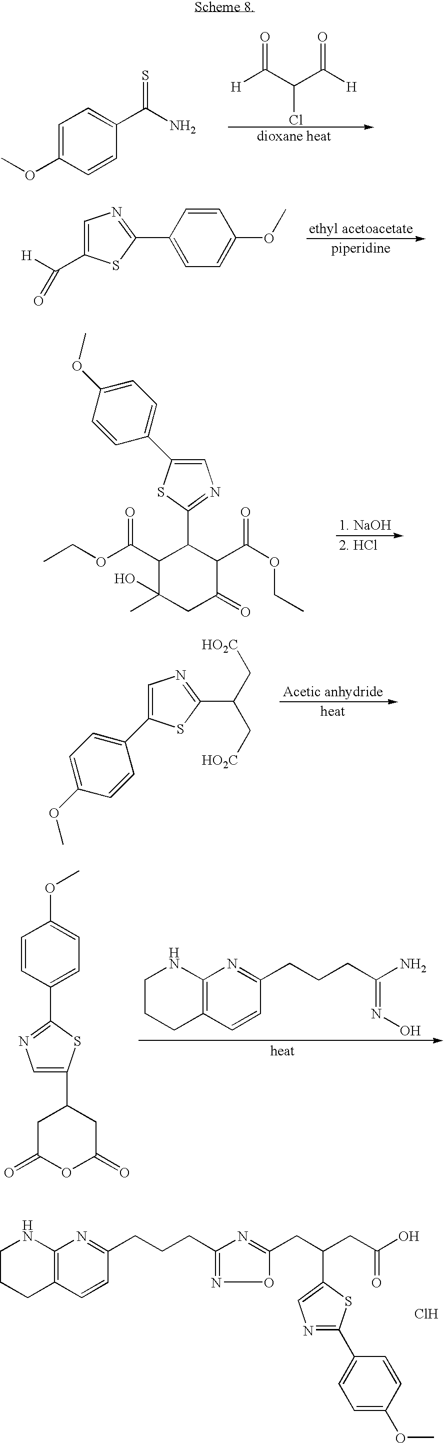

3-[2-(4-Methoxyphenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride was made according to the methods described for preparing 3-[2-(4-chlorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride starting from the appropriate thioamide: ¹H NMR (400 MHz) DMSO-d₆ δ 13.78 (br s, 1H), 7.88 (br s, 1H), 7.76-7.75 (m, 2H), 7.60 (s, 1H), 7.53 (d, 1H), 7.02-6.99 (m, 2H), 6.53-6.51 (d, 1H), 3.99-3.86 9m, 1H), 3.83 (s, 3H), 3.46-3.29 (m, 4H), 2.93-2.62 (m, 8H), 2.01-1.91 (m, 2H), 1.84-1.76 (m, 2H). Mass Spectrum: (MH⁺)=520.

### EXAMPLE 26

3-(2-Methyl-1,3-benzothiazol-5-yl)-4-{3-[3-(5,6,7,8-tetrahydro-1,8-naphthyridin-2-yl)propyl]-1,2,4-oxadiazol-5-yl}butanoic acid hydrochloride.

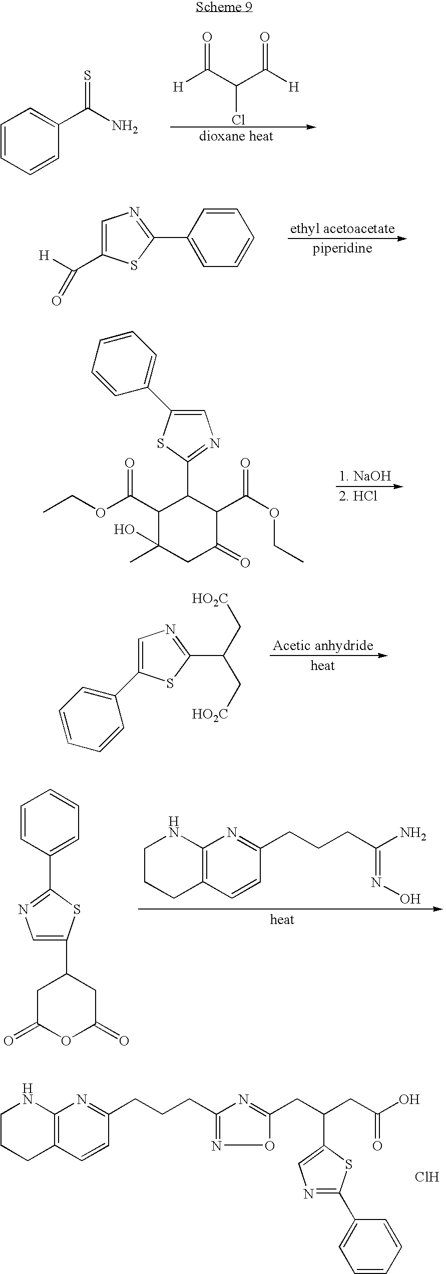

3-(2-Methyl-1,3-benzothiazol-5-yl)-4-{3-[3-(5,6,7,8-tetrahydro-1,8-naphthyridin-2-yl)propyl]-1,2,4-oxadiazol-5-yl}butanoic acid hydrochloride was made according to the methods described for preparing 3-[2-(4-chlorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride starting from the appropriate thioamide: ¹H NMR (400 MHz) DMSO-d₆ δ 7.77-7.75 (m, 3H), 7.63-7.61 (m, 2H), 7.42-7.39 (m, 3H), 6.42-6.37 (d, 1H), 3.95-3.88 (m, 1H), 3.34-3.28 (m, 4H), 2.89-2.53 (m, 8H), 1.94-1.87 (m, 2H), 1.76-1.70 (m, 2H) Mass Spectrum: (MH⁺)=490.6.

### EXAMPLE 27

3-[2-(4-Fluorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride. 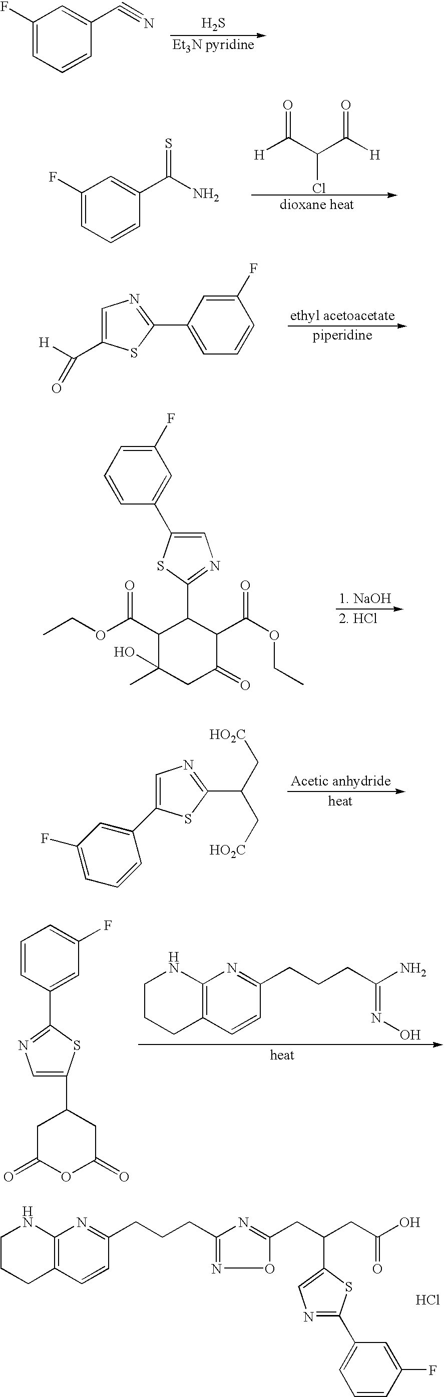
Step 1. 3-Fluorobenzenecarbothioamide.

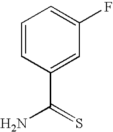

3-Fluorobenzonitrile (10 g, 82.6 mmol), triethylamine (6 mL), and pyridine (60 mL) were added to a flask and then hydrogen sulfide gas was bubbled through the reaction mixture for 5 minutes. The reaction flask was then capped and allowed to sit for 48 hours. The solvent was then dried off with nitrogen and placed under vacuum to give product (12.00 g, 94%). ¹H NMR (400 MHz) DMSO-d₆ [symbol] 10.05 (s, 1H), 9.60 (s, 1H), 7.72-7.71 (m, 2H), 7.48-7.32 (m, 2H).
Steps 2-6. 3-[2-(4-fluorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride.

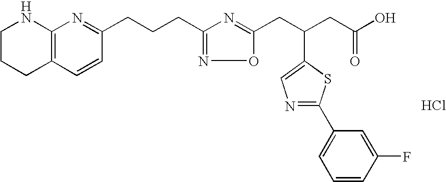

3-Fluorobenzenecarbothioamide was converted to 3-[2-(4-fluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride according to the method described for preparing 3-[2-(4-chlorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride: ¹H NMR (400 MHz) DMSO-d₆ [symbol] 7.87 (br s, 1H), 7.7 (s, 1H), 7.65-7.47 (m, 3H), 7.33-7.28 (m, 2H), 6.53-6.51 (d, 1H), 4.05-3.95 (m, 1H), 3.56-3.34 (m, 4H), 2.96-2.63 (m, 8H), 2.00-1.93 (m, 2H), 1.83-1.80 (m, 2H). Mass Spectrum: (MH⁺)=508.1.

### EXAMPLE 28

3-[2-(3,5-Difluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride.

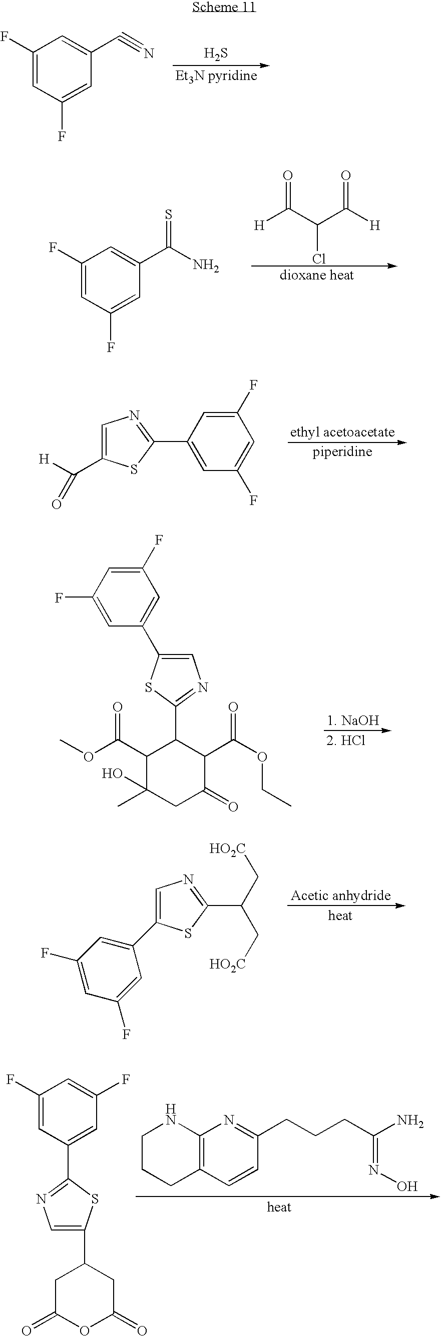

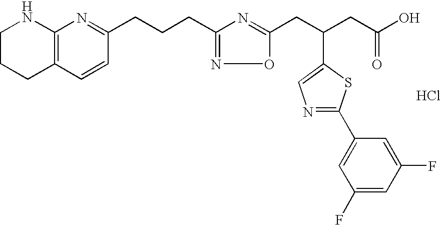

3-[2-(3,5-Difluorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride was made according to the method as described for preparing 3-[2-(4-fluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride starting from the appropriate benzonitrile: ¹H NMR (400 MHz) DMSO-d₆ [symbol] 7.95 (br s, 1H), 7.77 (s, 1H), 7.54-7.51 (m, 3H), 7.40-7.32 (m, 1H), 6.53-6.51 (d, 1H), 4.04-3.96 (m, 1H), 3.50-3.34 (m, 4H), 2.96-2.63 (m, 8H), 2.01-1.94 (m, 2H), 1.84-1.78 (m, 2H). Mass Spectrum: (MH⁺)=526.2.

### EXAMPLE 29

3-[2-(3,4-Difluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride.

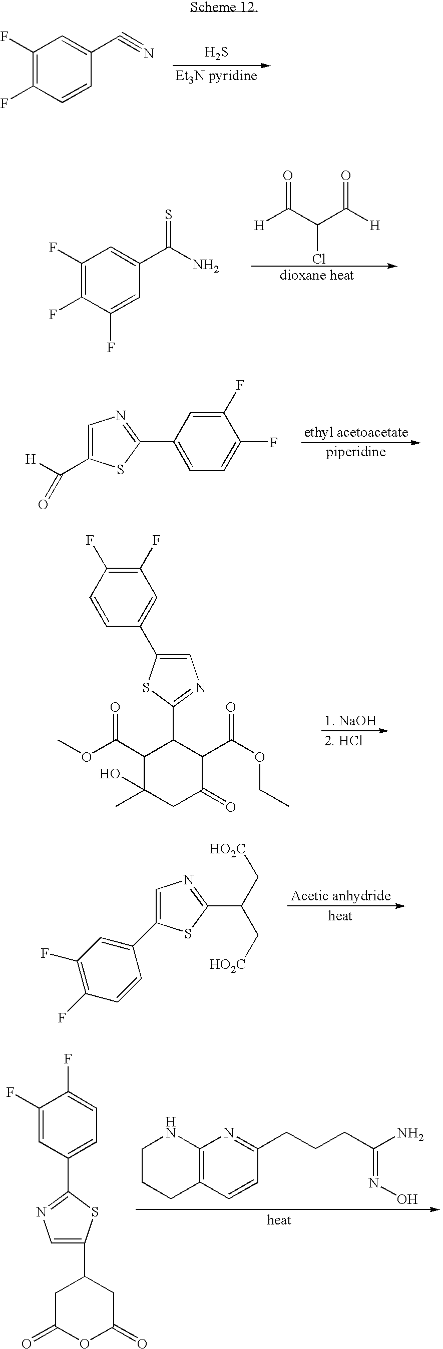

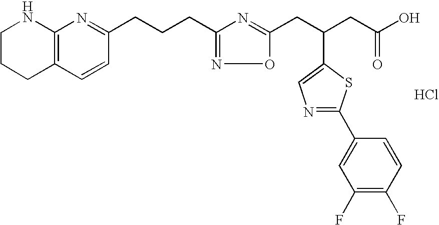

3-[2-(3,4-Difluorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride was made according to the method described for preparing 3-[2-(4-fluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride starting from the appropriate benzonitrile: ¹H NMR (400 MHz) DMSO-d₆ [symbol] 7.88-7.83 (m, 2H), 7.71 (s, 1H), 7.69-7.66 (m, 1H), 7.59-7.50 (m, 2H), 6.53-6.52 (d, 1H), 3.99-3.95 (m, 1H), 3.49-3.30 (m, 4H), 2.95-2.63 (m, 8H), 2.01-1.94 (m, 2H), 1.84-1.78 (m, 2H). Mass Spectrum: (MH⁺)=526.2.

### EXAMPLE 30

3-[2-(2-Furyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride.

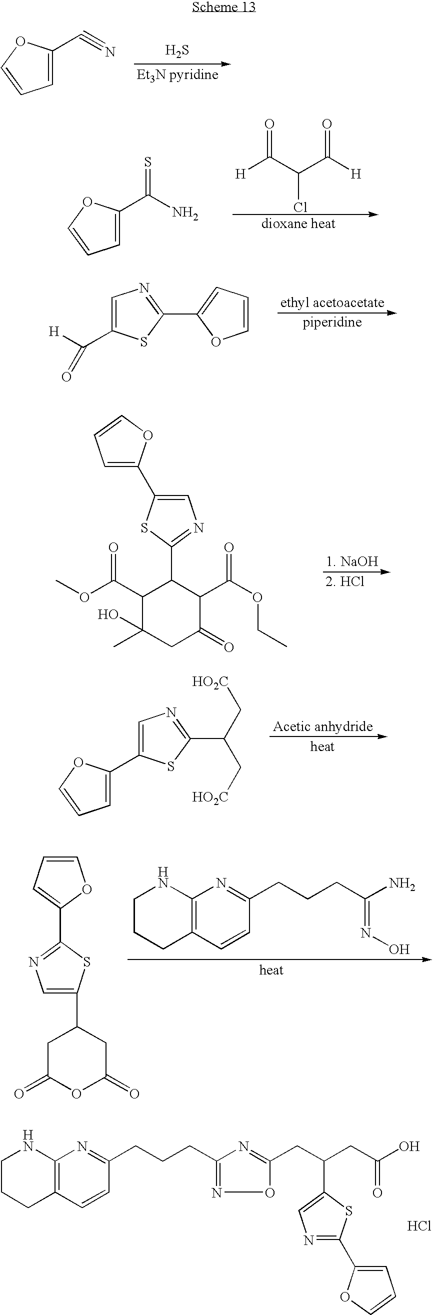

3-[2-(2-Furyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride was made according to the method described for preparing 3-[2-(4-fluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride starting from the appropriate nitrile: ¹H NMR (400 MHz) DMSO-d₆ [symbol] 7.87 (br s, 1H), 7.83 (m, 1H), 7.63 (s, 1H), 7.56 (d, 1H), 6.97 (m, 1H), 6.67-6.66 (m, 1H), 6.54 (d, 1H), 3.98-3.93 (m, 1H), 3.47-3.32 (m, 4H), 2.94-2.66 (m, 8H), 2.01-1.94 (m, 2H), 1.84-1.78 (m, 2H). Mass Spectrum: (MH⁺)=480.1.

### EXAMPLE 31

3-(3,4-Dimethoxyphenyl)-4-{3-[3-(5,6,7,8-tetrahydro-1,8-naphthyridin-2-yl)propyl]1,2,4-oxadiazol-5-yl}butanoic acid trifluoroacetate.

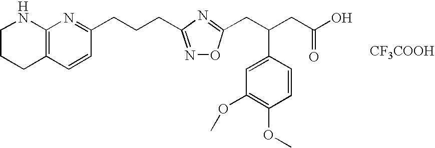

This and the following Examples (32-37) were synthesized as in Example 1, starting from the corresponding substituted aryl- or heteroaryl-aldehydes. ¹H NMR (DMSO-d₆) δ 7.74 (br s, 1H), 7.6 (d, J=7.5 Hz, 1H), 6.85-6.70 (m, 3H), 6.55 (d, J=7.5 Hz, 1H), 3.69 (s, 3H), 3.66 (s, 3H), 3.54-3.39 (m, 5H), 3.31-3.16 (m, 2H), 2.73-2.60 (m, 6H), 1.99-1.89 (m, 2H), 1.85-1.76 (m, 2H). Mass Spectrum: (MH⁺)=467.

### EXAMPLE 32

3-(3,5-Dimethoxyphenyl)-4-{3-[3-(5,6,7,8-tetrahydro-1,8-naphthyridin-2-yl)propyl] 1,2,4-oxadiazol-5-yl}butanoic acid trifluoroacetate.

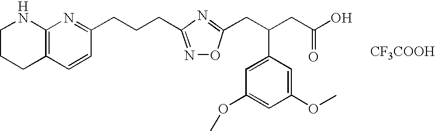

¹H NMR (DMSO-d₆) δ 12.20 (br s, 1H), 7.91 (br s, 1H), 7.59 (d, J=7.5 Hz, 1H), 6.55 (d, J=7.5 Hz, 1H), 6.39 (m, 2H), 6.30 (m, 1H), 3.69 (s, 6H), 3.54-3.39 (m, 5H), 3.31-3.16 (m, 2H), 2.73-2.60 (m, 6H), 1.99-1.89 (m, 2H), 1.85-1.76 (m, 2H). Mass Spectrum: (MH⁺)=467.

### EXAMPLE 33

3-(3,5-Dichlorophenyl)-4-(3-[3-(5,6,7,8-tetrahydro-1,8-naphthyridin-2-yl)propyl]-1,2,4-oxadiazol-5-yl}butanoic acid trifluoroacetate.

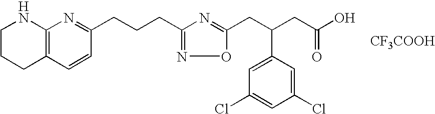

¹H NMR (DMSO-d₆) 12.3 (br s, 1H), 7.85 (br s, 1H), 7.6 (d, J=7.5 Hz, 1H), 7.42-7.35 (m, 3H), 6.55 (d, J=7.5 Hz, 1H), 3.62-3.5 (m, 2H), 3.4-3.22 (m, 4H), 2.85-2.6 (m, 7H), 1.99-1.89 (m, 2H), 1.85-1.76 (m, 2H). Mass Spectrum: (MH⁺)=476.

### EXAMPLE 34

3-(3,5-Difluorophenyl)-4-{3-[3-(5,6,7,8-tetrahydro-1,8-naphthyridin-2-yl)propyl]-1,2,4-oxadiazol-5-yl}butanoic acid trifluoroacetate.

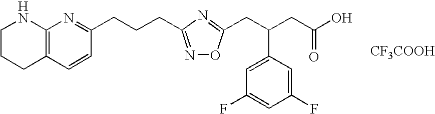

¹H NMR (DMSO-d₆) δ 7.70 (br s, 1H), 7.51 (d, J=7.5 Hz, 1H), 7.09-7.0 (m, 3H), 6.50 (d, J=7.5 Hz, 1H), 3.62-3.53 (m, 1H), 3.4-3.22 (m, 4H), 2.85-2.6 (m, 8H), 1.99-1.89 (m, 2H), 1.85-1.76 (m, 2H). Mass Spectrum: (MH⁺)=443.

### EXAMPLE 35

3-(3-Fluoro-4-methoxyphenyl)-4-{3-[3-(5,6,7,8-tetrahydro-1,8-naphthyridin-2-yl)propyl]-1,2,4-oxadiazol-5-yl}butanoic acid trifluoroacetate.

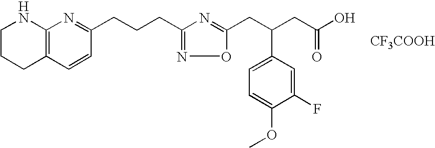

¹H NMR (DMSO-d₆) δ 12.20 (br s, 1H), 7.91 (br s, 1H), 7.59 (d, J=7.5 Hz, 1H), 7.25-7.20 (m, 2H), 7.1-7.0 (m, 2H), 6.60 (d, J=7.5 Hz, 2H), 3.80 (s, 3H), 3.63-3.41 (m, 3H), 3.38-3.21 (m, 2H), 2.82-2.63 (m, 6H), 1.99-1.89 (m, 2H), 1.85-1.76 (m, 2H). Mass Spectrum: (MH⁺)=455.

### EXAMPLE 36

4-{3-[3-(5,6,7,8-Tetrahydro-1,8-naphthyridin-2-yl)propyl]-1,2,4-oxadiazol-5-yl}-3-[4-(trifluoromethyl)phenyl]butanoic acid trifluoroacetate.

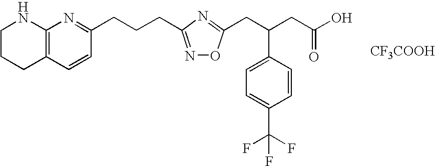

¹H NMR (DMSO-d₆) δ 12.3 (br s, 1H), 7.72 (br s, 1H), 7.61 (d, J=7.5 Hz, 2H), 7.57 (d, J=7.5 Hz, 1H), 7.49 (d, J=7.5 Hz, 2H), 6.52 (d, J=7.5 Hz, 1H), 3.70-3.60 (m, 1H), 3.4-3.27 (m, 4H), 2.85-2.55 (m, 8H), 1.99-1.89 (m, 2H), 1.85-1.76 (m, 2H). Mass Spectrum: (MH⁺)=475.

### EXAMPLE 37

3-(2-Methyl-1,3-thiazol-5-yl)-4-{3-[3-(5,6,7,8-tetrahydro-1,8-naphthyridin-2-yl)propyl]-1,2,4-oxadiazol-5-yl}butanoic acid trifluoroacetate.

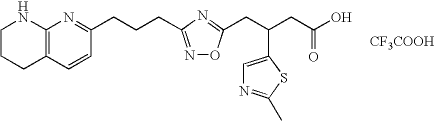

¹H NMR (DMSO-d₆) δ 12.45 (br s, 1H), 7.80 (br s, 1H), 7.61 (d, J=7.5 Hz, 1H), 7.34 (s, 1H), 6.58 (d, J=7.5 Hz, 1H), 3.91-3.80 (m, 1H), 3.45-3.22 (m, 4H), 2.90-2.64 (m, 8H), 2.53 (s, 3H), 2.01-1.92 (m, 2H), 1.85-1.76 (m, 2H). Mass Spectrum: (MH⁺)=427.

### EXAMPLE 38

3-(1-Phenyl-1H-pyrazol-4-yl)-4-{3-[3-(5,6,7,8-tetrahydro-1,8-naphthyridin-2-yl)propyl]-1,2,4-oxadiazol-5-yl}butanoic acid trifluoroacetate.

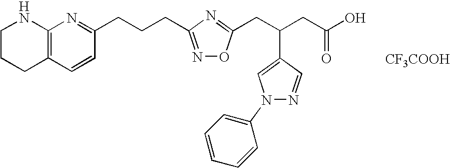

This compound was prepared from 1-phenyl-1H-pyrazole-4-carbaldehyde (Muri, Estela M. F.; Barreiro, Eliezer J.; Fraga, Carlos A. M.; Synth. Commun; 28; 7; 1998; 1299-1321) according to the procedure for Examples 32-37. ¹H NMR (DMSO-d₆) δ 12.3 (br s, 1H), 8.35 (s, 1H), 7.91 (br s, 1H), 7.73-7.70 (m, 2H), 7.59 (s, 1H), 7.54 (d, J=7.5 Hz, 1H), 7.44 (t, 2H), 7.25 (t, 1H), 6.52 (d, J=7.5 Hz, 1H), 3.65-3.55 (m, 1H), 3.43-3.20 (m, 4H), 2.81-2.60 (m, 8H), 1.99-1.89 (m, 2H), 1.85-1.76 (m, 2H). Mass Spectrum: (MH⁺)=473.

### EXAMPLE 39

3-(1-Benzofuran-6-yl)-4-{3-[3-(5,6,7,8-tetrahydro-1,8-naphthyridin-2-yl)propyl]-1,2,4-oxadiazol-5-yl}butanoic acid hydrochloride: 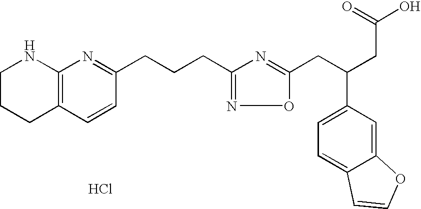
STEP 1. Triethyl 2-(1-benzofuran-6-yl)propane-1,1,3-tricarboxylate: Sodium ethoxide (7.5 mL, 19.9 mmols), diethyl malonate (3 mL, 19.9 mmols), and ether (50 mL) were stirred for 30 minutes. Then, 3-(benzofuran-6-yl)-acrylic acid ethyl ester (4.3 g, 19.9 mmols; prepared according to the procedure of Duggan, Mark, et al.; International Patent Application No. WO 99/30709) was added and the reaction mixture was refluxed for five hours. The mixture was cooled to room temperature, acidified with aqueous acetic acid, and extracted with ether. The ether was extracted with saturated bicarbonate, brine, dried (Na₂SO₄), and concentrated to give 6.6 grams of product (88%). NMR spectrum of the product was consistent for the proposed structure.
STEP 2. Diethyl 3-(1-benzofuran-6-yl)pentanedioate. Triethyl 2-(1-benzofuran-6-yl)propane-1,1,3-tricarboxylate (6.6 g., 17.5 mmols) was dissolved in DMSO (28 mL), H₂O (0.28 mL) and NaCl (520 mg., 8.8 mmols) was added. The mixture was heated to 160° C. for eight hours and then cooled to room temperature. The reaction mixture was extracted with ethyl acetate and water. The water layer was again extracted with ethyl acetate, the ethyl acetate fractions were combined and washed with water, brine, dried (Na₂SO₄), and concentrated. The residue was purified by flash chromatography 20% ethyl acetate: 80% hexane to give 4.4 grams of product (83%). NMR spectrum of the product was consistent for the proposed structure.

STEP 3. 4-(1-Benzofuran-6-yl)dihydro-2H-pyran-2,6(3H)-dione: Diethyl 3-(1-benzofuran-6-yl)pentanedioate (1.5 g., 4.9 mmols) was dissolved in a mixture of 15 mL methanol and 15 mL of THF and 15 mL of 1N NaOH solution was added. The reaction mixture was stirred at ambient temperature overnight. The volatile solvents were removed and the remaining aqueous solution was acidified with 1N HCl till pH of 1. The aqueous solution was extracted with ethyl acetate (2×). The ethyl acetate fractions were combined, dried (Na₂SO₄), and concentrated to give a quantitative yield of the desired product. NMR spectrum of the product was consistent for the proposed structure. This was converted to 4-(1-benzofuran-6-yl)dihydro-2H-pyran-2,6(3H)-dione as shown in earlier examples.

STEP 4. 3-(1-benzofuran-6-yl)-4-{3-[3-(5,6,7,8-tetrahydro-1,8-naphthyridin-2-yl)propyl]-1,2,4-oxadiazol-5-yl}butanoic acid hydrochloride: This was prepared starting from product in step 3 and using the procedures described in earlier examples. ¹H NMR (DMSO-d₆) δ 12.25 (br s, 1H), 8.02 (br s, 1H), 7.96-7.9 (m, 1H), 7.6-7.5 (m, 2H), 7.41-7.12 (m, 2H), 7.05-6.85 (m, 1H), 6.48-6.43 (m, 1H), 4.00-3.63 (m, 1H), 3.46-3.28 (m, 4H), 2.95-2.53 (m, 8H), 1.99-1.90 (m, 2H), 1.88-1.76 (m, 2H). Mass Spectrum: (MH⁺)=449. Mass Spectrum: (MH⁺)=447.

### EXAMPLE 40

3-(2,3-dihydro-1-benzofuran-6-yl)-4-{3-[3-(5,6,7,8-tetrahydro-1,8-naphthyridin-2-yl)propyl]-1,2,4-oxadiazol-5-yl}butanoic acid hydrochloride.

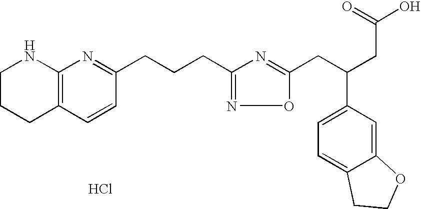

Diethyl 3-(1-benzofuran-6-yl)pentanedioate (2.63 g., 8.6 mmols) was dissolved in ethanol/water/acetic acid (52 mL/6 mL/2 mL) and the solution was degassed with argon and treated with Pd(OH)₂ (320 mg.). The mixture was placed under 1 atm of H₂ and stirred at room temperature overnight. The mixture was diluted with ethyl acetate and filtered through celite. The filtrate was concentrated and purified by flash chromatography 10% ethyl acetate: 90% hexane to give 2.53 grams of the desired product (96%). This was used as in Example 1 to synthesize the title compound. ¹H NMR (DMSO-d₆) δ 12.2 (br s, 1H), 7.85 (br s, 1H), 7.58 (d, J=7.5 Hz, 1H), 7.5-6.8 (m, 2H), 6.79 (d, J=7.5 Hz, 1H), 6.7-6.6 (m, 1H), 6.52 (t, 2H), 4.484.40 (m, 2H), 3.62-3.39 (m, 3H), 3.3-3.03 (m, 4H), 2.80-2.60 (m, 6H), 1.99-1.90 (m, 2H), 1.88-1.76 (m, 2H). Mass Spectrum: (MH⁺)=449.

Using the procedures described in earlier examples, the following compound was synthesized.

### EXAMPLE 41

3-(1,3-Benzodioxol-5-yl)-4-(3-{3-[(pyridin-2-ylamino)methyl]phenyl}-1,2,4-oxadiazol-5-yl)butanoic acid hydrochloride:

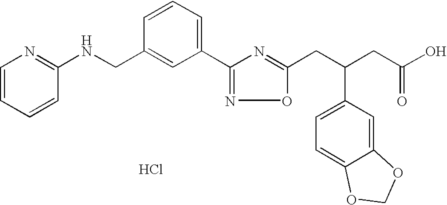

3-(1,3-Benzodioxol-5-yl)-4-(3-{3-[(pyridin-2-ylamino)methyl]phenyl}-1,2,4-oxadiazol-5-yl)butanoic acid hydrochloride: ¹H NMR (DMSO-d₆) δ 12.2 (br s, 1H), 9.06 (br s, 1H), 8.01-7.88 (m, 4H), 7.62-7.53 (m, 2H), 7.09 (d, J=7.5 Hz, 1H), 6.96 (m, 1H), 6.88 (t, 1H), 6.78-6.68 (m, 2H), 5.95 (s, 2H), 4.7 (m, 2H), 3.62-3.52 (m, 1H), 3.42-3.25 (m, 2H), 2.71-2.59 (m, 2H). Mass Spectrum: (MH⁺)=459.

### EXAMPLE 42

3-(7-Fluoro-1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate. 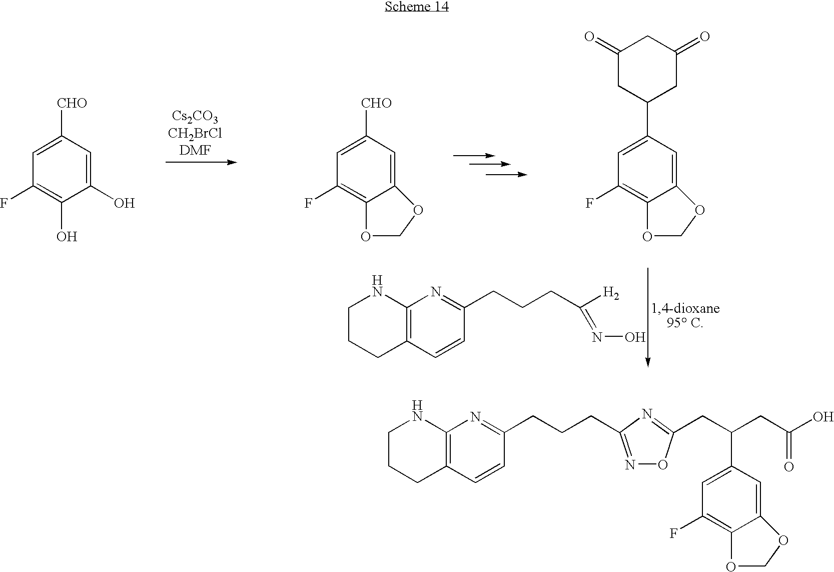
Step 1. 3-Fluoro-4,5-dihydroxybenzaldehyde:

Following the procedure of Kirk, K.; Cantacuzene, D.; Collins, B.; Chen, G.; Nimit, Y.; Creveling, C.; J. Med. Chem. 1982, 25, 680-684.

Step 2. 7-fluoro-1,3-benzodioxole-5-carbaldehyde:

Following the procedure of Zelle, R.; McClellan, W.; Tetrahedron Letters, 1991, 32, 2461-2464: To a stirred suspension of 3-fluoro-4,5-dihydroxybenzaldehyde (0.87 g, 0.00561 moles) and Cs₂CO₃ (2.74 g, 0.0842 moles) in anhydrous DMF (15 mL) was added BrCH₂Cl (0.55 mL, 0.00842 moles) and the resulting mixture was heated to 110° C. for 2 hours. The reaction was cooled to 25° C. and filtered through a pad of celite with EtOAc washing. The filtrate was concentrated and the residue diluted with water and extracted with EtOAc. The extracts were combined and washed with water, brine, dried over anhydrous MgSO₄, filtered and concentrated to provide a tan solid 0.67 g (71%).

Step 3. 4-(7-fluoro-1,3-benzodioxol-5-yl)dihydro-2H-pyran-2,6(3H)-dione.

This compound was synthesized starting from 7-fluoro-1,3-benzodioxole-5-carbaldehyde according to procedures outlined in the following references: Tokoroyama, Takashi; Kusaka, Hisashi; Can. J. Chem.; 74; 12; 1996; 2487-2502. Victory, P.; Alvarez-Larena, A.; Barbera, E.; J. Chem. Res. Miniprint; 4; 1989; 0631-0674. Hofman, S.; Baecke, G. D.; Kenda, B.; Clercq, P. J. De Synthesis; 1998; 479-489.

Step 4. 3-(7-Fluoro-1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate.

The title compound was prepared according to the method as described for preparing using the anhydride from STEP 3: ¹H NMR (CD₃OD) δ 7.52 (d, 1H), 6.61 (d, 1H), 6.57 (d, 1H), 6.53 (d, 1H), 5.91 (m, 2H), 3.60 (m, 1H), 3.48 (m, 2H), 3.29 (m, 2H), 3.25 (m, 2H), 2.80 (m, 2H), 2.70 (m, 4H), 2.04 (m, 2H), 1.93 (m, 2H). Mass Spectrum: (MH⁺)=469.20.

### EXAMPLE 43

3-(1,3-Benzoxazol-6-yl)-4-{3-[3-(5,6,7,8-tetrahydro-1,8-naphthyridin-2-yl)propyl]-1,2,4-oxadiazol-5-yl}butanoic acid hydrochloride.

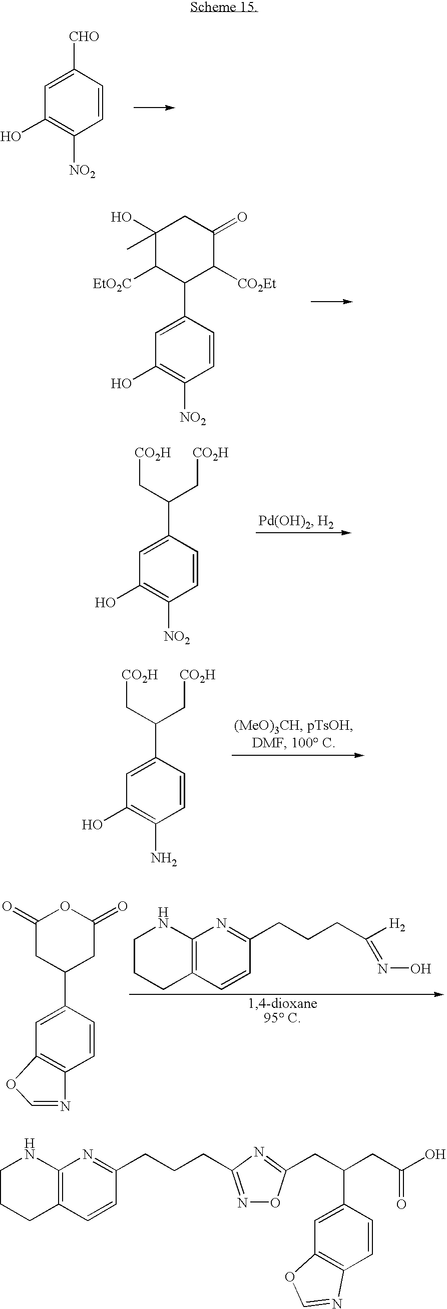

The title compound was prepared according to the methods outlined in scheme 15 and utilizing methods described for earlier examples. ¹H NMR (CD₃OD) 8.21 (s, 1H), 7.83 (d, 1H), 7.58 (d, 1H), 6.75 (d, 1H), 6.67(m, 1H), 6.52(d, 1H), 3.61(m, 1H), 3.50 (m, 2H), 3.30(m, 2H), 2.75 (m, 2H), 2.64 (m, 2H), 2.60 (m, 2H), 2.05 (m, 2H), 1.95 (m, 2H). Mass Spectrum: (MH⁺)=448.20.

### EXAMPLE 44

3-(3-Methyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate. 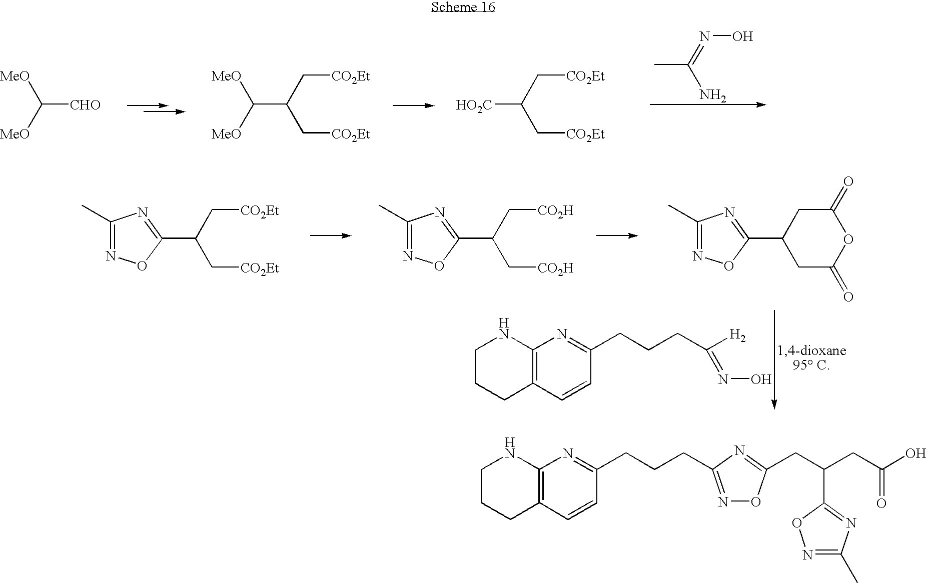
Step 1. Diethyl 3-(dimethoxymethyl)pentanedioate.

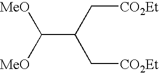

This compound was synthesized from dimethoxyacetaldehyde according to procedures outlined in: Tokoroyama, Takashi; Kusaka, Hisashi; Can. J. Chem.; 74; 12; 1996; 2487-2502. Victory, P.; Alvarez-Larena, A.; Barbera, E.; J. Chem. Res. Miniprint; 4; 1989; 0631-0674. Hofman, S.; Baecke, G. D.; Kenda, B.; Clercq, P. J. De Synthesis; 1998; 479-489.
Step 2. 4-Ethoxy-2-(2-ethoxy-2-oxoethyl)-4-oxobutanoic acid.

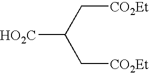

The compound was synthesized from Diethyl 3-(dimethoxymethyl)pentanedioate according to the procedures outlined in Eillison, R., Lukenbach, E., Chiu, C.; Tetrahedron Letters; 1975, 8, 499-502 and Bal, B. S.; Childers, W. E. Jr.; Pinnick, W. Tetrahedron 37, 1981, 2091-2096.
Step 3. N′-hydroxyethanimidamide.

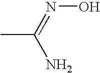

The compound was synthesized according to the procedures outlined in Bedford, C. D.; Howd, R. A.; Dailey, O. D.; Miller, A.; J. Med. Chem.; 1986, 29, 2174-2183.
Step 4 Diethyl 3-(3-methyl-1,2,4-oxadiazol-5-yl)pentanedioate.

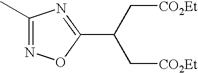

The compound was prepared according to the methods described in earlier examples, by coupling N′-hydroxyethanimidamide with 4-Ethoxy-2-(2-ethoxy-2-oxoethyl)-4-oxobutanoic acid.
Step 5. 3-(3-methyl-1,2,4-oxadiazol-5-yl)pentanedioic acid.

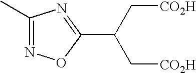

The compound was prepared from Diethyl 3-(3-methyl-1,2,4-oxadiazol-5-yl)pentanedioate according to the method as described for preparing EXAMPLE 1, STEP 2.
Step 6. 4-(3-methyl-1,2,4-oxadiazol-5-yl)dihydro-2H-pyran-2,6(3H)-dione.

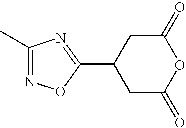

The compound was prepared from the 3-(3-methyl-1,2,4-oxadiazol-5-yl)pentanedioic acid according to the method as described for preparing EXAMPLE 1, STEP 3.
Step 7. 3-(3-methyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate.

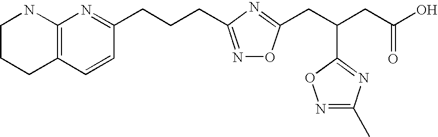

The title compound was prepared according to the method as described for preparing EXAMPLE 1, STEP 4 using the appropriate anhydride from STEP 6 (4-(3-methyl-1,2,4-oxadiazol-5-yl)dihydro-2H-pyran-2,6(3H)-dione): ¹H NMR (CDCl₃) 9.45 (s, br, 1H), 7.38(d, 1H), 6.43(d, 1H), 5.30(s, 1H), 4.08(m, 1H), 3.50(m, 2H), 3.45(m, 2H), 3.05(m, 2H), 2.79(m, 4H), 2.39(s, 3H), 2.15(m, 2H), 1.95(m, 2H). Mass Spectrum: (MH⁺)=413.2.

### EXAMPLE 45

3-(3-Ethyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate.

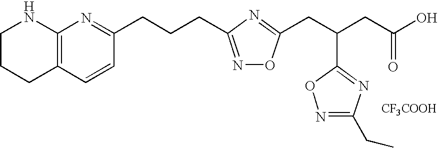

The title compound was prepared utilizing N′-hydroxypropanimidamide (Reference: Bedford, C. D.; Howd, R. A.; Dailey, O. D.; Miller, A.; J. Med. Chem.; 1986, 29, 2174-2183) in STEP 4 according to the methods described for preparing example 44, (3-(3-methyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate), STEPS 1-7. ¹H NMR (CD₃OD) 7.60 (d, 1H), 6.63(d, 1H), 4.05(m, 1H), 3.50(m, 4H), 3.00(m, 2H), 2.88 (m, 2H), 2.78 (m, 4H), 2.70 (m, 2H), 2.11(m, 2H), 1.96 (d, 2H), 1.25(m, 3H), Mass Spectrum: (MH⁺)=427.50.

### EXAMPLE 46

3-(3-Phenyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate.

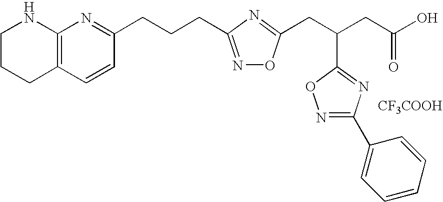

The title compound was prepared utilizing N′-hydroxybenzimidamide in STEP 4 according to the method as described for preparing example 45, (3-(3-ethyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate), STEPS 1-7. ¹H NMR (CD₃OD) 7.98 (d, 2H), 7.50(m, 4H), 6.52(d, 1H), 4.25(m, 1H), 3.58(m, 2H), 3.50(m, 2H), 3.08 (m, 2H), 2.80 (m, 4H), 2.65 (m, 2H), 2.05(m, 2H), 1.95 (d, 2H),

Mass Spectrum: (MH⁺)=475.60.

### EXAMPLES 47-67

Examples 47-67 were synthesized using anhydrides prepared by using one of the following methods: Vogel, A. I.; J. Chem. Soc.; 1934; pp1758-1765; McElvain, S. M.; Clemens, D. H.; J. Amer. Chem. Soc.; 80; 3915-3923 (1958); Diederich, F.; Dick, K.; Chem. Ber.; 118, 3817-3829 (1985).

### EXAMPLE 47

[1-Benzoyl-4-({3-[3-(5,6,7,8-tetrahydro-1,8-naphthyridin-2-yl)propyl]-1,2,4-oxadiazol-5-yl}methyl)piperidin-4-yl]acetic acid trifluoroacetate.

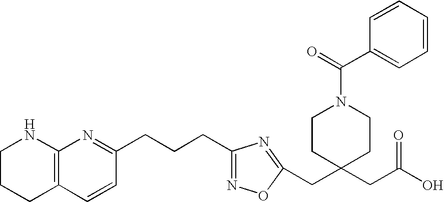

¹H NMR (CD₃OD) δ 1.70-1.96 (m, 4H), 2.03 (m, 2H), 2.21 (m, 2H), 2.64 (s, 2H), 2.88 (m, 6H), 3.44 (s, 2H), 3.58 (m, 4H), 3.83 (m, 1H), 3.98 (m, 1H), 6.71 (d, 1H), 7.48 (m, 2H), 7.55 (m, 3H), 7.65 (m, 1H); MS (ESI+) for C28H33N5O4 m/z 504.7 (M+H)+.

### EXAMPLE 48

[1-Benzoyl-4-({3-[4-(pyridin-2-ylamino)butyl]-1,2,4-oxadiazol-5-yl}methyl)piperidin-4-yl]acetic acid trifluoroacetate.

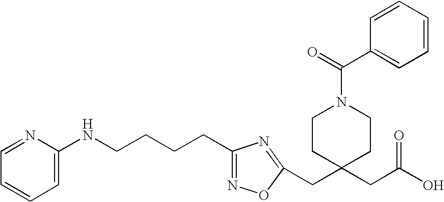

¹H NMR (CD₃OD) δ 1.61-1.96 (m, 10H), 2.57 (s, 2H), 2.84 (t, 2H), 3.38 (s, 2H), 3.42 (t, 2H), 3.51 (m, 1H), 3.78 (m, 1H), 3.90 (m, 1H), 6.89 (m, 1H), 7.05 (m, 1H), 7.42 (m, 2H), 7.48 (m, 3H), 7.81 (m, 1H), 7.89 (m, 1H); MS (ESI+) for C26H31N5O4 m/z 478.2466 (M+H)+.

### EXAMPLE 49

[1-(tert-Butoxycarbonyl)-4-({3-[3-(5,6,7,8-tetrahydro-1,8-naphthyridin-2-yl)propyl]-1,2,4-oxadiazol-5-yl}methyl)piperidin-4-yl]acetic acid trifluoroacetate.

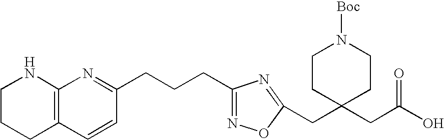

¹H NMR (CD₃OD) δ 1.47 (s, 9H), 1.57-1.73 (m, 3H), 1.98 (m, 3H), 2.14 (m, 2H), 2.50-2.69 (m, 3H), 2.83 (m, 6H), 3.27 (s, 2H), 3.40-3.62 (m, 4H), 6.64 (d, 1H), 7.60 (d, 1H); MS (ESI+) for C26H37N5O5 m/z 500.7 (M+H)⁺.

### EXAMPLE 50

[1-(tert-Butoxycarbonyl)-4-({3-[4-(pyridin-2-ylamino)butyl]-1,2,4-oxadiazol-5-yl}methyl)piperidin-4-yl]acetic acid trifluoroacetate.

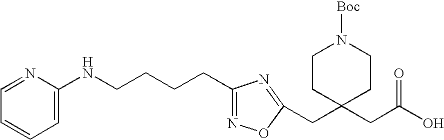

¹H NMR (CD₃OD) δ 1.48 (s, 9H), 1.5-2.0 (m, 7H), 2.46-2.62 (m, 2H), 2.82 (t, 2H), 3.25 (s, 2H), 3.38 (s, 2H), 3.39-3.6 (m, 4H), 6.83 (m, 1H), 6.98 (m, 1H), 7.84 (m, 2H); MS (ESI+) for C₂₄H₃₅N₅O₅ m/z 474.2736 (M+H)⁺.

### EXAMPLE 51

3-(4-Methylphenyl)-4-{3-[3-(5,6,7,8-tetrahydro-1,8-naphthyridin-2-yl)propyl]-1,2,4-oxadiazol-5-yl}butanoic acid trifluoroacetate.

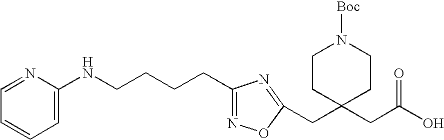

¹H NMR (CD₃OD) δ 1.97 (m, 2H), 2.08 (m, 2H), 2.26 (s, 3H), 2.65-2.86 (m, 8H), 3.20-3.38 (m, 2H), 3.51 (m, 2H), 3.68 (m, 1H), 6.58 (d, 1H), 7.07-7.18 (m, 4H), 7.57 (d, 1H); MS (ESI+) for C24H28N4O3 m/z 421 (M+H)+.

### EXAMPLE 52

3-(3-Chlorophenyl)-4-{3-[3-(5,6,7,8-tetrahydro-1,8-naphthyridin-2-yl)propyl]-1,2,4-oxadiazol-5-yl}butanoic acid hydrochloride.

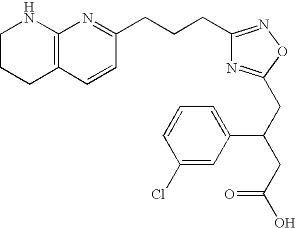

¹H NMR (CD₃OD) δ 1.98 (m, 2H), 2.08 (m, 2H), 2.60-2.90 (m, 8H), 3.25-3.40 (m, 2H), 3.51 (m, 2H), 3.72 (m, 1H), 6.58 (d, 1H), 7.19-7.32 (m, 4H), 7.57 (d, 1H); MS (ESI+) for C23H25ClN4O3 m/z 441 (M+H)+.

### EXAMPLE 53

3-(4-Methoxy-3-methylphenyl)-4-{3-[3-(5,6,7,8-tetrahydro-1,8-naphthyridin-2-yl)propyl]-1,2,4-oxadiazol-5-yl}butanoic acid hydrochloride.

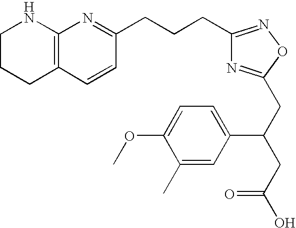

¹H NMR (CD₃OD) δ 1.98 (m, 2H), 2.07 (m, 2H), 2.13 (s, 3H), 2.64-2.85 (m, 8H), 3.19-3.35 (m, 2H), 3.52 (m, 2H), 3.62 (m, 1H), 3.77 (s, 3H), 6.56 (d, 1H), 6.78 (d, 1H), 7.00-7.10 (m, 2H), 7.58 (d, 1H); MS (ESI+) for C25H30N4O4 m/z 451 (M+H)+.

### EXAMPLE 54

3-[4-(Methylthio)phenyl]-4-{3-[3-(5,6,7,8-tetrahydro-1,8-naphthyridin-2-yl)propyl]-1,2,4-oxadiazol-5-yl}butanoic acid trifluoroacetate.

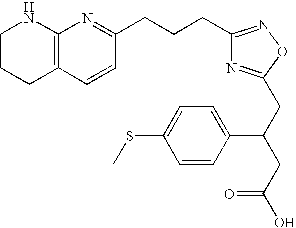

¹H NMR (CD₃OD) δ 1.95 (m, 2H), 2.06 (m, 2H), 2.42 (s, 3H), 2.62-2.88 (m, 8H), 3.22-3.38 (m, 2H), 3.52 (m, 2H), 3.68 (m, 1H), 6.55 (d, 1H), 7.17 (m, 4H), 7.58 (d, 1H); MS (ESI+) for C24H28N4O3S m/z 453 (M+H)+.

### EXAMPLE 55

3-(1-Methyl-1H-indol-3-yl)-4-{3-[3-(5,6,7,8-tetrahydro-1,8-naphthyridin-2-yl)propyl]-1,2,4-oxadiazol-5-yl}butanoic acid trifluoroacetate.

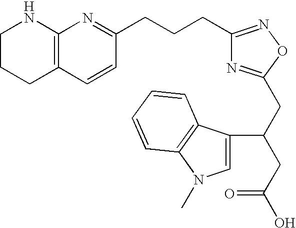

¹H NMR (CD₃OD) δ 1.97 (m, 4H), 2.55 (m, 2H), 2.68 (m, 2H), 2.80 (m, 2H), 2.90 (m, 2H), 3.35-3.46 (m, 2H), 3.50 (m, 2H), 3.72 (m, 3H), 4.05 (m, 1H), 6.43 (d, 1H), 6.97-7.15 (m, 3H), 7.27 (d, 1H), 7.51 (d, 1H), 7.53 (d, 1H); MS (ESI+) for C26H29N5O3 m/z 460 (M+H)+.

### EXAMPLE 56

3-(1,1′-Biphenyl-4-yl)-4-{3-[3-(5,6,7,8-tetrahydro-1,8-naphthyridin-2-yl)propyl]-1,2,4-oxadiazol-5-yl}butanoic acid hydrochloride.

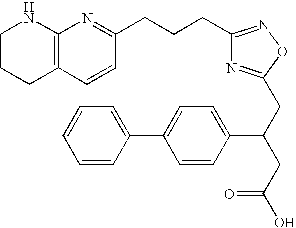

¹H NMR (CD₃OD) δ 1.93 (m, 2H), 2.05 (m, 2H), 2.58 (m, 2H), 2.75 (m, 5H), 2.85-2.92 (m, 1H), 3.28-3.43 (m, 2H), 3.46 (m, 2H), 3.79 (m, 1H), 6.47 (d, 1H), 7.30-7.43 (m, 6H), 7.53-7.57 (m, 4H); MS (ESI+) for C29H30N4O3 m/z 483 (M+H)+.

### EXAMPLE 57

3-(3-Bromophenyl)-4-{3-[3-(5,6,7,8-tetrahydro-1,8-naphthyridin-2-yl)propyl]-1,2,4-oxadiazol-5-yl}butanoic acid trifluoroacetate.

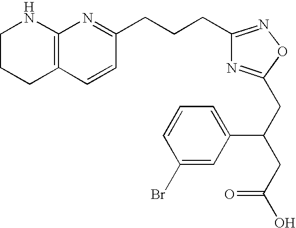

¹H NMR (CD₃OD) δ 1.96 (m, 2H), 2.08 (m, 2H), 2.68-2.91 (m, 8H), 3.24-3.42 (m, 2H), 3.52 (m, 2H), 3.71 (m, 1H), 6.58 (d, 1H), 7.20-7.24 (m, 1H), 7.26-7.30 (m, 1H), 7.32-7.38 (m, 1H), 7.46 (m, 1H), 7.59 (d, 1H); MS (ESI+) for C23H25BrN4O3 m/z 485 (M+H)+

### EXAMPLE 58

3-(4-Bromophenyl)-4-{3-[3-(5,6,7,8-tetrahydro-1,8-naphthyridin-2-yl)propyl]-1,2,4-oxadiazol-5-yl}butanoic acid hydrochloride.

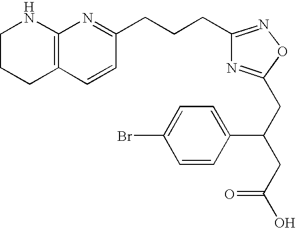

¹H NMR (CD₃OD) δ 1.96 (m, 2H), 2.05-2.11 (m, 2H), 2.65-2.77 (m, 5H), 2.80-2.88 (m, 3H), 3.23-3.39 (m, 2H), 3.53 (m, 2H), 3.71 (m, 1H), 6.57 (d, 1H), 7.22 (d, 2H), 7.42 (d, 2H), 7.58 (d, 1H); MS (ESI+) for C23H25BrN4O3 m/z 485 (M+H)+

### EXAMPLE 59

3-(3-Phenoxyphenyl)-4-{3-[3-(5,6,7,8-tetrahydro-1,8-naphthyridin-2-yl)propyl]-1,2,4-oxadiazol-5-yl}butanoic acid trifluoroacetate.

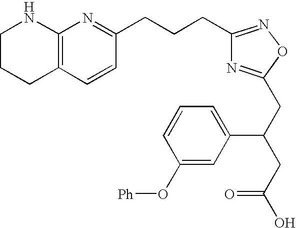

¹H NMR (CD₃OD) δ 1.96 (m, 2H), 2.08 (m, 2H), 2.67-2.89 (m, 8H), 3.23-3.29 (m, 1H), 3.48 (m, 2H), 3.71 (m, 1H), 6.58 (d, 1H), 6.89-6.92 (m, 3H), 7.03-7.15 (m, 2H), 7.25-7.37 (m, 3H), 7.54 (d, 1H); MS (ESI+) for C29H30N4O4 m/z 499 (M+H)+

### EXAMPLE 60

3-[3-(Benzyloxy)phenyl]-4-{3-[3-(5,6,7,8-tetrahydro-1,8-naphthyridin-2-yl)propyl]-1,2,4-oxadiazol-5-yl}butanoic acid hydrochloride.

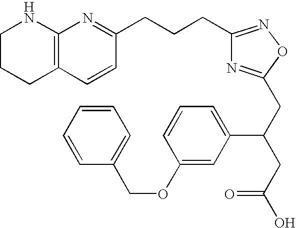

¹H NMR (CD₃OD) δ 1.94 (m, 2H), 2.09 (m, 2H), 2.64-2.83 (m, 8H), 3.22-3.32 (m, 2H), 3.46 (m, 2H), 3.71 (m, 1H), 5.08 (s, 2H), 6.53 (d, 1H), 6.83-6.95 (m, 3H), 7.20-7.23 (m, 1H), 7.30-7.49 (m, 6H); MS (ESI+) for C30H32N4O4 m/z 513 (M+H)+

### EXAMPLE 61

3-(3-Bromo-4-methoxyphenyl)-4-{3-[3-(5,6,7,8-tetrahydro-1,8-naphthyridin-2-yl)propyl]-1,2,4-oxadiazol-5-yl}butanoic acid trifluoroacetate.

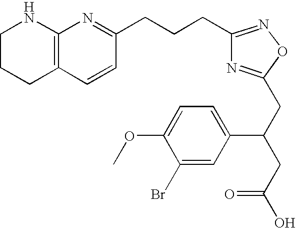

¹H NMR (CD₃OD) δ 1.95-2.09 (m, 4H), 2.65-2.76 (m, 4H), 2.82-2.87 (m, 4H), 3.21-3.28 (dd, 1H), 3.53 (m, 2H), 3.65 (m, 1H), 3.83 (s, 3H), 6.56 (d, 1H), 6.94 (d, 1H), 7.24 (dd, 1H), 7.43 (d, 1H), 7.59 (d, 1H); MS (ESI+) for C24H27BrN4O4 m/z 515 (M+H)+

### EXAMPLE 62

4-{3-[3-(5,6,7,8-Tetrahydro-1,8-naphthyridin-2-yl)propyl]-1,2,4-oxadiazol-5-yl}-3-(3,4,5-trimethoxyphenyl)butanoic acid trifluoroacetate.

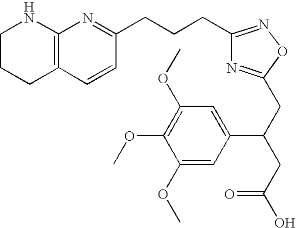

¹H NMR (CD₃OD) δ 1.95-2.11 (m, 4H), 2.67 (t, 2H), 2.72-2.79 (m, 3H), 2.82-2.88 (m, 3H), 3.25-3.39 (m, 2H), 3.52 (m, 2H), 3.69 (m, 1H), 3.71 (s, 3H), 3.82 (s, 6H), 6.54-6.57 (m, 3H), 7.57 (d, 1H); MS (ESI+) for C26H32N4O6 m/z 497 (M+H)+

### EXAMPLE 63

3-(2-Naphthyl)-4-{3-[3-(5,6,7,8-tetrahydro-1,8-naphthyridin-2-yl)propyl]-1,2,4-oxadiazol-5-yl}butanoic acid hydrochloride.

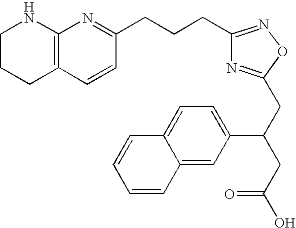

¹H NMR (CD₃OD) δ 1.93-2.00 (m, 4H), 2.46-2.52 (m, 2H), 2.68 (t, 2H), 2.78-2.88 (m, 3H), 2.94 (dd, 1H), 3.36-3.53 (m, 4H), 3.91 (m, 1H), 6.39 (d, 1H), 7.39-7.49 (m, 4H), 7.70-7.81 (m, 4H); MS (ESI+) for C₂₇H₂₈N₄O₃ m/z 457.6 (M+H)⁺.

### EXAMPLE 64

3-(3-Nitrophenyl)-4-{3-[3-(5,6,7,8-tetrahydro-1,8-naphthyridin-2-yl)propyl]-1,2,4-oxadiazol-5-yl}butanoic acid hydrochloride.

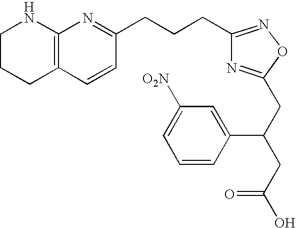

¹H NMR (CD₃OD) δ 1.96-2.05 (m, 4H), 2.69 (t, 2H), 2.74 (t, 2H), 2.80-2.86 (m, 3H), 2.95 (dd, 1H), 3.35 (dd, 1H), 3.94 (dd, 1H), 3.52 (m, 2H), 3.39 (m, 1H), 6.58 (d, 1H), 7.53-7.61 (m, 2H), 7.77 (m, 1H), 8.07 (m, 1H), 8.16 (m, 1H); MS (ESI+) for C₂₃H₂₅N₅O₅ m/z 452.5 (M+H)⁺.

### EXAMPLE 65

3-(3-Methylphenyl)-4-{3-[3-(5,6,7,8-tetrahydro-1,8-naphthyridin-2-yl)propyl]-1,2,4-oxadiazol-5-yl}butanoic acid hydrochloride.

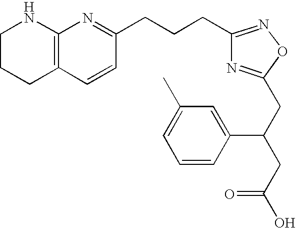

¹H NMR (CD₃OD) δ 1.97 (m, 2H), 2.08 (m, 2H), 2.29 (s, 3H), 2.67-2.76 (m, 5H), 2.80-2.85 (m, 3\ H), 3.25 (dd, 1H), 3.34 (dd, 1H), 3.51 (m, 2H), 3.69 (m, 1H), 6.59 (d, J=1 Hz, H), 7.00-7.18 (m, 4H), 7.57 (d, 1H); MS (ESI+) for C₂₄H₂₈N₄O₃ m/z 421.5 (M+H)⁺.

### EXAMPLE 66

3-(2-Furyl)-4-{3-[3-(5,6,7,8-tetrahydro-1,8-naphthyridin-2-yl)propyl]-1,2,4-oxadiazol-5-yl}butanoic acid hydrochloride.

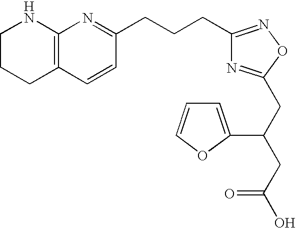

¹H NMR (CD₃OD) δ 1.97 (m, 2H), 2.12 (m, 2H), 2.67-2.85 (m, 8H), 3.24-3.38 (m, 2H), 3.52 (m, 2H), 3.83 (m, 1H), 6.13 (d, 1H), 6.30 (dd, 1H), 6.61 (d, 1H), 7.40 (d, 1H), 7.57 (d, 1H); MS (ESI+) for C₂₁H₂₄N₄O₄ m/z 397.1 (M+H)⁺.

### EXAMPLE 67

3-(2-Methylphenyl)-4-{3-[3-(5,6,7,8-tetrahydro-1,8-naphthyridin-2-yl)propyl]-1,2,4-oxadiazol-5-yl}butanoic acid hydrochloride.

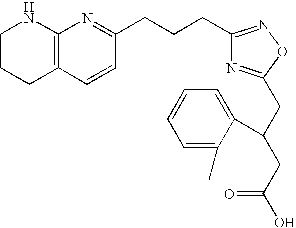

¹H NMR (CD₃OD) δ 1.97 (m, 2H), 2.05-2.13 (m, 2H), 2.41 (s, 3H), 2.66-2.87 (m, 8H), 3.22-3.35 (m, 2H), 3.52 (m, 2H), 4.06 (m, 1H), 6.57 (d, 1H), 7.05-7.20 (m, 3H), 7.29 (d, 1H), 7.58 (d, 1H); MS (ESI+) for C₂₄H₂₈N₄O₃ m/z 421.2 (M+H)⁺.

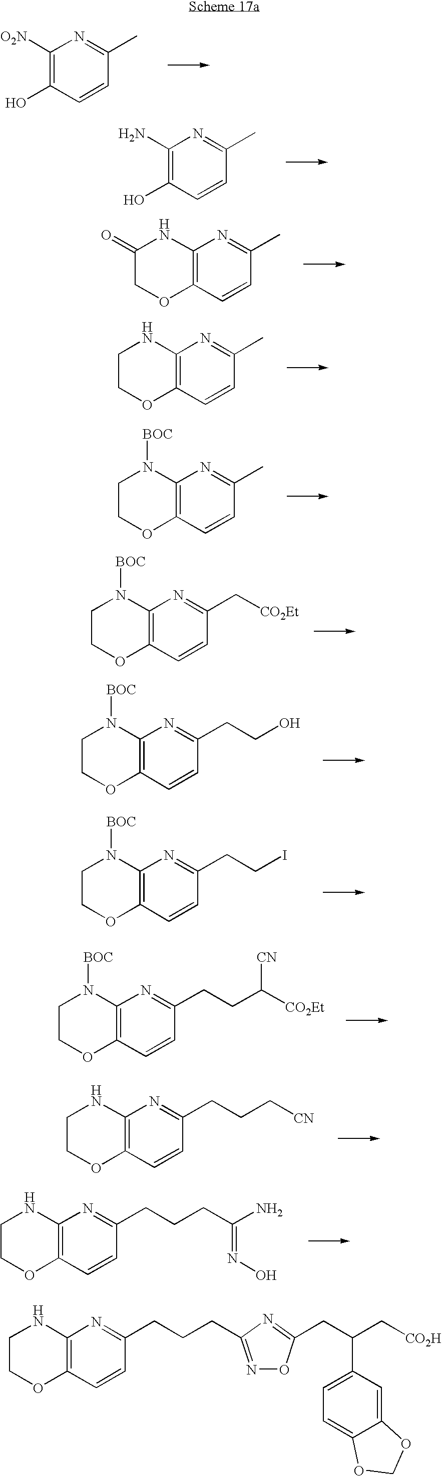

### EXAMPLE 68

Preparation of 3-(1,3-benzodioxol-5-yl)-4-{3-[3-(3,4-dihydro-2H-pyrido[3,2-b][1,4]oxazin-6-yl)propyl]-1,2,4-oxadiazol-5-yl}butanoic acid, TFA 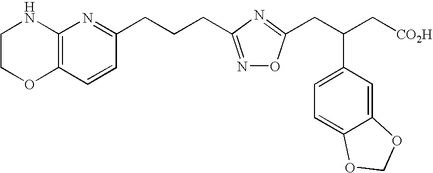
Step 1

3-Hydroxy-6-methyl-2-nitropyridine (30 g, 194.6 mmol) was hydrogenated in ethanol solution at 50° C. using H₂ at 5 psi and 20% Pd(OH)₂/C catalyst for 1 hour. Upon completion of the reaction, the catalyst was filtered off and the filtrate was concentrated under reduced pressure to get the desired product as a brown solid (23.68 g, 98%). NMR data was consistent with the proposed structure.

Step 2

0.37 mL chloroacetyl chloride was added dropwise to a stirred, cooled (0° C.) mixture of the product of step 1 (0.500 g), 0.810 g NaHCO₃, and 4 mL 2-butanone in 4 mL water. Once the addition was complete, the reaction mixture was warmed to room temp. and stirred for 30 minutes, then heated to 75° C. for 2 hours. The reaction mixture was cooled to room temp. and the 2-butanone was stripped off under reduced pressure. 1 mL water was added and the solids were filtered off and washed with water to get the crude product. The solid was dissolved in warmed (50° C.) ethyl acetate and filtered through a small plug of silica gel. The silica gel was washed with more warm ethyl acetate, combined with the filtrate, and concentrated under reduced pressure to get the desired product (0.250 g, 38%) as a deep orange solid. NMR data was consistent with the proposed structure. H NMR (400 MHz, CDCl₃) δ 2.45 (s, 3H), 4.64 (s, 2H), 6.78 (d, 1H), 7.15 (d, 1H).

Step 3

0.289 g LiAlH₄ was slowly added to 15 mL dry THF in a round-bottom flask fitted with a stirbar and a condenser. After stirring for 10 minutes, a solution of product of step 2 (1.00 g) in 15 mL dry THF was added dropwise. Upon completion of the addition, the reaction mixture was refluxed for 16 hours. The reaction was cooled to room temp. and quenched with 1 M NaOH solution until the mixture had become a milky yellow color. The precipitate was filtered off and washed 3 times with CH₂Cl₂. The filtrate and washings were combined, washed with brine, dried over MgSO₄, and concentrated under reduced pressure to get a pale yellow oil, which solidified on standing. NMR data was consistent with the proposed structure. H NMR (400 MHz, CDCl₃) δ 2.30 (s, 3H), 3.53 (m, 2H), 4.20 (t, 2H), 4.88 (s, 1H), 6.49 (d, 1H), 6.86 (d, 1H).

Step 4

A solution of the product of step 3 (2.96 g), di-tert-butyl dicarbonate (4.302 g) and triethylamine (2.75 mL) in 35 mL DMF was warmed to 50° C. with stirring for 16 hours. The reaction mixture was allowed to cool to room temp. and was concentrated under reduced pressure to get the crude product, which was purified by chromatography on silica gel (eluent: 30/70 ethyl acetate/hexane). The desired fractions were combined and concentrated under reduced pressure to get the desired product (1.46 g, 30%) as a yellow oil. NMR data was consistent with the proposed structure. H NMR (400 MHz, CDCl₃) δ 1.55 (s, 9H), 2.45 (s, 3H), 3.89 (t, 2H), 4.21 (t, 2H), 6.82 (d, 1H), 7.05 (d, 1H).

Step 5

8.17 mL lithium diisopropylamide solution (2.0 M in THF/ethylbenzene/heptane) was added dropwise to a chilled (−78° C.), stirred solution of product of step 4 (1.46 g) and diethyl carbonate (2.549 g) in 15 mL dry THF under nitrogen atmosphere. After 30 minutes the reaction was quenched with saturated NH₄Cl solution and warmed to room temp. The mixture was extracted three times with ethyl acetate and all organic extracts were combined, dried over MgSO₄, and concentrated under reduced pressure to get the crude product, which was purified by chromatography on silica gel (eluent: 40/60 ethyl acetate/hexane). The desired fractions were combined and concentrated under reduced pressure to get the desired product (1.48 g, 78%) as a yellow solid. NMR data was consistent with the proposed structure.

Step 6

To a solution of product of step 5 (1.48 g) in dry THF (20 mL) at room temp. was added a solution of LiBH₄ (2.0 M in THF, 2.75 mL), and the resulting mixture was heated to reflux. After 16 hours the mixture was cooled to 0° C. and carefully quenched with water (20 mL). After 10 minutes, the mixture was extracted three times with ethyl acetate. The combined organic extracts were washed with brine, dried over MgSO₄, filtered, and concentrated under reduced pressure to give a yellow solid. H NMR (400 MHz, CDCl₃) δ 1.55 (s, 9H), 2.90 (t, 2H), 3.93 (t, 2H), 3.96 (m, 2H), 4.23 (m, 2H), 6.78 (d, 1H), 7.11 (d, 1H).

Step 7

A mixture of the residue of step 6 and 4 M HCl in dioxane (6 mL) was stirred at room temperature for 4 hours, and then concentrated under reduced temperature. The residue was chromatographed on silica gel (eluent: 94.5/5/0.5 chloroform/ethanol/ammonium hydroxide) to afford a yellow solid. H NMR (400 MHz, CDCl₃) δ 2.77 (t, 3H), 3.56 (m, 2H), 3.91 (t, 2H), 4.21 (t, 2H), 4.73 (s, 1H), 6.39 (d, 1H), 6.90 (d, 1H).

Step 8

To a stirred, cooled (0° C.) solution of the product of step 6 (3.12 g, 11.13 mmol), triphenylphosphine (3.795 g, 14.47 mmol) and imidazole (1.084 mg, 15.92 mmol) in CH₃CN (10 mL) and dry ether (16 mL) was slowly added iodine (3.955 g, 15.58 mmol) and then stirred for 1 hour. The resulting mixture was added 150 mL ether, washed successively with saturated aqueous Na₂S₂O₃ and brine, dried over Na₂SO₄ and concentrated under reduced pressure. The residue was purified by flash chromatography (silica, 20% EtOAC/Hex) to afford a yellow solid. H NMR (400 MHz, CDCl₃) δ 1.54 (s, 9H), 3.23 (t, 2H), 3.49 (t, 2H), 3.91 (t, 2H), 4.23 (t, 2H), 6.84 (d, 1H), 7.10 (d, 1H).

Step 9

NaH (103 mg of a 60% weight dispersion in mineral oil, 2.564 mmol) was suspended in DMF (13 mL) at 0° C. under N₂. Ethyl cyanoacetate (0.27 mL, 2.564 mmol) was added and the resulting mixture stirred for 30 min at 0° C. The product of step 8 (1.00 g, 2.564 mmol) in DMF (2 mL) was introduced to the reaction mixture and stirred for 1 hour at room temperature. The mixture was cooled to 0 and quenched with water and extracted with EtOAc (3×). The organic layers were washed with brine, dried over Na₂SO₄ and concentrated in vacuo. The residue was purified by flash chromatography (silica, 50% EtOAC/Hex) to afford a colorless oil. H NMR (400 MHz, CDCl₃) δ 1.32 (t, 3H), 1.54 (s, 9H), 2.30 (m, 1H), 2.45 (m, 1H), 2.90 (m, 2H), 3.90 (m, 2H), 3.97 (m, 1H), 4.25 (m, 4H), 6.36 (d, 1H), 7.09 (d, 1H).

Step 10

A mixture of the product of step 9 (460 mg, 1.225 mmol) and KOH (powder, 123 mg, 1.838 mmol) in ethylene glycol (2 mL) under N₂ was heated at 150 for 3 hours. The mixture was cooled to 0° C. and portioned between water and EtOAc. The organic phase was washed with brine, dried over Na₂SO₄ and concentrated in vacuo. Flash chromatography (silica, 100% EtOAc) yielded a colorless oil. H NMR (400 MHz, CDCl₃) δ 2.03 (m, 2H), 2.34 (t, 2H), 2.67 (t, 2H), 3.54 (m, 2H), 4.20 (t, 2H), 6.41 (d, 1H), 6.89 (d, 1H).

Step 11

A mixture of the product of step 10 (460 mg, 2.263 mmol) and hydroxylamine (0.329 mL of a 50% weight solution in water, 4.98 mmol) in ethanol (6 mL) under N₂ was heated at 60° C. overnight. The mixture was cooled to room temperature and concentrated in vacuo to yield a white solid.

Step 12

A mixture of the product of step 11 (550 mg, 2.263 mmol) and the product from Example 1, Step 3 (529 mg, 2.263 mmol) in 1,4-dioxane (3 mL) was heated at 90° C. overnight. The reaction mixture was allowed to cool to room temperature and concentrated. The residue was purified on HPLC using acetonitrile gradient 15-50% in 30 min to yield 30 mg desired product as an yellow oil. FAB-MS:(MH+)=453. H NMR (400 MHz, CD₃OD) [symbol] 2.05 (m, 2H), 2.74 (m, 6H), 3.27 (m, 2H), 3.65 (m, 3H), 4.30 (t, 2H), 5.86 (s, 2H), 6.61 (d, 1H), 6.69 (s, 2H), 6.78 (s, 1H), 7.34 (d, 1H).

### EXAMPLE 69

Preparation of 4-{3-[3-(3,4-dihydro-2H-pyrido[3,2-b][1,4]oxazin-6-yl)propyl]-1,2,4-oxadiazol-5-yl}-3-(3,5-dimethoxyphenyl)butanoic acid, TFA

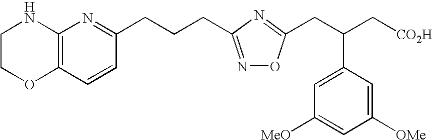

A mixture of the product of step 11, Example 68 (190 mg, 0.542 mmol) and the appropriate anhydride (135 mg, 0.542 mmol) in 1,4-dioxane (4 mL) was heated at 90° C. overnight. The reaction mixture was allowed to cool to room temperature and concentrated. The residue was purified on HPLC using acetonitrile gradient 15-50% in 30 min to yield 20 mg desired product as a yellow oil. FAB-MS:(MH+)=469. H NMR (400 MHz, CD₃OD) [symbol] 2.00 (m, 2H), 2.70 (m, 6H), 3.25 (m, 2H), 3.61 (m, 3H), 3.66 (s, 6H), 4.24 (t, 2H), 6.26 (t, 1H), 6.36 (d, 2H), 6.54 (d, 1H), 7.27 (d, 1H).

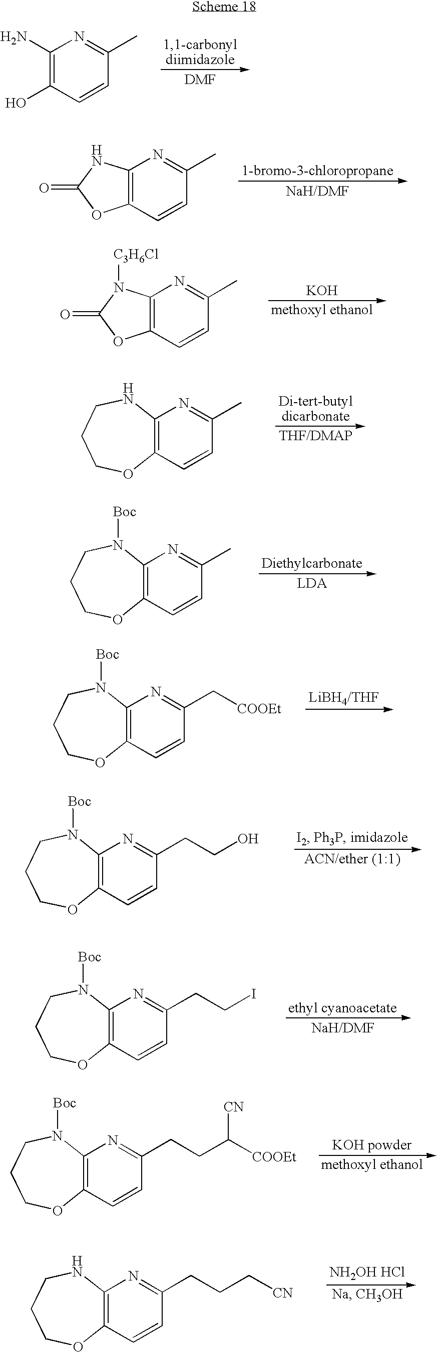

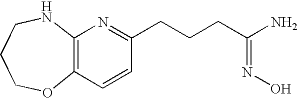

### EXAMPLE 70

3-Benzo[1,3]dioxol-5-yl-4-{3-[3-(6,7,8,9-tetrahydro-5-oxa-1,9-diaza-benzocyclohepten-2-yl)-propyl]-[1,2,4]oxadiazol-5-yl}-butyric acid. 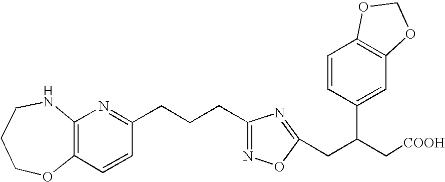
Step 1. 5-Methyl-3H-oxazolo[4,5-b]pyridin-2-one

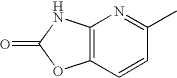

The solution of 2-amino-6-methyl-pyridin-3-ol (0.2 g, 1.6 mmol), 1,1-carbonyldiimidazole (0.39 g, 2.4 mmol), and DMF (10 mL) was stirred at room temperature overnight, and then was diluted with water (50 mL), extracted with ethyl acetate (3×50 mL). The ethyl acetate solution was washed with water and brine, concentrated, purified on a silica gel column, eluting with 10% ethyl acetate/hexane to afford 120 mg of the desired product as light yellow solid. ¹H-NMR (CD₃OD): δ 2.42 (3H, s, CH₃), δ 6.92 (1H, d, Py-H), δ7.38 (1H, d, Py-H).
Step 2. 3-(3-Chloro-propyl)-5-methyl-3H-oxazolo[4,5-b]pyridin-2-one.

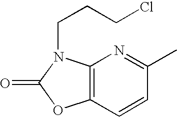

The solution of NaH 60% (0.021 g, 0.51 mmol), DMF (10 mL), and 5-methyl-3H-oxazolo[4,5-b]pyridin-2-one (70 mg, 0.47 mmol) was stirred at room temperature for 0.5 hour. 1-Bromo-3-chloro propane (0.056 mL, 0.56 mmol) was added. The resulting solution was stirred at room temperature overnight. The solution was diluted water (50 mL) and then extracted with ethyl acetate, washed with water, brine, concentrated to give 110 mg of the desired product as yellow oil. ¹H-NMR (CDCl₃): δ 2.35 (2H, m, CH₂), 2.53 (3H, s, CH₃) 3.60 (2H, t, CH₂), 4.08 (2H, t, CH₂), 6.90 (1H, d, Py-H), 7.28 (1H, d, Py-H).
Step 3. 2-Methyl-6,7,8,9-tetrahydro-5-oxa-1,9-diaza-benzocycloheptene.

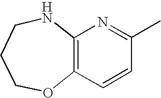

The mixture of 3-(3-chloro-propyl)-5-methyl-3H-oxazolo[4,5-b]pyridin-2-one (15 g), methoxyl ethanol (200 mL), and KOH (18.24 g) was heated to reflux for two days under nitrogen. The solution was cooled to room temperature and then solvent was removed. The residue was dissolved in the mixture of water and ethyl acetate (1:1, 1 L). The aqueous portion was extracted well with ethyl acetate and the combined organic extract was washed with water, brine and dried with MgSO₄. Solvent was removed to give the crude product that was purified on a silica gel column, eluting with 70% ethyl acetate/hexane to afford 7.7 g of the desired product as colorless oil. ¹H-NMR (CDCl₃): δ 2.025 (2H, m, CH₂), 2.35 (3H, s, CH₃) 3.32 (2H, m, CH₂), 4.10 (2H, t, CH₂), 4.65 (1H, br s, NH), 6.50 (1H, d, Py-H), 7.05 (1H, d, Py-H).
Step 4. 2-Methyl-7,8-dihydro-6H-5-oxa-1,9-diaza-benzocycloheptene-9-carboxylic acid tert-butyl ester.

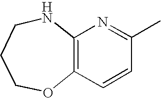

A solution of 2-methyl-6,7,8,9-tetrahydro-5-oxa-1,9-diaza-benzocycloheptene (25.44 g, 0.155 mole), tetrahydrofuran (100 mL), di-tert-butyldicarbonate (67.61 g, 0.31 mole), and dimethylaminopyridine (5 g) was heated up to 40° C. for 3 hours. The solution was cooled to room temperature and then concentrated to give crude product that was crystallized with methanol to offer 23 g of the desired product as yellow solid. ¹H-NMR (CDCl₃): δ 1.45 (9H, s, 3×CH₃), 2.05 (2H, m, CH₂), 2.50 (3H, s, CH₃) 3.70 (2H, br s, CH₂), 4.10 (2H, t, CH₂), 6.95 (1H, d, Py-H), 7.25 (1H, d, Py-H).
Step 5. 2-Ethoxycarbonylmethyl-7,8-dihydro-6H-5-oxa-1,9-diaza-benzocycloheptene-9-carboxylic acid tert-butyl ester.

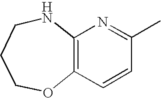

The solution of 2-methyl-7,8-dihydro-6H-5-oxa-1,9-diaza-benzocycloheptene-9-carboxylic acid tert-butyl ester (3.79 g, 14.34 mmol) and THF (30 mL) was cooled to −78° C. Lithium diisopropylamide (10.75 mL, 21.51 mmol) was added. The mixture was stirred at −78° C. for 0.5 hour, and then diethylcarbonate was added. The resulting mixture was stirred at −78° C. for 2 hours. Saturate aqueous NH4Cl solution was added to quench the reaction. The solution was extracted with ethyl acetate, washed with brine, dried with MgSO₄, concentrated, and purified on a silica gel column (10% ethyl acetate/hexane) to afford 1.15 g of the desired product. ¹H-NMR (CDCl₃): δ 1.45 (9H, s, 3×CH₃), 2.025 (2H, m, CH₂), 2.35 (3H, s, CH₃) 3.32 (2H, m, CH₂), 4.10 (2H, t, CH₂), 4.65 (1H, br s, NH), 6.50 (1H, d, Py-H), 7.05 (1H, d, Py-H).
Step 6. 2-(2-Hydroxy-ethyl)-7,8-dihydro-6H-5-oxa-1,9-diaza-benzocyclohept-ene-9-carboxylic acid tert-butyl ester.

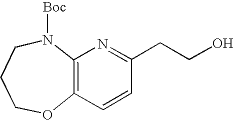

The solution of 2-ethoxycarbonylmethyl-7,8-dihydro-6H-5-oxa-1,9-diaza-benzocycloheptene-9-carboxylic acid tert-butyl ester (1.67 g, 5.00 mmol) and THF (50 mL) and Lithium borohydride (4.96 mL, 9.93 mmol) was heated to reflux overnight. The reaction was quenched with water, extracted with ethyl acetate, washed with brine, concentrated, and purified on a silica gel column (40% ethyl acetate/hexane) to afford 1.07 g of the desired product. ¹H-NMR (CD₃OD): δ 1.45 (9H, s, 3×CH₃), 2.08 (2H, m, CH₂), 2.95 (2H, t, CH₂), 3.70 (2H, br s, CH₂), 3.85 (2H, t, CH₂), 4.10 (2H, br s, CH₂), 7.20 (1H, d, Py-H), 7.45 (1H, d, Py-H).
Step 7. 2-(2-Iodo-ethyl)-7,8-dihydro-6H-5-oxa-1,9-diaza-benzocycloheptene-9-carboxylic acid tert-butyl ester.

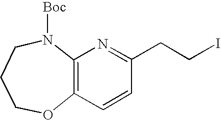

The solution of 2-(2-hydroxy-ethyl)-7,8-dihydro-6H-5-oxa-1,9-diaza-benzocycloheptene-9-carboxylic acid tert-butyl ester (0.20 g, 0.68 mmol) and acetonitrile/ether (1:1, 30 mL), triphenylphosphine (0.23 g, 0.88 mmol), imidazole (0.06 g, 0.98 mmol) was cooled to 0° C. Iodine (0.24 g, 0.95 mmol) was added. The mixture was stirred at −78° C. for 0.5 hour, and then diethylcarbonate was added. The resulting mixture was stirred at 0° C. for 2 hours. Saturate aqueous Na₂S₂O₄ solution was added to quench the reaction. The solution was extracted with ethyl acetate, washed with brine, dried with MgSO₄, concentrated, and purified on a silica gel column (30% ethyl acetate/hexane) to afford 0.223 g of the desired product. ¹H-NMR (CDCl₃): δ 1.39 (9H, s, 3×CH₃), δ 2.03 (2H, m, CH₂), δ 3.23 (2H, t, CH₂), δ 3.42 (2H, t, CH₂), δ 3.68 (2H, br s, CH₂), δ 4.08 (2H, m, CH₂), δ 6.92 (1H, d, Py-H), δ 7.22 (1H, d, Py-H).
Step 8. 2-(3-Cyano-3-ethoxycarbonyl-propyl)-7,8-dihydro-6H-5-oxa-1,9-diaza-benzocycloheptene-9-carboxylic acid tert-butyl ester.

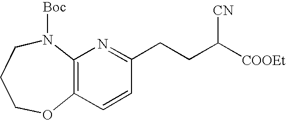

The solution of NaH (0.1 g, 2.52 mmol) and DMF (15 mL) was cooled to 0° C. Ethyl cyanoacetate (0.27 mL, 2.52 mmol) was added. The mixture was stirred at 0° C. for 0.5 hour, and then 2-(2-iodo-ethyl)-7,8-dihydro-6H-5-oxa-1,9-diaza-benzocycloheptene-9-carboxylic acid tert-butyl ester in DMF (0.68 g, 1.68 mmol) was added. The resulting mixture was stirred at 0° C. for one hour. Water was added to quench the reaction. The solution was extracted with ethyl acetate, washed with brine, dried with MgSO₄, concentrated, and purified on a silica gel column (10% ethyl acetate/hexane) to afford 0.2 g of the desired product. ¹H-NMR (CDCl₃): δ 1.30 (3H, t, CH₃), δ 1.45 (9H, s, 3×CH₃), δ 2.15 (2H, m, CH₂), δ 2.45 (2H, m, CH₂), δ 3.05 (2H, m, CH₂), δ 3.70 (2H, br s, CH₂), δ 3.80 (2H, m, CH₂), δ 4.15 (2H, m, CH₂), δ 4.30 (2H, q, CH₂), δ 6.95 (1H, d, Py-H), 67.30 (1H, d, Py-H).
Step 9. 4-(6,7,8,9-Tetrahydro-5-oxa-1,9-diaza-benzocyclohepten-2-yl)-butyronitrile.

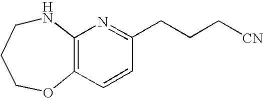

The solution of 2-(3-Cyano-3-ethoxycarbonyl-propyl)-7,8-dihydro-6H-5-oxa-1,9-diaza-benzocycloheptene-9-carboxylic acid tert-butyl ester (0.27 g, 0.58 mmol), ethylene glycol (20 mL) and KOH powder (0.058 g, 1.04 mmol) was heated to 150° C. for 3 hours. The reaction was quenched with water, extracted with ethyl acetate, washed with brine, concentrated, and purified on a silica gel column (ethyl acetate) to afford 0.146 g of the desired product. ¹H-NMR (CDCl₃): δ 1.45 (9H, s, 3×CH₃), δ 2.08 (2H, m, ¹H-NMR (CD₃OD): δ 2.04 (2H, m, CH₂), δ 2.06 (2H, t, CH₂), δ 2.36 (2H, t, CH₂), δ 2.70 (2H, t, CH₂), δ 3.35 (2H, m, CH₂), δ 4.10 (2H, t, CH₂), δ 4.84 (1H, s, NH), δ 6.52 (1H, d, Py-H), δ 7.08 (1H, d, Py-H).
Step 10. N-Hydroxy-4-(6,7,8,9-tetrahydro-5-oxa-1,9-diaza-benzocyclohepten-2-yl)-butyramidine.

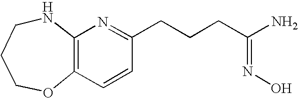

To a methanol (7 mL) at room temperature under N₂ was added Na (154 mg, 6.7 mmol). After the Na was dissolved in methanol NH₂OH hydrochloride salt was added (465 mg, 6.7 mmol). The resulting solution was stirred under N₂ for 2 hours and then filtered. The filtrate was added to 4-(6,7,8,9-Tetrahydro-5-oxa-1,9-diaza-benzocyclohepten-2-yl)-butyronitrile and then the mixture was heated to 40° C. overnight. The solvent was removed. The crude product was purified on a reverse phase HPLC using acetonitrile/water (1%) gradient to give 168 mg of the desired product as white solid. ¹H-NMR (CD₃OD): δ 2.08 (2H, m, CH₂), 2.25 (2H, m, CH₂), 2.50 (2H, t, CH₂), 2.80 (2H, t, CH₂), 3.70 (2H, m, CH₂), 3.85 (2H, t, CH₂), 6.67 (1H, d, Py-H), 7.48 (1H, d, Py-H).
Step 11. 3-Benzo[1,3]dioxol-5-yl-4-{3-[3-(6,7,8,9-tetrahydro-5-oxa-1,9-diaza-benzocyclohepten-2-yl)-propyl]-[1,2,4]oxadiazol-5-yl}-butyric acid.

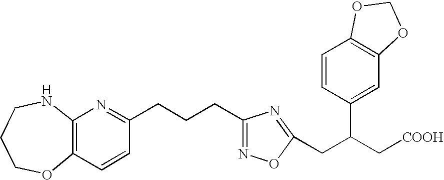

A solution of N-hydroxy-4-(6,7,8,9-tetrahydro-5-oxa-1,9-diaza-benzocyclohepten-2-yl)-butyramidine (168 mg, 0.46 mmol), 4-benzo[1,3]dioxol-5-yl-dihydro-pyran-2,6-dione (129 mg, 0.55 mmol), dioxane (15 mL), and triethylamine (0.13 mL, 0.92 mmol) was headed to 95° C. under N₂ overnight. The solvent was removed. The crude product was purified on a reverse phase HPLC using acetonitrile/water (5%) gradient to give 0.093 g of the desired product as colorless oil. ¹H-NMR (CD₃CN): δ 1.93 (2H, m, CH₂), 2.58 (2H, m, CH₂), 2.50 (2H, m, CH₂), 2.60 (2H, m, CH₂), 2.70 (2H, m, CH₂), 3.24 (2H, m, CH₂), 3.49 (1H, m, CH), 3.58 (2H, t, CH₂), 4.23 (2H, t, CH₂), 5.93 (2H, s, CH₂), 6.60 (1H, d, Py-H), 6.68 (1H, d, Ar—H), 6.74 (1H, d, Ar—H), 6.90 (1H, s, Ar—H), 7.42 (1H, d, Py-H). Calcd. for C₂₄H₂₆N₄O₆. 1.5 TFA, 1.0H₂O: C, 49.47; H, 4.54; N, 8.55. Found: C, 49.47; H, 4.62; N, 8.78.

### EXAMPLE 71

3-(3-Fluoro-4-methoxyphenyl)-4-{3-[3-(6,7,8,9-tetrahydro-5-oxa-1,9-diaza-benzocyclohepten-2-yl)-propyl]-[1,2,4]oxadiazol-5-yl}-butyric acid.

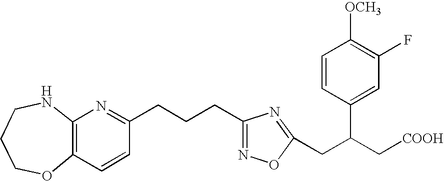

This was prepared utilizing the procedure for the preparation of Example 70 using the appropriate anhydride in step 11. ¹H-NMR (CD₃CN): δ 1.93 (2H, m, CH₂), 2.00 (2H, m, CH₂), 2.18 (2H, m, CH₂), 2.65 (2H, m, CH), 2.78 (2H, m, CH₂), 3.20 (2H, m, CH₂), 3.58 (1H, m, CH), 3.61 (2H, t, CH₂), 3.80 (3H, s, CH₃), 4.28 (2H, t, CH₂), 6.48 (1H, d, Py-H), 7.00 (3H, m, Ar—H), 7.32 (1H, s, Py-H). Calcd. for C₂₄H₂₇FN₄O₅ 1.3 TFA, 1.0H₂O: C, 50.18; H, 4.80; N, 8.80. Found: C, 50.27; H, 4.48; N, 9.13.

### EXAMPLE 72

3-(3,5-Difluorophenyl)-4-{3-[3-(6,7,8,9-tetrahydro-5-oxa-1,9-diaza-benzocyclohepten-2-yl)-propyl]-[1,2,4]oxadiazol-5-yl}-butyric acid.

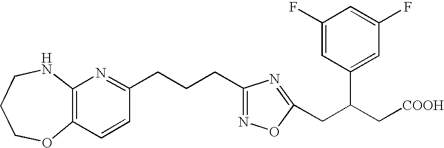

This was prepared utilizing the procedure for the preparation of Example 70 using the appropriate anhydride in step 11. ¹H-NMR (CD₃CN): δ 1.93 (2H, m, CH₂), 2.00 (2H, m, CH₂), 2.18 (2H, m, CH₂), 2.67 (2H, m, CH), 2.75 (2H, m, CH₂), 3.22 (2H, m, CH₂), 3.60 (2H, t, CH₂), 3.68 (1H, m, CH), 4.28 (2H, t, CH₂), 6.50 (1H, d, Pyr-H), 6.78 (1H, m, Ar—H), 6.90 (2H, m, Ar—H), 7.35 (1H, s, Py-H). Calcd. for C₂₃H₂₄F₂N₄O₄.1.4 TFA, 1.0H₂O: C, 48.72; H, 4.07; N, 9.02. Found: C, 48.47; H, 4.07; N, 9.02.

### EXAMPLE 73

3-(3,5-Dimethoxyphenyl)-4-{3-[3-(6,7,8,9-tetrahydro-5-oxa-1,9-diaza-benzocyclohepten-2-yl)-propyl]-[1,2,4]oxadiazol-5-yl}-butyric acid.

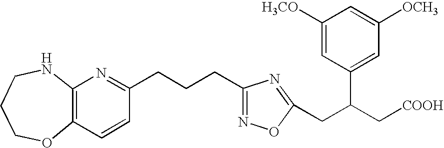

This was prepared utilizing the procedure for the preparation of Example 70 using the appropriate anhydride in step 11. ¹H-NMR (CD₃CN): δ 1.93 (2H, m, CH₂), 2.00 (2H, m, CH₂), 2.18 (2H, m, CH₂), 2.65 (2H, m, CH), 2.75 (2H, m, CH₂), 3.20 (2H, m, CH₂), 3.58 (1H, m, CH), 3.61 (2H, t, CH₂), 3.70 (6H, s, 2×CH₃), 4.28 (2H, t, CH₂), 6.30 (1H, m, Ar—H), 6.38 (2H, d, Ar—H), 6.50 (1H, d, Py-H), 7.32 (1H, s, Py-H). Calcd. for C₂₅H₃₀N₄O₆.1.4 TFA, 0.8H₂O: C, 50.86; H, 5.07; N, 8.53. Found: C, 50.77; H, 4.98; N, 8.89.

### EXAMPLE 74

3-(2-Methylbenzothiazol-5-yl)-4-{3-[3-(6,7,8,9-tetrahydro-5-oxa-1,9-diaza-benzocyclohepten-2-yl)-propyl]-[1,2,4]oxadiazol-5-yl}-butyric acid.

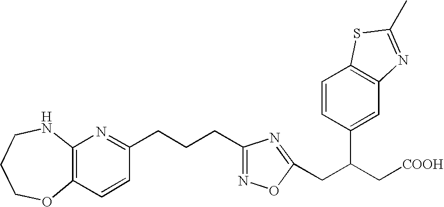

This was prepared utilizing the procedure for the preparation of Example 70 using the appropriate anhydride in step 11. ¹H-NMR (CD₃CN): δ 1.93 (2H, m, CH₂), 1.95 (2H, m, CH₂), 2.18 (2H, m, CH₂), 2.60 (2H, m, CH), 2.72 (3H, s, CH₃), 2.85 (2H, m, CH₂), 3.30 (2H, m, CH₂), 3.60 (2H, t, CH₂), 3.78 (1H, m, CH), 4.28 (2H, t, CH₂), 6.42 (1H, d, Py-H), 7.25 (1H, d, Ar—H), 7.30 (1H, d, Ar—H), 7.78 (1H, s, Ar—H), 7.80 (1H, s, Py-H). Calcd. for C₂₅H₂₇N₅O₄S. 1.3 TFA, 1.0H₂O: C, 50.24; H, 4.63; N, 10.61. Found: C, 50.34; H, 4.37; N, 10.56.

### EXAMPLE 75

Preparation of 3-(1,3-benzodioxol-5-yl)-4-{3-[3-(1,2,3,5-tetrahydropyrido[2,3-e][1,4]oxazepin-8-yl)propyl]-1,2,4-oxadiazol-5-yl}butanoic acid, TFA.

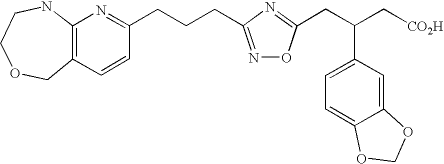

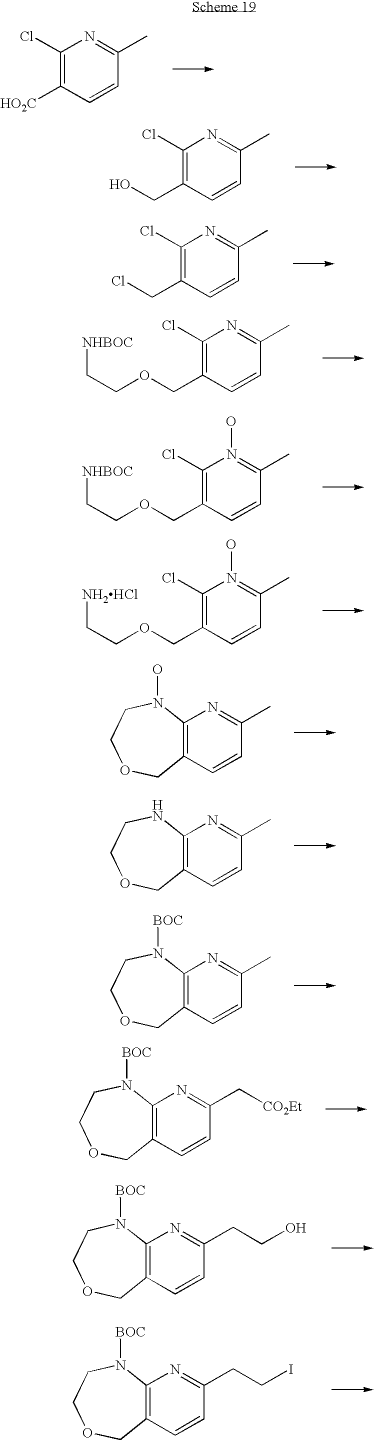

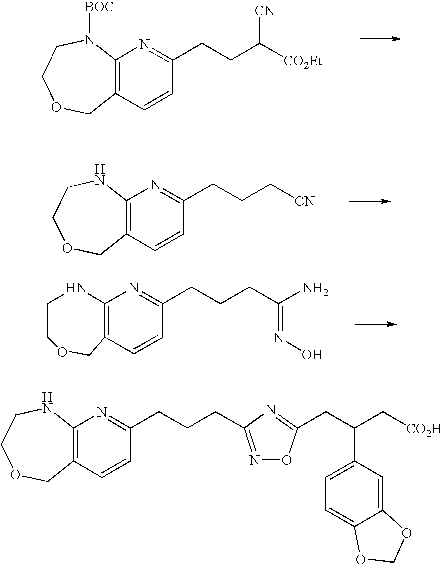

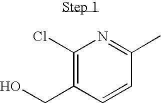

To a 1M solution of borane-tetrahydrofuran complex (200 mL, 200 mmol) at 0° C. under N₂ was added 2-chloro-6-methylnicotinic acid (15 g, 87.42 mmol) in dry THF (45 mL) using a dropping funnel. The ice bath was removed after completion of the addition and the reaction stirred overnight. A mixture of acetic acid (12 mL) and methanol (12 mL) was added dropwise to the reaction flask at 0° C. and stirred for 1 hour. The volatiles were removed in vacuo and the residue was dissolved in water. The solution was neutralized with 1N NaOH and then extracted with EtOAc (3×). The organic layers were washed with brine, dried over Na₂SO₄ and concentrated in vacuo to afford the title compound as a white solid. H NMR (400 MHz, CDCl₃) d 2.53 (s, 3H), 4.75 (s, 2H), 7.13 (d, 1H), 7.74 (d, 2H).

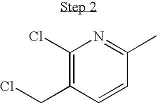

To a solution of the product of step 1 (10 g, 63.7 mmol) in CH₂Cl₂ (150 mL) at room temperature under Ar was added thionyl chloride (16.3 mL, 223 mmol) and the mixture stirred for 4 hours. The mixture was poured into an ice cold water very slowly. The layers were separated and the water layer was extracted with CH₂Cl₂ (2×). The combined organic layers was washed with brine, dried over Na₂SO₄ and concentrated in vacuo to afford the title compound as a white solid. H NMR (400 MHz, CDCl₃) d 2.55 (s, 3H), 4.68 (s, 2H), 7.13 (d, 1H), 7.71 (d, 2H).

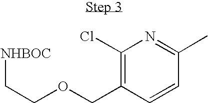

To a solution of t-butyl N-(−2-hydroxyethyl)-carbamate (8.41 g, 52.2 mmol) in DMSO (30 mL) at room temperature was added powder KOH (5.86 g, 104.4 mmol) and followed by the product of step 2 (6 g, 34 mmol). After stirring at room temperature for 2 hours the reaction was quenched with water. After extraction with Et₂O(3×), the organic layers were washed partitioned between water and EtOAc, washed with brine, dried over Na₂SO₄ and concentrated in vacuo to afford the title compound as a yellow oil. H NMR (400 MHz, CDCl₃) d 1.45 (s, 9H), 2.56 (s, 3H), 3.40 (m, 2H), 3.64 (t, 2H), 4.57 (s, 2H), 7.20 (d, 1H), 7.29 (d, 2H).

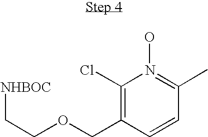

The pyridine from step 3 (8.8 g, 29.3 mmol) and mCPBA (7.6 g, 43.98 mmol) were dissolved in CHCl₃ and stirred at 50° C. overnight. The solution was concentrated in vacuo and purified by flash chromatography (silica, 98:2:0.5, CH₂Cl₂: MeOH: NH₄OH) to yield a yellow oil. H NMR (400 MHz, CDCl₃) d 1.44 (s, 9H), 2.56 (s, 3H), 3.40 (m, 2H), 3.64 (t, 2H), 4.56 (s, 2H), 7.22 (d, 1H), 7.32 (d, 2H).

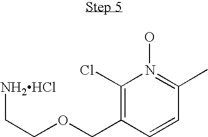

The product of step 4 (5.3 g, 16.8 mmol) was dissolved in HCl-EtOH solution (35 mL) at room temperature and stirred overnight. The reaction was concentrated and dried to yield a white solid.

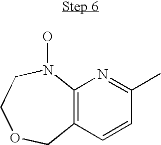

To a solution of the product of step 5 (4.23 g, 16.8 mmol) in t-amyl alcohol (30 mL) at room temperature under N₂ was added NaHCO₃ (7.05 g, 84 mmol) and the mixture was heated to reflux overnight. The reaction was cooled, diluted with CH₂Cl₂ and filtered. The filtrate was concentrated in vacuo and purified by flash chromatography (silica, 98:2:0.5, CH₂Cl₂: MeOH: NH₄OH) to yield a light yellow crystals. H NMR (400 MHz, CDCl₃) δ 2.54 (s, 3H), 3.40 (m, 2H), 3.90 (t, 2H), 4.60 (s, 2H), 6.63 (d, 1H), 6.93 (d, 2H).

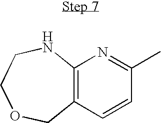

A solution of the product of step 6 (3.47 g, 19.28 mmol), ion powder (1.62 g, 28.9 mmol), triphenylphosphine (5.06 g, 19.28 mmol) and acetic acid (50 ml) was heated to reflux for 1 hour. The solution was cooled, filtrated through a celite bed, and washed with ethyl acetate. The filtrate was concentrated and purified on a silica gel column, eluting with dichloromethane/methanol/ammonium hydroxide (97.5:2:0.5) to afford a light yellow crystals. H NMR (400 MHz, CDCl₃) δ 2.40 (s, 3H), 3.26 (m, 2H), 3.84 (t, 2H), 4.53 (s, 2H), 6.59 (d, 1H), 7.82 (d, 2H).

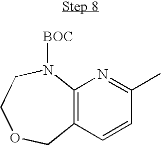

A solution of the product of step 7 (3.4 g, 20.7 mmol), di-tert-butyl dicarbonate (9.05 g, 41.46 mmol), and DMAP (251 mg) in THF (100 mL) was heated to reflux overnight. The reaction mixture was allowed to cool to room temperature and concentrated in vacuo. The residue was crystallized from 20% EtOAc/Hex to afford a brown solid. H NMR (400 MHz, CDCl₃) δ 1.45 (s, 9H), 2.54 (s, 3H), 3.90 (m, 2H), 4.55 (s, 2H), 7.01 (d, 1H), 7.50 (d, 2H).

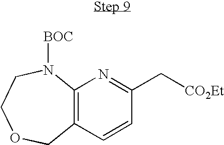

Lithium diisopropylamide solution (4.7 mL, 9.54 mmol, 2.0 M in THF/ethylbenzene/heptane) was added dropwise to a chilled (−78° C.), stirred solution of the product of step 8 (2.1 g, 7.95 mmol) in dry THF (30 mL) under N₂ and the resulting solution stirred for 20 min at −78° C. Diethyl carbonate (3.6 mL, 29.41 mmol) was introduced to the mixture. After 1 hour the reaction was quenched with saturated NH₄Cl solution and warmed to room temperature. The mixture was extracted three times with ethyl acetate and all organic extracts were combined, washed with brine, dried over MgSO₄, and concentrated under reduced pressure to get the crude product, which was purified by chromatography on silica gel (eluent: 25% ethyl acetate/hexane). The desired product is a yellow solid. H NMR (400 MHz, CDCl₃) δ1.27 (t, 3H), 1.44 (s, 9H), 3.83 (s, 2H), 4.16 (q, 2H), 4.57 (s, 2H), 7.19 (d, 1H), 7.60 (d, 2H).

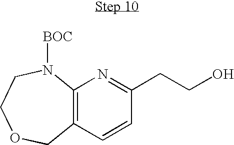

To a solution of the product of step 9 (1.9 g, 5.7 mmol) in dry THF (25 mL) at room temperature was added a solution of LiBH₄ (2.0 M in THF, 3.4 mL, 6.78 mmol), and the resulting mixture was heated to reflux. After 16 hours the mixture was cooled to 0° C. and carefully quenched with water (20 mL). After 10 minutes, the mixture was extracted three times with ethyl acetate. The combined organic extracts were washed with brine, dried over MgSO₄, filtered, and concentrated under reduced pressure to give a yellow solid. H NMR (400 MHz, CDCl₃) δ 1.46 (s, 9H), 3.01 (t, 2H), 3.90 (m, 2H), 4.00 (m, 2H), 4.57 (s, 2H), 7.03 (d, 1H), 7.56 (d, 1H).

Step 11

A mixture of the product of step 10 and 4 M HCl in dioxane (6 mL) was stirred at room temperature for 4 hours, and then concentrated under reduced temperature. The residue was chromatographed on silica gel (eluent: 94.5/5/0.5 chloroform/ethanol/ammonium hydroxide) to afford a yellow oil. H NMR (400 MHz, CDCl₃) δ 3.11 (t, 2H), 3.63 (m, 2H), 3.96 (m, 2H), 4.05 (t, 3H), 4.67 (s, 2H), 6.74 (d, 1H), 7.60 (d, 1H).

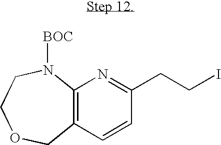

To a stirred, cooled (0° C.) solution of the product of step 10 (1.26 g, 4.28 mmol), triphenylphosphine (1.46 g, 5.56 mmol) and imidazole (417 mg, 6.12 mmol) in CH₃CN (6 mL) and dry ether (8 mL) was slowly added iodine (1.52 g, 6 mmol) and then stirred for 1 hour. The resulting mixture was added 100 mL ether, washed successively with saturated aqueous Na₂S₂O₃ and brine, dried over Na₂SO₄ and concentrated under reduced pressure. The residue was purified by flash chromatography (silica, 20% EtOAC/Hex) to afford a yellow solid. H NMR (400 MHz, CDCl₃) δ 1.45 (s, 9H), 3.35 (t, 2H), 3.50 (t, 2H), 3.92(s, 2H), 4.57 (s, 2H), 7.04 (d, 1H), 7.57 (d, 1H).

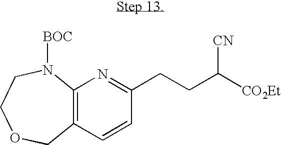

NaH (208 mg of a 60% weight dispersion in mineral oil, 5.19 mmol) was suspended in DMF (15 mL) at 0° C. under N₂. Ethyl cyanoacetate (0.405 mL, 3.8 mmol) was added and the resulting mixture stirred for 30 min at 0° C. The product of step 12 (1.4 g, 3.46 mmol) in DMF (2 mL) was introduced to the reaction mixture and stirred for 1 hour at room temperature. The mixture was cooled to 0 and quenched with water and extracted with EtOAc (3×). The organic layers were washed with brine, dried over Na₂SO₄ and concentrated in vacuo. The residue was purified by flash chromatography (silica, 50% EtOAC/Hex) to afford a colorless oil. H NMR (400 MHz, CDCl₃) δ 1.32 (t, 3H), 1.44 (s, 9H), 2.31 (m, 1H), 2.50 (m, 1H), 3.00 (m, 2H), 3.66 (m, 1H), 3.90 (s, 2H), 4.26 (q, 2H), 4.57 (s, 2H), 7.05 (d, 1H), 7.55 (d, 1H).

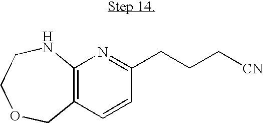

A mixture of the product of step 13 (700 mg, 1.8 mmol) and KOH (powder, 152 mg, 2.7 mmol) in ethylene glycol (8 mL) under N₂ was heated at 150° C. for 3 hours. The mixture was cooled to 0° C. and portioned between water and EtOAc. The organic phase was washed with brine, dried over Na₂SO₄ and concentrated in vacuo. Flash chromatography (silica, EtOAc) yielded a light yellow oil. H NMR (400 MHz, CDCl₃) δ 2.07 (t, 3H), 2.37 (t, 1H), 2.77 (t, 2H), 3.24 (m, 2H), 3.85 (m, 2H), 4.53 (s, 2H), 4.98 (s, 1H), 6.61 (d, 1H), 7.33 (d, 1H).

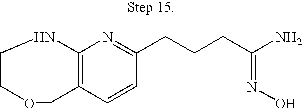

A mixture of the product of step 14 (270 mg, 1.24 mmol) and hydroxylamine (0.18 mL of a 50% weight solution in water, 2.73 mmol) in ethanol (4 mL) under N₂ was heated at 60° C. overnight. The mixture was cooled to room temperature and concentrated in vacuo to yield a white solid.

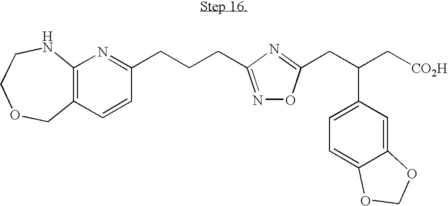

A mixture of the product of step 15 (110 mg, 0.44 mmol) and the anhydride from Example 1, step 1-3, (93 mg, 0.4 mmol) in 1,4-dioxane (3 mL) was heated at 90° C. overnight. The reaction mixture was allowed to cool to room temperature and concentrated. The residue was purified on HPLC using acetonitrile gradient 15-50% in 30 min to yield 56 mg desired product as a yellow oil. FAB-MS:(MH+)=467. H NMR (500 MHz, CD₃OD) δ 2.08 (m, 2H), 2.72 (m, 5H), 3.26 (m, 2H), 3.61(m, 1H), 3.68 (t, 2H), 3.98 (t, 2H), 4.25 (s, 2H), 5.84 (m, 2H), 6.67 (m, 2H), 6.76 (s, 2H), 6.79 (d, 1H), 7.78 (d, 1H). Anal Calcd. for C₂₄H₂₆N₄O₆ plus 1.3 CF₃COOH: C, 51.98; H, 4.48; N, 9.11. Found: 51.67; H, 4.54; N, 9.24.

### EXAMPLE 76

Preparation of 3-(3,5-dimethoxyphenyl)-4-{3-[3-(1,2,3,5-tetrahydropyrido[2,3-e][1,4]oxazepin-8-yl)propyl]-1,2,4-oxadiazol-5-yl}butanoic acid, TFA.

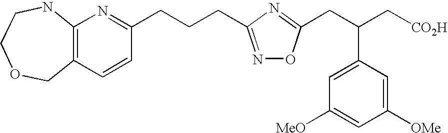

A mixture of the product of Example 75, step 15 (176 mg, 0.70 mmol) and the appropriate anhydride prepared from 3,5-dimethoxybenzaldehyde as in Example 1, step 1-3 (160 mg, 0.63 mmol) in 1,4-dioxane (4 mL) was heated at 90° C. overnight. The reaction mixture was allowed to cool to room temperature and concentrated. The residue was purified on HPLC using acetonitrile gradient 15-50% in 30 min to yield 35 mg desired product as a yellow oil. FAB-MS:(MH+)=483. H NMR (500 MHz, CD₃OD) δ 2.08 (m, 2H), 2.74 (m, 5H), 3.28 (m, 2H), 3.62 (m, 1H), 3.69 (m, 8H), 3.78 (m, 1H), 4.00 (t, 2H), 4.84 (s, 2H), 6.27, (t, 1H), 6.37 (d, 2H), 6.77 (d, 1H), 7.77 (d, 1H).

### EXAMPLE 77

3-(1,3-Benzodioxol-5-yl)-4-(3-{3-[6-(methylamino)pyridin-2-yl]propyl}-1,2,4-oxadiazol-5-yl)butanoic acid hydrochloride 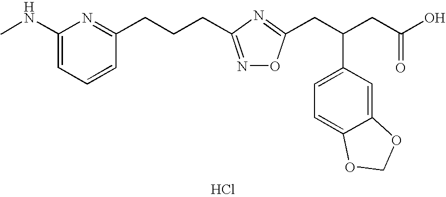 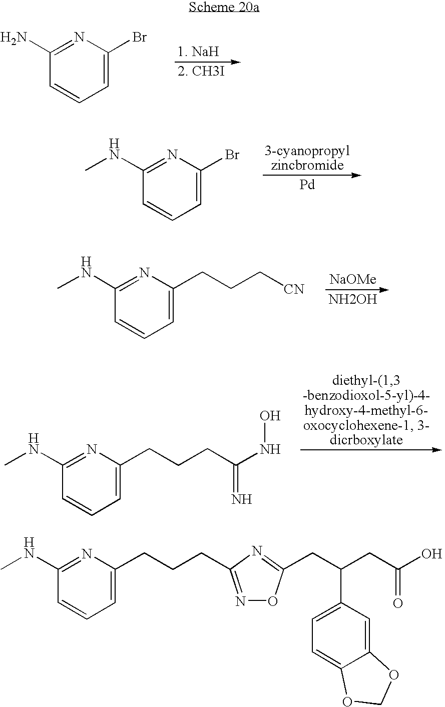
Step 1: 6-Bromo-N-methylpyridin-2-amine.

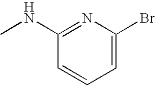

Sodium hydride (95%, 1.0108 g) was suspended in THF (25 mL) under nitrogen atmosphere. A solution of 2-amino-6-bromopyridine (7.3 g) in THF (50 mL) was added slowly to this mixture at room temperature. This mixture was stirred at an ambient temperature for 30 minutes. The mixture was quenched with Iodomethane (5.0 mL). The reaction mixture was stirred at room temperature for 30 minutes before quenching with water and extracting with ethyl acetate. The ethyl acetate extract was washed with brine, dried (Na₂SO₄) and concentrated to afford crude oil, which was purified with silica gel chromatography (Biotage Flash 40M) to afford 6.1 g of the title compound as pale yellow oil. ¹HNMR(CD₃OD): δ 7.95(t, 1H), 7.65(m, 1H), 7.42(d, 1H), 7.05(d, 1H), 3.55(d, 3H).
Step 2: 4-[6-(Methylamino)pyridin-2-yl]butanenitrile.

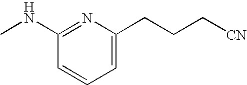

The product of step 1 (3.45 g, 20 mmol) was dissolved in THF (50 mL) and was treated with tetrakis(triphenylphosphine)palladium (0). The mixture was stirred at an ambient temperature for 15 minutes under nitrogen atmosphere and was treated with 0.4M solution of 3-cyanopropylzincbromide in THF. The mixture was stirred at room temperature under nitrogen atmosphere for 20 hours. The mixture was quenched with a saturated solution of sodium bicarbonate and extracted with ethyl acetate. The ethyl acetate extract was washed with brine, dried (Na₂SO₄) and concentrated to afford crude oil, which was purified using reversed phase HPLC (water with 2% TFA/CH₃CN mobile phase) to afford 1.4 g of the title compound as TFA salt. ¹HNMR(CD₃OD): δ7.75(t, 1H), 6.65(m, 2H), 3.05(s, 3H), 2.82 (t, 2H), 2.45 (t, 2H), 2.1 (m, 2H).
Step 3: N-hydroxy-4-[6-(methylamino)pyridin-2-yl]butanimidamide.

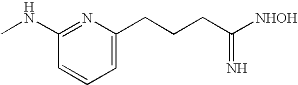

Sodium metal (0.715 g, 31 mmol) was dissolved in MeOH (30 mL) under nitrogen atmosphere and hydroxylamine hydrochloride (2.16 g) was added to the mixture. The mixture was stirred at an ambient temperature under nitrogen atmosphere for 2 hours. The solid was filtered under vacuum and the filtrate was added to the free base of the product from step 2 and was heated to 41° C. and was stirred at 41° C. for 48 hours and the mixture was quenched with water (1 mL) and was concentrated to afford crude oil, which was purified using reversed phase HPLC (water with 2% TFA/CH₃CN mobile phase) to afford 0.520 g of the title compound as TFA salt. ¹HNMR(CD₃OD): δ7.55(m, 1H), 6.55(m, 2H), 3.45(s, 3H), 2.72 (t, 2H), 2.35 (t, 2H), 2.1 (m, 2H).
Step 4: 3-(1,3-Benzodioxol-5-yl)-4-(3-{3-[6-(methylamino)pyridin-2-yl]propyl}-1,2,4-oxadiazol-5-yl)butanoic acid hydrochloride.

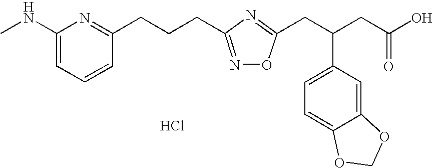

The product from step 3 (322 mg) was dissolved in dioxane (5 mL) containing triethylamine (200 mg) and was treated with the appropriate anhydride (as in Example 1) (210 mg) in a scintillation vial and was heated to 90° C. for 16 hours. The mixture was concentrated to afford crude oil, which was purified using reversed phase HPLC (water with 2% HCl/CH₃CN mobile phase) to afford 0.120 g of the title compound as HCl salt.

¹HNMR(CD₃OD): δ 7.85(t, 1H), 6.92(d, 1H), 6.78(s, 1H), 6.68(m, 3H), 5.87(s, 2H), 3.62(m, 1H), 3.25(m, 1H), 3.05(s, 3H), 2.75(m, 6H), 2.10(m, 2H). Mass Spectrum: (MH+): 425.2.

### EXAMPLE 78

3-(3-Fluorophenyl)-4-(3-{3-[6-(methylamino)pyridin-2-yl]propyl}-1,2,4-oxadiazol-5-yl)butanoic acid trifluoroacetate.

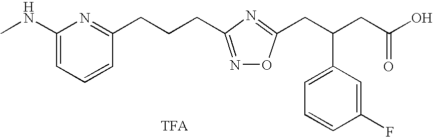

This compound was prepared by following the procedure described in Example 77, step 4 using the appropriate anhydride. ¹HNMR(CD₃OD): δ 7.85(t, 1H), 7.28(m, 1H), 7.10(d, 1H), 7.05(m, 1H), 6.88(m, 2H), 6.7(d, 1H), 3.72(m, 1H), 3.35(m, 2H), 3.05(s, 3H), 2.75(m, 6H), 2.10(m, 2H). Mass Spectrum: (MH+): 399.2.

### EXAMPLE 79

3-(1,3-benzodioxol-5-yl)-4-(3-{3-[6-(ethylamino)pyridin-2-yl]propyl}-1,2,4-oxadiazol-5-yl)butanoic acid trifluoroacetate. 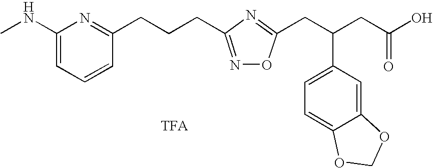
Step 1: 6-bromo-N-ethylpyridin-2-amine.

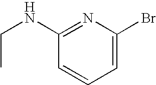

This compound was prepared following the procedure described in Example 77, step: 1 and replacing iodomethane with iodoethane. The compound was isolated as pale yellow oil. ¹HNMR(CD₃OD): δ 7.45(t, 1H), 6.45(d, 1H), 6.22(d, 1H), 3.25(m, 2H), 1.25(t, 3H),
Step 2: 4-[6-(ethylamino)pyridin-2-yl]butanenitrile.

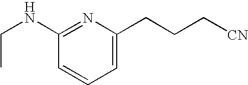

This compound was prepared following the procedure described in Example 77, Step 2. The compound was isolated as TFA salt. ¹HNMR(CD₃OD): δ 7.45(t, 1H), 6.45(d, 1H), 6.22(d, 1H), 4.5(m, 1H), 3.25(m, 2H), 2.72 (t, 2H), 2.35 (t, 2H), 2.1 (m, 2H), 1.25(t, 3H),
Step 3: 4-[6-(Ethylamino)pyridin-2-yl]-N-hydroxybutanimidamide.

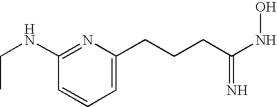

This compound was prepared following the procedure described in Example 77, step 3 using the product of step 2 of this example. The compound was isolated as TFA salt. ¹HNMR(CD₃OD): δ 7.55(m, 1H), 6.55(m, 2H), 3.45(m, 2H), 2.72 (t, 2H), 2.35 (t, 2H), 2.1 (m, 2H), 1.25(t, 3H),
Step 4: 3-(3-Fluorophenyl)-4-(3-{3-[6-(methylamino) pyridin-2-yl]propyl-1,2,4-oxadiazol-5-yl)butanoic acid trifluoroacetate.

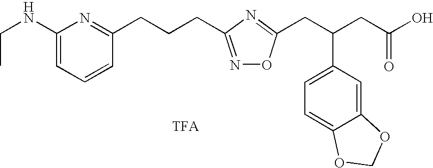

The product from step 3 (336 mg) was dissolved in dioxane (5 mL) containing triethylamine (200 mg) and was treated with the appropriate anhydride (as in Example 1) (210 mg) in a scintillation vial and was heated to 90° C. for 16 hours. The mixture was concentrated to afford crude oil, which was purified using reversed phase HPLC (water with 2% TFA/CH₃CN mobile phase) to afford 0.290 g of the title compound as TFA salt. ¹HNMR(CD₃OD): 7.85(m, 1H), 6.92(d, 1H), 6.78 (s, 1H), 6.68 (m, 3H), 5.87(s, 2H), 3.62(m, 1H), 3.45(q, 2H), 3.25(m, 2H), 2.75(m, 6H), 2.10(m, 2H), 1.35(t, 3H). Mass Spectrum: (MH+): 439.17.

### EXAMPLE 80

3-(3-Fluorophenyl)-4-(3-{3-[6-(methylamino) pyridin-2-yl]propyl-1,2,4-oxadiazol-5-yl)butanoic acid trifluoroacetate.

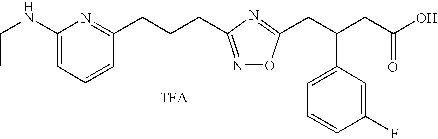

The product from Example 79, step 3 (336 mg) was dissolved in dioxane (5 mL) containing triethylamine (200 mg) and was treated with the appropriate anhydride (200 mg) in a scintillation vial and was heated to 90° C. for 16 hours. The mixture was concentrated to afford crude oil, which was purified using reversed phase HPLC (water with 2% TFA/CH₃CN mobile phase) to afford 0.130 g of the title compound as TFA salt. ¹HNMR(CD₃OD): δ7.85 (m, 1H), 7.3(m, 1H), 7.1(d, 1H), 7.05(m, 1H), 6.92(d, 1H), 6.88 (d, 1H), 6.68 (d, 2H), 5.87, 3.72(m, 1H), 3.45(q, 2H), 3.25(m, 2H), 2.75(m, 6H), 2.10(m, 2H), 1.35 (t, 3H). Mass Spectrum: (MH+): 439.17.

### EXAMPLE 81

3-(1,3-Benzodioxol-5-yl)-4-(3-{4-[(4-methylpyridin-2-yl)amino]butyl}-1,2,4-oxadiazol-5-yl)butanoic acid. 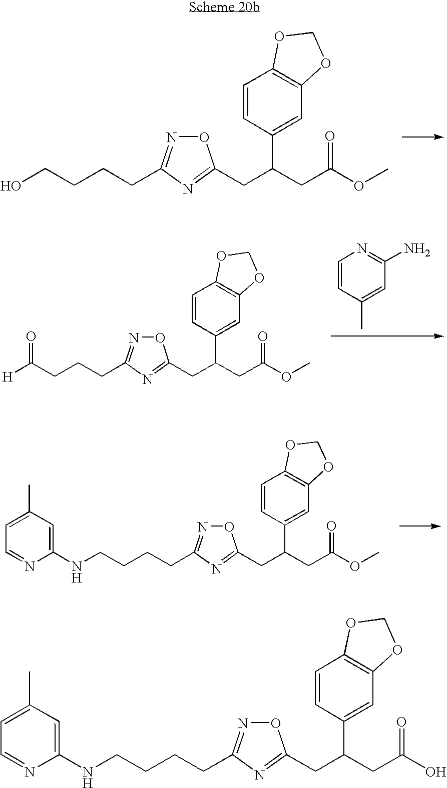
Step 1. Methyl 3-(1,3-benzodioxol-5-yl)-4-[3-(4-oxobutyl)-1,2,4-oxadiazol-5-yl]butanoate.

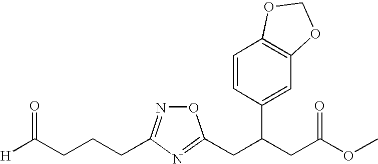

A mixture of methyl 3-(1,3-benzodioxol-5-yl)-4-[3-(4-hydroxybutyl)-1,2,4-oxadiazol-5-yl]butanoate (described in earlier examples) (2.4 g, 6.5 mmol), N-methylmorpholine-N-oxide (1.1 g, 9.7 mmol), molecular sieves (3.3 g), acetonitrile (20 mL), and dichloromethane (20 mL) was stirred at room temperature. After 10 min, tetrapropylammoium perruthenate (0.12 g, 0,32 mmol) was added. The resulting reaction was stirred for 2 h, filtered through a pad of Celite (2″), and washed with dichloromethane (30 mL). The filtrate was concentrated. The residue was purified by chromatography (on silica gel, Toluene/ethyl acetate=6/4) to give a clear oil in 1.5 g (60%). The NMR spectra were consistent for the proposed structure.
Step 2. Methyl 3-(1,3-benzodioxol-5-yl)-4-(3-{4-[(4-methylpyridin-2-yl)amino]butyl}-1,2,4-oxadiazol-5-yl)butanoate.

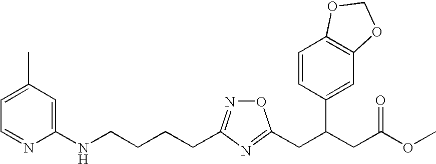

A mixture of the product of step 1 (0.28 g, 0.78 mmol), 2-amino-4-picoline (0.10 g, 0.93 mmol), sodium triacetoxyborohydride (0.26 g, 1.2 mmol), and dichloromethane (20 mL) was stirred at room temperature. After 18 h, the reaction was diluted with ethyl acetate (150 mL). The organic layer was washed with H₂O (50 mL), brine (50 mL), dried over MgSO₄ and concentrated. The residue was purified by chromatography (on silica gel, toluene/ethyl acetate85/15) to afford viscous oil in 0.20 g (57%). The NMR spectra were consistent for the proposed structure.
Step 3. 3-(1,3-Benzodioxol-5-yl)-4-(3-{4-[(4-methylpyridin-2-yl)amino]butyl}-1,2,4-oxadiazol-5-yl)butanoic acid.

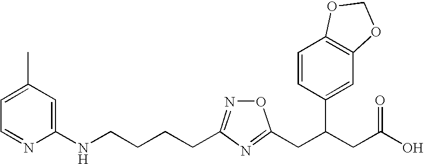

A solution of the product of step 2 (0.2 g, 0.44 mmol) in sodium hydroxide (15 mL, 1 N) and methanol (25 mL) was stirred at room temperature. After 18 h, the reaction was acidified with trifluoroacetic acid (1.5 mL), and concentrated. The residue was purified on HPLC using acetonitrile gradient 10-50% in 30 min to give a gummy solid in 95 mg (37%). ¹H NMR (CDCl₃) δ 9.13 (1H, br. s); 7.61 (1H, d); 6.60-6.66 (3H, m); 6.49-6.52 (2H, m); 5.86 (1H, s); 5.28 (3H, s); 3.63 (1H, m); 3.08-3.25 (4H, m); 2.63-2.79 (4H, m); 1.79 (2H, m); 1.66 (2H, m). Anal. Calcd for C₂₃H₂₆N₄O₅. 1.25 CF₃COOH.0.25H₂O C, 52.31; H, 4.78; N, 9.57; found C, 52.01; H, 4.78; N, 9.53.

### EXAMPLE 82

3-(1,3-benzodioxol-5-yl)-4-(3-{4-[(6-methylpyridin-2-yl)amino]butyl}-1,2,4-oxadiazol-5-yl)butanoic acid.

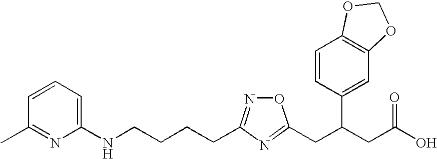

This compound was prepared as for Example 81 using 2-amino-6-picoline in place of 2-amino-4-picoline in step 2. ¹H NMR (MeOD) δ 7.78 (1H, t, J=8 Hz); 6.85 (1H, dd, J=8,2); 6.70 (1H, d, J=2 Hz); 6.68 (2H, dd, J=8,2 Hz); 7.65 (1H, s); 5.85 (2H, s); 3.57-3.64 (1H, m); 3.37 (2H, t, 8 Hz); 3.15-3.32 (2H, m); 2.64-2.81(4H, m); 2.50 (3H, s); 1.76-1.84 (2H, m); 1.62-1.70 (2H, m). Anal. Calcd for C₂₃H₂₆N₄O₅.1.6 CF₃COOH C, 50.68; H, 4.48; N, 9.02; found C, 50.83; H, 4.56; N, 8.86.

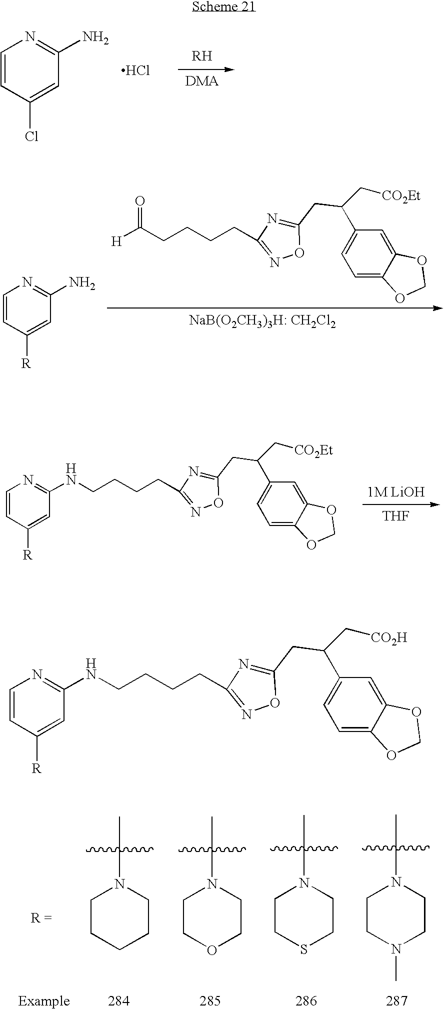

### EXAMPLE 83

3-(1,3-Benzodioxol-5-yl)-4-(3-{4-[(4-piperidin-1-ylpyridin-2-yl)amino]butyl}-1,2,4-oxadiazol-5-yl)butanoic acid hydrochloride. 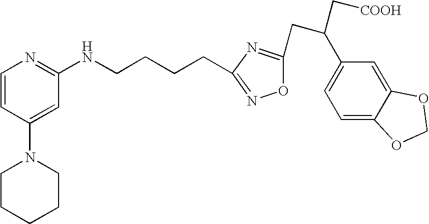
Step 1. Preparation of 4-piperidin-1-ylpyridin-2-amine.

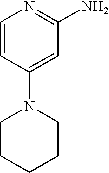

4-Chloropyridin-2-amine 4-chloropyridin-2-amine, synthesized according to procedures outlined in Sundberg, Richard; Jiang, Songchun; Organic Preparation and Procedure; 29 (1), 1997, 117-122, (300 mg, 1.786 mmol) was combined with piperidine (2.5 mL) in 2 mL DMA in a sealed vessel. This mixture was heated in the microwave (CSA Discover) for 5 minutes at 200° C. Upon cooling, the reaction was concentrated in vacuo and purified via silica gel chromatography (eluent: 95/5/0.5 CH₂Cl₂/MeOH/NH₄OH) to give the product as a yellow solid. Yield: 114 mg (45%). ¹H NMR (DMSO-d₆) δ 7.63-7.54 (m, 1H), 6.56-6.52 (m, 1H), 6.01-5.96 (m, 1H), 3.50-3.42, (m, 4H), 1.68-1.60 (m, 2H), 1.59-1.50 (m, 4H).
Step 2. Preparation of 3-(1,3-benzodioxol-5-yl)-4-(3-{4-[(4-piperidin-1-ylpyridin-2-yl)amino]butyl}-1,2,4-oxadiazol-5-yl)butanoic acid hydrochloride.

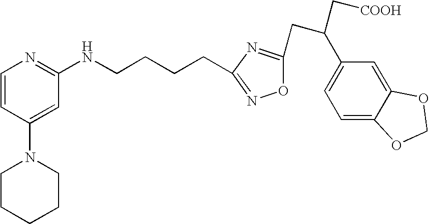

A mixture of 4-piperidin-1-ylpyridin-2-amine (114 mg, 0.643 mmol) and ethyl(3R)-3-(1,3-benzodioxol-5-yl)-4-[3-(5-oxopentyl)-1,2,4-oxadiazol-5-yl]butanoate (0.578 mmol) was dissolved in CH₂Cl₂ under argon. After stirring at room temperature for 30 minutes, sodium triacetoxyborohydride was added and the reaction was stirred at room temperature for 18 hours. The reaction was quenched with H₂O and extracted with ethyl acetate 3×. The organic layers were dried over MgSO4, filtered, and concentrated in vacuo. The residue was purified via reverse phase HPLC using a gradient of 10-60% CH₃CN/H₂O/0.5% HCl over 30 minutes to obtain the crude product. The crude product was dissolved in THF (2 mL) at room temperature. 1M LiOH (2.5 mL) was added, and the reaction was allowed to stir at room temp. for 20 hours. The reaction was acidified to pH=1 with conc. HCl and concentrated in vacuo. The residue was purified via reverse phase HPLC using a gradient of 10-50% CH₃CN/H₂O/0.5% HCl over 30 minutes to obtain the desired product. Yield: 16% over 2 steps. ¹H NMR (DMSO-d₆) δ 7.60-7.53 (m, 1H), 6.92-6.87 (m, 1H), 6.27-6.22 (m, 1H), 6.65-6.61 (m, 1H), 6.56-6.51 (m, 1H), 5.97-5.90 (m, 3H), 3.55-3.43 (m, 5H), 3.30-3.13 (m, 4H), 2.77-2.53 (m, 4H), 1.74-1.61 (m, 4H), 1.59-1.47 (m, 6H). Analysis Calculated for C₂₇H₃₃N₅O₅.3.4 HCl: C, 51.35; H, 5.81; N, 11.09. Found C, 51.80; H, 6.23; N, 11.07.

### EXAMPLE 84

3-(1,3-benzodioxol-5-yl)-4-(3-{4-[(4-morpholin-4-ylpyridin-2-yl)amino]butyl}-1,2,4-oxadiazol-5-yl)butanoic acid hydrochloride

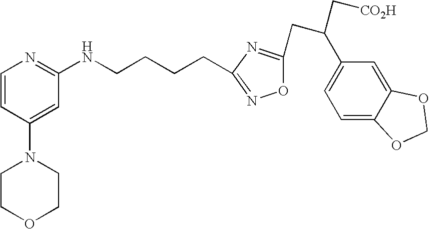

This compound was prepared according to the method described for in Example 83 using morpholine in place of piperidine. Yield: 12% over 2 steps. ¹H NMR (DMSO-d₆) δ 7.67-7.60 (m, 1H), 6.92-6.88 (m, 1H), 6.78-6.75 (m, 1H), 6.67-6.62 (m, 1H), 6.68-6.54 (m, 1H), 6.02-5.97 (m, 1H), 5.95 (s, 2H), 3.73-3.67 (m, 4H), 3.52-3.38 (m, 5H), 3.31-3.15 (m, 4H), 2.78-2.54 (m, 4H), 1.74-1.64 (m, 2H), 1.58-1.48 (m, 2H).

Analysis Calculated for C₂₆H₃₁N₅O₆.2.4 HCl. C, 52.30; H, 5.64; N, 11.73. Found C, 52.15; H, 6.25; N, 11.72. Calculated Mass: 509.55. Found Mass: 510.23 (for MH⁺)

### EXAMPLE 85

3-(1,3-benzodioxol-5-yl)-4-(3-{4-[(4-thiomorpholin-4-ylpyridin-2-yl)amino]butyl}-1,2,4-oxadiazol-5-yl)butanoic acid hydrochloride.

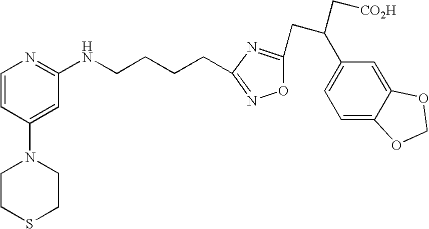

This compound was prepared according to the method described in Example 83, using thiomorpholine in place of piperidine. Yield: 17% over 2 steps. ¹H NMR (DMSO-d₆) δ 7.58-7.65 (m, 1H), 6.93-6.88 (m, 1H), 6.75-6.72 (m, 1H), 6.65-6.62 (m, 1H), 6.58-6.55 (m, 1H), 5.98-5.92 (m, 3H), 3.90-3.82 (m, 4H), 3.52-3.41 (m, 1H), 3.32-3.14 (m, 4H), 2.76-2.54 (m, 8H), 1.73-1.64 (m, 2H), 1.57-1.48 (m, 2H). Analysis Calculated for C₂₆H₃₁N₅O₅S.2.4 HCl: C, 50.93; H, 5.49; N, 11.42. Found: C, 50.93; H, 6.11; N, 11.17. Calculated Mass: 525.62. Found Mass: 526.21 (for MH⁺)

### EXAMPLE 86

3-(1,3-Benzodioxol-5-yl)-4-[3-(4-{[4-(4-methylpiperazin-1-yl)pyridin-2-yl]amino}butyl)-1,2,4-oxadiazol-5-yl]butanoic acid hydrochloride.

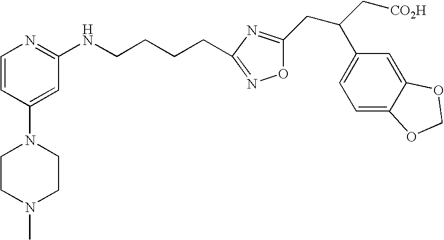

The compound was prepared according to the method described in Example 83, using N-methylpiperidine in place of piperidine. Yield: 8% over 2 steps. ¹H NMR (DMSO-d₆) δ 7.77-7.67 (m, 2H), 6.92-6.88 (m, 1H), 6.78-6.74 (m, 1H), 6.67-6.58 (m, 2H), 6.13-6.09 (m, 1H), 5.95 (s, 1H), 3.58-3.04 (m, 13H), 2.84-2.78 (m, 3H), 2.80-2.55 (m, 4H), 1.75-1.65 (m, 2H), 1.59-1.50 (m, 2H). Elemental Analysis Calculated for C₂₇H₃₄N₆O₅.3.7 HCl: C, 49.32; H, 5.78; N, 12.78. Found: C, 49.37; H, 6.72; N, 12.29. Calculated Mass: 522.60. Found Mass: 523.26 (for MH⁺).

### EXAMPLE 87

(3S)-3-(1,3-benzodioxol-5-yl)-4-{3-[4-(ethanimidoylamino)butyl]-1,2,4-oxadiazol-5-yl}butanoic acid hydrochloride. 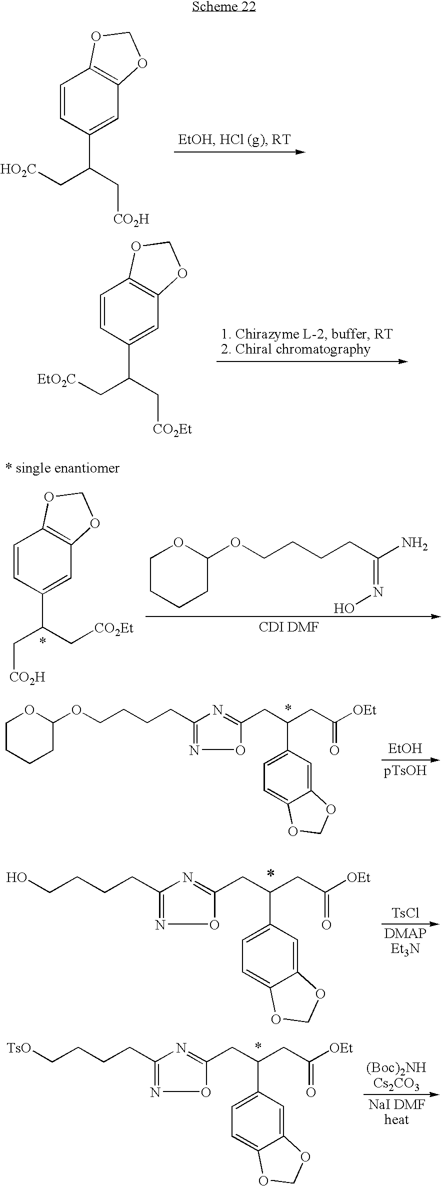 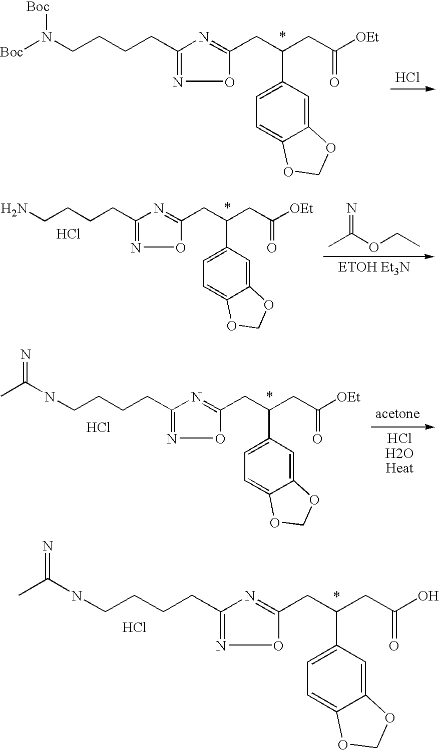
Step 1. Diethyl 3-(1,3-benzodioxol-5-yl)pentanedioate

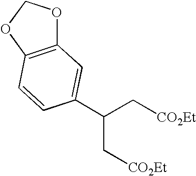

To 3-(1,3-benzodioxol-5-yl)pentanedioic acid (3.86 g, 15.3 mol) was added absolute ethanol (19 mL). The reaction mixture was cooled to −10° C. and HCl gas was passed to saturate the ethanolic reaction mixture at between −10° C. to 20° C. The mixture was then stirred at RT for 2 h resulting in the formation of a solution. The solution was concentrated to give 4.79 g (quantitative yield) of liquid, which was 97% pure by HPLC. ¹H NMR (400 MHz, CDCl₃) δ 1.18 (6H), 2.56 (2H, dd), 2.67 (2H, dd), 3.52-3.62 (1H, m), 4.15 (4H, two overlapping q), 5.92 (2H, s), 6.67-6.72 (3H, m).
Step 2. (3S)-3-(1,3-benzodioxol-5-yl)-5-ethoxy-5-oxopentanoic acid.

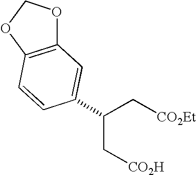

Diethyl 3-(1,3-benzodioxol-5-yl)pentanedioate (4.12 g, 13.4 mol) and 28 mM potassium phosphate buffer pH 7.4 (120 mL) were mixed with stirring to form a dispersion. Chirazyme L-2 enzyme (200 mg, 4.85 wt %) was added and the mixture was stirred at RT while the pH was maintained around 7.28 using an automatic titrator. After 24 h, another portion of Chirazyme L-2 enzyme (200 mg, 4.85 wt %) was added. The mixture was stirred for a total of 140 h. A total of ca. 20 mL of 1 N NaOH solution was automatically added during the addition. The reaction mixture was filtered on a Whatman no.1 filter paper. The filtrate was acidified with 3 N HCl (6 mL) and saturated with sodium chloride. The sodium chloride was filtered off and the filtrate was extracted with ethyl acetate (2×60 mL). The ethyl acetate extracts were washed with brine (2×150 mL), dried with sodium sulfate, and then concentrated to give 3.50 g (93% mass recovery) of a pale yellow oil. Chiral HPLC analysis showed a 93:7 ratio of desired and undesired enantiomers (i.e., 86% ee). The crude product was further purified by chiral stationary phase chromatography to give a total of 2.86 g (76% yield) of the desired product. ¹H NMR (400 MHz, CDCl₃) δ 1.18 (3H, t), 2.65 (4H, four overlapping dd), 3.67 (1H, quintet), 4.15 (4H, q), 5.91 (2H, s), 6.70 (3H, m). [α]₅₈₉ −6.9 (c 1.017, CHCl₃); [α]₃₆₅ −29.5 (1.017, CHCl₃).
Step 3. Ethyl 3-(1,3-benzodioxol-5-yl)-4-{3-[4-(tetrahydro-2H-pyran-2-yloxy)butyl]-1,2,4-oxadiazol-5-yl}butanoate.

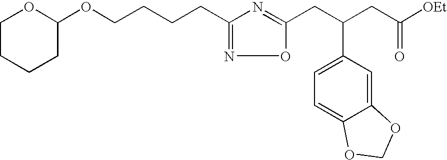

(3S)-3-(1,3-Benzodioxol-5-yl)-5-ethoxy-5-oxopentanoic acid (30.6 g, 104 mmol), CDI (16.9 g, 104 mmol) and DMF (200 mL) were added to a 3-N RBF and allowed to stir under nitrogen for a half hour. (1E)-N′-hydroxy-5-(tetrahydro-2H-pyran-2-yloxy)pentanimidamide (22.5 g 104 mmol) was then added with more DMF (200 mL) and the reaction was allowed to stir for twenty-four hours. The reaction was then heated to 90° C. The DMF was then removed and ethyl acetate and water were added. The water layer was washed three times with ethyl acetate. The ethyl acetate was dried and condensed to give pure product weighing 40 g (84% yield).
Step 4. Ethyl 3-(1,3-benzodioxol-5-yl)-4-[3-(4-hydroxybutyl)-1,2,4-oxadiazol-5-yl]butanoate.

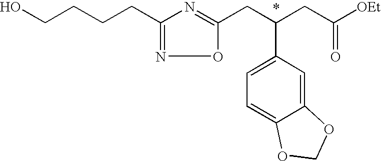

P-toluenesulfonic acid (165 mg, 0.87 mmol), ethyl 3-(1,3-benzodioxol-5-yl)-4-{3-[4-(tetrahydro-2H-pyran-2-yloxy)butyl]-1,2,4-oxadiazol-5-yl}butanoate (40 g, 87 mmol), and ethanol (300 mL) were allowed to stir for twenty-four hours. Methylene chloride (300 mL) and PS-DIEA resin (0.921 g, 3.18 mmol) were added and the reaction was again allowed to stir for twenty-four hours. The reaction mixture was then passed through a filter and the solvent was removed to give ethyl 3-(1,3-benzodioxol-5-yl)-4-[3-(4-hydroxybutyl)-1,2,4-oxadiazol-5-yl]butanoate (32 g, 85%)
Step 5. Ethyl 3-(1,3-benzodioxol-5-yl)-4-[3-(4-{[(4-methylphenyl)sulfonyl]oxy}butyl)-1,2,4-oxadiazol-5-yl]butanoate.

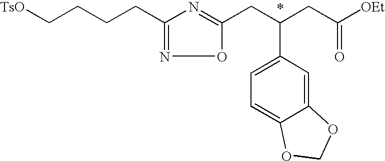

Ethyl 3-(1,3-benzodioxol-5-yl)-4-[3-(4-hydroxybutyl)-1,2,4-oxadiazol-5-yl]butanoate (30 g, 80 mmol) in methylene chloride (300 mL) was cooled to 0° C. Then tosyl chloride (20 g, 104 mmol), DMAP (1 g, 8 mmol), and triethylamine (33 mL, 239 mmol) were added and the reaction was placed in the refrigerator for thirty-six hours. The reaction was not complete so the reaction was allowed to stir at room temperature for 18 hours. The solvent was stripped off. The crude product was put through a silica column using 3:2 ethyl acetate to hexane as the eluent. The column did not purify all of the product so another column was run using a gradient that started at 5:1 hexane to ethyl acetate and ended at 1:1 hexane to ethyl acetate. The collected fractions yielded 30.5 g (72%) of ethyl 3-(1,3-benzodioxol-5-yl)-4-[3-(4-{[(4-methylphenyl)sulfonyl]oxy}butyl)-1,2,4-oxadiazol-5-yl]butanoate
Step 6. Ethyl 3-(1,3-benzodioxol-5-yl)-4-(3-{4-[bis(tert-butoxycarbonyl)amino]-butyl}-1,2,4-oxadiazol-5-yl)butanoate.

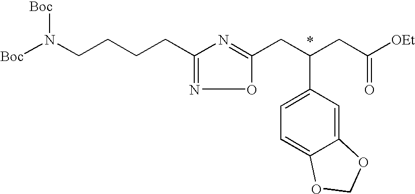

Ethyl 3-(1,3-benzodioxol-5-yl)-4-[3-(4-{[(4-methylphenyl)sulfonyl]oxy}butyl)-1,2,4-oxadiazol-5-yl]butanoate (30.5 g, 57.5 mmol), Cesium Carbonate (28.1 g, 86 mmol), and Sodium Iodide (0.86 g, 5.75 mmol), di-tert-butyl iminodicarboxylate (13.7 g, 63 mmol), and DMF (300 mL) were stirred at 80° C. for two hours. The reaction was allowed to cool and then water and ethyl acetate were added. The aqueous layer was washed three times with ethyl acetate. The ethyl acetate fractions were combined and washed with water, and brine. The ethyl acetate was then dried and condensed to give ethyl 3-(1,3-benzodioxol-5-yl)-4-(3-{4-[bis(tert-butoxycarbonyl)amino]butyl}-1,2,4-oxadiazol-5-yl)butanoate (29 g, 86%).
Step 7. Ethyl 4-[3-(4-aminobutyl)-1,2,4-oxadiazol-5-yl]-3-(1,3-benzodioxol-5-yl)butanoate hydrochloride.

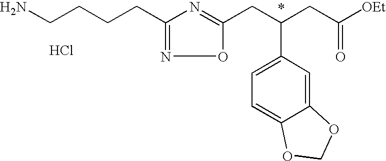

Ethyl 3-(11,3-benzodioxol-5-yl)-4-(3-{4-[bis(tert-butoxycarbonyl)amino]butyl}-1,2,4-oxadiazol-5-yl)butanoate (29 g, 50 mmol) was cooled to 5° C. HCl in ethyl acetate (392.2 mL of 1.9 molar solution) was then added. The reaction vessel was capped and placed in the refrigerator and allowed to sit for 18 hours. The reaction was then allowed to stir at room temperature for two hours. The solvent was removed under a stream on nitrogen and then the reaction residue was condensed under vacuum. The reaction gave 17.5 g (42%) of ethyl 4-[3-(4-aminobutyl)-1,2,4-oxadiazol-5-yl]-3-(1,3-benzodioxol-5-yl)butanoate hydrochloride.
Step 8. Ethyl 3-(1,3-benzodioxol-5-yl)-4-{3-[4-(ethanimidoylamino)butyl]-1,2,4-oxadiazol-5-yl}butanoate hydrochloride.

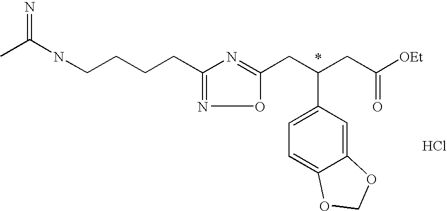

Ethyl 4-[3-(4-aminobutyl)-1,2,4-oxadiazol-5-yl]-3-(1,3-benzodioxol-5-yl)butanoate hydrochloride (200 mg, 0.49 mmol), ethyl ethanimidoate (60 mg, 0.49 mmol), triethylamine (0.2 mL, 1.5 mmol), and EtOH (5 mL) were placed in a vial, capped and allowed to stir at 60° C. for six hours. The solvent was then removed under a stream of nitrogen and then purified by reverse phase HPLC. The reaction yielded 150 mg (85%) of ethyl 3-(1,3-benzodioxol-5-yl)-4-{3-[4-(ethanimidoylamino)butyl]-1,2,4-oxadiazol-5-yl}butanoate hydrochloride.
Step 9. 3-(1,3-Benzodioxol-5-yl)-4-{3-[4-(ethanimidoylamino)butyl]-1,2,4-oxadiazol-5-yl}butanoic acid hydrochloride.

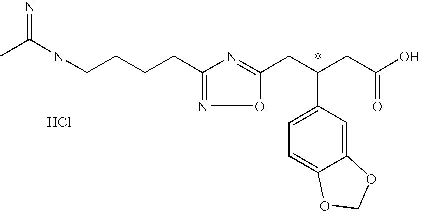

Ethyl 3-(1,3-benzodioxol-5-yl)-4-{3-[4-(ethanimidoylamino)butyl]-1,2,4-oxadiazol-5-yl}butanoate hydrochloride (150 mg, 0.4 mmol), acetone (3 mL), conc. HCl (0.18 mL, 2.2 mmol), and water (0.18 mL), were allowed to heat to 57° C. for five hours. The reaction was then dried under a stream of nitrogen and purified on a reverse phase HPLC. The reaction yielded 81 mg (52%) of 3-(1,3-benzodioxol-5-yl)-4-{3-[4-(ethanimidoylamino)butyl]-1,2,4-oxadiazol-5-yl}butanoic acid hydrochloride. ¹H NMR (400 MHz) DMSO-d₆ [symbol] 9.45 (br s, 1H), 9.1 (br s, 1H), 8.55 (br s, 1H), 7.1 (ds, 1H), 6.75 (d, 1H), 6.6 (dd 1H), 5.95 (s, 2H), 3.45 (m, 1H), 3.3-3.1 (m, 4H), 2.7-2.5 (m, 4H), 2.12 (s, 3H), 1.65 (m, 2H), 1.5 (m, 2H). Mass Spectrum: (MH⁺)=389.2.

### EXAMPLE 88

3-(1,3-Benzodioxol-5-yl)-4-{3-[4-(3,4,5,6-tetrahydro-2H-azepin-7-ylamino)butyl]-1,2,4-oxadiazol-5-yl}butanoic acid hydrochloride.

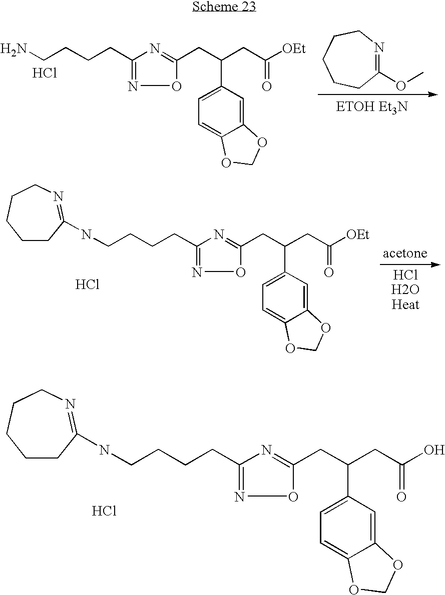

3-(1,3-Benzodioxol-5-yl)-4-{3-[4-(3,4,5,6-tetrahydro-2H-azepin-7-ylamino)butyl]-1,2,4-oxadiazol-5-yl} butanoic acid hydrochloride was made according to the method as described for preparing 3-(1,3-benzodioxol-5-yl)-4-{3-[4-(ethanimidoylamino) butyl]-1,2,4-oxadiazol-5-yl}butanoic acid hydrochloride using 7-methoxy-3,4,5,6-tetrahydro-2H-azepine: ¹H NMR (400 MHz) DMSO-d₆ [symbol] 9.55 (br s, 1H), 9.15 (br s, 1H), 6.9 (ds, 1H), 6.75 (d, 1H), 6.65 (dd, 1H), 5.95 (s, 2H), 3.55-3.1 (m, 7H), 2.8-2.55 (m, 6H), 1.8-1.5 (m, 10H) Mass Spectrum: (MH⁺)=443.1.

### EXAMPLE 89

3-(1,3-Benzodioxol-5-yl)-4-{3-[4-(3,4,5,6-tetrahydropyridin-2-ylamino)butyl]-1,2,4-oxadiazol-5-yl}butanoic acid hydrochloride. 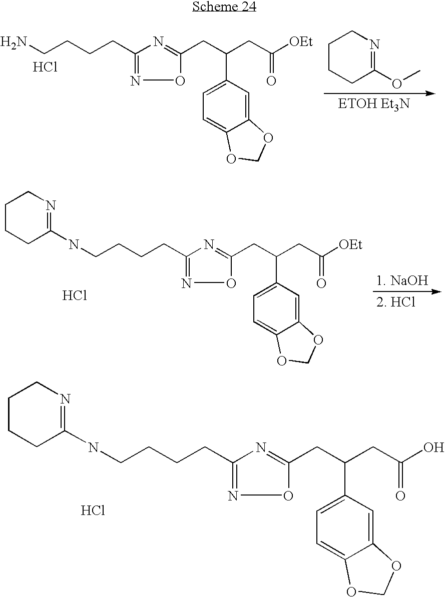
Step 1. 3-(1,3-Benzodioxol-5-yl)-4-{3-[4-(3,4,5,6-tetrahydropyridin-2-ylamino)butyl]-1,2,4-oxadiazol-5-yl}butanoate hydrochloride.

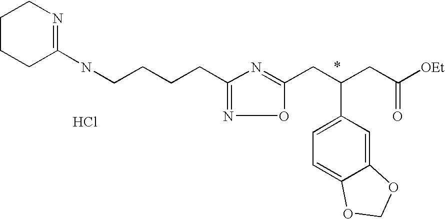

3-(1,3-Benzodioxol-5-yl)-4-{3-[4-(3,4,5,6-tetrahydropyridin-2-ylamino)butyl]-1,2,4-oxadiazol-5-yl}butanoate hydrochloride was made according to the method as described for preparing 3-(1,3-benzodioxol-5-yl)-4-{3-[4-(ethanimidoylamino)butyl]-1,2,4-oxadiazol-5-yl}butanoic acid hydrochloride using 6-methoxy-2,3,4,5-tetrahydropyridine.
Step 2. 3-(1,3-Benzodioxol-5-yl)-4-{3-[4-(3,4,5,6-tetrahydropyridin-2-ylamino)butyl]-1,2,4-oxadiazol-5-yl}butanoic acid hydrochloride.

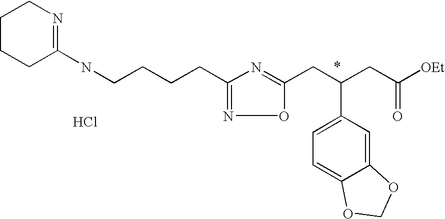

3-(1,3-Benzodioxol-5-yl)-4-{3-[4-(3,4,5,6-tetrahydropyridin-2-ylamino)butyl]-1,2,4-oxadiazol-5-yl}butanoate hydrochloride (180 mg, 0.4 mmol), conc. HCl (0.2 mL, 2.4 mmol), water (0.2 mL, 2.4 mmol) and acetone (1.5 mL) were placed in a vial capped and allowed to stir at room temperature for 18 hours. The solvent was then removed under a stream of nitrogen and purified on a reverse phase HPLC. The reaction yielded 92 mg (75%) of 3-(1,3-benzodioxol-5-yl)-4-{3-[4-(3,4,5,6-tetrahydropyridin-2-ylamino)butyl]-1,2,4-oxadiazol-5-yl}butanoic acid hydrochloride. ¹H NMR (400 MHz) DMSO-d₆ [symbol] 9.55 (br s, 1H), 9.15 (br s, 1H), 6.9 (ds, 1H), 6.75 (d, 1H), 6.65 (dd, 1H), 5.95 (s, 2H), 3.55-3.1 (m, 5H), 2.8-2.55 (m, 6H), 1.8-1.5 (m, 8H) Mass Spectrum: (MH⁺)=429.2.

### EXAMPLE 90

3-(1,3-Benzodioxol-5-yl)-4-[3-(4-{[imino(phenyl)methyl]-amino}butyl)-1,2,4-oxadiazol-5-yl]butanoic acid hydrochloride.

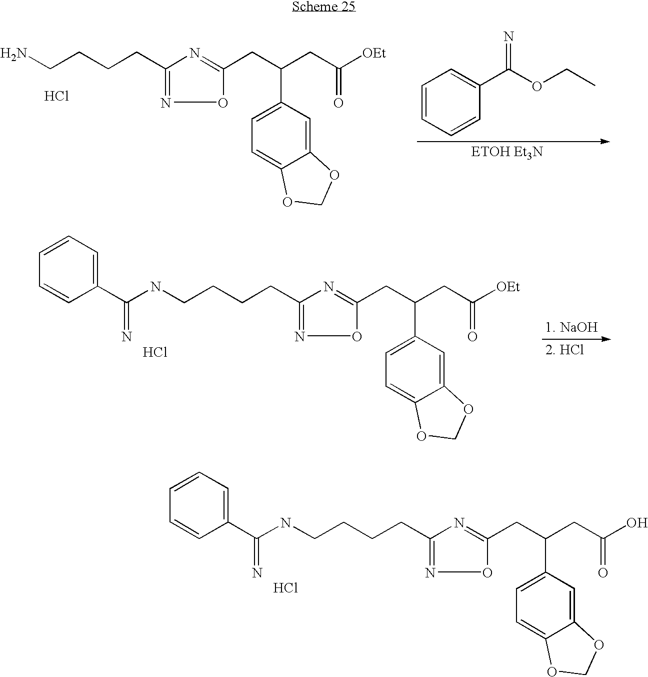

3-(1,3-Benzodioxol-5-yl)-4-[3-(4-{[imino(phenyl)methyl]amino}butyl)-1,2,4-oxadiazol-5-yl]butanoic acid hydrochloride was prepared according to the method described to prepare 3-(1,3-benzodioxol-5-yl)-4-{3-[4-(3,4,5,6-tetrahydropyridin-2-ylamino)butyl]-1,2,4-oxadiazol-5-yl}butanoic acid hydrochloride using ethyl benzenecarboximidoate. ¹H NMR (400 MHz) DMSO-d₆ [symbol] 9.65 (br s, 1H), 9.45 (br s, 1H), 9.05 (br s, 1H), 7.73 (m, 3H), 7.6 (m, 2H), 6.9 (sd, 1H), 6.75 (d, 1H), 6.65 (dd, 1H), 5.95 (s, 2H), 3.55-3.1 (m, 5H), 2.8-2.55 (m, 4H), 1.8-1.6 (m, 4H) Mass Spectrum: (MH⁺)=451.1.

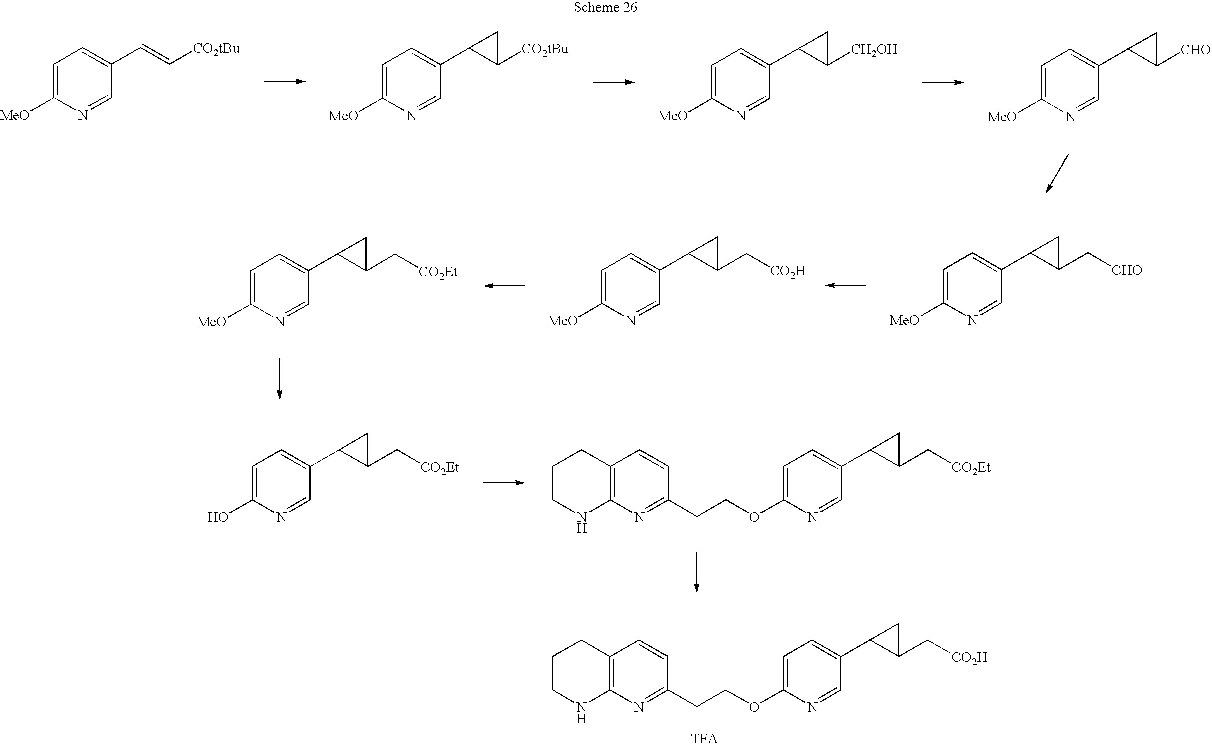

### EXAMPLE 91

(2-{6-[2-(5,6,7,8-Tetrahydro-1,8-naphthyridin-2-yl)ethoxy]pyridin-3-yl}cyclopropyl)acetic acid. 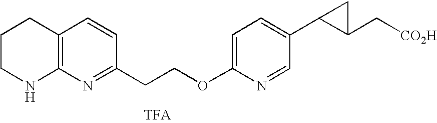
Step 1. Synthesis of 2-methoxy-5-(2-methylcyclopropyl)pyridine.

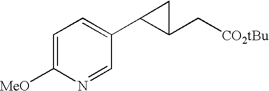

A solution of trimethylsulfoxonium iodide (4.4 g, 0.02 mol) in DMSO (20 mL) was stirred at room temperature. To this solution was added sodium hydride, 60% in mineral oil (0.84 g, 0.021 mol) over a period of 20 minutes; the suspension was stirred for one hour at room temperature. A solution of 2-methoxy-5-[(1E)-prop-1-enyl]pyridine (2.35 g, 0.01 mol) in DMSO (20 mL) was added drop wise and the reaction mixture stirred for 3-6 hours. The reaction was quenched by addition of sat. NH₄Cl, the mixture was diluted with four-fold amount of water and extracted with CH₂Cl₂ (3 times). The combined organic layer was washed with brine, dried over Na₂SO₄, filtered and concentrated. The concentrated residue was chromatographed on silica gel using 5% ethyl acetate/hexane to give 1.5 g (51%) white powder. ¹H NMR (CDCl₃) δ 7.97 (m, 1H), 7.2 (m, 1H), 6.63 (m, 1H), 3.8 (s, 3H), 2.35 (m, 1H), 1.71 (m, 1H), 1.48 (m, 1H), 1.45 (s, 9H), 1.15 (m, 1H).
Step 2. Synthesis of [2-(6-methoxypyridin-3-yl)cyclopropyl]methanol.

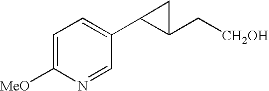

To the ester from step 1 (1.3 gm, 5.2 mmol) in THF (20 mL) at −78° C. was added DIBAL (1M in hexane, 48 mL, 13 mmol). The reaction was stirred at −78° C. for 30 minutes and then at 0° C. for 2-3 hours. The reaction mixture was cooled to −78° C. and quenched by slow addition of sat. aqueous potassium sodium tartrate. The reaction mixture was stirred for 2 hours at room temperature. The solution was extracted with ethyl acetate (3 times), washed with brine, dried over Na₂SO₄ and concentrated to give oil (0.8 g, 85%). ¹H NMR (CDCl₃) δ 7.90 (m, 1H), 7.12 (m, 1H), 6.63 (m, 1H), 3.8 (s, 3H), 3.57 (m, 2H), 1.71 (m, 1H), 1.32 (m, 1H), 0.83 (m, 1H).
Step 3. Synthesis of 2-(6-methoxypyridin-3-yl)cyclopropanecarbaldehyde.

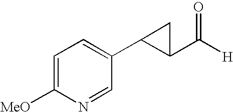

A solution of oxalyl chloride (2M in CH₂Cl₂, 1.67 mL, 3.4 mmol) in methylene chloride (10 mL) was cooled to −78° C. under nitrogen and a solution of DMSO (0.47 mL, 6.7 mmol) in methylene chloride (10 mL) was added dropwise and stirring was continued for 5 minutes. The product from step 2 (0.3 g, 1.6 mmol) in CH₂Cl₂ (10 mL) was added drop wise over 5 minutes and the resultant mixture was stirred for 15 minutes at −78° C. Triethylamine (1.8 mL, 13 mmol) was added rapidly and the mixture stirred at −78° C. for an additional 5 minutes followed by 30 minutes at room temperature. The reaction was diluted with water and extracted several times with CH₂Cl₂. The combined organic extracts were washed with 1.5 N aqueous. HCl. The aqueous layer after acidic extraction was neutralized with 2.5N NaOH and back extracted with CH₂Cl₂. The combined organic layers were washed with sat. NaHCO₃ solution, brine, dried over Na₂SO₄ and concentrated to give yellow oil. ¹H NMR (CDCl₃) δ 9.32 (d, 1H), 7.94 (m, 1H), 7.22 (m, 1H), 6.63 (m, 1H), 3.8 (s, 3H), 2.57 (m, 1H), 2.12 (m, 1H), 1.71 (m, 1H), 1.32 (m, 1H).
Step 4. Synthesis of [2-(6-methoxypyridin-3-yl)cyclopropyl]acetic acid.

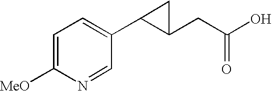

To a solution of methoxymethyl(triphenyl)phosphonium chloride (4.5 g, 0.02 mol) in dry THF (25 mL) at 0° C. was added lithiumbis(trimethylsilyl)amide (1M in THF, 21.5 mL, 0.022 mol). The reaction was stirred for 30 minutes at 0° C. To the solution was added aldehyde from step 3 (2.31 g, 0.013 mol) in THF (40 mL). Ice bath was removed 30 minutes later and reaction was stirred for one hour at room temperature. The reaction mixture was poured into water and extracted with ether. The organic layer was washed with brine, dried over Na₂SO₄ and concentrated. To the resulting methoxy olefin in THF (30 mL) was added HCl (1.5N, 30 mL) and refluxed for 2 hours. The reaction was cooled to room temperature, neutralized by adding saturated aqueous sodium bicarbonate and extracted with ether. The organic layer washed with brine, dried over sodium sulfate and concentrated to give oil used without further purification. A solution of aldehyde (6.0 g, 0.032 mol) in ethanol (40 mL) was cooled to 0° C. To this solution was added silver nitrate (10.88 g, 0.064 mol in 25 mL distilled water), followed by sodium hydroxide (5.12 g, 0.128 mol in 25 mL distilled water) over a period of 10 minutes. Ice bath was removed 20 minutes later. The black solution was stirred at room temperature for additional 2 hours, filtered through pad of celite and washed with excess 2.5N aqueous NaOH solution. Filtrate was concentrated to remove ethanol. The aqueous solution was extracted with ether followed by ethyl acetate. The combined organic layer was discarded and aqueous solution was acidified using 5% citric acid. The aqueous layer was extracted with dichloromethane (3 times), the organic solution washed with brine, dried over sodium sulfate and concentrated to give 1.5 g oil. ¹H NMR (CDCl₃) δ 7.94 (m, 1H), 7.42 (m, 1H), 6.73 (m, 1H), 3.8 (s, 3H), 2.2-2.62 (m, 2H), 1.79 (m, 1H), 1.2 (m, 1H), 0.92 (m, 1H), 0.85 (m, 1H).
Step 5. Synthesis of ethyl [2-(6-methoxypyridin-3-yl)cyclopropyl]acetate.

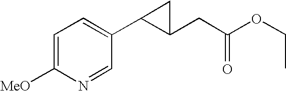

To the solution of the acid from step 4 (3 g, 14.5 mmol) in ethanol (30 mL) was added 4N HCl in dioxane (11 mL, 43.4 mmol). The reaction mixture was stirred for 3 hours at room temperature. The solvent was concentrated and residue dissolved in ethyl acetate. The organic layer was washed with saturated aqueous sodium bicarbonate, brine, dried over sodium sulfate and concentrated to give brown oil. ¹H NMR (CDCl₃) δ 7.94 (m, 1H), 7.32 (m, 1H), 6.63 (m, 1H), 4.08 (q, 2H), 3.8 (s, 3H), 2.2-2.62 (m, 2H), 1.73 (m, 1H), 1.24 (t, 3H), 1.2 (m, 1H), 0.92 (m, 1H), 0.85 (m, 1H).
Step 6. Synthesis of ethyl [2-(6-hydroxypyridin-3-yl)cyclopropyl]acetate.

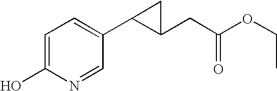

A solution of methoxy ethyl ester from step 5 (1.6 g, 6.8 mmol), sodium iodide (102 g, 0.68 mol) and acetonitrile (200 mL) was cooled at 0° C. To this solution trimethylsilyl chloride (86 mL. 0.68 mol) was added dropwise followed by additional acetonitrile (100 mL) and water (0.5 mL). The solution was heated at 60° C. for 5 hours. The reaction was cooled to 0° C. and quenched by slow addition of ethanol (100 mL). The reaction was stirred at room temperature for 1 hour, solvent concentrated and residue dissolved in ethyl acetate. The organic layer washed with brine and Na₂S₂O₃ (1:1) mixture. The aqueous layer was extracted with ethyl acetate (4 times). Combined organic layer was dried over sodium sulfate, filtered and concentrated to give 1.4 g white powder. ¹H NMR (CDCl₃) δ 7.4 (m, 1H), 7.12 (m, 1H), 6.43 (m, 1H), 4.08 (q, 2H), 2.1-2.24 (m, 1H), 2.4-2.6 (m, 1H), 1.63 (m, 1H), 1.14 (m, 1H), 0.92 (m, 1H), 0.85 (m, 1H).
Step 7. Synthesis of ethyl(2-{6-[2-(5,6,7,8-tetrahydro-1,8-naphthyridin-2-yl)ethoxy]pyridin-3-yl}cyclopropyl)acetate.

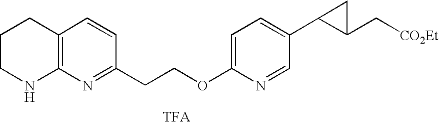

A solution of 2-(5,6,7,8-tetrahydro-1,8-napthyridin-2-yl)-1-ethanol (WO 0033838, 0.211 g, 1.19 mmol), polymer bound PPh3(0.403 g, 1.19 mmol) in dry THF (20 mL) was stirred at room temperature. A solution of cyclopropyl intermediate from step 6 (0.175 g, 0.79 mmol) in dry THF (20 mL) was added followed by DIAD (0.24 mL, 1.19 mmol) over 5 minutes. The reaction mixture was stirred for 4 days at room temperature, filtered through celite and washed with excess THF. The solvent was concentrated, residue dissolved in (10 mL) 50% acetonitrile in water and acidified by adding TFA. The residue was purified on reverse phase HPLC to give the title compound as yellow solid. ¹H NMR (CD₃OD) δ 7.92 (m, 1H), 7.65 (m, 1H), 7.42 (m, 1H), 6.79 (m, 2H), 4.5 (t, 2H), 4.1 (q, 2H), 3.45 (t, 2H), 3.12 (t, 2H), 2.91 (t, 2H), 2.2-2.4 (m, 1H), 2.5-2.6 (m, 1H), 1.93 (m, 2H), 1.62-1.8 (m, 1H), 1.23(t, 3H), 0.95 (m, 1H), 0.84 (m, 1H).
Step 8. Synthesis of (2-{6-[2-(5,6,7,8-tetrahydro-1,8-naphthyridin-2-yl)ethoxy]pyridin-3-yl}cyclopropyl)acetic acid.

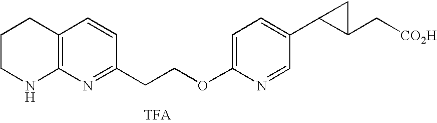

The ethyl ester from step 7 (0.1 g) was dissolved in ethanol (5 mL) and water (1 mL). LiOH (0.05 g) was added and the reaction heated at 50° C. for 3 hours. The solvent was concentrated to remove ethanol. The residue was dissolved in (10 mL) 50% acetonitrile in water and acidified by adding TFA. The residue was purified on reverse phase HPLC to give the title compound as white solid (0.02 g). ¹H NMR (CD₃OD) δ 7.92 (m, 1H), 7.65 (m, 1H), 7.42 (m, 1H), 6.79 (m, 2H), 4.1 (q, 2H), 3.45 (t, 2H), 3.12 (t, 2H), 2.91 (t, 2H), 2.2-2.4 (m, 1H), 2.5-2.6 (m, 1H), 1.93 (m, 2H), 1.62-1.8 (m, 1H), 0.95 (m, 1H), 0.84 (m, 1H). Anal. Calcd for C₂₀H₂₃N₃O₃: Mol. Wt. 353.42. Found: 354.1971 (M+H, HRMS).

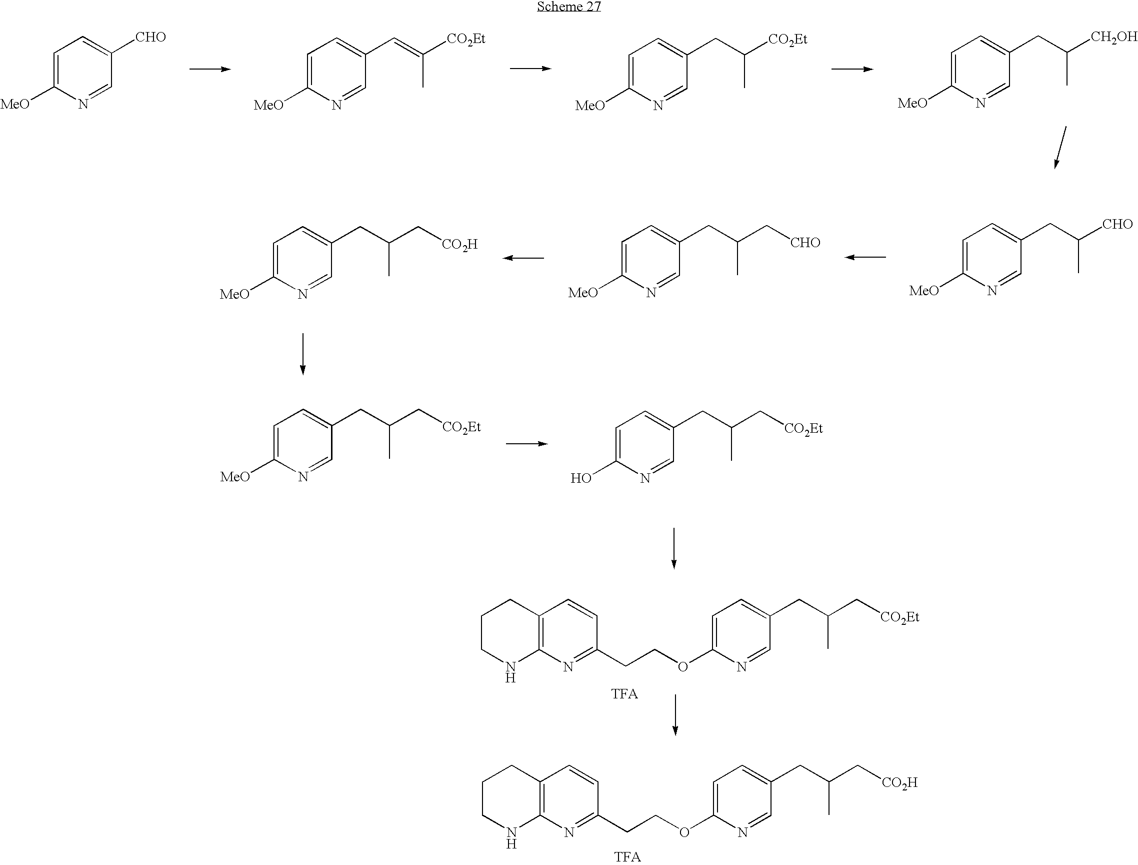

### EXAMPLE 92

3-Methyl-4-{6-[2-(5,6,7,8-tetrahydro-1,8-naphthyridin-2-yl)ethoxy]pyridin-3-yl}butanoic acid 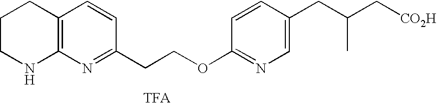
Step 1. Synthesis of ethyl(2E)-3-(6-methoxypyridin-3-yl)-2-methylprop-2-enoate

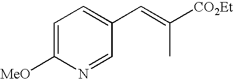

A solution of 6-methoxynictonialdehyde (1.0 g, 7.2 mmol) and carbethoxy-ethylidiene triphenylphosphorane (3.34 g, 9.48 mmol) was dissolved in dichloromethane (70 mL). The solution was refluxed for 3 hours, cooled to room temperature and washed with brine. The aqueous layer was extracted three times with dichloromethane, combined organic layers dried over sodium sulfate, filtered, and concentrated to give yellow solid. The residue chromatographed on silica gel using 20% ethyl acetate/hexane to give oil (1.3 g, 80%). NMR (CDCl₃) δ 8.24 (m, 1H), 7.62 (m, 1H), 7.52 (m, 1H), 6.73 (m, 1H), 4.21 (q, 2H), 3.8 (s, 3H), 2.08 (s, 3H), 1.24 (t, 3H).
Step 2. Synthesis of ethyl 3-(6-methoxypyridin-3-yl)-2-methylpropanoate.

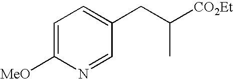

A solution of olefin from step 1 (1.2 g, 5.4 mmol) was dissolved in ethanol (50 mL) and stirred with 5% Pd/C (0.13 g) under an atmosphere of hydrogen (30 psi) at room temperature. After 2 days the reaction was complete. The catalyst was removed by filtration through celite and washed with 10 mL ethanol. Evaporation of the combined filtrate and washing yielded oil (1.1 g, 91%). NMR (CDCl₃) δ 7.92 (m, 1H), 7.55 (m, 1H), 6.78 (m, 1H), 4.1 (q, 2H), 3.8 (s, 3H), 2.8 (m, 1H), 2.64 (m, 2H), 1.16 (m, 6H).
Step 3. Synthesis of 3-(6-methoxypyridin-3-yl)-2-methylpropan-1-ol.

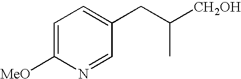

To the ester from step 2 (1.3 g, 5.2 mmol) in THF (20 mL) at −78° C. was added DIBAL (1M in hexane, 48 mL, 13 mmol). The reaction was stirred at −78° C. for 30 minutes and then at 0° C. for 2-3 hours. The reaction mixture was cooled to −78° C. and quenched by slow addition of sat. aqueous potassium sodium tartrate. The reaction mixture was stirred for 2 hours at room temperature. The solution was extracted with ethyl acetate (3 times), washed with brine, dried over Na₂SO₄ and concentrated to give oil (0.8 g, 85%). ¹H NMR (CDCl₃) δ 7.90 (m, 1H), 7.4 (m, 1H), 6.68 (m, 1H), 3.8 (s, 3H), 3.47 (m, 2H), 2.6-2.7 (m, 1H), 2.3-2.4 (m, 1H), 0.94 (m, 4H).
Step 4. Synthesis of 3-(6-methoxypyridin-3-yl)-2-methylpropanal.

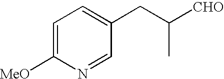

A solution of oxalyl chloride (2M in CH₂Cl₂, 27 mL, 0.054 mol) in methylene chloride (30 mL) was cooled to −78° C. under nitrogen and a solution of DMSO (7.6 mL, 0.108 mol) in methylene chloride (30 mL) was added dropwise and stirring continued for 5 minutes. The product from step 3 (4.8 g, 0.027 mol) in CH₂Cl₂ (40 mL) was added dropwise over 5 minutes and the resultant mixture was stirred for 15 minutes at −78° C. Triethylamine (30 mL, 0.216 mol) was added rapidly and the mixture stirred at −78° C. for an additional 5 minutes followed by 30 minutes at room temperature. The reaction was diluted with water and extracted several times with CH₂Cl₂. The combined organic extracts were washed with aqua. 1.5 N HCl. The aqueous layer after acidic extraction was neutralized with 2.5N NaOH and back extracted with CH₂Cl₂. The combined organic layers were washed with sat. NaHCO₃ solution, brine, dried over Na₂SO₄ and concentrated to give yellow oil (4.6 g). ¹H NMR (CDCl₃) δ 9.32 (d, 1H), 7.90 (m, 1H), 7.4 (m, 1H), 6.68 (m, 1H), 3.8 (s, 3H), 3.47 (m, 2H), 2.6-2.7 (m, 1H), 2.3-2.4 (m, 1H), 0.94 (m, 4H).
Step 5. Synthesis of 4-(6-methoxypyridin-3-yl)-3-methylbutanal.

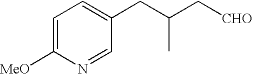

To a solution of methoxymethyl(triphenyl)phosphonium chloride (18.11 g, 0.0528 mol) in dry THF (50 mL) at 0° C. was added lithiumbis(trimethylsilyl)amide (1M in THF, 58 mL, 0.058 mol). The reaction was stirred for 30 minutes at 0° C. To the solution was added aldehyde from step 4 (4.6 g, 0.0264 mol) in THF (50 mL). Ice bath was removed 30 minutes later and reaction was stirred for one hour at room temperature. The reaction mixture was poured into water and extracted with ether. The organic layer was washed with brine, dried over Na₂SO₄ and concentrated. To the resulting methoxy olefin in THF (50 mL) was added HCl (1.5 N, 50 mL) and refluxed overnight. The reaction was cooled to room temperature, neutralized by adding saturated aqueous sodium bicarbonate and extracted with ether. The organic layer washed with brine, dried over sodium sulfate and concentrated to give oil used without further purification.
Step 6. Synthesis of 4-(6-methoxypyridin-3-yl)-3-methylbutanoic acid.

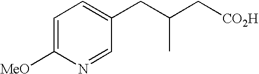

A solution of aldehyde from step 5 (4.0 g, 0.021 mol) in ethanol (30 mL) was cooled at 0° C. To this solution was added silver nitrate (6.93 g, 0.042 mol in 25 mL distilled water), followed by sodium hydroxide (3.36 g, 0.084 mol in 25 mL distilled water) over a period of 10 minutes. Ice bath was removed 20 minutes later. The black solution was stirred at room temperature for additional 2 hours, filtered through pad of celite and washed with excess 2.5N aqueous NaOH solution. Filtrate was concentrated to remove ethanol. The aqueous solution was extracted with ether followed by ethyl acetate. The combined organic layer was discarded and aqueous solution was acidified using 5% citric acid and extracted with dichloromethane (3 times), the combined organic layer washed with brine, dried over sodium sulfate and concentrated to give oil (3.5 g, 80%). ¹H NMR (CDCl₃) δ 7.9 (m, 1H), 7.4 (m, 1H), 6.68 (m, 1H), 3.8 (s, 3H), 2.52-2.6 (m, 1H), 2.38-2.48 (m, 1H), 2.3-2.35 (m, 1H), 2.1-2.2 (m, 2H), 0.94 (d, 3H).
Step 7. Synthesis of ethyl 4-(6-methoxypyridin-3-yl)-3-methylbutanoate.

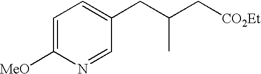

To the solution of the acid from step 6 (3.5 g, 14.75 mmol) in ethanol (30 mL) was added 4N HCl in dioxane (11 mL, 43.4 mmol). The reaction mixture was stirred for 3 hours at room temperature. The solvent was concentrated and residue dissolved in ethyl acetate. The organic layer was washed with saturated aqueous sodium bicarbonate, brine, dried over sodium sulfate and concentrated to give brown oil. ¹H NMR (CDCl₃) δ 7.9 (m, 1H), 7.4 (m, 1H), 6.68 (m, 1H), 4.2 (q, 2H), 3.8 (s, 3H), 2.52-2.6 (m, 1H), 2.38-2.48 (m, 1H), 2.3-2.35 (m, 1H), 2.1-2.2 (m, 2H), 1.2 (t, 3H), 0.94 (d, 3H).
Step 8. Synthesis of ethyl 4-(6-hydroxypyridin-3-yl)-3-methylbutanoate.

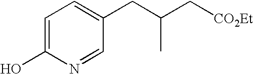

To a solution of methoxy ethyl ester from step 7 (1.5 g, 6.3 mmol) in anhydrous chloroform (20 mL) was added trimethylsilyl iodide (9.0 mL, 63 mmol). The solution was heated overnight at 50° C. under atmosphere of nitrogen, cooled down to 0° C. and quenched by 10 mL of ethanol. The reaction was stirred at room temperature for 1 hour, solvent concentrated and residue dissolved in ethyl acetate. The organic layer washed with brine:Na₂S₂O₃ (1:1) mixture. The aqueous layer extracted with ethyl acetate (4 times). Combined organic layer dried over sodium sulfate, filtered and concentrated to give 1.2 g oil. ¹H NMR (CDCl₃) δ 7.92 (m, 1H), 7.45 (m, 1H), 6.64 (m, 1H), 4.1 (q, 2H), 2.52-2.6 (m, 1H), 2.38-2.48 (m, 1H), 2.3-2.35 (m, 1H), 2.1-2.2 (m, 2H), 1.1 (t, 3H), 0.94 (d, 3H).
Step 9. Synthesis of ethyl 3-methyl-4-{6-[2-(5,6,7,8-tetrahydro-1,8-naphthyridin-2-yl)ethoxy]pyridin-3-yl}butanoate.

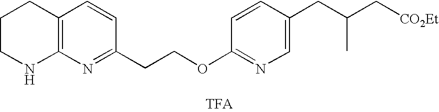

A solution of 2-(5,6,7,8-tetrahydro-1,8-napthyridin-2-yl)-1-ethanol (WO 0033838, 01.44 g, 8.07 mmol), polymer bound PPh₃ (3.26 g, 8.07 mmol) in dry THF (40 mL) was stirred at room temperature. A solution of compound from step 8 (1.2 g, 5.38 mmol) in dry THF (40 mL) was added followed by DIAD (1.76 mL, 8.07 mmol) over 15 minutes. The reaction mixture was stirred for overnight at room temperature, filtered through celite and washed with excess THF. The solvent was concentrated, residue dissolved in (10 mL) 50% acetonitrile in water and acidified by adding TFA. The residue was purified on reverse phase HPLC to give the title compound as white solid. ¹H NMR (CD₃OD) δ 7.62 (m, 1H), 7.45 (m, 1H), 7.36 (m, 1H), 6.4-6.5 (m, 2H), 4.3 (t, 2H), 4.1 (q, 2H), 3.5 (t, 2H), 3.2 (t, 2H), 2.72 (t, 2H), 2.4 (m, 1H), 2.2-2.4 (m, 2H), 2.0-2.2 (m, 2H), 1.93 (m, 2H), 1.2 (t, 3H), 0.9 (d, 3H).
Step 10. Synthesis of 3-methyl-4-{6-[2-(5,6,7,8-tetrahydro-1,8-naphthyridin-2-yl)ethoxy]pyridin-3-yl}butanoic acid.

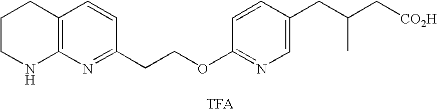

The ethyl ester from step 9 (1.0 g) was dissolved in ethanol (10 mL) and water (1 mL). LiOH (0.21 g) was added and the reaction heated at 50° C. for 3 hours. The solvent was concentrated to remove ethanol. The residue was dissolved in (10 mL) 50% acetonitrile in water and acidified by adding TFA. The residue was purified on reverse phase HPLC to give the title compound as white solid (0.22 g). ¹H NMR (CD₃OD) δ 7.64 (m, 1H), 7.46 (m, 1H), 7.38 (m, 1H), 6.4-6.5 (m, 2H), 4.3 (t, 2H), 3.5 (t, 2H), 3.2 (t, 2H), 2.72 (t, 2H), 2.4 (m, 1H), 2.2-2.4 (m, 2H), 2.0-2.2 (m, 2H), 1.93 (m, 2H), 0.9 (d, 3H). Anal. Calcd for C₂₀H₂₅N₃O₃: Mol. Wt. 355.43. Found. 356.1973 (M+H, HRMS).

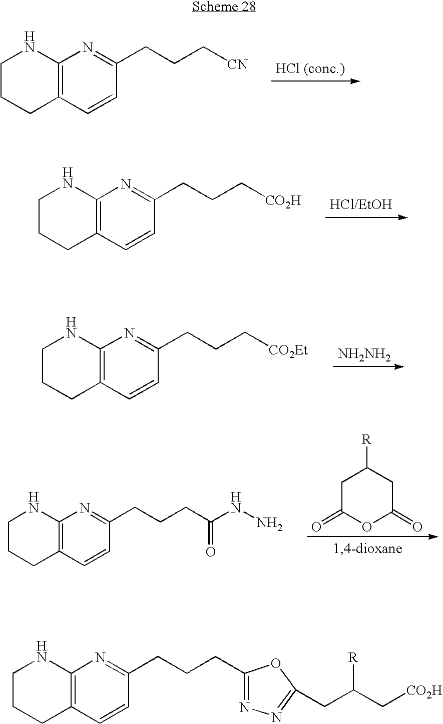

### EXAMPLE 93

Preparation of 3-(1,3-benzodioxol-5-yl)-4-{5-[3-(5,6,7,8-tetrahydro-1,8-naphthyridin-2-yl)propyl]-1,3,4-oxadiazol-2-yl}butanoic acid trifluoroacetate. 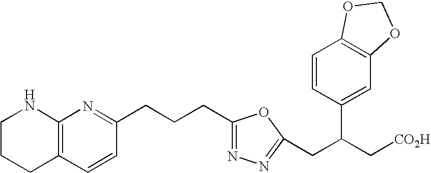
Step 1. Preparation of 4-(5,6,7,8-tetrahydro-1,8-naphthyridin-2-yl)butanoic acid.

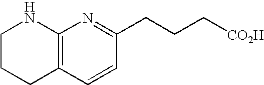

Chill a flask containing 4-(5,6,7,8-tetrahydro-1,8-naphthyridin-2-yl)butanenitrile (800 mg, 3.97 mmol) to 0° C. Slowly add conc. HCl (25 mL), warm to room temperature, and let stir for 16 hrs. Concentrate reaction in vacuo and lyophilize. Quantitative yield; go directly to esterification. ¹H NMR (DMSO-d₆) δ 7.61 (d, 1H), 6.60 (d, 1H), 3.45-3.36 (m, 2H), 2.74 (t, 2H), 2.65 (t, 2H), 2.08 (t, 2H), 1.88-1.78 (m, 4H).
Step 2. Preparation of ethyl 4-(5,6,7,8-tetrahydro-1,8-naphthyridin-2-yl)butanoate.

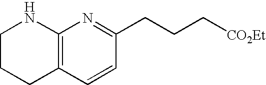

A mixture of 4-(5,6,7,8-tetrahydro-1,8-naphthyridin-2-yl)butanoic acid (0.80 g, 3.97 mmol) in HCl/EtOH solution (25 mL) was heated to reflux for 16 hrs. The reaction was concentrated in vacuo and purified via reverse phase HPLC using a gradient of 5-40% acetonitrile/H₂O/2% TFA over 30 min to obtain a yellow solid. Yield: 350 mg of TFA salt (24% over 2 steps). ¹H NMR (DMSO-d₆) δ 7.60 (d, 1H), 6.60 (d, 1H), 4.04 (q, 2H), 3.45-3.36 (m, 2H), 2.75 (t, 2H), 2.66 (t, 2H), 2.33 (t, 2H), 1.93-1.78 (m, 4H), 1.16 (t, 3H).
Step 3. Preparation of 4-(5,6,7,8-tetrahydro-1,8-naphthyridin-2-yl)butanohydrazide.

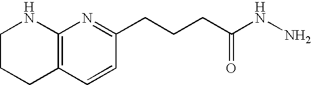

Ethyl 4-(5,6,7,8-tetrahydro-1,8-naphthyridin-2-yl)butanoate (300 mg, 0.83 mmol) was dissolved in hydrazine hydrate (neat) and heated to 100° C. for 4 hrs. The reaction was cooled and concentrated in vacuo to give 305 mg of the desired crude product as a white sticky solid. ¹H NMR (DMSO-d₆) δ 7.33 (d, 1H), 6.42, (d, 1H), 3.03 (t, 2H), 2.67 (t, 2H), 2.55-2.48 (m, 2H), 2.04 (t, 2H), 1.88-1.73 (m, 4H).
Step 4. Preparation of 3-(1,3-benzodioxol-5-yl)-4-{5-[3-(5,6,7,8-tetrahydro-1,8-naphthyridin-2-yl)propyl]-1,3,4-oxadiazol-2-yl}butanoic acid trifluoroacetate.

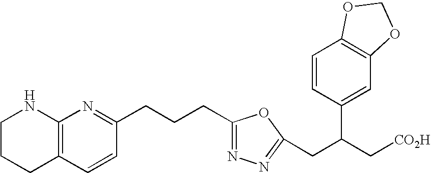

Dissolve 4-(5,6,7,8-tetrahydro-1,8-naphthyridin-2-yl)butanohydrazide (305 mg) and the anhydride, 4-(1,3-benzodioxol-5-yl)dihydro-2H-pyran-2,6(3H)-dione (334.8 mg) in 1,4-dioxane under argon. Heat to 100° C. for 16 hrs. The reaction mixture allowed to cool and concentrated in vacuo. The residue was purified via reverse phase HPLC using a gradient of 10-50% acetonitrile/H₂O/2% TFA over 30 min to obtain a white solid. Yield: 153 mg (19%). ¹H NMR (DMSO-d₆) δ 7.65-7.60(d, 1H), 7.10-7.00 (m, 1H), 6.90-6.79(m, 2H), 6.67-6.61 (m, 1H), 6.12-5.97 (m, 2H), 3.45-3.38 (m, 2H), 3.16-2.99 (m, 2H), 2.94-2.83 (2H), 2.77-2.65 (m, 4H), 2.33-2.24 (m, 2H), 1.95-1.77 (m, 4H). Analysis Calculated for C₂₄H₂₆N₄O₅.1.4 TFA: Expected: C, 52.76; H, 4.53; N, 9.18. Found: C, 52.59; H, 4.80; N, 9.29. Calculated Mass: 450.50. Found Mass: 451.0 (for MH⁺).

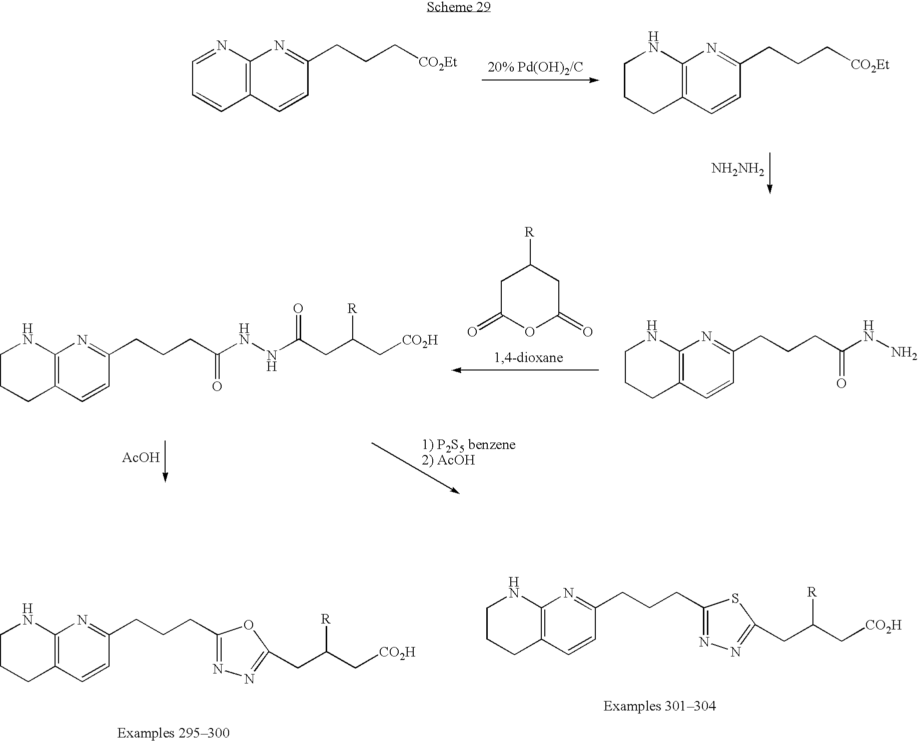

### EXAMPLE 94

Preparation of 3-(3-fluorophenyl)-4-{5-[3-(5,6,7,8-tetrahydro-1,8-naphthyridin-2-yl)propyl]-1,3,4-oxadiazol-2-yl}butanoic acid trifluoroacetate.

Step 1. Preparation of ethyl 4-(5,6,7,8-tetrahydro-1,8-naphthyridin-2-yl)butanoate.

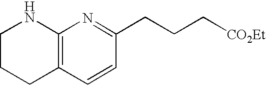

Ethyl 4-(1,8-naphthyridin-2-yl)butanoate (5.45 g) was hydrogenated in EtOH using 20% Pd(OH)₂/C at 5 psi and room temperature for 6 hrs. The reaction was filtered and concentrated in vacuo to give the crude desired product as a yellow oil. Yield: 5.12 g (92%). ¹H NMR (DMSO-d₆) δ 7.02 (d, 1H), 6.22 (d, 1H), 4.05 (q, 2H), 3.25-3.20 (m, 2H), 2.59 (t, 2H), 2.44 (t, 2H), 2.25 (t, 2H), 1.87-1.80 (m, 2H), 1.79-1.71 (m, 2H), 1.17 (t, 3H).
Step 2. Preparation of 3-(3-fluorophenyl)-5-oxo-5-{2-[4-(5,6,7,8-tetrahydro-1,8-naphthyridin-2-yl)butanoyl]hydrazino}pentanoic acid.

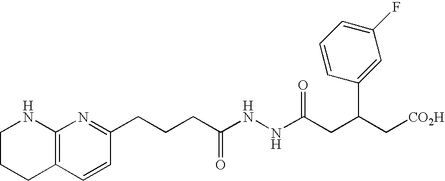

A mixture of 4-(5,6,7,8-tetrahydro-1,8-naphthyridin-2-yl)butanohydrazide, prepared as in Scheme 29, (510 mg, 2.18 mmol) and the anhydride 4-(3-fluorophenyl)dihydro-2H-pyran-2,6(3H)-dione (500 mg, 2.40 mmol) were combined in 1,4-dioxane and heated to 70° C. for 16 hrs. The resulting mixture was cooled and concentrated in vacuo to give a beige solid of the crude desired product. ¹H NMR (DMSO-d₆) δ 7.35-7.25 (m, 1H), 7.12-6.95 (m, 4H), 6.28-6.21 (m, 1H), 3.55-3.43 (m, 1H), 3.28-3.18 (m, 2H), 2.78-2.38 (m, 8H), 2.13-2.04 (t, 2H), 1.75-1.69 (m, 4H).
Step 3. Preparation of 3-(3-fluorophenyl)-4-{5-[3-(5,6,7,8-tetrahydro-1,8-naphthyridin-2-yl)propyl]-1,3,4-oxadiazol-2-yl}butanoic acid trifluoroacetate.

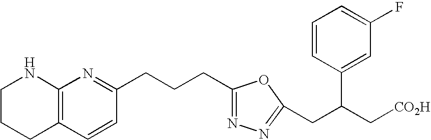

Dissolve 3-(3-fluorophenyl)-5-oxo-5-{2-[4-(5,6,7,8-tetrahydro-1,8-naphthyridin-2-yl)butanoyl]hydrazino}pentanoic acid (200 mg, 0.45 mmol) in AcOH (3 mL) in a sealed tube. Heat to 85° C. for 20 hr. The reaction was cooled and concentrate and in vacuo. The residue was purified via reverse phase HPLC using a gradient of 5-40% acetonitrile/H₂O/2% TFA over 30 min to obtain a yellow solid. Yield: 81 mg (32%). ¹H NMR (DMSO-d₆) δ7.65-7.60 (m, 1H), 7.48-7.21(m, 3H), 7.15-7.05 (m, 1H), 6.65-6.60 (m, 1H), 3.55-3.38 (m, 3H), 3.24-3.07 (m, 2H), 3.03-2.92 (m, 2H), 2.79-2.68 (m, 4H), 3.34-2.25 (t, 2H), 1.95-1.68 (m, 4H)., Analysis Calculated for C₂₃H₂₅FN₄O₃.1.2 TFA, 1.2H₂O: Expected: C, 54.18; H, 4.73; N, 9.95. Found: C, 53.96; H, 4.85; N, 10.17. Calculated Mass: 424.47. Found Mass: 425.14 (for MH⁺).

### EXAMPLE 95

3-(3-Fluoro-4-methoxyphenyl)-4-{5-[3-(5,6,7,8-tetrahydro-1,8-naphthyridin-2-yl)propyl]-1,3,4-oxadiazol-2-yl}butanoic acid trifluoroacetate.

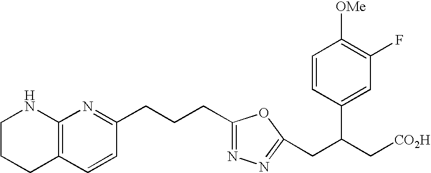

This compound was prepared by starting from 4-(3-fluoro-4-methoxyphenyl)-dihydro-2H-pyran-2,6(3H)-dione and the procedures described in example 94 above in steps 2 and 3: ¹H NMR (DMSO-d₆) δ7.67-7.60 (m, 1H), 7.27-0.17 (m, 1H), 7.11-7.08 (m, 2H), 6.68-6.60 (m, 1H), 3.83 (s, 1H), 3.47-3.38 (m, 3H), 3.18-3.01 (m, 2H), 2.98-2.89 (m, 2H), 2.78-2.65 (m, 4H), 2.33-2.25 (m, 2H), 1.92-1.78 (m, 4H), Analysis Calculated for C₂₄H₂₇FN₄O₄.2.5 TFA. Expected: C, 47.10; H, 4.02; N, 7.58. Found: C, 47.22; H, 4.06; N, 7.60. Calculated Mass: 454.49. Found Mass: 455.14 (for MH⁺).

### EXAMPLE 96

3-(3,5-Dimethoxyphenyl)-4-{5-[3-(5,6,7,8-tetrahydro-1,8-naphthyridin-2-yl)propyl]-1,3,4-oxadiazol-2-yl}butanoic acid trifluoroacetate

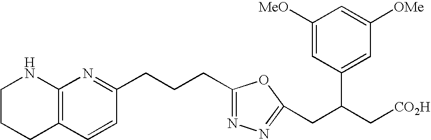

This was prepared by starting from 4-(3,5-dimethoxyphenyl)-dihydro-2H-pyran-2,6(3H)-dione and using the procedures described above as in Example 94. ¹H NMR (DMSO-d₆) δ 7.65-7.59 (m, 1H), 6.68-6.53 (m, 3H), 6.42-6.35 (m, 1H), 3.73 (s, 6H), 3.45-3.38 (m, 3H), 3.19-3.05 (m, 2H), 2.98-2.85 (m, 2H), 2.34-2.24 (m, 2H), 1.95-1.78 (m, 4H), Analysis Calculated for C₂₅H₃₀N₄O₅.1.5 TFA. Expected: C, 52.75; H, 4.98; N, 8.79. Found: C, 52.60; H, 5.40; N, 8.85. Calculated Mass: 466.53. Found Mass: 467.00 (for MH⁺)

### EXAMPLE 97

3-(2-Methyl-1,3-thiazol-5-yl)-4-{5-[3-(5,6,7,8-tetrahydro-1,8-naphthyridin-2-yl)propyl]-1,3,4-oxadiazol-2-yl}butanoic acid trifluoroacetate.

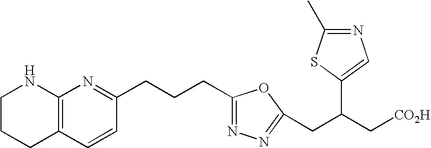

This was prepared by starting from 4-(2-methyl-1,3-thiazol-5-yl)-dihydro-2H-pyran-2,6(3H)-dione and using the procedures described above as in Example 94. ¹H NMR (DMSO-d₆) δ 7.65-7.48 (m, 2H), 6.65-6.58 (m, 1H), 3.90-3.70 (m, 1H), 3.47-3.39 (m, 2H), 3.22-2.98 (m, 4H), 2.78-2.65 (m, 4H), 2.62 (s, 3H), 2.32-2.22 (m, 2H), 1.94-1.75 (m, 4H), Analysis Calculated for C₂₁H₂₅N₅O₃S.3.2 TFA. Expected: C, 41.53; H, 3.59; N, 8.84. Found: C, 41.46; H, 3.97; N, 9.12. Calculated Mass: 427.52. Found Mass: 428.00 (for MH⁺)

### EXAMPLE 98

3-(4-Fluorophenyl)-4-{5-[3-(5,6,7,8-tetrahydro-1,8-naphthyridin-2-yl)propyl]-1,3,4-oxadiazol-2-yl}butanoic acid trifluoroacetate.

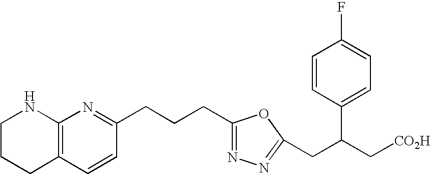

This was prepared by starting from 4-(4-fluorophenyl)-dihydro-2H-pyran-2,6(3H)-dione and using the procedures described above as in Example 94. ¹H NMR (DMSO-d₆) δ 7.65-7.59 (m, 1H), 7.50-7.39 (m, 2H), 7.25-7.12 (m, 2H), 3.62-3.38 (m, 3H), 3.20-3.04 (m, 2H), 3.00-2.88 (m, 2H), 2.78-2.65 (m, 4H), 2.35-2.25 (m, 2H), 1.97-1.67 (m, 4H), Analysis Calculated for C₂₃H₂₅FN₄O₃.1.9TFA. Expected: C, 50.21; H, 4.23; N, 8.74. Found: C, 50.49; H, 4.64; N, 8.32. Calculated Mass: 424.47. Found Mass: 425.14 (for MH⁺)

### EXAMPLE 99

3-(3,5-Difluorophenyl)-4-{5-[3-(5,6,7,8-tetrahydro-1,8-naphthyridin-2-yl)propyl]-1,3,4-oxadiazol-2-yl}butanoic acid trifluoroacetate

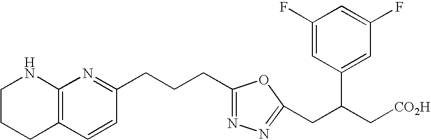

This was prepared by starting from 4-(3,5-difluorophenyl)dihydro-2H-pyran-2,6(3H)-dione and using the procedures described above as in Example 94. ¹H NMR (DMSO-d₆) δ 7.65-7.58-(m, 1H), 7.25-7.10 (m, 3H), 6.67-6.61 (m, 1H), 3.54-3.38 (m, 3H), 3.22-3.06 (m, 2H), 3.04-2.93 (m, 2H), 2.78-2.68 (m, 4H), 2.34-2.25 (m, 2H), 1.95-1.78 (m, 4H). Analysis Calculated for C₂₃H₂₄F₂N₄O₃.1.5 TFA. Expected: C, 50.90; H, 4.19; N, 9.13. Found: C, 50.08; H, 4.54; N, 8.51. Calculated Mass: 442.46. Found Mass: 443.07 (for MH⁺)

### EXAMPLE 100

3-(3,5-Difluorophenyl)-4-(5-[3-(5,6,7,8-tetrahydro-1,8-naphthyridin-2-yl)propyl]-1,3,4-thiadiazol-2-yl}butanoic acid trifluoroacetate

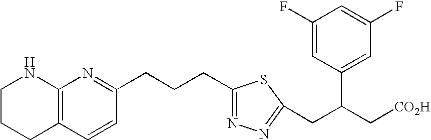

A mixture of 3-(3,5-difluorophenyl)-5-oxo-5-{2-[4-(5,6,7,8-tetrahydro-1,8-naphthyridin-2-yl)butanoyl]hydrazino}pentanoic acid (992 mg, 2.24 mmol), was stirred in benzene (10 mL) under argon. Add P₂S₅ (1.60 g, 3.60 mmol) and heat to reflux for 16 hrs. The reaction mixture was concentrated in vacuo and triturated with acetonitrile/H₂O mixture. The supernatant was decanted and concentrated in vacuo. This procedure was repeated several times and the resulting residue was dissolved in AcOH (25 mL) and heated to reflux overnight. The reaction mixture was concentrated in vacuo and purified via reverse phase HPLC using a gradient of 10-50% % acetonitrile/H₂O/2% NH₄OAC over 30 min. To gain the TFA salt of the desired product, another reverse phase HPLC was done using the gradient of 10-50% acetonitrile/H₂O/0.05% TFA over 30 min. Yield: 80 mg (6%). ¹H NMR (DMSO-d₆) δ 7.61-7.52 (m, 1H), 7.07-6.98 (m, 3H), 6.59-6.53 (m, 1H), 3.52-3.32(m, 5H), 3.04-2.95 (m, 2H), 2.79-2.58 (m, 4H), 2.03-1.93 (m, 2H), 1.84-1.74 (m, 2H). Analysis Calculated for C₂₃H₂₄F₂N₄O₂S.2.5 TFA. Expected: C, 45.23; H, 3.59; N, 7.53. Found: C, 45.33; H, 3.72; N, 8.01. Calculated Mass: 458.53. Found Mass: 459.16 (for MH⁺)

### EXAMPLE 101

3-(4-Fluorophenyl)-4-{5-[3-(5,6,7,8-tetrahydro-1,8-naphthyridin-2-yl)propyl]-1,3,4-thiadiazol-2-yl}butanoic acid trifluoroacetate

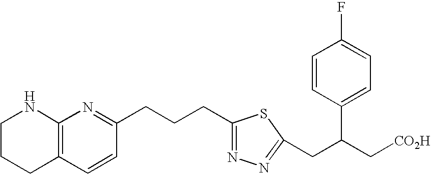

This compound was prepared starting from 3-(4-fuorophenyl)-5-oxo-5-{2-[4-(5,6,7,8-tetrahydro-1,8-naphthyridin-2-yl)butanoyl]hydrazino}pentanoic acid (992 mg, 2.24 mmol), and using the procedure described for Example 100. ¹H NMR (DMSO-d₆) δ 7.54-7.48 (m, 1H), 7.24-7.15(m, 2H), 7.06-6.94 (m, 2H), 6.52-6.45 (m, 1H), 2.42-3.24 (m, 5H), 2.95-2.88(m, 2H), 2.74-2.47(m, 6H), 1.97-1.83(m, 2H), 1.78-1.67(m, 2H). Analysis Calculated for C₂₃H₂₅FN₄O₂S.1.7 TFA. Expected: C, 49.98; H, 4.24; N, 8.83. Found: C, 49.83; H, 4.52; N, 9.21. Calculated Mass: 440.53. Found Mass: 441.17 (for MH⁺)

### EXAMPLE 102

3-(2-Methyl-1,3-thiazol-5-yl)-4-{5-[3-(5,6,7,8-tetrahydro-1,8-naphthyridin-2-yl)propyl]-1,3,4-thiadiazol-2-yl}butanoic acid trifluoroacetate

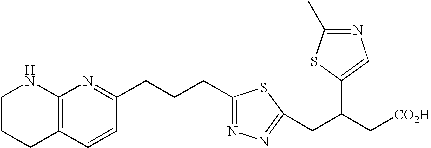

This compound was prepared starting from 3-(2-methyl-1,3-thiazol-5-yl)-5-oxo-5-{2-[4-(5,6,7,8-tetrahydro-1,8-naphthyridin-2-yl)butanoyl]hydrazino}pentanoic acid (992 mg, 2.24 mmol), and using the procedure described for Example 100. ¹H NMR (DMSO-d₆) δ 7.68-7.62 (m, 1H), 6.69-6.63(m, 1H), 3.90-3.77(m, 1H), 3.59-3.38 (m, 2H), 3.15-3.07(m, 2H), 2.91-2.63 (m, 8H), 2.62 (s, 3H), 2.15-2.02(m, 2H), 1.94-1.83(m, 2H). Analysis Calculated for C₂₁H₂₅N₅O₂S₂.4.5 TFA. Expected: C, 37.66; H, 3.11; N, 7.32. Found: C, 37.66; H, 3.73; N, 9.56. Calculated Mass: 443.59. Found Mass: 444.15 (for MH⁺)

### EXAMPLE 103

3-(1,3-Benzodioxol-5-yl)-4-{5-[3-(5,6,7,8-tetrahydro-1,8-naphthyridin-2-yl)propyl]-1,3,4-thiadiazol-2-yl}butanoic acid trifluoroacetate.

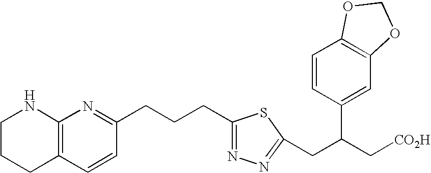

This compound was prepared starting from 3-(1,3-benzodioxol-5-yl)-5-oxo-5-{2-[4-(5,6,7,8-tetrahydro-1,8-naphthyridin-2-yl)butanoyl]hydrazino}pentanoic acid (992 mg, 2.24 mmol), and using the procedure described for Example 100. ¹H NMR (DMSO-d₆) δ 8.00 (br s, 1H), 7.60 (d, 1H), 6.93 (d, 1H), 6.77 (d, 1H), 6.67 (dd, 1H), 6.57 (d, 1H), 5.94 (d, 2H), 3.43-3.30(m, 4H), 3.02 (t, 2H), 2.77-2.66-(m, 4H), 2.60-2.45 (m, 2H), 2.04-1.95 (m, 2H), 1.87-1.78 (m, 2H). Calculated Mass: 467.1746. Found Mass: 467.1745 (for MH⁺).

### EXAMPLE 104

Synthesis of 3-(1,3-benzodioxol-5-yl)-4-{3-[2-(5,6,7,8-tetrahydro-1,8-naphthyridin-2-yl)ethoxy]isoxazol-5-yl}butanoic acid. 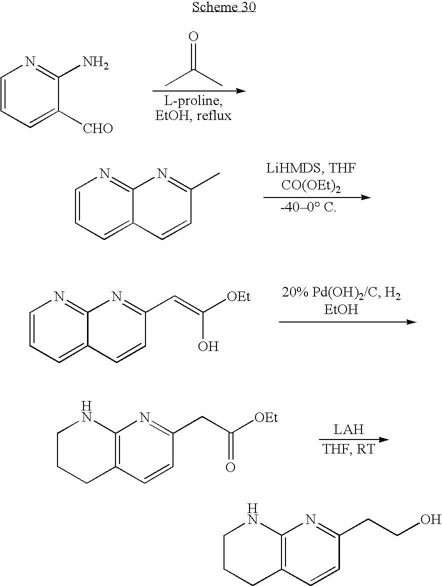
Step 1. Synthesis of 2-methyl-1,8-naphthyridine.

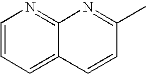

To 2-amino-3-nicotinaldehyde (50.0 g, 0.41 mol) in EtOH (600 mL) was added L-proline (51 g, 0.45 mol) and acetone (90 mL, 1.23 mol). The reaction mixture was refluxed overnight. The reaction mixture was cooled to room temperature and the white solid filtered. The filtrate was concentrated to a yellowish solid, redissolved in CH₂Cl₂ (500 mL), and the insolubles filtered. The filtrate was washed with water (2×100 mL), the organic layer was separated and the aqueous layers combined and washed with CH₂Cl₂ (4×75 mL). The organic layers were combined, washed with brine, dried over Na₂SO₄ and concentrated to a yellow solid (57.2 g, 0.40 mol, 97%).
Step 2. Synthesis of (E)-1-ethoxy-2-(1,8-naphthyridin-2-yl)ethanol.

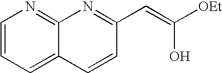

To the product from step 1, (81.5 g, 0.57 mol) in anhydrous THF (1.9 L) at −40° C. under Ar gas was added lithium bis(trimethylsilyl)amide (1 M in THF, 1.2 L, 1.2 mol). After stirring for 30 min at −40° C., diethylcarbonate (72.5 mL, 0.60 mol) was added. The temperature of the reaction mixture was warmed up to 0° C. and stirred for 2 h. The reaction mixture was quenched into saturated aq. NH₄Cl (700 mL) and the THF removed under reduced pressure. The resulting mixture was extracted with EtOAc (3×700 mL). The organic layers were combined, washed with brine, dried over Na₂SO₄, and concentrated under reduced pressure. The resulting residue was purified by flash column chromatography using 50% EtOAc/hexane to give a yellow solid (81.2 g, 0.38 mol, 66%). ¹H NMR (400 MHz, DMSO-d6) δ 1.22 (t, 3H), 4.11(q, 2H), 4.89 (s, 1H), 6.78 (d, 1H), 7.15 (dd, 1H), 7.47 (d, 1H), 7.80 (d, 1H), 8.36 (d, 1H), 11.8 (bs, 1H). LC-MS (MH+)=217.
Step 3. Synthesis of ethyl 5,6,7,8-tetrahydro-1,8-naphthyridin-2-ylacetate.

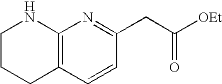

Compound from step 2, (51.4 g, 0.24 mol) in EtOH was hydrogenated using 20% Pd(OH)₂/C at room temperature under a pressure of 5 psi. After 2 h, the reaction was complete. The Pd(OH)₂/C was filtered and the filtrate concentrated to a yellow solid (50.3 g, 0.23 mol, 96%). ¹H NMR (400 MHz, DMSO-d6) δ 1.17 (t, 3H), 1.74 (m, 2H), 2.61 (t, 2H), (3.23, 2H), 3.47 (s, 2H), 4.04 (q, 2H), 6.32 (d, 1H), 6.41 (bs, 1H), 7.07 (d, 1H). LC-MS (MH+)=221.
Step 4. Synthesis of 2-(5,6,7,8-tetrahydro-1,8-naphthyridin-2-yl)ethanol.

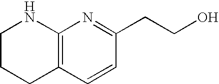

To anhydrous THF (910 mL) under Ar gas at room temperature was added a 1M solution of lithium aluminum hydride in THF (910 mL, 0.91 mol). The temp of the reaction mixture was lowered to 15° C. and a solution of product from step 3, (50.3 g, 0.23 mol) in anhydrous THF (500 mL) was slowly added over 30 min. The resulting reaction was stirred at room temperature for 3.5 h. The temperature was lowered to 0° C. and the reaction was slowly quenched with brine (260 mL). Additional THF (300 mL) was added during the quench to break-up the emulsions. After complete addition of brine, the reaction mixture was stirred at RT overnight. Na₂SO₄ was added and the mixture stirred for 15 min and filtered. The residue was washed with EtOAc (3×300). The organics were combined, concentrated to about 1.5 L, dried with Na₂SO₄, and concentrated under reduced pressure. The resulting residue was purified by flash column chromatography using 100% EtOAc, followed by 5% MeOH/EtOAc as eluents. The desired product was obtained as solid (34.9 g, 85%). 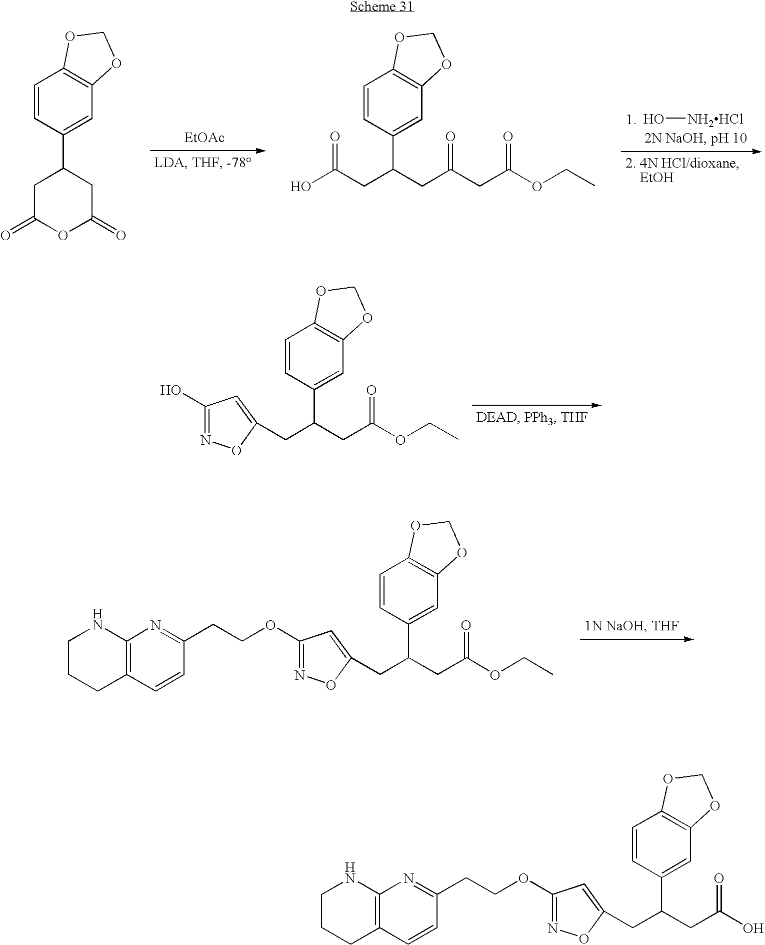
Step 5. Synthesis of 3-(1,3-benzodioxol-5-yl)-7-ethoxy-5,7-dioxoheptanoic acid.

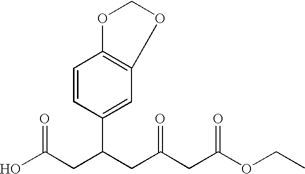

To a solution of anhydrous EtOAc (4.38 mL, 44.8 mmol) in anhydrous THF (25 mL) at −78° C. under Ar gas was slowly added lithium diisopropylamide (2M in heptane/THF/ethylbenzene, 22.4 mL, 44.8 mmol). The resulting solution was stirred at −78° C. for 25 min and added dropwise via cannula to a solution of the anhydride (5.0 g, 21.3 mmol) in anhydrous THF (170 mL) at −78° C. under Ar gas. The reaction mixture was stirred at −78° C. for 1.5 h. The reaction mixture was quenched with 2N HCl in ether (80 mL) and allowed to warm up to room temperature. To the reaction mixture was added water (100 mL) and extracted with EtOAc (3×100 mL). The organic layers were combined, washed with brine, dried over Na₂SO₄, and concentrated under reduced pressure. The resulting residue was purified by flash column chromatography using 40% EtOAc/hexane to give a white solid (5.61 g, 17.4 mmol, 82%). ¹H NMR (400 MHz, CDCl₃) δ 1.25 (t, 3H), 2.55-2.73 (m, 2H), 2.90 (m, 2H), 3.34 (s, 2H), 3.60 (m, 1H), 4.15 (q, 2H), 5.93 (s, 2H), 6.70 (m, 3H). LC-MS (M+Na)=345.
Step 6. Synthesis of ethyl 3-(1,3-benzodioxol-5-yl)-4-(3-hydroxyisoxazol-5-yl)butanoate.

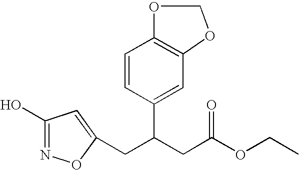

Hydroxylamine hydrochloride (1.1 g, 16.4 mmol) was dissolved in approximately 4.3 mL of 2N NaOH to achieve a solution of pH 10.0±0.3 (pH meter used). The solution was cooled to 0° C. and stirred vigorously while a solution of product from step 5 (4.8 g, 14.9 mmol) in 2N NaOH (approximately 8.5 mL) was added slowly while maintaining the pH of the reaction mixture at 10.0+0.3 by dropwise addition of 2N NaOH. After complete addition, the reaction mixture was stirred at 0° C. for 1.5 h and quenched into ice cold concentrated HCl (20 mL). The reaction mixture was warmed up to room temperature and stirred for 4 h. The resulting mixture was poured into ice water (200 mL) and extracted with EtOAc (3×200 mL). The organic layers were combined, washed with brine, dried over Na₂SO₄, and concentrated under reduced pressure. The resulting residue (3.8 g) was dissolved in EtOH (15 mL), and 4N HCl in dioxane (15 mL) was added. The reaction mixture was stirred at room temperature overnight. The reaction mixture was concentrated under reduced pressure and the residue purified by flash column chromatography using 50% EtOAc/hexane as eluent. Obtained was a yellow oil (1.22 g, 3.8 mmol, 26%). ¹H NMR (400 MHz, DMSO-d6) δ 1.08 (t, 3H), 2.54-2.72 (m, 2H), 2.93 (m, 2H), 3.33 (m, 1H), 3.95 (q, 2H), 5.58 (s, 1H), 5.97 (s, 2H). 6.68 (dd, 1H), 6.78 (d, 1H), 6.91 (d, 1H), 10.95 (s, 1H). LC-MS (MH+)=320.
Step 7. Synthesis of ethyl 3-(1,3-benzodioxol-5-yl)-4-{3-[2-(5,6,7,8-tetrahydro-1,8-naphthyridin-2-yl)ethoxy]isoxazol-5-yl}butanoate.

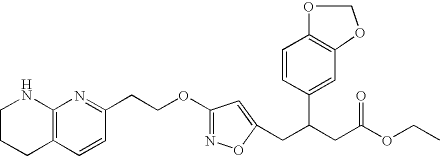

To 2-(5,6,7,8-tetrahydro-1,8-naphthyridin-2-yl)ethanol (400 mg, 1.2 mmol), ethyl 3-(1,3-benzodioxol-5-yl)-4-(3-hydroxyisoxazol-5-yl)butanoate (246 mg, 1.4 mmol), and triphenylphosphine (393 mg, 1.5 mmol) in anhydrous THF under Ar gas at room temperature was added diethyl azodicarboxylate (236 μL, 1.5 mmol). The reaction mixture was stirred overnight. The reaction mixture was quenched into saturated aqueous NH₄Cl (5 mL) and extracted with EtOAc (3×5 mL). The organic layers were combined, washed with brine, dried over Na₂SO₄, and concentrated under reduced pressure. The residual oil was purified by flash column chromatography using 100% EtOAc as eluent. Obtained was an oil (466 mg) containing the product and a triphenylposphine oxide impurity. ¹H NMR (400 MHz, DMSO-d6) δ 1.06 (t, 3H), 1.75 (m, 2H), 2.53-2.73 (m, 4H), 2.84 (t, 2H), 2.97 (m, 2H), 3.23 (m, 2H), 3.35 (m, 1H), 3.94 (m, 2H), 4.35 (t, 2H), 5.79 (s, 1H), 5.97 (s, 2H), 6.29 (d, 1H), 6.34 (bs, 1H), 6.68 (dd, 1H), 6.77 (d, 1H), 6.91 (d, 1H), 7.03 (d, 1H). LC-MS (MH+)=480.
Step 8. Synthesis of 3-(1,3-benzodioxol-5-yl)-4-{3-[2-(5,6,7,8-tetrahydro-1,8-naphthyridin-2-yl)ethoxy]isoxazol-5-yl}butanoic acid.

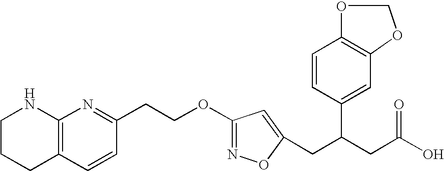

To the impure product from step 7 (466 mg) in THF (5 mL) was added 1N NaOH (5 mL). The reaction mixture was stirred at room temperature overnight. The reaction mixture was acidified, concentrated, and purified by reverse phase HPLC using (H₂O/TFA)/CH₃CN as eluent (2.5 mL TFA in 4 L H₂O). Obtained was a white solid (141, 0.24 mmol, 2 step yield 19%). ¹H NMR (400 MHz, DMSO-d6) δ 1.82 (m, 2H), 2.45-2.64 (m, 2H), 2.74 (t, 2H), 2.97 (m, 2H), 3.08 (t, 2H), 3.30 (m, 1H), 3.40 (m, 2H), 4.38 (t, 2H), 5.81 (s, 1H), 5.96 (s, 2H), 6.65 (m, 2H), 6.77 (d, 1H), 6.90 (d, 1H), 7.58 (d, 1H), 8.07 (bs, 1H), 12.14 (bs, 1H). LC-MS (MH+)=451. Anal. Cald. for C₂₄H₂₅N₃O₆.1.2TFA.0.25H₂O: C, 53.49; H, 4.54; N, 7.09. Found: C, 53.37; H, 4.40; N, 7.07.

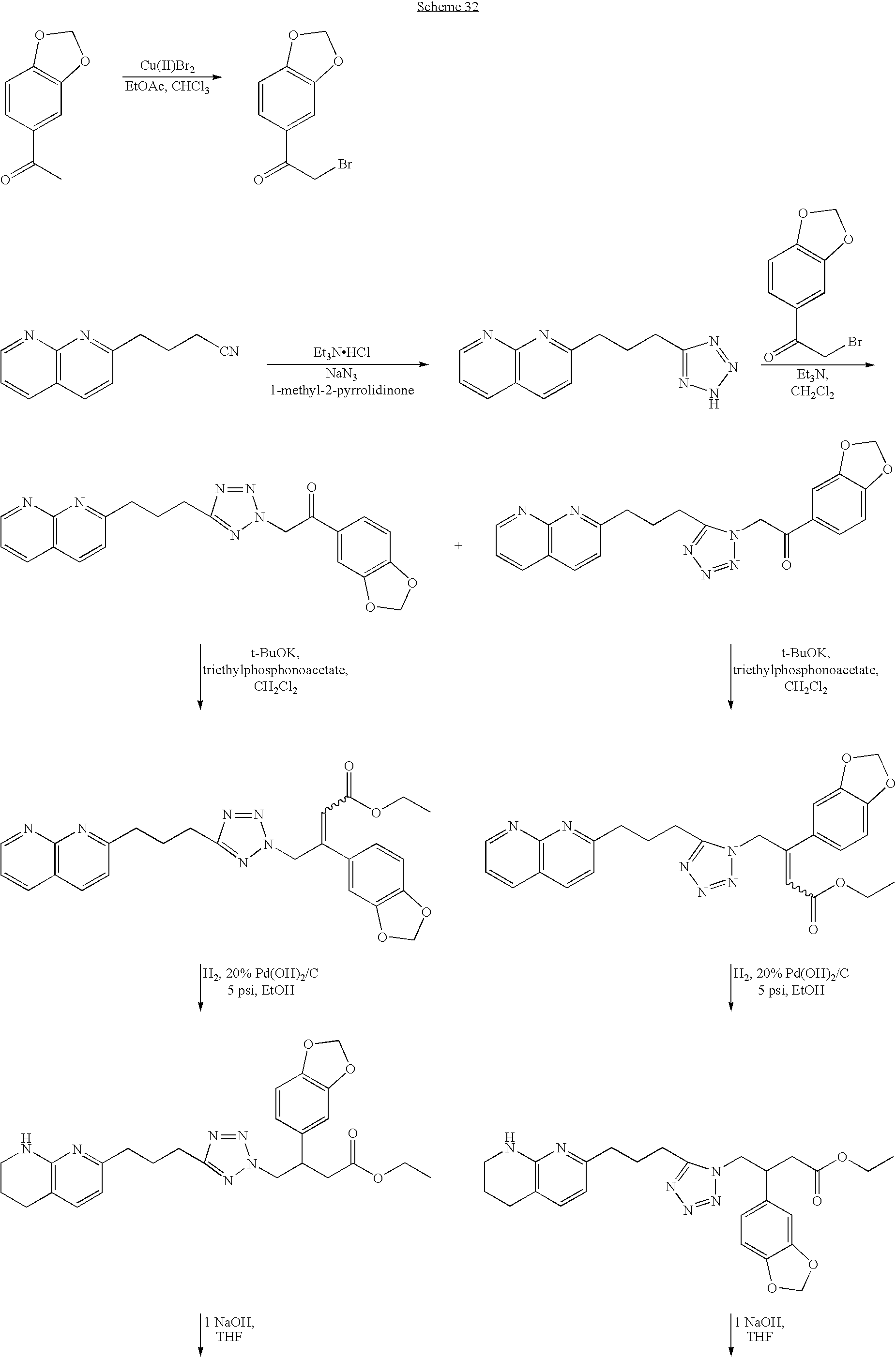

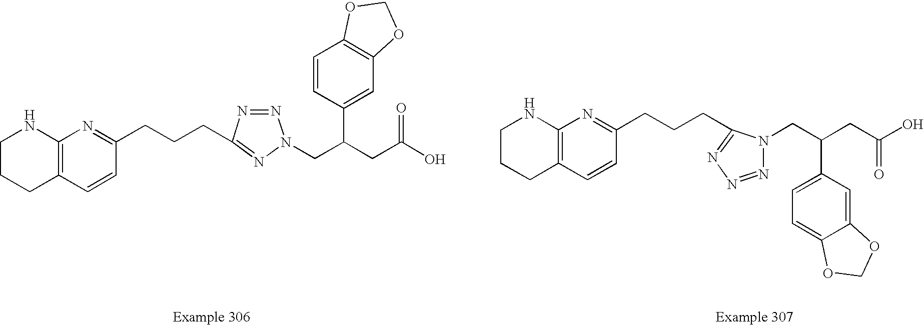

### EXAMPLE 105

Synthesis of 3-(1,3-benzodioxol-5-yl)-4-{5-[3-(5,6,7,8-tetrahydro-1,8-naphthyridin-2-yl)propyl]-2H-tetraazol-2-yl}butanoic acid.

Step 1. Synthesis of 1-(1,3-benzodioxol-5-yl)-2-bromoethanone.

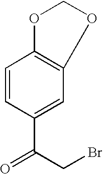

3′,4′-(methylenedioxy)acetophenone (10.0 g, 61 mmol) in CHCl₃ (360 mL) was added to a mixture of Cu(II)Br₂ (27.0 g, 122 mmol) in EtOAc (360 mL) at 65° C. The resulting mixture was refluxed for 4 h. The reaction mixture was cooled to room temperature and Norite was added. The mixture was stirred for 1 h and filtered through celite. The filtrate was concentrated under reduced pressure. The resulting residue was purified by flash column chromatography using 15% EtOAc/hexane as eluent. Obtained was a white solid (8.44 g, 57%). ¹H NMR (400 MHz, DMSO-d6) δ 4.85 (s, 2H), 6.15 (s, 2H), 7.07 (d, 1H), 7.47 (d, 1H), 7.65 (dd, 1H). LC-MS (MH+)=244.
Step 2. Synthesis of 2-[3-(2H-tetraazol-5-yl)propyl]-1,8-naphthyridine.

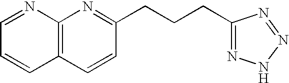

To the nitrile [synthesis described previously] (1.0 g, 5.1 mmol) in 1-methyl-2-pyrrolidinone (15 mL) was added NaN₃ (1.0 g, 15 mmol) and Et₃N.HCl (1.1 g, 8.1 mmol). The reaction was heated to 160° C. for 8 h and allowed to cool to room temperature overnight. The precipitate was filtered. To the filtrate was added 2N HCl in ether (70 mL). After stirring for 30 min, the resulting brown solid was filtered, washed with EtOAc (3×5 mL) and CH₂Cl₂ (3×5 mL). Obtained was a brown solid (1.26 g, 4.0 mmol, 79%). ¹H NMR (400 MHz, DMSO-d6) δ 2.32 (m, 2H), 3.04 (t, 2H), 3.24 (t, 2H), 8.0 (m, 2H), 8.87 (d, 1H), 9.0 (dd, 1H), 9.30 (dd, 1H). LC-MS (MH+)=241. Anal. Cald. for C₁₂H₁₂N₆: 2HCl: C, 46.02; H, 4.51; N, 26.83. Found: C, 45.76; H, 4.23; N, 26.60.
Step 3. Synthesis of 1-(1,3-benzodioxol-5-yl)-2-{5-[3-(1,8-naphthyridin-2-yl)propyl]-2H-tetraazol-2-yl}ethanone and 1-(1,3-benzodioxol-5-yl)-2-{5-[3-(1,8-naphthyridin-2-yl)propyl]-1H-tetraazol-1-yl}ethanone.

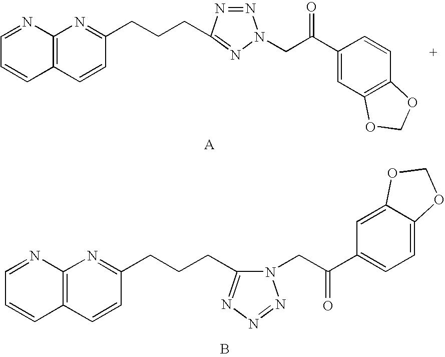

To 2-[3-(2H-tetraazol-5-yl)propyl]-1,8-naphthyridine (918 mg, 3.6 mmol) in anhydrous CH₂Cl₂ (35 mL) at room temperature was added anhydrous Et₃N (5 mL, 36 mmol). After 45 min at room temperature, compound 1-(1,3-benzodioxol-5-yl)-2-bromoethanone (1.1 g, 4.4 mmol) was added and the reaction mixture stirred overnight. The reaction mixture was concentrated under reduced pressure. The resulting residue was purified by flash column chromatography using 5% MeOH/EtOAc as eluent. Obtained were isomers A (400 mg, 1.0 mmol, 28%) and B (488 mg, 1.2 mmol, 34%). Compound A: ¹H NMR (400 MHz, DMSO-d6) δ 2.28 (m, 2H), 3.00 (t, 2H), 3.07 (t, 2H), 6.19 (s, 2H), 6.49 (s, 2H), 7.15 (d, 1H), 7.52 (d, 1H), 7.57 (m, 2H), 7.73 (dd, 1H), 8.40 (d, 1H), 8.44 (dd, 1H), 9.03 (dd, 1H). LC-MS (MH+)=403. Compound B: ¹H NMR (400 MHz, DMSO-d6) δ 2.24 (m, 2H), 2.91 (t, 2H), 3.05 (t, 2H), 6.19 (s, 2H), 6.33 (s, 2H), 7.14 (d, 1H), 7.50 (d, 1H), 7.54 (d, 1H), 7.58 (dd, 1H), 7.73 (dd, 1H), 8.35 (d, 1H), 8.41 (dd, 1H), 9.02 (dd, 1H). LC-MS (MH+)=403.
Step 4. Synthesis of ethyl(2E)-3-(1,3-benzodioxol-5-yl)-4-{5-[3-(1,8-naphthyridin-2-yl)propyl]-2H-tetraazol-2-yl}but-2-enoate.

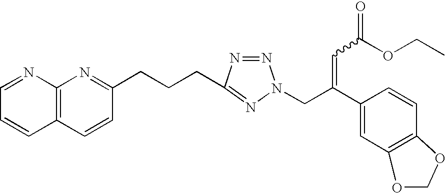

To a solution of t-BuOK (1M in THF, 1.4 mL, 1.4 mmol) in anhydrous CH₂Cl₂ (6 mL) at room temperature under Ar gas was slowly added triethylphosphonoacetate (277 μL, 1.4 mmol). After stirring for 30 min, a solution of 1-(1,3-benzodioxol-5-yl)-2-{5-[3-(1,8-naphthyridin-2-yl)propyl]-2H-tetraazol-2-yl}ethanone (469 mg, 1.2 mmol) in anhydrous CH₂Cl₂ (10 mL) was added. The reaction mixture was stirred at room temperature overnight. The reaction mixture was loaded onto a flash silica gel column and purified using 5% MeOH/EtOAc to give a mixture of cis and trans isomers (480 mg). LC-MS (MH+)=473.
Step 5. Synthesis of ethyl(2E)-3-(1,3-benzodioxol-5-yl)-4-{5-[3-(1,8-naphthyridin-2-yl)propyl]-1H-tetraazol-1-yl}but-2-enoate.

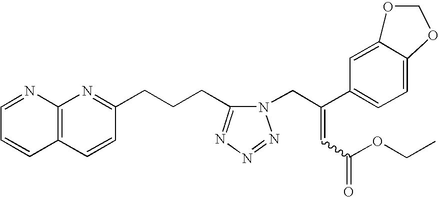

Same synthetic procedure as for ethyl(2E)-3-(1,3-benzodioxol-5-yl)-4-{5-[3-(1,8-naphthyridin-2-yl)propyl]-2H-tetraazol-2-yl}but-2-enoate, using 1-(1,3-benzodioxol-5-yl)-2-{5-[3-(1,8-naphthyridin-2-yl)propyl]-1H-tetraazol-1-yl}ethanone as starting material. LC-MS (MH+)=473
Step 6. Synthesis of ethyl 3-(1,3-benzodioxol-5-yl)-4-{5-[3-(5,6,7,8-tetrahydro-1,8-naphthyridin-2-yl)propyl]-2H-tetraazol-2-yl}butanoate.

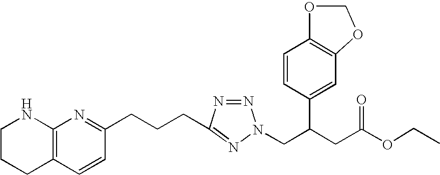

Ethyl(2E)-3-(1,3-benzodioxol-5-yl)-4-{5-[3-(1,8-naphthyridin-2-yl)propyl]-2H-tetraazol-2-yl}but-2-enoate (480 mg) in EtOH was hydrogenated using 20% Pd(OH)₂/C at room temperature under a pressure of 5 psi. After 6 h, the reaction was complete. The Pd(OH)₂/C was filtered and the filtrate concentrated to the desired product (435 mg). LC-MS (MH+)=479.
Step 7. Synthesis of ethyl 3-(1,3-benzodioxol-5-yl)-4-{5-[3-(5,6,7,8-tetrahydro-1,8-naphthyridin-2-yl)propyl]-1H-tetraazol-1-yl}butanoate.

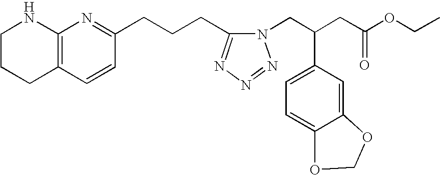

Same synthetic procedure as for ethyl 3-(1,3-benzodioxol-5-yl)-4-{5-[3-(5,6,7,8-tetrahydro-1,8-naphthyridin-2-yl)propyl]-2H-tetraazol-2-yl}butanoate, using ethyl (2E)-3-(1,3-benzodioxol-5-yl)-4-{5-[3-(1,8-naphthyridin-2-yl)propyl]-1H-tetraazol-1-yl}but-2-enoate as starting material. LC-MS (MH+)=479
Step 8. Synthesis of 3-(1,3-benzodioxol-5-yl)-4-{5-[3-(5,6,7,8-tetrahydro-1,8-naphthyridin-2-yl)propyl]-2H-tetraazol-2-yl}butanoic acid

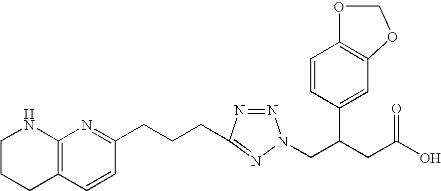

Same procedure as a previous example using ethyl 3-(1,3-benzodioxol-5-yl)-4-{5-[3-(5,6,7,8-tetrahydro-1,8-naphthyridin-2-yl)propyl]-2H-tetraazol-2-yl}butanoate as starting material. ¹H NMR (500 MHz, DMSO-d6) δ 1.83 (m, 2H), 1.98 (m, 2H), 2.64 (m, 3H), 2.73 (m, 3H), 2.81 (t, 2H), 3.42 (m, 2H), 3.64 (m, 1H), 4.85 (m, 2H), 5.93 (d, 2H), 6.55 (m, 2H), 6.70 (d, 1H), 6.88 (d, 1H), 7.59 (d, 1H), 8.37 (bs, 1H). Anal. Cald. for C₂₃H₂₆N₆O₄.1.1TFA.0.25H₂O: C, 52.15; H, 4.79; N, 14.48. Found: C, 52.20; H, 4.68; N, 14.49.

### EXAMPLE 106

Synthesis of 3-(1,3-benzodioxol-5-yl)-4-{5-[3-(5,6,7,8-tetrahydro-1,8-naphthyridin-2-yl)propyl]-1H-tetraazol-1-yl}butanoic acid

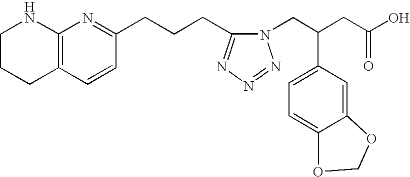

Same procedure as a previous example using ethyl 3-(1,3-benzodioxol-5-yl)-4-{5-[3-(5,6,7,8-tetrahydro-1,8-naphthyridin-2-yl)propyl]-1H-tetraazol-1-yl}butanoate as starting material. ¹H NMR (400 MHz, DMSO-d6) δ 1.84 (m, 4H), 2.58-2.85 (m, 8H), 3.41 (m, 2H), 3.50 (m, 1H), 4.43-4.63 (m, 2H), 5.92 (d, 2H), 6.50 (d, 1H), 6.55 (d, 1H), 6.72 (d, 1H), 6.79 (s, 1H), 7.61 (d, 1H), 8.15 (bs, 1H). LC-MS (MH+)=451. Anal. Cald. for C₂₃H₂₆N₆O₄.1.6TFA.0.25H₂O: C, 49.37; H, 4.44; N, 13.18. Found: C, 49.38; H, 4.27; N, 13.39.

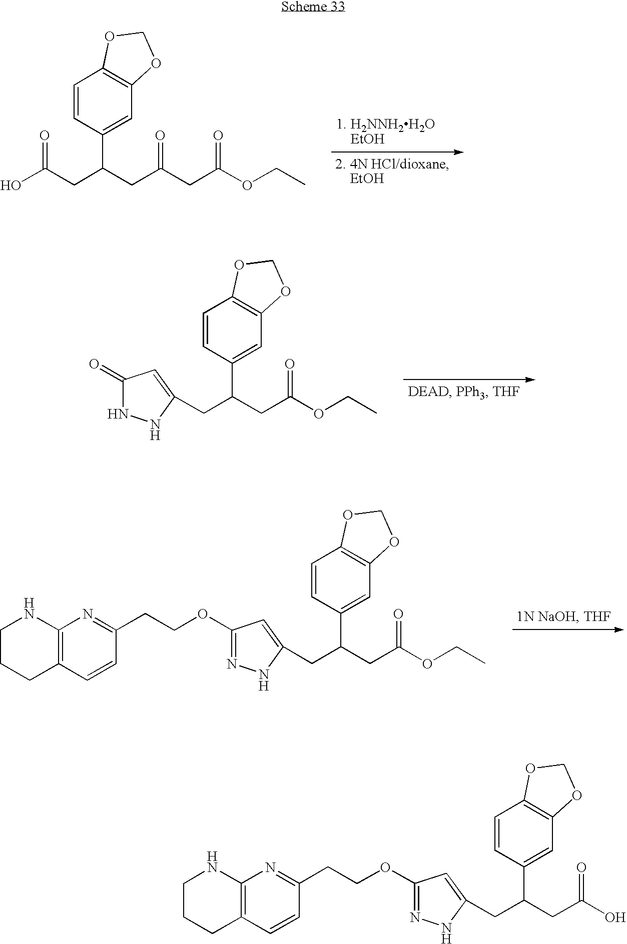

### EXAMPLE 107

Synthesis of 3-(1,3-benzodioxol-5-yl)-4-{3-[2-(5,6,7,8-tetrahydro-1,8-naphthyridin-2-yl)ethoxy]-1H-pyrazol-5-yl}butanoic acid. 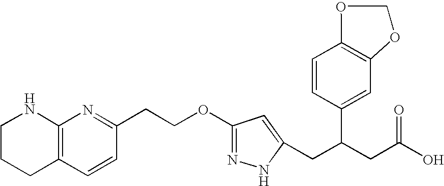
Step 1. Synthesis of ethyl 3-(1,3-benzodioxol-5-yl)-4-(5-oxo-2,5-dihydro-1H-pyrazol-3-yl)butanoate.

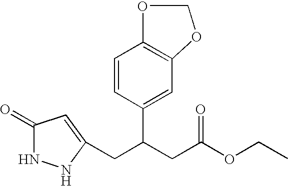

To 3-(1,3-benzodioxol-5-yl)-7-ethoxy-5,7-dioxoheptanoic acid (1.0 g, 3.1 mmol) in absolute EtOH (6 mL) at 45° C. was added hydrazine monohydrate (165 μL, 3.4 mmol). The resulting solution was heated at 45° C. for 7 h and allowed to cool to room temperature overnight. The solvent was removed under reduced pressure. The residue was dissolved in absolute EtOH (7 mL) and 4N HCl in dioxane (7 mL) was added. The reaction mixture was stirred at room temperature overnight. The reaction mixture was concentrated under reduced pressure and partitioned between saturated aqueous NaHCO₃ (20 mL) and EtOAc (20 mL). The organic layers were separated and the aqueous extracted with EtOAc (2×15 mL). The organic layers were combined, washed with brine, dried over Na₂SO₄, and concentrated under reduced pressure. Obtained was a brown solid (880 mg, 2.8 mmol, 89%). ¹H NMR (400 MHz, DMSO-d6) δ 1.04 (t, 3H), 2.51-2.63 (m, 2H), 2.72 (m, 2H), 3.25 (m, 1H), 3.93 (m, 2H), 5.09 (s, 1H), 5.95 (s, 2H), 6.65 (dd, 1H), 6.77 (d, 1H), 6.85 (d, 1H). LC-MS (MH+)=319.
Step 2. Synthesis of ethyl 3-(1,3-benzodioxol-5-yl)-4-{3-[2-(5,6,7,8-tetrahydro-1,8-naphthyridin-2-yl)ethoxy]-1H-pyrazol-5-yl}butanoate.

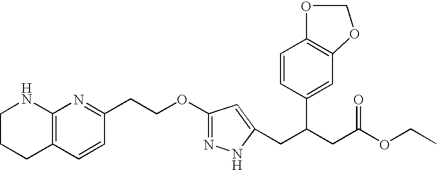

To ethyl 3-(1,3-benzodioxol-5-yl)-4-(5-oxo-2,5-dihydro-1H-pyrazol-3-yl)butanoate (300 mg, 0.94 mmol), 2-(5,6,7,8-tetrahydro-1,8-naphthyridin-2-yl)ethanol (178 mg, 1.0 mmol), and triphenylphosphine (296 mg, 1.1 mmol) in anhydrous THF (4 mL) under Ar gas at room temperature was added DEAD (173 μL, 1.1 mmol). The reaction mixture was stirred overnight. The reaction mixture was quenched into H₂O (5 mL) and extracted with EtOAc (3×5 mL). The organic layers were combined, washed with brine, dried over Na₂SO₄, and concentrated under reduced pressure. The resulting residue was purified by flash column chromatography using 10% MeOH/EtOAc as eluent. Obtained was an oil (117 mg, 0.24 mmol, 26%). ¹H NMR (400 MHz, DMSO-d6) δ 1.05 (t, 3H), 1.74 (m, 2H), 2.53-2.64 (m, 4H), 2.78 (m, 4H), 3.20-3.31 (m, 3H), 3.93 (dq, 2H), 4.21 (t, 2H), 5.27 (s, 1H), 5.95 (s, 2H), 6.29 (m, 2H), 6.64 (dd, 1H), 6.77 (d, ₁H), 6.84 (d, 1H), 7.03 (d, 1H), 11.5 (bs, 1H). LC-MS (MH+)=479.
Step 3. Synthesis of 3-(1,3-benzodioxol-5-yl)-4-{3-[2-(5,6,7,8-tetrahydro-1,8-naphthyridin-2-yl)ethoxy]-1H-pyrazol-5-yl}butanoic acid.

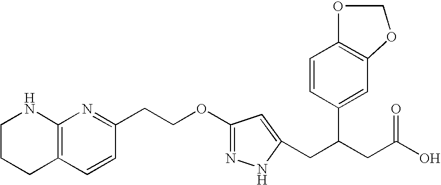

Same synthetic procedure as a previous example using ethyl 3-(1,3-benzodioxol-5-yl)-4-{3-[2-(5,6,7,8-tetrahydro-1,8-naphthyridin-2-yl)ethoxy]-1H-pyrazol-5-yl}butanoate as starting material. ¹H NMR (400 MHz, DMSO-d6) δ 1.82 (m, 2H), 2.41-2.56 (m, 2H), 2.75 (m, 4H), 3.04 (t, 2H), 3.24 (m, 1H), 3.41 (m, 2H), 4.26 (t, 2H), 5.29 (s, 1H), 5.95 (s, 2H), 6.65 (m, 2H), 6.77 (d, 1H), 6.83 (d, 1H), 7.61 (d, 1H), 8.09 (bs, 1H). LC-MS (MH+)=451. Anal. Cald. for C₂₄H₂₆N₄O₅.2.6TFA: C, 46.95; H, 3.86; N, 7.50. Found: C, 46.74; H, 3.74; N, 7.78.

### EXAMPLE 108

Synthesis of 3-(1,3-benzodioxol-5-yl)-4-{3-[3-(4,5-dihydro-1H-imidazol-2-ylamino)propoxy]isoxazol-5-yl}butanoic acid. 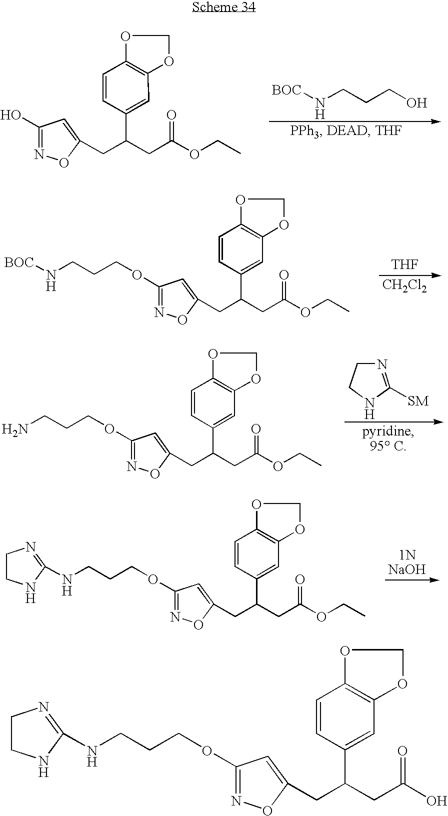
Step 1. Synthesis of ethyl 3-(1,3-benzodioxol-5-yl)-4-{3-[3-(N-BOC amino)propoxy]isoxazol-5-yl}butanoate.

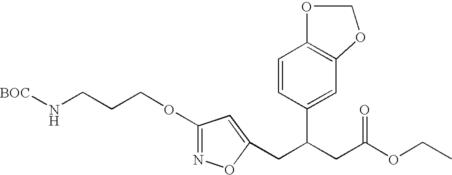

To ethyl 3-(1,3-benzodioxol-5-yl)-4-(3-hydroxyisoxazol-5-yl)butanoate.

(890 mg, 2.8 mmol) in anhydrous THF (11 mL) was added triphenylphosphine (892 mg, 3.4 mmol) and t-butyl N-(3-hydroxypropyl)-carbamate (581 μL, 3.4 mmol). The temperature of the reaction mixture was lowered to 0° C. and DEAD (535 μL, 3.4 mmol) was added. The reaction mixture was allowed to warm up to room temperature and stirred for 4 h. The reaction mixture was concentrated under reduced pressure and the resulting residue purified by flash column chromatography using 50% EtOAc/hexane as eluent. Obtained was a yellow oil (890 mg, 1.9 mmol, 67%). ¹H NMR (400 MHz, DMSO-d6) δ 1.07 (t, 3H), 1.35 (s, 9H), 1.77 (m, 2H), 2.54-2.73 (m, 2H), 2.99 (m, 4H), 3.33 (m, 1H), 3.95 (dq, 2H), 4.07 (t, 2H), 5.81 (s, 1H), 5.97 (s, 2H), 6.68 (dd, 1H), 6.77 (d, 1H), 6.87 (t, 1H), 6.92 (d, 1H).
Step 2. Synthesis of ethyl 4-[3-(3-aminopropoxy)isoxazol-5-yl]-3-(1,3-benzodioxol-5-yl)butanoate.

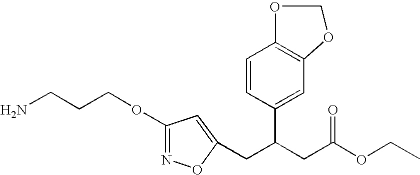

To ethyl 3-(1,3-benzodioxol-5-yl)-4-{3-[3-(N-BOCamino)propoxy]isoxazol-5-yl}butanoate (880 mg, 1.8 mmol) in CH₂Cl₂ (7 mL) was added TFA (7 mL). The reaction mixture was stirred at room temperature for 2 h. The reaction mixture was concentrated under reduced pressure and partitioned between saturated aqueous NaHCO₃ (20 mL) and EtOAc (20 mL). The organic layers were separated and the aqueous extracted with EtOAc (2×15 mL). The organic layers were combined, washed with brine, dried over Na₂SO₄, and concentrated under reduced pressure. Obtained was a yellow oil (677 mg, 1.8 mmol, 99%). ¹H NMR (400 MHz, DMSO-d6) δ 1.07 (t, 3H), 1.83 (m, 2H), 2.55-2.77 (m, 5H), 2.98 (m, 2H), 3.34 (m, 1H), 3.95 (dq, 2H), 4.14 (t, 2H), 5.83 (s, 1H), 5.97 (s, 2H), 6.68 (dd, 1H), 6.77 (d, 1H), 6.92 (d, 1H). LC-MS (MH+)=377.
Step 3. Synthesis of ethyl 3-(1,3-benzodioxol-5-yl)-4-{3-[3-(4,5-dihydro-1H-imidazol-2-ylamino)propoxy]isoxazol-5-yl}butanoate.

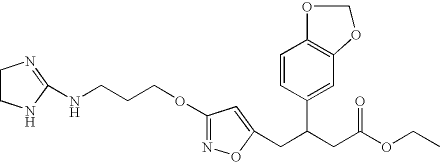

To ethyl 4-[3-(3-aminopropoxy)isoxazol-5-yl]-3-(1,3-benzodioxol-5-yl)butanoate (670 mg, 1.8 mmol) in anhydrous pyridine (8 mL) at 95° C. under Ar gas was added 2-methylthio-2-imidazoline hydriodide (537 mg, 2.2 mmol). After 5 h at 95° C., the solvent was removed under reduced pressure. The resulting residue was purified by flash column chromatography using 10% MeOH/CH₂Cl₂/NH₄OH as eluent. Obtained was a yellow oil (621 mg, 1.4 mmol, 78%). ¹H NMR (400 MHz, DMSO-d6) δ 1.07 (t, 3H), 1.90 (m, 2H), 2.55-2.73 (m, 2H), 2.99 (m, 2H), 3.23 (m, 2H), 3.35 (m, 1H), 3.56 (s, 4H), 3.95 (dq, 2H), 4.12 (t, 2H), 5.82 (s, 1H), 5.97 (s, 2H), 6.68 (dd, 1H), 6.78 (d, 1H), 6.93 (d, ₁H), 8.21 (bs, 1H). LC-MS (MH+)=445.
Step 4. Synthesis of 3-(1,3-benzodioxol-5-yl)-4-{3-[3-(4,5-dihydro-1H-imidazol-2-ylamino)propoxy]isoxazol-5-yl}butanoic acid.

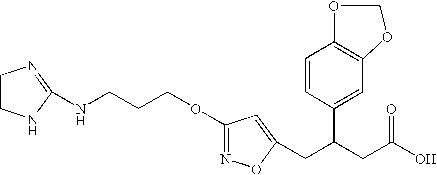

Same synthetic procedure as a previous example using ethyl 3-(1,3-benzodioxol-5-yl)-4-{3-[3-(4,5-dihydro-1H-imidazol-2-ylamino)propoxy]isoxazol-5-yl}butanoate as starting material. ¹H NMR (400 MHz, DMSO-d6) δ 1.90 (m, 2H), 2.49-2.65 (m, 2H), 2.98 (m, 2H), 3.23 (q, 2H), 3.33 (m, 1H), 3.56 (s, 4H), 4.12 (t, 2H), 5.80 (s, 1H), 5.97 (s, 2H), 6.68 (dd, 1H), 6.78 (d, 1H), 6.91 (d, 1H), 8.31 (t, 1H), 12.14 (bs, 1H). LC-MS (MH+)=417. Anal. Cald. for C₂₀H₂₄N₄O₆.1.3TFA. 0.25H₂O: C, 47.69; H, 4.57; N, 9.84. Found: C, 47.66; H, 4.46; N, 10.06.

### EXAMPLE 109

Example 116. Synthesis of 3-[2-(4-chlorophenyl)-1,3-thiazol-5-yl]4-{3-[2-(5,6,7,8-tetrahydro-1,8-naphthyridin-2-yl)ethoxy]isoxazol-5-yl}butanoic acid. 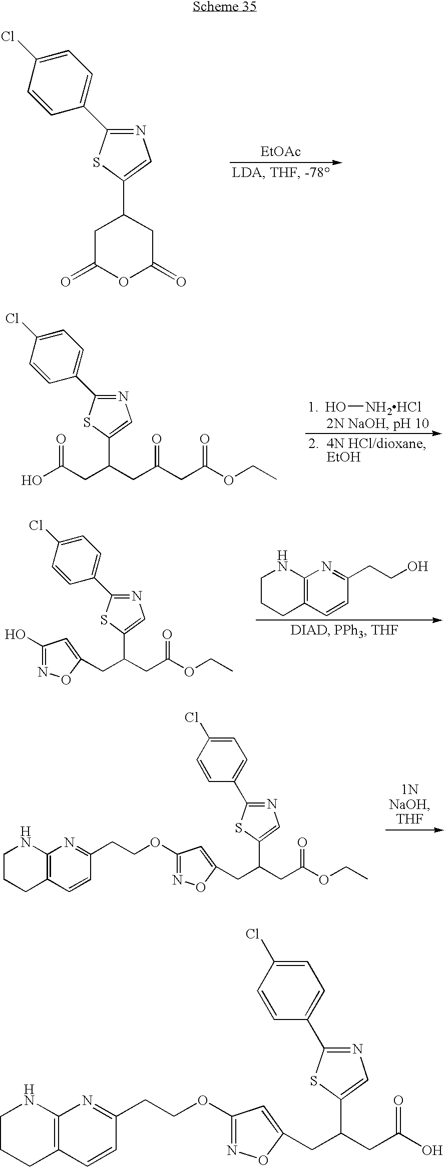
Step 1. Synthesis of 3-[2-(4-chlorophenyl)-1,3-thiazol-5-yl]-7-ethoxy-5,7-dioxoheptanoic acid.

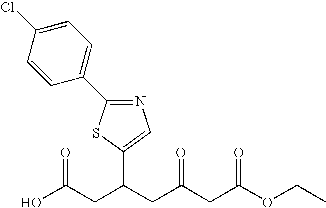

Same synthetic procedure as a previous example using 3-[2-(4-chlorophenyl)-1,3-thiazol-5-yl]dihydro-2H-pyran-2,6-dione as starting material. Compound purified by supercritical fluid chromatography using the cyano column. ¹H NMR (400 MHz, DMSO-d6) δ 1.15 (t, 3H), 2.56-2.76 (m, 2H), 3.08 (d, 2H), 3.60 (s, 2H), 3.86 (m, 1H), 4.05 (q, 2H), 7.54 (d, 2H), 7.70 (s, 1H), 7.89 (d, 2H), 12.35 (bs, 1H). LC-MS (MH+)=397.
Step 2. Synthesis of ethyl 3-[2-(4-chlorophenyl)-1,3-thiazol-5-yl]4-(3-hydroxyisoxazol-5-yl)butanoate.

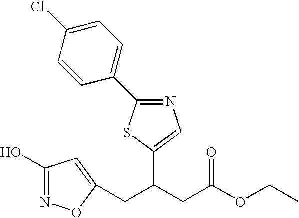

Same synthetic procedure as a previous example using 3-[2-(4-chlorophenyl)-1,3-thiazol-5-yl]-7-ethoxy-5,7-dioxoheptanoic acid as starting material. ¹H NMR (400 MHz, DMSO-d6) δ 1.12 (t, 3H), 2.70-2.89 (m, 2H), 3.01-3.17 (m, 2H), 3.85 (m, 1H), 4.03 (q, 2H), 5.77 (s, 1H), 7.55 (d, 2H), 7.71 (s, 1H), 7.88, (d, 2H), 11.05 (s, 1H). LC-MS (MH+) 393.
Step 3. Synthesis of ethyl 3-[2-(4-chlorophenyl)-1,3-thiazol-5-yl]-4-{3-[2-(5,6,7,8-tetrahydro-1,8-naphthyridin-2-yl)ethoxy]isoxazol-5-yl}butanoate.

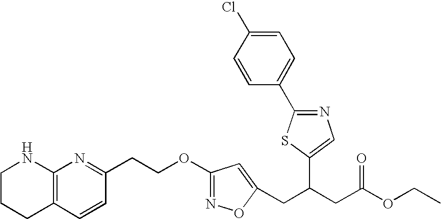

To a solution of ethyl 3-[2-(4-chlorophenyl)-1,3-thiazol-5-yl]4-(3-hydroxyisoxazol-5-yl)butanoate (195 mg, 0.50 mmol) in anhydrous THF (4.5 mL) under Ar gas was added 2-(5,6,7,8-tetrahydro-1,8-naphthyridin-2-yl)ethanol (98 mg, 0.55 mmol) and triphenylphosphine (157 mg, 0.60 mmol). The temperature of the reaction mixture was lowered to 0° C. and diisopropyl azodicarboxylate (119 μL, 0.60 mmol) was added. The reaction mixture was stirred at room temperature overnight. The reaction was quenched into saturated aq. NH₄Cl (15 mL) and extracted with EtOAc (3×15 mL). The organic layers were combined, washed with brine, dried over Na₂SO₄, and concentrated to an oil. The oil was purified by flash column chromatography using EtOAc as the eluent. Obtained was ethyl 3-[2-(4-chlorophenyl)-1,3-thiazol-5-yl]-4-{3-[2-(5,6,7,8-tetrahydro-1,8-naphthyridin-2-yl)ethoxy]isoxazol-5-yl}butanoate, contaminated with the triphenylphosphine oxide byproduct (226 mg, approximately 1:1 ratio by NMR).
Step 4. Synthesis of 3-[2-(4-chlorophenyl)-1,3-thiazol-5-yl]-4-{3-[2-(5,6,7,8-tetrahydro-1,8-naphthyridin-2-yl)ethoxy]isoxazol-5-yl}butanoic acid.

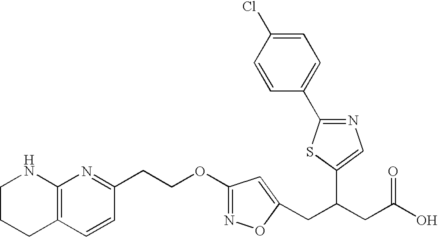

Impure compound ethyl 3-[2-(4-chlorophenyl)-1,3-thiazol-5-yl]-4-{3-[2-(5,6,7,8-tetrahydro-1,8-naphthyridin-2-yl)ethoxy]isoxazol-5-yl}butanoate (220 mg) was dissolved in THF (1 mL). To the solution was added 1N NaOH (0.8 mL). The reaction mixture was stirred vigorously overnight. The reaction mixture was concentrated and the resulting residue partitioned between H₂O (10 mL) and EtOAc (10 mL). The organic layer was removed and the aqueous extracted with EtOAc (1×4 mL). The aqueous was concentrated to a smaller volume and acidified with 1N HCl. Acetonitrile was added to form a solution which was purified by reverse phase HPLC using (H₂O/HCl)/CH₃CN as eluent (0.5 mL conc. HCl in 4 L H₂O). Obtained was a yellow solid 3-[2-(4-chlorophenyl)-1,3-thiazol-5-yl]-4-{3-[2-(5,6,7,8-tetrahydro-1,8-naphthyridin-2-yl)ethoxy]isoxazol-5-yl}butanoic acid (105 mg) as an HCl salt. ¹H NMR (400 MHz, DMSO-d6) δ 1.80 (m, 2H), 2.63-2.84 (m, 4H), 3.03-3.21 (m, 4H), 3.41 (m, 2H), 3.84 (m, 1H), 4.45 (t, 2H), 6.03 (s, 1H), 6.63 (d, 1H), 7.54 (m 3H), 7.71 (s, 1H), 7.88 (d, 2H), 8.12 (bs, 1H). LC-MS (MH+) 526. Anal. Cald. for C₂₆H₂₅ClN₄O₄S.1.75HCl.5H₂O: C, 46.00; H, 5.46; N, 8.25. Found: C, 46.07; H, 5.55; N, 8.32.

### EXAMPLE 110 & 111

3-Benzo[1,3]dioxol-5-yl-4-{3-[2-(6,7,8,9-tetrahydro-5-oxa-1,9-diaza-benzocyclohepten-2-yl)-ethoxy]-isoxazol-5-yl}-butyric acid, and 3-Benzo[1,3]dioxol-5-yl-4-{3-oxo-2-[2-(6,7,8,9-tetrahydro-5-oxa-1,9-diaza-benzocyclohepten-2-yl)-ethyl]-2,3-dihydro-isoxazol-5-yl}-butyric acid.

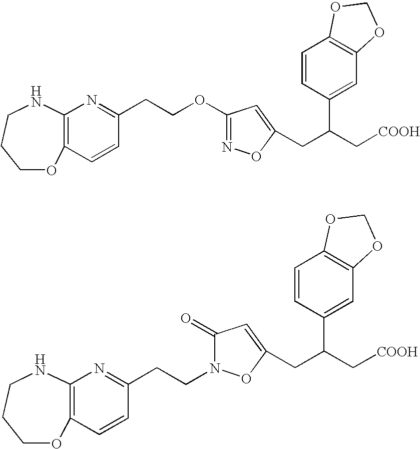

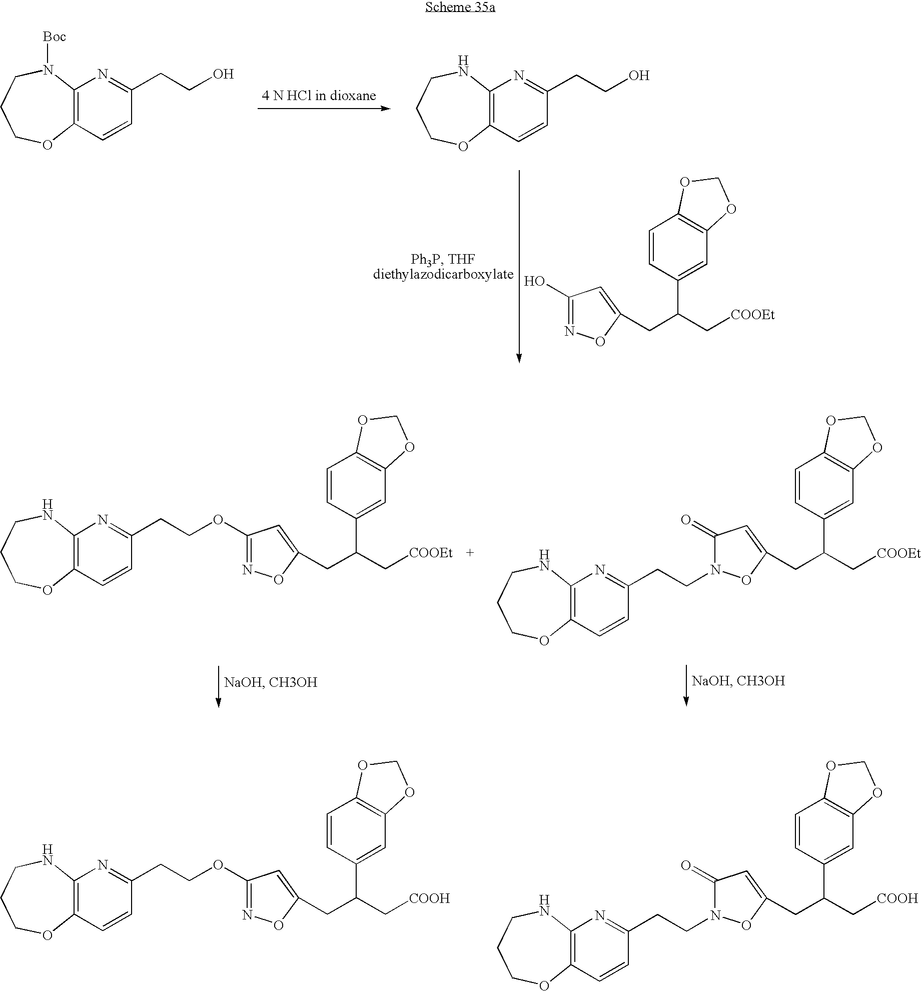

Step 1. 2-(6,7,8,9-Tetrahydro-5-oxa-1,9-diaza-benzocyclohepten-2-yl)-ethanol.

The solution of appropriate intermediate (0.86 g, 2.92 mmol) and 4 N HCl in dioxane (5 mL) was stirred at room temperature for 2 hours. The solvent was removed. The crude product was purified on silica gel column, eluting with dichloromethane/ethanol/ammonium hydroxide (95:4.5:0.5) to give 0.466 g of 2-(6,7,8,9-tetrahydro-5-oxa-1,9-diaza-benzocyclohepten-2-yl)-ethanol as yellow oil. ¹H-NMR (CD₃OD): δ 2.00 (2H, m, CH₂), δ 2.78 (2H, t, CH₂), δ3.85 (2H, t, CH₂), δ 4.10 (2H, t, CH₂), δ 6.58 (1H, d, Py-H), δ 7.10 (1H, d, Py-H).

Step 2

3-Benzo[1,3]dioxol-5-yl-4-{3-[2-(6,7,8,9-tetrahydro-5-oxa-1,9-diaza-benzocyclohepten-2-yl)-ethoxy]-isoxazol-5-yl}-butyric acid ethyl ester and 3-Benzo[1,3]dioxol-5-yl-4-{3-oxo-2-[2-(6,7,8,9-tetrahydro-5-oxa-1,9-diaza-benzocyclohepten-2-yl)-ethyl]-2,3-dihydro-isoxazol-5-yl}-butyric acid ethyl ester.

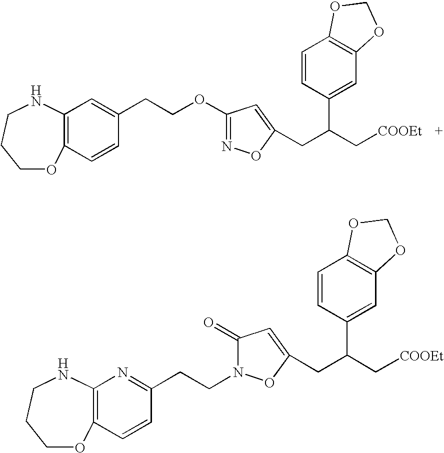

To a solution of 3-benzo[1,3]dioxol-5-yl-4-(3-hydroxy-isoxazol-5-yl)-butyric acid ethyl ester (0.41 g, 1.29 mmol) and tetrahydrofuran (5 mL) was added triphenylphosphine (0.44 g, 1.67 mmol) and diethyl azodicarboxylate (0.28 mL, 1.67 mmol) at 0° C. The solution was stirred for 30 minutes. The solution appropriate intermediate (0.26 g, 1.32 mmol) and tetrahydrofuran (5 mL) was added. The resulting solution was stirred at room temperature overnight. Solvent was removed. The crude product was purified on a reverse phase HPLC using acetonitrile/water (5%) gradient to give 0.380 mg of the mixture of two products. This mixture was carried out to next step without further purification.

Step 2

3-Benzo[1,3]dioxol-5-yl-4-{3-[2-(6,7,8,9-tetrahydro-5-oxa-1,9-diaza-benzocyclohepten-2-yl)-ethoxy]-isoxazol-5-yl}-butyric acid and 3-Benzo[1,3]dioxol-5-yl-4-{3-oxo-2-[2-(6,7,8,9-tetrahydro-5-oxa-1,9-diaza-benzocyclohepten-2-yl)-ethyl]-2,3-dihydro-isoxazol-5-yl}-butyric acid.

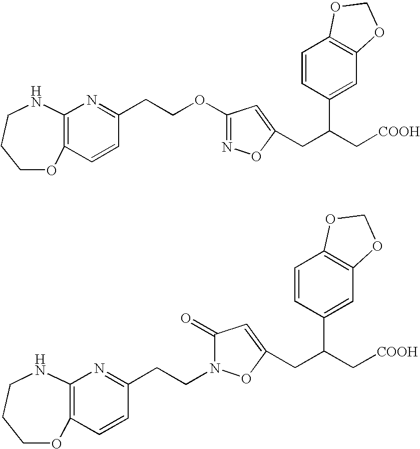

A solution of the mixture from the above step (0.38 g), and 1 N sodium hydroxide in ethanol (50 mL) was stirred overnight. Solvent was removed. The crude product was purified on a reverse phase HPLC using acetonitrile/water (5%) gradient to give 0.27 g of and 0.053 g of two products respectively: First product: ¹H-NMR (CD₃CD): δ 2.18 (2H, m, CH₂), 2.60 (2H, m, CH₂), 2.95 (2H, m, CH₂), 3.08(2H, t, CH₂), 3.38 (1H, m, CH), 3.60 (2H, t, CH₂), 4.28 (2H, t, CH₂), 4.90 (2H, t, CH₂), 5.59 (1H, s, CH), 5.90 (2H, s, CH₂), 6.56(1H, d, Py-H), 6.68 (1H, d, Ar—H), 6.75 (2H, m, Ar—H), 7.30 (1H, d, Py-H). Calcd. for C₂₄H₂₅N₃O_{7.}1.0 TFA, 1.0H₂O: C, 52.09; H, 4.71; N, 7.01. Found: C, 52.16; H, 4.35; N, 7.28. Second Product: ¹H-NMR (CD₃CD): δ 2.18 (2H, m, CH₂), 2.60 (2H, m, CH₂), 2.85 (2H, m, CH₂), 2.95(2H, t, CH₂), 3.32 (1H, m, CH), 3.60 (2H, t, CH₂), 4.08 (2H, t, CH₂), 4.28 (2H, t, CH₂), 5.30 (1H, s, CH), 5.93 (2H, s, CH₂), 6.32 (1H, d, Py-H), 6.70 (1H, m, Ar—H), 6.78 (2H, m, Ar—H), 7.30 (1H, d, Py-H). Calcd. for C₂₄H₂₅N₃O_{7.}2.0 TFA, 1.4H₂O: C, 46.66; H, 4.17; N, 5.83. Found: C, 46.32; H, 3.88; N, 6.25.

### EXAMPLE 112

Preparation of 3-(1,3-benzodioxol-5-yl)-4-{3-[2-(3,4-dihydro-2H-pyrido[3,2-b][1,4]oxazin-6-yl)ethoxy]isoxazol-5-yl}butanoic acid, TFA. 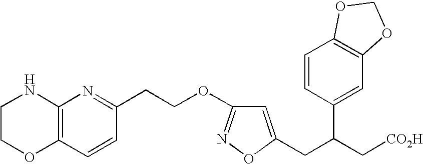 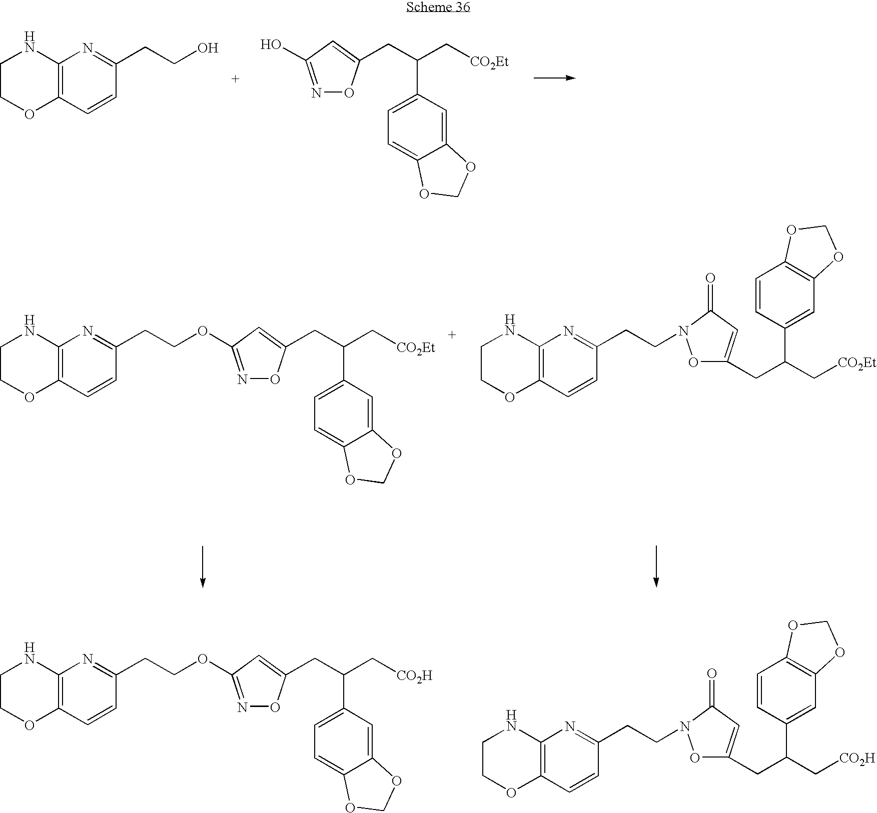
Step 1

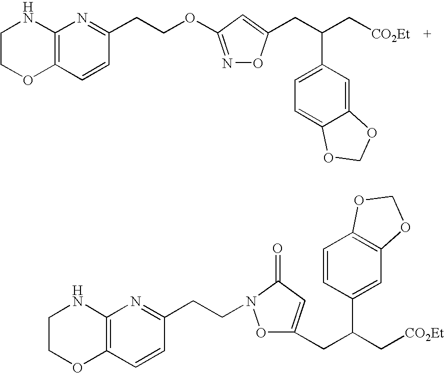

To a solution of the appropriate intermediate (568 mg, 1.78 mmol) and triphenylphosphine (447 mg, 1.78 mmol) in 10 mL THF under N₂ at room temperature was added a solution of diethyl azodicarboxylate (292 mg, 1.78 mmol) in THF (6 mL) and stirred for 15 min. The product of step 6, example 68, (320 mg, 1.78 mmol) was added. The resulting reaction mixture was stirred at room temperature for 3 h. THF was evaporated and the residue was purified on HPLC using acetonitrile gradient 15-50% in 30 mm to yield 80 mg product A and 150 mg product B, both as yellow oils.

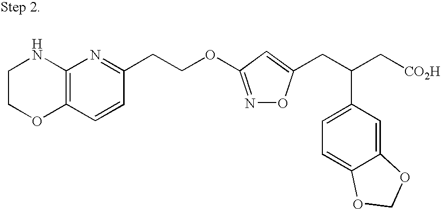

The product B of step 1 (150 mg, 0.31 mmol) was dissolved in 2 mL methanol and 2 mL 1N sodium hydroxide solution. The reaction was stirred at room temperature for 18 h, acidified with 1 mL trifluoroacetic acid, and concentrated. The residue was purified on HPLC using acetonitrile gradient 15-50% in 30 min to yield 110 mg desired product as an yellow oil. FAB-MS:(MH⁺)=454. H NMR (400 MHz, CD₃OD) [symbol] 2.61 (m, 2H), 3.00 (m, 2H), 3.12 (t, 2H), 3.40 (m, 1H), 3.64 (t, 2H), 4.28 (t, 2H), .4.41 (t, 2H), 5.60 (s, 1H), 5.89 (s, 2H), 6.68 (m, 4H), 7.31 (d, 1H). Anal Calcd. for C₂₃H₂₃N₃O₇ plus 1.5 CF₃COOH: C, 50.01; H, 3.95; N, 6.73. Found: 49.98; H, 4.29; N, 7.02.

### EXAMPLE 113

Preparation of 3-(1,3-benzodioxol-5-yl)-4-{2-[2-(3,4-dihydro-2H-pyrido[3,2-b][1,4]oxazin-6-yl)ethyl]-3-oxo-2,3-dihydroisoxazol-5-yl}butanoic acid, TFA

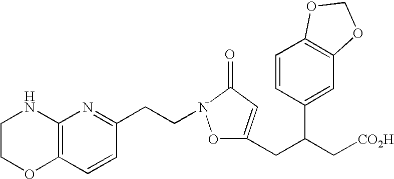

The appropriate intermediate (80 mg, 0.17 mmol) was dissolved in 1.5 mL methanol and 1.5 mL 1N sodium hydroxide solution. The reaction was stirred at room temperature for 18 h, acidified with 1 mL trifluoroacetic acid, and concentrated. The residue was purified on HPLC using acetonitrile gradient 15-50% in 30 min to yield 37 mg desired product as a yellow oil. FAB-MS:(MH⁺)=454. H NMR (500 MHz, CD₃OD) [symbol] 2.65 (m, 2H), 2.98 (m, 4H), 3.41(m, 1H), 3.64 (t, 2H), 4.17 (t, 2H), 4.28 (t, 2H), 5.41 (s, 1H), 5.90 (d, 2H), 6.28 (d, 1H), 6.74 (m, 2H), 6.80 (d, 1H), 7.18 (d, 1H).

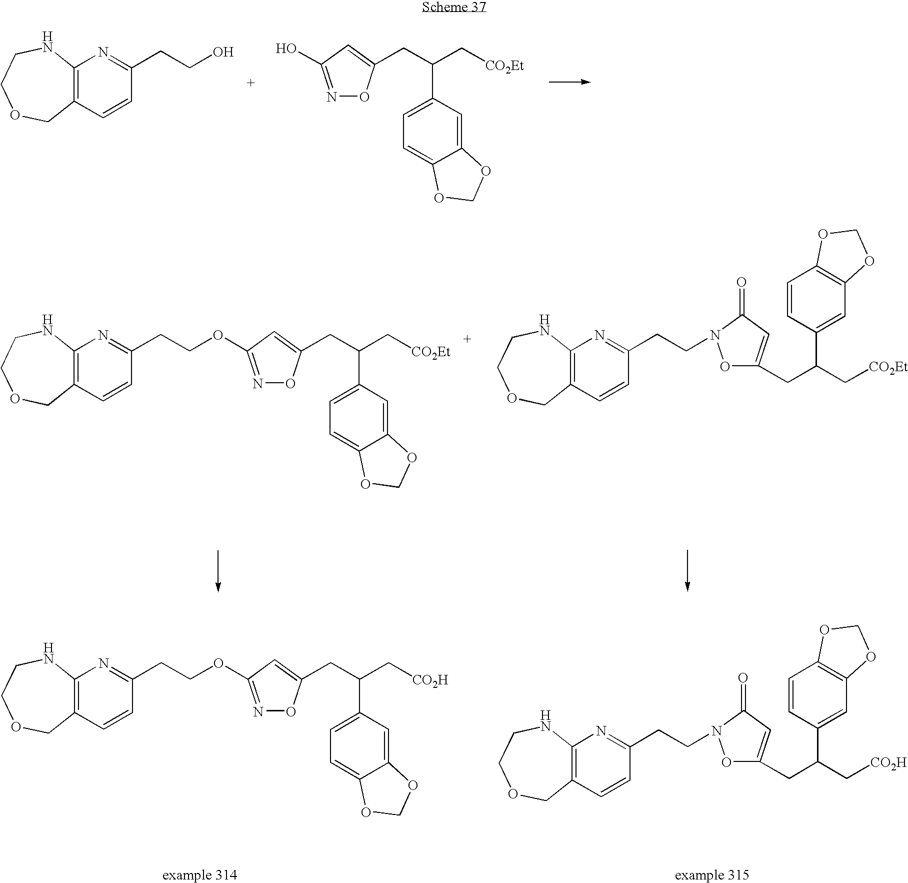

### EXAMPLE 114

Preparation of 3-(1,3-benzodioxol-5-yl)-4-{3-[2-(1,2,3,5-tetrahydropyrido[2,3-e][1,4]oxazepin-8-yl)ethoxy]isoxazol-5-yl}butanoic acid, TFA.

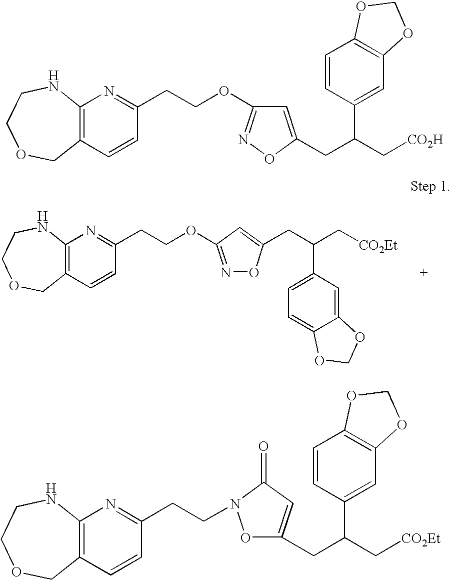

To a solution of the appropriate intermediate shown in a previous example (408 mg, 1.28 mmol) and triphenylphosphine (336 mg, 1.28 mmol) in 10 mL THF under N₂ at room temperature was added a solution of diethyl azodicarboxylate (222 mg, 1.28 mmol) in THF (6 mL) and stirred for 15 min. The appropriate intermediate (360 mg, 2.0 mmol) was added. The resulting reaction mixture was stirred at room temperature for 3 h. THF was evaporated and the residue was purified on HPLC using acetonitrile gradient 15-50% in 30 min to yield 70 mg product A and 135 mg product B, both as yellow oils.

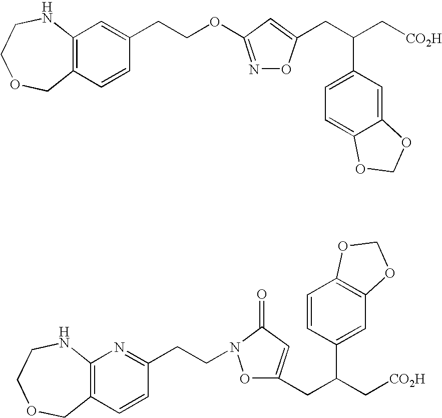

The product B of step 1 (135 mg, 0.27 mmol) was dissolved in 2 mL methanol and 2 mL 1N sodium hydroxide solution. The reaction was stirred at room temperature for 18 h, acidified with 1 mL trifluoroacetic acid, and concentrated. The residue was purified on HPLC using acetonitrile gradient 15-50% in 30 min to yield 97 mg desired product as an yellow oil. FAB-MS:(MH+)=468. H NMR (400 MHz, CD₃OD) δ 2.61 (m, 2H), 3.00 (m, 2H), 3.20 (t, 2H), 3.40 (m, 1H), 3.67 (t, 2H), 3.98 (t, 2H), 4.46 (t, 2H), 4.74 (s, 2H), 5.60 (s, 1H), 5.89 (s, 2H), 6.69 (m, 3H), 6.86 (d, 1H), 7.78 (d, 1H). Anal Calcd. for C₂₄H₂₅N₃O₇ plus 1.5 CF₃COOH: C, 50.79; H, 4.18; N, 6.58. Found: 50.87; H, 4.38; N, 6.69.

### EXAMPLE 115

Preparation of 3-(1,3-benzodioxol-5-yl)-4-{3-oxo-2-[2-(1,2,3,5-tetrahydropyrido[2,3-e][1,4]oxazepin-8-yl)ethyl]-2,3-dihydroisoxazol-5-yl}butanoic acid, TFA.

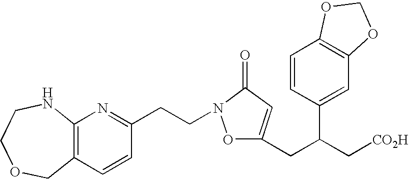

The product A of step 1 (70 mg, 0.14 mmol) was dissolved in 1.5 mL methanol and 1.5 mL 1N sodium hydroxide solution. The reaction was stirred at room temperature for 18 h, acidified with 1 mL trifluoroacetic acid, and concentrated. The residue was purified on HPLC using acetonitrile gradient 15-50% in 30 min to yield mg desired product as an yellow oil. FAB-MS:(MH+)=468. H NMR (400 MHz, CD₃OD) [symbol] 2.64 (m, 2H), 2.97 (m, 2H), 3.09 (t, 2H), 3.39 (m, 1H), 3.70 (t, 2H), 4.00 (t, 2H), 4.21 (t, 2H), 4.75 (s, 2H), 5.41 (s, 1H), 5.90 (dd, 2H), 6.54 (d, 1H), 6.75 (m, 3H), 7.70 (d, 1H).

### EXAMPLE 116

Preparation of 3-(1,3-benzodioxol-5-yl)-4-(3-{2-[5-(methoxymethyl)-6-(methylamino)pyridin-2-yl]ethoxy}isoxazol-5-yl)butanoic acid, TFA.

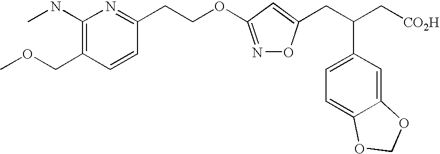

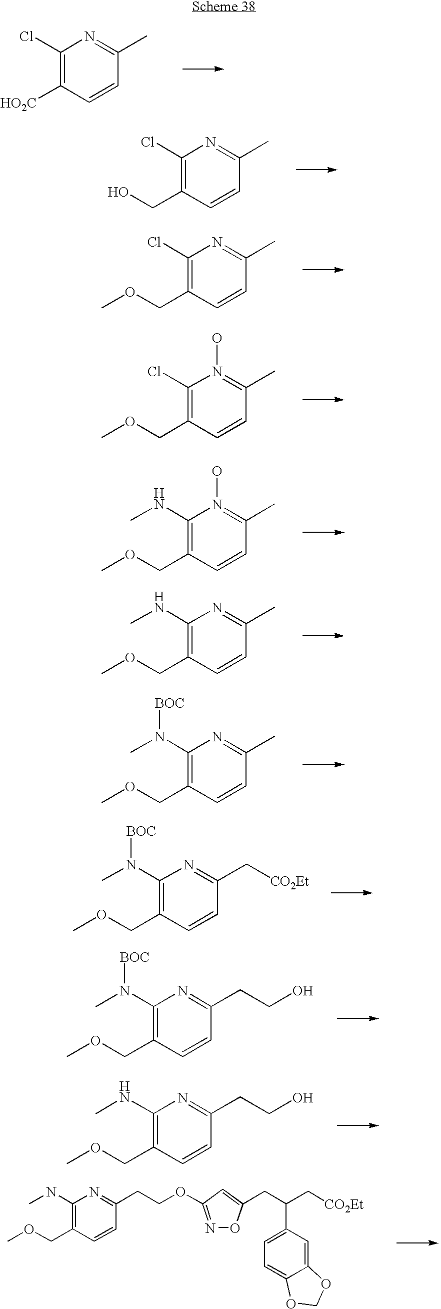

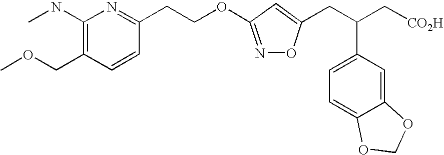

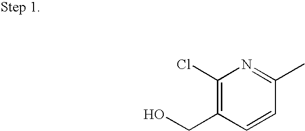

As described in a previous example.

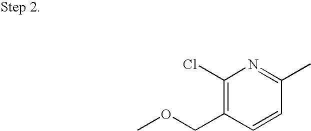

To a solution the product of step 1 (7 g, 44.58 mmol) in DMSO (35 mL) at room temperature was added powder KOH (7.5 g, 133.74 mmol) and followed by MeI (4.2 mL, 66.87 mmol). After stirring at room temperature for 2 hours the reaction was quenched with water. After extraction with Et₂O(3×), the organic layers were washed partitioned between water and EtOAc, washed with brine, dried over Na₂SO₄ and concentrated in vacuo to afford the title compound as a yellow crystalline solid. H NMR (400 MHz, CDCl₃) δ 2.54 (s, 3H), 3.47 (s, 3H), 4.50 (s, 2H), 7.10 (d, 1H), 7.70 (d, 1H).

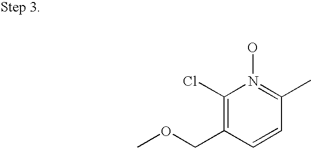

The product of step 2 (7.38 g, 43.03 mmol) and mCPBA (14.85 g, 86.06 mmol) were dissolved in CHCl₃ (50 mL) and stirred at 50° C. overnight. The solution was concentrated in vacuo and purified by flash chromatography (silica, 98:2:0.5 CH₂Cl₂: MeOH: NH₄OH) to yield white crystals. H NMR (400 MHz, CDCl₃) δ 2.57 (s, 3H), 3.48 (s, 3H), 4.50 (s, 2H),) 7.20 (d, 1H), 7.30 (d, 1H).

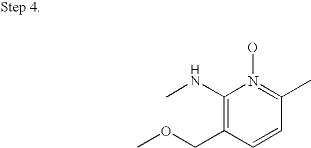

A mixture of the product of step 3 (5.8 g, 30.85 mmol), methylamine (30 mL, 2M solution in THF, 60 mmol) and NaHCO₃ (13 g, 154 mmol) in t-amyl alcohol (70 mL) was heated to 90 in a pressure tube for 48 hours. The reaction was cooled, diluted with CH₂Cl₂ and filtered. The filtrate was concentrated in vacuo and purified by flash chromatography (silica, 98:2:0.5, CH₂Cl₂: MeOH: NH₄OH) to yield a light yellow crystals. H NMR (400 MHz, CDCl₃) δ 2.50 (s, 3H), 3.26 (d, 3H), 3.37 (s, 3H), 4.50 (s, 2H),) 6.50 (d, 1H), 7.02 (d, 1H).

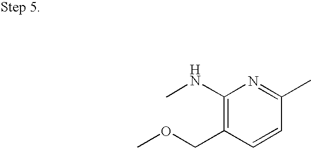

A solution of the product of step 4 (7.38 g, 40.55 mmol), ion powder (3.4 g, 60.82 mmol), triphenylphosphine (10.64 g, 40.55 mmol) and acetic acid (100 ml) was heated to reflux for 1 hour. The solution was cooled, filtrated through a celite bed, and washed with ethyl acetate. The filtrate was concentrated and purified on a silica gel column, eluting with 50% EtOAc/Hex to afford a yellow oil. H NMR (400 MHz, CDCl₃) δ 2.41 (s, 3H), 3.00 (s, 3H), 3.29 (s, 3H), 4.36 (s, 2H),) 6.36 (d, 1H), 7.02 (d, 1H).

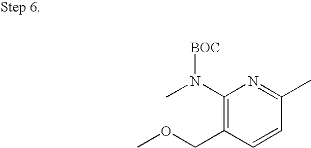

A solution of the product of step 5 (3 g, 18.07 mmol), di-tert-butyl dicarbonate (7.86 g, 36 mmol), and DMAP (250 mg) in THF (5 mL) was heated to reflux overnight. The reaction mixture was allowed to cool to room temperature and was concentrated under reduced pressure to get the crude product. Purified by chromatography on silica gel (eluent: 30/70 ethyl acetate/hexane) to afford a yellow oil.

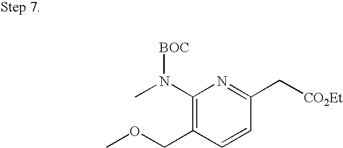

Lithium diisopropylamide solution (2.6 mL, 5.26 mmol, 2.0 M in THF/ethylbenzene/heptane) was added dropwise to a chilled (−78° C.), stirred solution of the product of step 6 (0.5 g, 1.88 mmol) in dry THF (30 mL) under N₂ and the resulting solution stirred for 20 min at −78° C. Diethyl carbonate (0.843 mL, 6.95 mmol) was introduced to the mixture. After 1 hour the reaction was quenched with saturated NH₄Cl solution and warmed to room temperature. The mixture was extracted three times with ethyl acetate and all organic extracts were combined, washed with brine, dried over MgSO₄, and concentrated under reduced pressure to get the crude product, which was purified by chromatography on silica gel (eluent: 25% ethyl acetate/hexane). The desired product is a yellow oil. MHz, CDCl₃) δ 1.19 (t, 3H), 1.35 (s, 9H), 3.16 (s, 3H), 3.32 (s, 3H), 3.74 (s, 2H), 4.10 (q, 2H), 4.22 (s, 2H),) 7.15 (d, 1H), 7.76 (d, 1H).

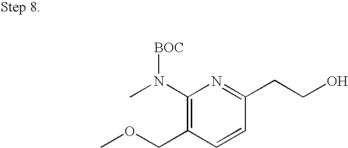

To a solution of the product of step 7 (660 mg, 1.95 mmol) in dry THF (10 mL) at room temperature was added a solution of LiBH₄ (2.0 M in THF, 1.17 mL, 2.34 mmol), and the resulting mixture was heated to reflux. After 16 hours the mixture was cooled to 0° C. and carefully quenched with water. After 10 minutes, the mixture was extracted three times with ethyl acetate. The combined organic extracts were washed with brine, dried over Na₂SO₄, filtered, and concentrated under reduced pressure to give an yellow oil.

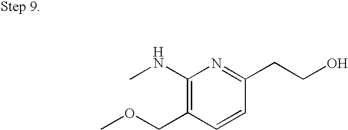

A mixture of the product of step 7 and 4 M HCl in dioxane (4 mL) was stirred at room temperature for 4 hours, and then concentrated under reduced temperature. The residue was dissolved in 4 mL of MeOH, to this was added 1 g NaHCO₃. The resulting mixture was stirred at room temperature for 1 hour, filtered and concentrated in vacuo to afford a yellow oil. H NMR (400 MHz, CD₃OD) [symbol] 2.94 (t, 2H), 3.03 (s, 3H), 3.30 (s, 3H), 3.84 (t, 2H), 4.35 (t, 2H), 6.67 (d, 1H), 7.70 (d, 1H).

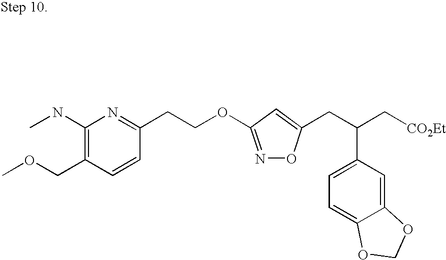

To a solution of product from Example 111, Step 6 (652 mg, 2.04 mmol) and triphenylphosphine (525 mg, 2.04 mmol) in 8 ml THF under N2 at room temperature was added a solution of diethyl azodicarboxylate (329 mg, 2.04 mmol) in THF (3 mL) and stirred for 15 min. The product of step 9 (400 mg, 2.04 mmol) was added. The resulting reaction mixture was stirred at room temperature for 3 h. THF was evaporated and the residue was purified on HPLC using acetonitrile gradient 15-50% in 30 min to yield a yellow oils.
Step 11. 3-(1,3-benzodioxol-5-yl)-4-(3-{2-[5-(methoxymethyl)-6-(methylamino)-pyridin-2-yl]ethoxy}isoxazol-5-yl)butanoic acid

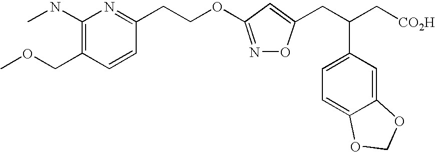

The product of step 9 (100 mg, 0.21 mmol) was dissolved in 2 ml methanol and 2 ml 1N sodium hydroxide solution. The reaction was stirred at room temperature for 18 h, acidified with 1 ml trifluoroacetic acid, and concentrated. The residue was purified on HPLC using acetonitrile gradient 15-50% in 30 min to yield 57 mg desired product as a yellow oil. FAB-MS:(MH+)=466. H NMR (500 MHz, CD₃OD) [symbol] 2.63 (m, 2H), 3.00 (m, 2H), 3.13 (s, 3H), 3.29 (m, 2H), 3.40 (m, 4H), 4.42 (s, 2H), 4.48 (t, 2H), 5.62 (s, 1H), 5.88 (s, 2H), 6.70 (m, 4H), 7.76 (d, 1H).

### EXAMPLE 117

3-(1,3-Benzodioxol-5-yl)-4-{3-[3-(5,5-dimethyl-5,6,7,8-tetrahydro-1,8-naphthyridin-2-yl)propyl]-1,2,4-oxadiazol-5-yl}butanoic acid. 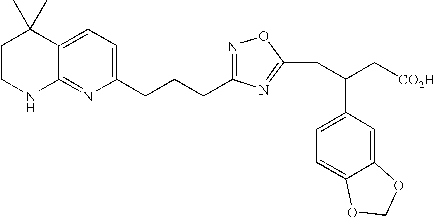 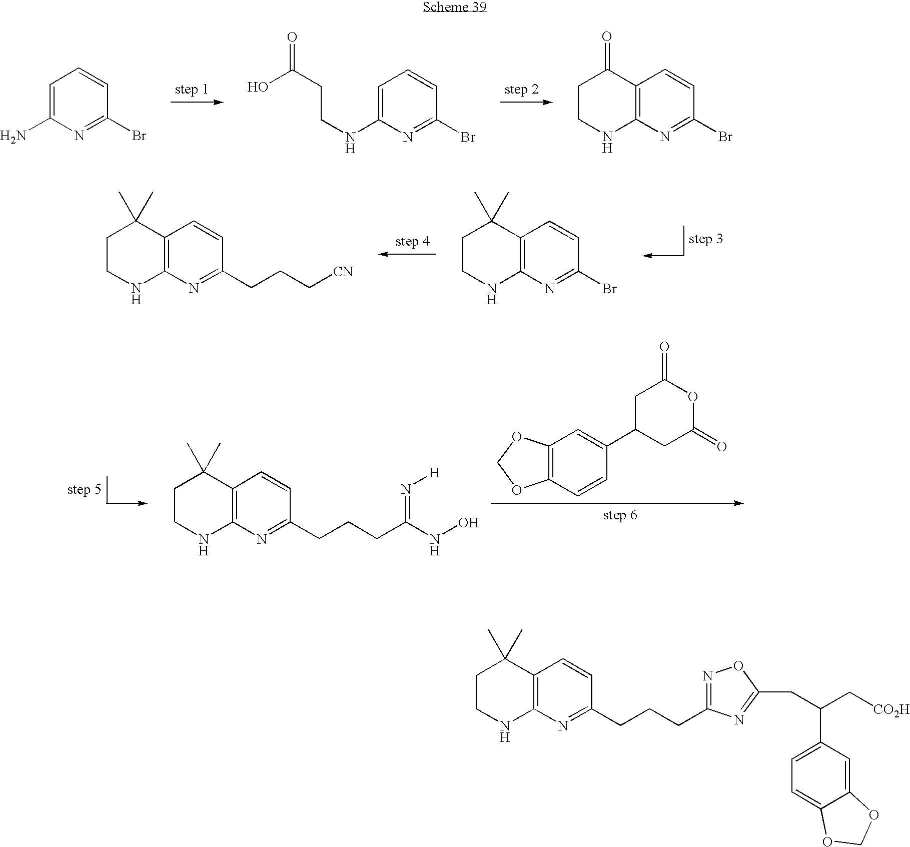
Step 1. Preparation of N-(6-bromopyridin-2-yl)-beta-alanine

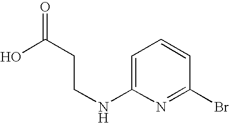

Commercially available 2-Bromo-5-aminopyridine (25.46 g) was combined with 18.9 mL of ethylacrylate and to this solution was added 4.41 mL of glacial acetic acid. The solution was heated to 130° C. under an atmosphere of argon for 3 days. The reaction was cooled and then 58 mL of 6N aqueous sodium hydroxide was added and the reaction was heated to 100° C. for 40 minutes. After the reaction was cooled, the pH was adjusted to 5 with concentrated hydrochloric acid. The precipitate was collected and washed with fresh water and then hexane. The mother liquors were washed with ethyl acetate or methylene chloride to give 6.6 g of product. ¹H NMR, 400 MHz, DMSO δ 12.23 (1H, br. s); 7.27 (1H, dd, J=8, 7 Hz); 7.00 (1H, br. t, J=6 Hz); 6.63 (1H, d, J=7 Hz); 6.46 (1H, d, J=8 Hz); 3.39 (2H, q, J=6.5 Hz); 2.49 (2H, t, J=7 Hz
Step 2. Preparation of 17-bromo-2,3-dihydro-1,8-naphthyridin-4(1H)-one

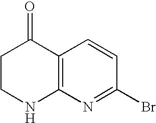

The product (6.6 g) from the previous step was suspended in 99 g of polyphosphoric acid and heated for 40 minutes at 120° C. The reaction mixture was transferred to a glass beaker to cool down and then added portion-wise to ice then stirring till the viscous oil completely dissolved. The solution was kept at 0° C. at all times. The pH of the resulting solution was adjusted between 8 and 9 with cold concentrated ammonium hydroxide. The resulting solid was filtered from the solution then washed with water then dissolved in methylene chloride. This solution was washed with brine, dried (MgSO₄) and the solvent was removed under reduced pressure. The resulting solid was dried under high vacuum and then washed with absolute ethanol to give the desired product (1.1 g) whose purity was acceptable for use in the next

Step 3.

¹H NMR, 300 MHz, CDCl₃δ 7.88 (1H, d, J=8 Hz); 6.87 (1H, d, J=8 Hz); 5.54 (1H, br. s); 3.67 (2H, td, J=7, 2 Hz); 2.72 (2H, t, J=7 Hz).
Step 4. Preparation of 17-bromo-4,4-dimethyl-1,2,3,4-tetrahydro-1,8-naphthyridine

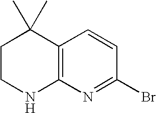

To a solution of 26 mL of methylene chloride was added 15.08 ml of a 1M solution of titanium tetrachloride in methylene chloride and cooled to −30° C. A solution of dimethylzinc (7.54 mL; 2.0 M in toluene) was added drop-wise and after the addition, the solution was stirred for 10 minutes. The ketone, generated from step 2, was added all at once and the reaction mixture was allowed to warm to 25° C. over a 2 hr period. The solution was poured into 50 ml of ice water and then extracted with methylene chloride. The organic extracts were dried (MgSO₄) then evaporated to dryness to provide a yellow oil, which started to solidify upon standing. The crude reaction mixture was purified by column chromatography to provide 261 mg of the desired product. ¹H NMR, 300 MHz, CDCl₃δ 7.19 (1H, d, J=8 Hz); 6.67 (1H, d, J=8 Hz); 5.25 (1H, br. s); 3.43 (2H, m); 1.68 (2H, distorted t, J˜7 Hz); 1.25 (6H, s).
Step 5. Preparation of 14-(5,5-dimethyl-5,6,7,8-tetrahydro-1,8-naphthyridin-2-yl)butanenitrile

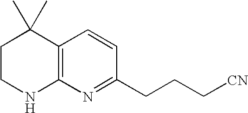

A flask equipped with an inert atmosphere was charged with 4.5 mL of a 0.5 M solution of 3-cyanopropylzinc bromide and to this solution was added 131.7 mg of tetrakis triphenylphoshine palladium followed by the bromonapthyridine generated from step 3. The reaction was very sluggish and was warmed to 50° C. Additional catalyst was added portion wise, in intervals, in an attempt to further drive the reaction. The reaction was quenched with a saturated solution of sodium chloride, and then extracted with methylene chloride. The solution was purified by flash chromatography (SiO₂; 10% MeOH/CH₂Cl₂) then purified by reverse phase chromatography to give 47 mg of the desired product. ¹H NMR, 300 MHz, CD₃CN δ 9.57 (1H, br. s); 7.70 (1H, d, J=8 Hz); 6.55 (1H, d, J=8 Hz); 3.50 (2H, br. t, J=7 Hz); 2.79 (2H, t, J=7 Hz); 2.46 (2H, t, J=7 Hz); 2.02 (2H, p, J=7 Hz); 1.73 (2H, distorted t, J=7 Hz); 1.29 (6H, s).
Step 6. Preparation of (1 Z)-4-(5,5-dimethyl-5,6,7,8-tetrahydro-1,8-naphthyridin-2-yl)-N-hydroxybutanimidamide

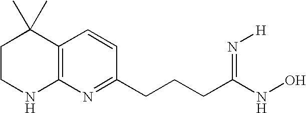

The product (47 mg; 0.13 mmol) from the previous step was dissolved in 1-2 mL of absolute ethanol and to this solution was added 17.96 mg of potassium carbonate. The solution was stirred a 25° C. After 1 hr, 7.9 mL of a 50% wt aqueous solution of hydroxylamine was added at regular intervals while monitoring the reaction by LCMS until most of the starting material was converted into product. The reaction mixture was evaporated to dryness and pumped under high vacuum and used as is in the next step without further purification. ¹H NMR, 300 MHz, CD₃ODδ 7.32 (1H, d, J=8 Hz); 6.37 (1H, d, J=8 Hz); 3.35 (2H, t, J=7 Hz); 2.50 (2H, t, J=7 Hz); 2.07 (2H, t, J=7 Hz); 1.83 (2H, p, J=7 Hz); 1.62 (2H, t, J=7 Hz); 1.20 (6H, s).
Step 7. Preparation of 3-(1,3-benzodioxol-5-yl)-4-{3-[3-(5,5-dimethyl-5,6,7,8-tetrahydro-1,8-naphthyridin-2-yl)propyl]-1,2,4-oxadiazol-5-yl}butanoic acid

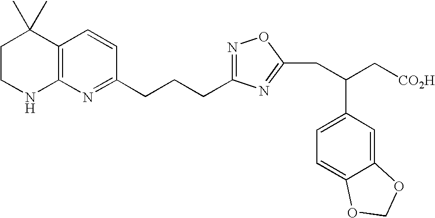

The material obtained from the previous step was dissolved in 1 mL of dioxane and to this solution was added 30 mg of the anhydride. The reaction was heated to 60° C. for one day then the temperature was raised to 100° C. and stirred overnight. The solvent was evaporated to dryness and purified by reverse phase chromatography (gradient H₂O/acetonitrile 0.1% HCl) to give 3.56 mg of the desired compound as the HCl salt which was 16-20% pure. ¹H (CD₃CN): d 8.44 (1H, br. s); 7.68 (1H, d, J=8 Hz); 6.82 (1H, d, J=1 Hz); 6.76 (1H, d, J=8 Hz); 6.73 (1H, dd, J=8, 1 Hz); 6.52 (1H, d, J=8 Hz); 5.93 (2H, s); 5.47 (1H, s); 3.61 (1H, p, J=7 Hz); 3.53 (2H, br. t, J=6 Hz); 3.23 (1H, dd, J=15, 7 Hz); 3.15 (1H, dd, J=15, 7 Hz); 2.82 (1H, dd, J=16, 7 Hz); 2.72 (4H, t, J=7 Hz); 2.70 (1H, dd, J=16, 7 Hz); 2.14 (2H, p, J=7 Hz); 1.74 (1H, distorted t, J=6 Hz); 1.28 (6H, s).

### EXAMPLE 118

3-(1,3-benzodioxol-5-yl)-4-{3-[3-(1-methyl-1,2,3,4-tetrahydropyrido[2,3-b]pyrazin-6-yl)propyl]-1,2,4-oxadiazol-5-yl}butanoic acid 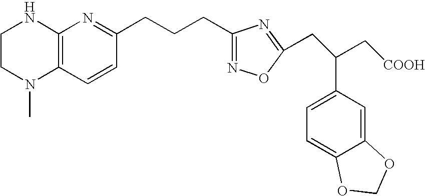 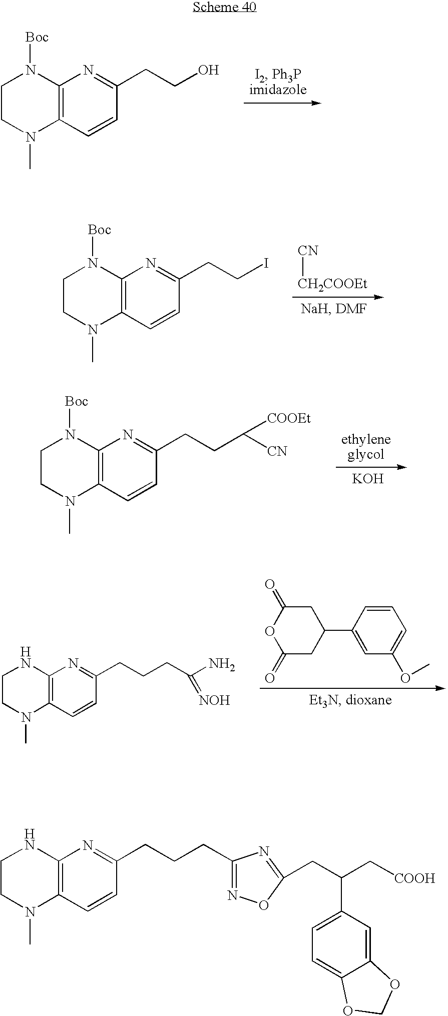
Step 1. Synthesis of 6-methyl-2-nitropyridin-3-yl trifluoromethanesulfonate

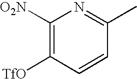

To a solution of 3-hydroxy-6-methyl-2-nitropyridine (2 g, 12.97 mmol, 1 eq) in CH₂Cl₂ (150 mL) at 0° C. under N₂ was added triethylamine (2.68 mL, 19.27 mmol, 1.48 eq) and followed by trifluoromethanesulfonic anhydride (2.62 mL, 15.57 mmol, 1.2 eq). The mixture was stirred for 2 hours at 0° C. and then quenched with water. The organic layer was separated, washed with water and dried over MgSO4. After filtration and concentration at reduced pressure, the crude mixture was purified by flash chromatography on silica gel (15% EA/Hex) to afford the desired product (3.65 g, 98% yield) as yellow oil. H NMR (400 MHz, CDCl₃) δ 2.70 (s, 3H), 7.59 (d, 1H), 7.81(d, 2H).
Step 2. Synthesis of ethyl N-methyl-N-(6-methyl-2-nitropyridin-3-yl)glycinate

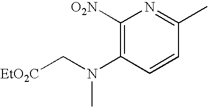

To a solution of 6-methyl-2-nitropyridin-3-yl trifluoromethanesulfonate (7 g, 24.47 mmol, 1 eq) in toluene (40 mL) at room temperature under N₂ was added sarcosine ester hydrochloride (9.4 g, 61.2 mmol, 2.5 eq) and followed by triethylamine (8.51 mL, 61.2 mmol, 2.5 eq). The mixture was refluxed overnight under N₂. The reaction was cooled to room temperature and quenched with water. The mixture was extracted three times with ethyl acetate and all organic extracts were combined, washed with brine, dried over Na₂SO₄. After filtration and concentration at reduced pressure, the crude mixture was purified by flash chromatography on silica gel (20% EA/Hex) to afford the desired product (4.3 g, 69% yield) as brown oil. H NMR (400 MHz, CDCl₃) δ 1.026 (t, 3H), 2.50 (s, 3H), 2.95 (s, 3H), 3.88 (s, 2H), 4.20 (q, 2H), 7.27 (d, 1H), 7.49(d, 2H).
Step 3. Synthesis of 1,6-dimethyl-1,4-dihydropyrido[2,3-b]pyrazin-3(2H)-on

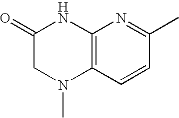

6-Methyl-2-nitropyridin-3-yl trifluoromethanesulfonate (4.3 g, 17 mmol) was hydrogenated in ethanol solution at room temperature using H₂ at 5 psi and 20% Pd(OH)₂/C catalyst for 2 hour. Upon completion of the reaction, the catalyst was filtered off and the filtrate was concentrated under reduced pressure. The product was crystallized out from 50% EA/Hex solution as yellow crystalline solid. The mother liquid was concentrated and purified by flash chromatography on silica gel (50% EA/Hex). (1.44 g, 46% yield) H NMR (400 MHz, CDCl₃) δ 2.26 (s, 3H), 2.70 (s, 3H), 3.18 (t, 2H), 3.58 (m, 2H), 6.34 (d, 1H), 6.57(d, 2H).
Step 4. Synthesis of 1,6-dimethyl-1,2,3,4-tetrahydropyrido[2,3-b]pyrazine

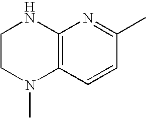

LiAlH₄ (214 mg, 5.64 mmol) was slowly added to 10 mL anhydrous THF in a round-bottom flask fitted with a stirbar and a condenser. After stirring for 10 minutes, a solution of 1,6-dimethyl-1,4-dihydropyrido[2,3-b]pyrazin-3(2H)-one (500 mg, 2.82 mmol) in 5 mL anhydrous THF was added drop wise. Upon completion of the addition, the reaction mixture was refluxed for 16 hours. The reaction was cooled to room temperature and quenched with 1 M NaOH solution until the mixture had become a milky yellow color. The precipitate was filtered off and washed 3 times with CH₂Cl₂. The filtrate and washings were combined, washed with brine, dried over MgSO₄. Filtered and concentrated under reduced pressure to give the desired product as light yellow oil, which solidified on standing. (420 mg, 91% yield). H NMR (400 MHz, CDCl₃) δ2.27 (s, 3H), 2.80 (s, 3H), 3.17 (t, 2H), 3.58 (m, 2H), 6.36 (d, 1H), 6.56(d, 2H)
Step 5. Synthesis of tert-butyl 1,6-dimethyl-2,3-dihydropyrido[2,3-b]pyrazine-4(1H)-carboxylate.

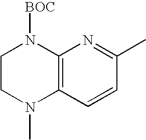

A solution of 1,6-dimethyl-1,2,3,4-tetrahydropyrido[2,3-b]pyrazine (1.14 g, 7 mmol), di-tert-butyl dicarbonate (2.29 g, 10.5 mmol), DMAP (100 mg) and triethylamine (1.46 mL, 10.5 mmol) in 30 mL THF was refluxed 72 hours under N₂. The reaction mixture was allowed to cool to room temperature and diluted with ethyl acetate. The mixture was washed with brine, dried over Na₂SO₄. After filtration and concentration at reduced pressure, the crude mixture was purified by flash chromatography on silica gel (40% EA/Hex) to afford the desired product (1.6 g, 90% yield) as yellow oil. H NMR (400 MHz, CDCl₃) δ 1.51 (s, 9H), 2.40 (s, 3H), 2.90 (s, 3H), 3.28 (t, 2H), 3.83 (m, 2H), 6.78 (d, 1H), 6.83(d, 2H).
Step 6. Synthesis of tert-butyl 6-(2-ethoxy-2-oxoethyl)-1-methyl-2,3-dihydropyrido[2,3-b]pyrazine-4(1H)-carboxylate

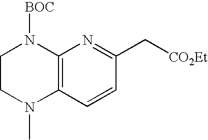

Lithium diisopropylamide solution (5 mL, 10 mmol, 2.0 M in THF/ethylbenzene/heptane) was added drop wise to a chilled (−78° C.), stirred solution of tert-butyl 1,6-dimethyl-2,3-dihydropyrido[2,3-b]pyrazine-4(1H)-carboxylate (950 mg, 3.61 mmol) and diethyl carbonate (1.62 mL, 13.36 mmol) in 20 mL dry THF under nitrogen atmosphere. After 1 hour the reaction was quenched with saturated NH₄Cl solution and warmed to room temperature. The mixture was extracted three times with ethyl acetate and all organic extracts were combined, dried over Na₂SO₄, and concentrated under reduced pressure to get the crude product, which was purified by chromatography on silica gel (eluent: 30% ethyl acetate/hexane). The desired fractions were combined and concentrated under reduced pressure to get the desired product F (1.05 g, 87% yield) as a yellow solid. H NMR (400 MHz, CDCl₃) δ 1.25 (t, 3H), 1.50 (s, 9H), 2.78 (s, 3H), 3.38 (t, 2H), 3.68(s, 2H), 3.84 (t, 2H), 4.14 (q, 2H), 6.86 (d, 1H), 6.95(d, 2H).
Step 7. Synthesis of tert-butyl 6-(2-hydroxyethyl)-1-methyl-2,3-dihydropyrido[2,3-b]pyrazine-4(1H)-carboxylate

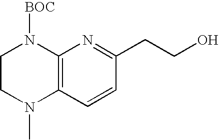

To a solution of tert-butyl 6-(2-ethoxy-2-oxoethyl)-1-methyl-2,3-dihydropyrido[2,3-b]pyrazine-4(1H)-carboxylate (26.5 g, 79.01 mmol)) in dry THF (50 mL) at room temperature was added a solution of LiBH₄ (2.0 M in THF, 59.26 mL), and the resulting mixture was heated to reflux. After 16 hours the mixture was cooled to 0° C. and carefully quenched with water. The mixture was extracted three times with ethyl acetate. The combined organic extracts were dried over MgSO₄, filtered, and concentrated under reduced pressure to get the crude product, which was chromatographed on silica gel (eluent: (1:1) hexane/ethyl acetate) to afford the desired product (17.3 g) H NMR (400 MHz, CDCl₃) δ 1.55 (s, 9H), 2.73 (t, 2H), 2.80 (s, 3H), 3.30 (t, 2H), 3.78(t, 2H), 3.85 (t, 2H), 6.76 (d, 1H), 6.85(d, 2H), 7.28 (s, 1H).
Step 8. Synthesis of tert-butyl 6-(2-iodoethyl)-1-methyl-2,3-dihydropyrido[2,3-b]pyrazine-4(1H)-carboxylate

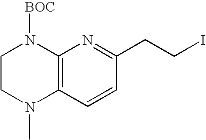

To a stirred, cooled (0° C.) solution of the product of step (5.6 g, 19.09 mmol), triphenylphosphine (6.51 g, 24.82 mmol) and imidazole (1.82 g, 26.72 mmol) in a mixture of CH₃CN and dry ether (1:1) was slowly added iodine (6.78 g, 26.72 mmol) and then stirred for 2 hour. The resulting mixture was added 150 mL ether, washed successively with saturated aqueous Na₂S₂O₃ and brine, dried over Na₂SO₄ and concentrated under reduced pressure. The residue was purified by flash chromatography (silica, 20% EtOAC/Hex) to afford a yellow solid. H NMR (400 MHz, CDCl₃) δ 1.55 (s, 9H), 2.93 (s, 3H), 3.18 (t, 2H), 3.30 (t, 2H), 3.458(t, 2H), 3.85 (t, 2H), 6.85(q, 2H). LC-MS (M+H) 404.
Step 9. Synthesis of tert-butyl 6-(3-cyano-4-ethoxy-4-oxobutyl)-1-methyl-2,3-dihydropyrido[2,3-b]pyrazine-4(1H)-carboxylate

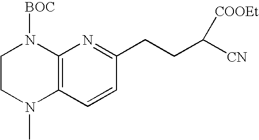

NaH (620 mg, 24.55 mmol) was suspended in DMF (203 mL) at 0° C. under N₂. Ethyl cyanoacetate (2.6 mL, 24.55 mmol) was added and the resulting mixture stirred for 30 min at 0° C. The product of step 8 (6.6 g, 16.37 mmol) in DMF (10 mL) was introduced to the reaction mixture and stirred for 2 hours at room temperature. The mixture was cooled to 0 and quenched with water and extracted with EtOAc (3×). The organic layers were washed with brine, dried over Na₂SO₄ and concentrated in vacuo. The residue was purified by flash chromatography (silica, 90% EtOAC/Hex) to afford colorless oil (5.78 g). H NMR (400 MHz, CDCl₃) δ 1.35 (q, 3H), 1.55 (s, 9H), 2.38 (m, 2H), 2.89 (t, 2H), 2.93 (s, 3H), 3.32 (t, 2H), 3.85 (t, 2H), 3.90(m, 1H), 4.30 (q, 2H), 6.85(d, 2H). LC-MS (M+H) 389.
Step 10. Synthesis of 4-(1-methyl-1,2,3,4-tetrahydropyrido[2,3-b]pyrazin-6-yl)butanenitrile

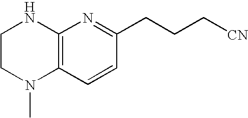

A mixture of the product of step 9 (5.78 g, 14.88 mmol) and KOH (powder, 1.25 g, 22.32 mmol) in ethylene glycol (30 mL) under N₂ was heated at 150 for 3 hours. The mixture was cooled to 0° C. and portioned between water and EtOAc. The organic phase was washed with brine, dried over Na₂SO₄ and concentrated in vacuo. Flash chromatography (silica, 100% EtOAc) yielded a colorless oil). H NMR (400 MHz, CDCl₃) δ 2.00 (t, 2H), 2.32 (t, 2H), 2.63 (t, 23H), 2.82 (s, 3H), 3.20 (t, 2H), 3.56 (t, 2H), 6.40 (d, 1H), 6.58(d, 1H).
Step 11. (1E)-N′-hydroxy-4-(1-methyl-1,2,3,4-tetrahydropyrido[2,3-b]pyrazin-6-yl)butanimidamide

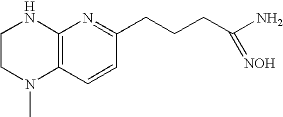

A mixture of the product of step 10 (500 mg, 2.31 mmol) and hydroxylamine (0.56 mL of a 50% weight solution in water, 8.50 mmol) in ethanol (20 mL) under N₂ was heated at 60° C. overnight. The mixture was cooled to room temperature and concentrated in vacuo to yield a white solid). H NMR (400 MHz, CD₃OD) δ 1.80 (m, 2H), 2.05 (t, 2H), 2.45 (t, 2H), 2.75 (s, 3H), 3.15 (t, 2H), 3.55 (t, 2H), 6.35 (d, 1H), 6.60 (d, 1H).
Step 12. 3-(1,3-benzodioxol-5-yl)-4-{3-[3-(1-methyl-1,2,3,4-tetrahydropyrido[2,3-b]pyrazin-6-yl)propyl]-1,2,4-oxadiazol-5-yl}butanoic acid

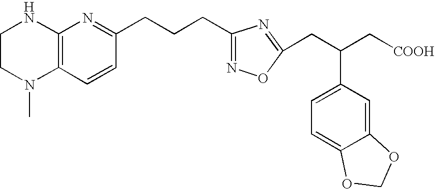

A mixture of the product of step 11 (260 mg, 1.04 mmol) and Example 1 Step 3 (290 mg, 1.25 mmol) in 1,4-dioxane (20 mL) was heated at 90° C. overnight. The reaction mixture was allowed to cool to room temperature and concentrated. The residue was purified on HPLC using acetonitrile gradient 5-50% in 30 min to yield 200 mg desired product. H NMR (400 MHz, DMSO-d6) [symbol] 1.95 (m, 2H), 2.58-2.80 (m, 6H), 3.85 (s, 3H), 3.10-3.35 (m, 5H), 3.60 (t, 2H), 5.86 (s, 2H), 6.54 (d, 1H), 6.63 (d, 1H), 6.75 (d, 1H), 7.90 (m, 2H). FAB-MS:(MH⁺)=466, Calcd. for C₂₄H₂₇N₅O₅. 1.5 TFA. 1.0H₂O: C, 49.54; H, 4.70; N, 10.70. Found: C, 49.29; H, 4.58; N, 11.06.

### EXAMPLE 119

3-(2-methyl-1,3-benzothiazol-5-yl)-4-{3-[3-(1-methyl-1,2,3,4-tetrahydropyrido[2,3-b]pyrazin-6-yl)propyl]-1,2,4-oxadiazol-5-yl}butanoic acid

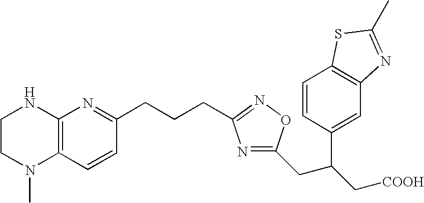

This prepared by starting from 4-(2-methyl-2,3-dihydro-1,3-benzothiazol-5-yl)dihydro-2H-pyran-2,6(3H)-dione and using the procedure described in example 118. ¹H NMR (400 MHz, DMSO-d6) [symbol] 1.95 (m, 2H), 2.58-2.80 (m, 6H), 3.85 (s, 3H), 3.10-3.35 (m, 5H), 3.60 (t, 2H), 5.86 (s, 2H), 6.54 (d, 1H), 6.63 (d, 1H), 6.75 (d, 1H), 7.90 (m, 2H). FAB-MS:(MH+)=493, Calcd. for C₂₅H₂₈N₆O₃S. 1.8 TFA. 0.5H₂O: C, 48.60; H, 4.39; N, 12.15. Found: C, 48.84; H, 4.71; N, 12.15.

### EXAMPLE 120

3-(3-fluoro-4-methoxyphenyl)-4-{3-[3-(1-methyl-1,2,3,4-tetrahydropyrido[2,3-b]pyrazin-6-yl)propyl]-1,2,4-oxadiazol-5-yl}butanoic acid

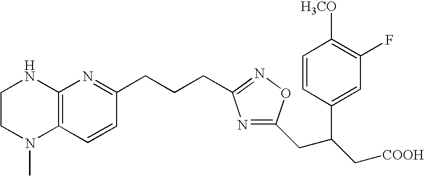

This prepared by starting from 4-(3-fluoro-4-methoxyphenyl)dihydro-2H-pyran-2,6(3H)-dione and using the procedure described in example 118. ¹H NMR (400 MHz, DMSO-d6) [symbol] 1.95 (m, 2H), 2.40-2.80 (m, 6H), 3.85 (s, 3H), 3.10-3.35 (m, 4H), 3.58 (m, 1H), 3.60 (t, 2H), 3.80 (s, 3H), 6.40 (d, 1H), 6.75 (d, 1H), 6.95 (s, 1H), 6.95-7.05 (dd, 2H). FAB-MS:(MH+)=493, Calcd. for C₂₄H₂₈FN₅O_{4.}1.3 TFA. 0.9H₂O: C, 51.12; H, 4.85; N, 11.21. Found: C, 51.16; H, 4.98; N, 11.28.

### EXAMPLE 121

3-(6-methoxypyridin-3-yl)-4-{3-[3-(1-methyl-1,2,3,4-tetrahydropyrido[2,3-b]pyrazin-6-yl)propyl]-1,2,4-oxadiazol-5-yl}butanoic acid

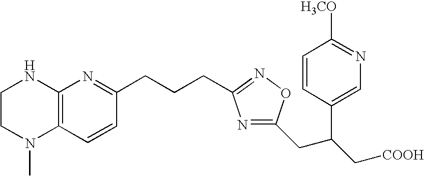

This prepared by starting from 4-(6-methoxypyridin-3-yl)dihydro-2H-pyran-2,6(3H)-dione and using the procedure described in example 118. ¹H NMR (400 MHz, DMSO-d6) [symbol] 1.95 (m, 2H), 2.60 (t, 2H), 2.70 (d, 2H), 2.80 (m, 2H), 2.85 (s, 3H), 3.20-3.40 (m, 4H), 3.65 (t, 2H), 3.680 (m, 1H), 3.82 (s, 3H), 6.560 (d, 1H), 6.78 (d, 1H), 6.90 (d, 1H), 7.70 (q, 1H), 7.96 (d, 1H). FAB-MS:(MH+)=453, Calcd. for C₂₃H₂₈N₆O_{4.}3.1 TFA: C, 43.51; H, 3.89; N, 10.43. Found: C, 43.36; H, 4.198; N, 10.19.

### EXAMPLE 122

Synthesis of 3-(1,3-benzodioxol-5-yl)-4-(3-{[2-(5,6,7,8-tetrahydro-1,8-naphthyridin-2-yl)ethyl]thio}-1H-1,2,4-triazol-5-yl)butanoic acid. 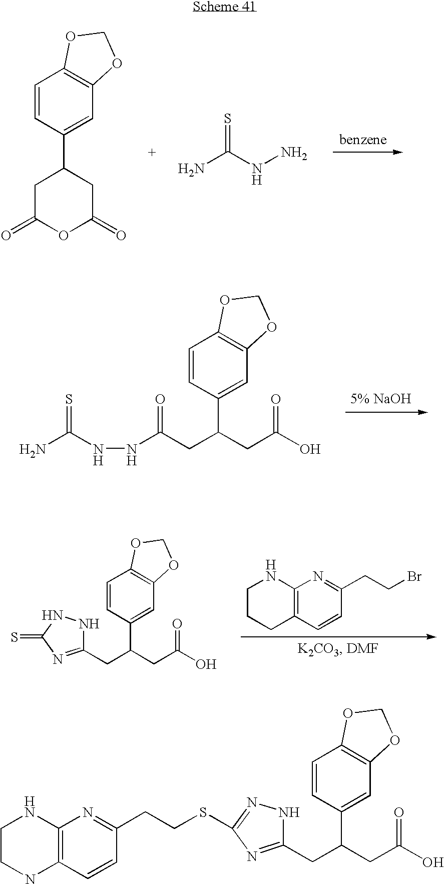
Step 1. Synthesis of 5-[2-(aminocarbonothioyl)hydrazino]-3-(1,3-benzodioxol-5-yl)-5-oxopentanoic acid

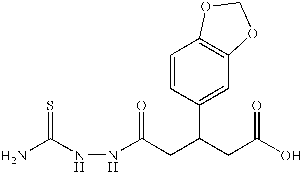

To 4-(1,3-benzodioxol-5-yl)dihydro-2H-pyran-2,6(3H)-dione (1.0 g, 4.3 mmol) in benzene (17 mL) was added thiosemicarbazide (428 mg, 4.7 mmol). The reaction was refluxed (7 mL of THF was added to help with solubility). After 3 h at reflux, the reaction mixture was concentrated to the desired product as a white solid (1.4 g). Compound had minor impurities and was used as is in the next step. LC-MS (MH+)=326.
Step 2. Synthesis of 3-(1,3-benzodioxol-5-yl)-4-(5-thioxo-2,5-dihydro-1H-1,2,4-triazol-3-yl)butanoic acid.

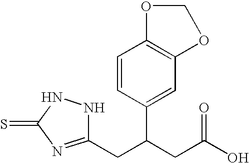

To 5-[2-(aminocarbonothioyl)hydrazino]-3-(1,3-benzodioxol-5-yl)-5-oxopentanoic acid (1.4 g, 4.3 mmol) was added 5% aq. NaOH (20 mL). The reaction mixture was heated at 100° C. for 1.5 h. The reaction mixture was cooled to room temp and acidified to pH≈4. The mixture was extracted with EtOAc (3×40 mL). The organic layers were combined, washed with brine, dried over Na₂SO₄, and concentrated to the desired product as a brown solid (156 mg). LC-MS (MH+)=308.
Step 3. Synthesis of 3-(1,3-benzodioxol-5-yl)-4-(3-{[2-(5,6,7,8-tetrahydro-1,8-naphthyridin-2-yl)ethyl]thio}-1H-1,2,4-triazol-5-yl)butanoic acid.

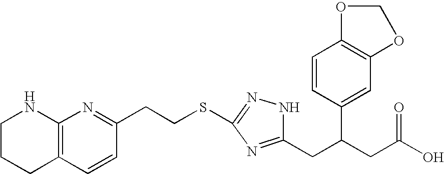

To a mixture of 3-(1,3-benzodioxol-5-yl)-4-(5-thioxo-2,5-dihydro-1H-1,2,4-triazol-3-yl)butanoic acid (155 mg, 0.50 mmol) and K₂CO₃ (145 mg, 1.1 mmol) in anhydrous DMF (9 mL) under Ar gas at 60° C. was added a solution of 7-(2-bromoethyl)-1,2,3,4-tetrahydro-1,8-naphthyridine (133 mg, 0.55 mmol) in anhydrous DMF (3 mL). After stirring for 4 h at 60° C., the reaction mixture was concentrated. The residue was acidified with 1N HCl and purified by reverse phase HPLC using (H₂O/HCl)/CH₃CN as eluent (0.5 mL conc. HCl in 4 L H₂O). Obtained was the HCl salt desired product (47 mg). ¹H NMR (400 MHz, DMSO-d₆) δ 1.81 (m, 2H), 2.50-2.71 (m, 2H), 2.73 (t, 2H), 2.96 (m, 2H), 3.02 (t, 2H), 3.43 (m, 5H), 5.93 (s, 2H), 6.52 (d, 1H), 6.64 (dd, 1H), 6.75 (d, 1H), 6.85 (d, 1H), 7.58 (d, 1H), 8.01 (bs, 1H); MS (ESI+) for C₂₃H₂₅N₅O₄S m/z 468.1721 (M+H)⁺. Anal. Cald. for C₂₃H₂₅N₅O₄S.2HCl.1.3H₂O: C, 48.99; H, 5.29; N, 12.42. Found: C, 48.69; H, 5.31; N, 12.34.

### EXAMPLE 123

Synthesis of 3-(1,3-benzodioxol-5-yl)-4-(1-methyl-5-{[2-(5,6,7,8-tetrahydro-1,8-naphthyridin-2-yl)ethyl]thio}-1H-1,2,4-triazol-3-yl)butanoic acid 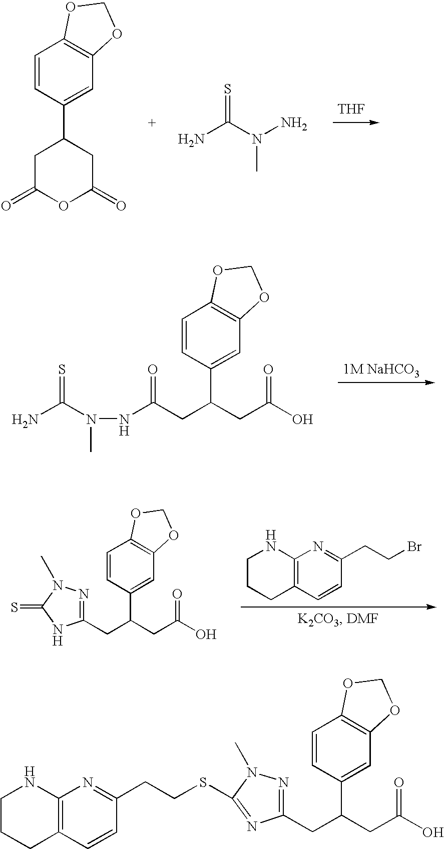
Step 1. Synthesis of 5-[2-(aminocarbonothioyl)-2-methylhydrazino]-3-(1,3-benzodioxol-5-yl)-5-oxopentanoic acid

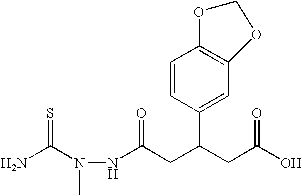

To 4-(1,3-benzodioxol-5-yl)dihydro-2H-pyran-2,6(3H)-dione (1.0 g, 4.3 mmol) in anhydrous THF (20 mL) was added 2-methyl-3-thiosemiarbazide (494 mg, 4.7 mmol). The reaction mixture was refluxed for 3 h. The reaction mixture was concentrated to give the desired product (1.5 g). LC-MS (MH+)=340.
Step 2. Synthesis of 3-(1,3-benzodioxol-5-yl)-4-(1-methyl-5-thioxo-4,5-dihydro-1H-1,2,4-triazol-3-yl)butanoic acid

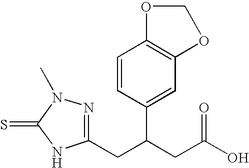

To 5-[2-(aminocarbonothioyl)-2-methylhydrazino]-3-(1,3-benzodioxol-5-yl)-5-oxopentanoic acid (1.5 g) was added a 1 M solution of NaHCO₃ (43 mL, 43 mmol). The reaction mixture was heated at 80° C. overnight. The reaction mixture was cooled to room temp and acidified to pH 3. The resulting mixture was extracted with EtOAc (3×40 mL). The organic layers were combined, washed with brine, dried over Na₂SO₄, and concentrated to the desired product (950 mg). ¹H NMR (400 MHz, DMSO-d₆) δ 2.50-2.64 (m, 2H), 2.75-2.92 (m, 2H), 3.43 (m, 1H), 3.51 (s, 3H), 5.96 (s, 2H), 6.67 (dd, 1H), 6.79 (d, 1H), 6.88 (d, 1H), 12.08 (bs, 1H). LC-MS (MH+)=322.
Step 3. Synthesis of 3-(1,3-benzodioxol-5-yl)-4-(1-methyl-5-{[2-(5,6,7,8-tetrahydro-1,8-naphthyridin-2-yl)ethyl]thio}-1H-1,2,4-triazol-3-yl)butanoic acid

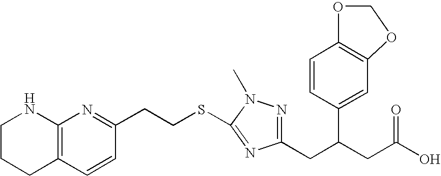

Same procedure as Example 122, step 3, using 3-(1,3-benzodioxol-5-yl)-4-(1-methyl-5-thioxo-4,5-dihydro-1H-1,2,4-triazol-3-yl)butanoic acid as starting material. ¹H NMR (400 MHz, DMSO-d₆) δ 1.81 (m, 2H), 2.46-2.68 (m, 2H), 2.73 (t, 2H), 2.75-2.90 (m, 2H), 3.03 (t, 2H), 3.41 (m, 3H), 3.49 (t, 2H), 3.59 (s, 3H), 5.92 (s, 2H), 6.54 (d, 1H), 6.67 (dd, 1H), 6.75 (d, 1H), 6.87 (d, 1H), 7.58 (d, 1H), 7.97 (bs, 1H). LC-MS (MH+)=482. Anal. Cald. for C₂₄H₂₇N₅O₄S.HCl.1.75H₂O: C, 52.45; H, 5.78; N, 12.74. Found: C, 52.46; H, 5.73; N, 12.71.

### EXAMPLE 124

Synthesis of 3-(1,3-benzodioxol-5-yl)-4-(4-methyl-5-{[2-(5,6,7,8-tetrahydro-1,8-naphthyridin-2-yl)ethyl]thio}-4H-1,2,4-triazol-3-yl)butanoic acid 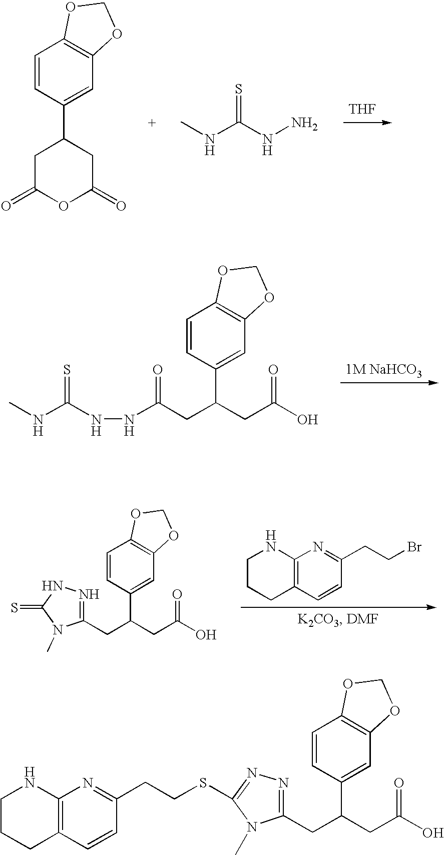
Step 1. Synthesis of 3-(1,3-benzodioxol-5-yl)-5-{2-[(methylamino)carbonothioyl]hydrazino}-5-oxopentanoic acid

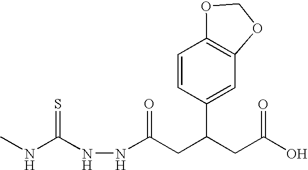

Same procedure as Example 123, step 1, using 4-methyl-3-thiosemicarbazide as starting material. LC-MS (MH+)=340.
Step 2. Synthesis of 3-(1,3-benzodioxol-5-yl)-4-(4-methyl-5-thioxo-4,5-dihydro-1H-1,2,4-triazol-3-yl)butanoic acid

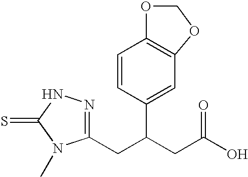

Same procedure as Example 123, step 2, using 3-(1,3-benzodioxol-5-yl)-5-{2-[(methylamino)carbonothioyl]hydrazino}-5-oxopentanoic acid as starting material.

¹H NMR (400 MHz, DMSO-d₆) δ 2.51-2.77 (m, 2H), 2.89-3.07 (m, 2H), 3.31 (s, 3H), 3.39 (m, 1H), 5.97 (s, 2H), 6.67 (dd, 1H), 6.78 (d, 1H), 6.92 (d, 1H), 12.12 (bs, 1H). LC-MS (MH+)=322.
Step 3. Synthesis of 3-(1,3-benzodioxol-5-yl)-4-(4-methyl-5-{[2-(5,6,7,8-tetrahydro-1,8-naphthyridin-2-yl)ethyl]thio}-4H-1,2,4-triazol-3-yl)butanoic acid

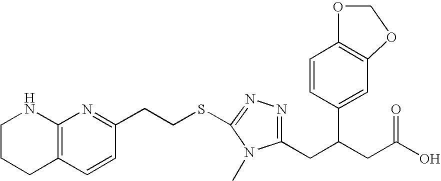

Same procedure as Example 122, step 3, using 3-(1,3-benzodioxol-5-yl)-4-(4-methyl-5-thioxo-4,5-dihydro-1H-1,2,4-triazol-3-yl)butanoic acid as starting material. ¹H NMR (400 MHz, DMSO-d₆) δ 1.81 (m, 2H), 2.60-2.89 (m, 4H), 3.08 (t, 2H), 3.20-3.41 (m, 2H), 3.43 (m, 2H), 3.54 (m, 1H), 3.58 (s, 3H), 3.62 (t, 2H), 5.97 (d, 2H), 6.67 (d, 1H), 6.78 (m, 2H), 7.01 (s, 1H), 7.61 (d, 1H), 8.15 (bs, 1H). MS (ESI+) for C₂₄H₂₇N₅O₄S m/z 482.1885 (M+H)⁺. Anal. Cald. for C₂₄H₂₇N₅O₄S.3HCl.H₂O: C, 47.34; H, 5.30; N, 11.50. Found: C, 47.33; H, 5.58; N, 11.49.

### EXAMPLE 125

3-(1,3-benzodioxol-5-yl)-4-{3-[2-(1-methyl-1,2,3,4-tetrahydropyrido[2,3-b]pyrazin-6-yl)ethoxy]isoxazol-5-yl}butanoic acid 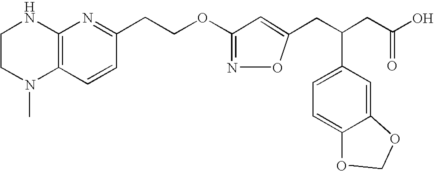 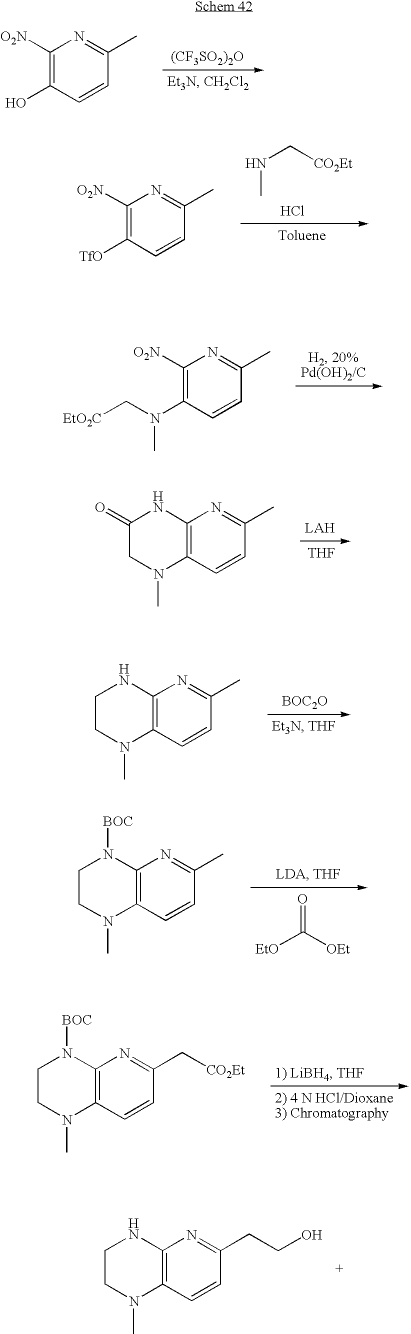 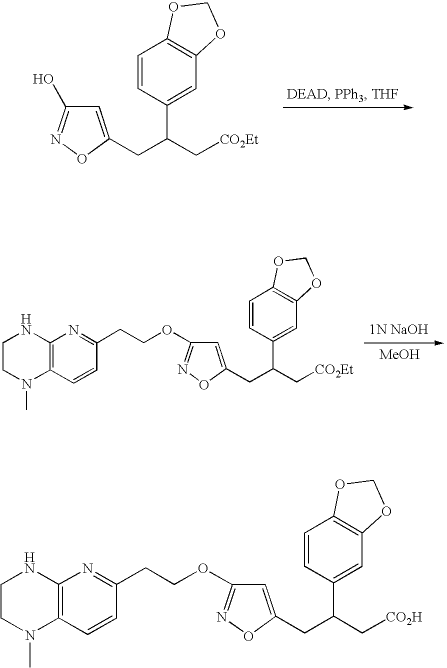
Step 1. Synthesis of 6-methyl-2-nitropyridin-3-yl trifluoromethanesulfonate.

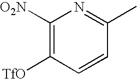

To a solution of 3-hydroxy-6-methyl-2-nitropyridine (2 g, 12.97 mmol, 1 eq) in CH₂Cl₂ (150 mL) at 0° C. under N₂ was added triethylamine (2.68 mL, 19.27 mmol, 1.48 eq) and followed by trifluoromethanesulfonic anhydride (2.62 mL, 15.57 mmol, 1.2 eq). The mixture was stirred for 2 hours at 0° C. and then quenched with water. The organic layer was separated, washed with water and dried over MgSO4. After filtration and concentration at reduced pressure, the crude mixture was purified by flash chromatography on silica gel (15% EA/Hex) to afford the desired product (3.65 g, 98% yield) as a yellow oil. H NMR (400 MHz, CDCl₃) δ 2.70 (s, 3H), 7.59 (d, 1H), 7.81 (d, 2H).

Step 2. Synthesis of ethyl N-methyl-N-(6-methyl-2-nitropyridin-3-yl)glycinate.

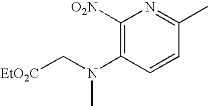

To a solution of 6-methyl-2-nitropyridin-3-yl trifluoromethanesulfonate (7 g, 24.47 mmol, 1 eq) in toluene (40 mL) at room temperature under N₂ was added sarcosine ester hydrochloride (9.4 g, 61.2 mmol, 2.5 eq) and followed by triethylamine (8.51 mL, 61.2 mmol, 2.5 eq). The mixture was refluxed overnight under N₂. The reaction was cooled to room temperature and quenched with water. The mixture was extracted three times with ethyl acetate and all organic extracts were combined, washed with brine, dried over Na₂SO₄. After filtration and concentration at reduced pressure, the crude mixture was purified by flash chromatography on silica gel (20% EA/Hex) to afford the desired product (4.3 g, 69% yield) as brown oil. H NMR (400 MHz, CDCl₃) δ 1.026 (t, 3H), 2.50 (s, 3H), 2.95 (s, 3H), 3.88 (s, 2H), 4.20 (q, 2H), 7.27 (d, 1H), 7.49 (d, 2H).
Step 3. Synthesis of 1,6-dimethyl-1,4-dihydropyrido[2,3-b]pyrazin-3(2H)-one.

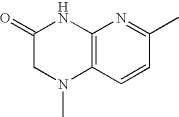

6-Methyl-2-nitropyridin-3-yl trifluoromethanesulfonate (4.3 g, 17 mmol) was hydrogenated in ethanol solution at room temperature using H₂ at 5 psi and 20% Pd(OH)₂/C catalyst for 2 hours. Upon completion of the reaction, the catalyst was filtered off and the filtrate was concentrated under reduced pressure. The product was crystallized out from 50% EA/Hex solution as yellow crystalline solid. The mother liquid was concentrated and purified by flash chromatography on silica gel (50% EA/Hex). (1.44 g, 46% yield) H NMR (400 MHz, CDCl₃) δ 2.26 (s, 3H), 2.70 (s, 3H), 3.18 (t, 2H), 3.58 (m, 2H), 6.34 (d, 1H), 6.57(d, 2H).
Step 4. Synthesis of 1,6-dimethyl-1,2,3,4-tetrahydropyrido[2,3-b]pyrazine.

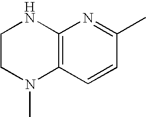

LiAlH₄ (214 mg, 5.64 mmol) was slowly added to 10 mL anhydrous THF in a round-bottom flask fitted with a stirbar and a condenser. After stirring for 10 minutes, a solution of 1,6-dimethyl-1,4-dihydropyrido[2,3-b]pyrazin-3(2H)-one (500 mg, 2.82 mmol) in 5 mL anhydrous THF was added dropwise. Upon completion of the addition, the reaction mixture was refluxed for 16 hours. The reaction was cooled to room temperature and quenched with 1 M NaOH solution until the mixture had become a milky yellow color. The precipitate was filtered off and washed 3 times with CH₂Cl₂. The filtrate and washings were combined, washed with brine, dried over MgSO₄. Filtered and concentrated under reduced pressure to give the desired product as light yellow oil, which solidified on standing. (420 mg, 91% yield). H NMR (400 MHz, CDCl₃) δ 2.27 (s, 3H), 2.80 (s, 3H), 3.17 (t, 2H), 3.58 (m, 2H), 6.36 (d, 1H), 6.56(d, 2H).
Step 5. Synthesis of tert-butyl 1,6-dimethyl-2,3-dihydropyrido[2,3-b]pyrazine-4(1H)-carboxylate.

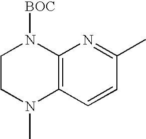

A solution of 1,6-dimethyl-1,2,3,4-tetrahydropyrido[2,3-b]pyrazine (1.14 g, 7 mmol), di-tert-butyl dicarbonate (2.29 g, 10.5 mmol), DMAP (100 mg) and triethylamine (1.46 mL, 10.5 mmol) in 30 mL THF was refluxed 72 hours under N₂. The reaction mixture was allowed to cool to room temperature and diluted with ethyl acetate. The mixture was washed with brine, dried over Na₂SO₄. After filtration and concentration at reduced pressure, the crude mixture was purified by flash chromatography on silica gel (40% EA/Hex) to afford the desired product (1.6 g, 90% yield) as yellow oil. H NMR (400 MHz, CDCl₃) δ 1.51 (s, 9H), 2.40 (s, 3H), 2.90 (s, 3H), 3.28 (t, 2H), 3.83 (m, 2H), 6.78 (d, 1H), 6.83(d, 2H).
Step 6. Synthesis of tert-butyl 6-(2-ethoxy-2-oxoethyl)-1-methyl-2,3-dihydropyrido[2,3-b]pyrazine-4(1H)-carboxylate.

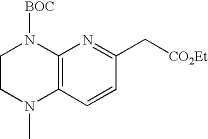

Lithium diisopropylamide solution (5 mL, 10 mmol, 2.0 M in THF/ethylbenzene/heptane) was added dropwise to a chilled (−78° C.), stirred solution of tert-butyl 1,6-dimethyl-2,3-dihydropyrido[2,3-b]pyrazine-4(1H)-carboxylate (950 mg, 3.61 mmol) and diethyl carbonate (1.62 mL, 13.36 mmol) in 20 mL dry THF under nitrogen atmosphere. After 1 hour the reaction was quenched with saturated NH₄Cl solution and warmed to room temperature. The mixture was extracted three times with ethyl acetate and all organic extracts were combined, dried over Na₂SO₄, and concentrated under reduced pressure to get the crude product, which was purified by chromatography on silica gel (eluent: 30% ethyl acetate/hexane). The desired fractions were combined and concentrated under reduced pressure to get the desired product F (1.05 g, 87% yield) as a yellow solid. H NMR (400 MHz, CDCl₃) δ 1.25 (t, 3H), 1.50 (s, 9H), 2.78 (s, 3H), 3.38 (t, 2H), 3.68(s, 2H), 3.84 (t, 2H), 4.14 (q, 2H), 6.86 (d, 1H), 6.95(d, 2H).
Step 7. Synthesis of 2-(1-methyl-1,2,3,4-tetrahydropyrido[2,3-b]pyrazin-6-yl)ethanol.

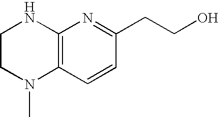

To a solution of tert-butyl 6-(2-ethoxy-2-oxoethyl)-1-methyl-2,3-dihydropyrido[2,3-b]pyrazine-4(1H)-carboxylate 1.05 g, 3.13 mmol)) in dry THF (15 mL) at room temperature was added a solution of LiBH₄ (2.0 M in THF, 1.88 mL), and the resulting mixture was heated to reflux. After 16 hours the mixture was cooled to 0° C. and carefully quenched with water (20 mL). After 10 minutes, the mixture was extracted three times with ethyl acetate. The combined organic extracts were dried over MgSO₄, filtered, and concentrated under reduced pressure. This residue was dissolved in CH₂Cl₂ (3 mL), and to this solution was added 4 M HCl in dioxane (6 mL) all at once at room temp. After 4 hours, the mixture was concentrated under reduced pressure to get the crude product, which was chromatographed on silica gel (eluent: 98/2/0.5 dichloromethane/methanol/-ammonium hydroxide) to afford the desired product as a gray solid. (390 mg) H NMR (400 MHz, CDCl₃) δ 2.73 (t, 2H), 2.72 (s, 3H), 3.20 (t, 2H), 3.58(m, 2H), 3.89 (t, 2H), 6.36 (d, 1H), 6.58(d, 2H).
Step 8. Synthesis of 3-(1,3-benzodioxol-5-yl)-7-ethoxy-5,7-dioxoheptanoic acid.

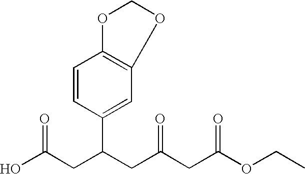

To a solution of anhydrous EtOAc (4.38 mL, 44.8 mmol) in anhydrous THF (25 mL) at −78° C. under Ar gas was slowly added lithium diisopropylamide (2M in heptane/THF/ethylbenzene, 22.4 mL, 44.8 mmol). The resulting solution was stirred at −78° C. for 25 min and added dropwise via cannula to a solution of 4-(1,3-benzodioxol-5-yl)dihydro-2H-pyran-2,6(3H)-dione (synthesis described in Example 1 Step 3) (5.0 g, 21.3 mmol) in anhydrous THF (170 mL) at −78° C. under Ar gas. The reaction mixture was stirred at −78° C. for 1.5 h. The reaction mixture was quenched with 2N HCl in ether (80 mL) and allowed to warm up to room temperature. To the reaction mixture was added water (100 mL) and extracted with EtOAc (3×100 mL). The organic layers were combined, washed with brine, dried over Na₂SO₄, and concentrated under reduced pressure. The resulting residue was purified by flash column chromatography using 40% EtOAc/hexane to give a white solid (5.61 g, 17.4 mmol, 82%). ¹H NMR (400 MHz, CDCl₃) δ 1.25 (t, 3H), 2.55-2.73 (m, 2H), 2.90 (m, 2H), 3.34 (s, 2H), 3.60 (m, 1H), 4.15 (q, 2H), 5.93 (s, 2H), 6.70 (m, 3H). LC-MS (M+Na)=345.
Step 9. Synthesis of ethyl 3-(1,3-benzodioxol-5-yl)-4-(3-hydroxyisoxazol-5-yl)butanoate.

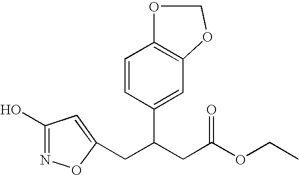

Hydroxylamine hydrochloride (1.1 g, 16.4 mmol) was dissolved in approximately 4.3 mL of 2N NaOH to achieve a solution of pH 10.0±0.3 (pH meter used). The solution was cooled to 0° C. and stirred vigorously while a solution of 3-(1,3-benzodioxol-5-yl)-7-ethoxy-5,7-dioxoheptanoic acid (4.8 g, 14.9 mmol) in 2N NaOH (approximately 8.5 mL) was added slowly while maintaining the pH of the reaction mixture at 10.0±0.3 by dropwise addition of 2N NaOH. After complete addition, the reaction mixture was stirred at 0° C. for 1.5 h and quenched into ice cold concentrated HCl (20 mL). The reaction mixture was warmed up to room temperature and stirred for 4 h. The resulting mixture was poured into ice water (200 mL) and extracted with EtOAc (3×200 mL). The organic layers were combined, washed with brine, dried over Na₂SO₄, and concentrated under reduced pressure. The resulting residue (3.8 g) was dissolved in EtOH (15 mL), and 4N HCl in dioxane (15 mL) was added. The reaction mixture was stirred at room temperature overnight. The reaction mixture was concentrated under reduced pressure and the residue purified by flash column chromatography using 50% EtOAc/hexane as eluent. Obtained was a yellow oil (1.22 g, 3.8 mmol, 26%). ¹H NMR (400 MHz, DMSO-d6) δ 1.08 (t, 3H), 2.54-2.72 (m, 2H), 2.93 (m, 2H), 3.33 (m, 1H), 3.95 (q, 2H), 5.58 (s, 1H), 5.97 (s, 2H). 6.68 (dd, 1H), 6.78 (d, 1H), 6.91 (d, 1H), 10.95 (s, 1H). LC-MS (MH+)=320.
Step 10. Synthesis of ethyl 3-(1,3-benzodioxol-5-yl)-4-{3-[2-(1-methyl-1,2,3,4-tetrahydropyrido[2,3-b]pyrazin-6-yl)ethoxy]isoxazol-5-yl}butanoate.

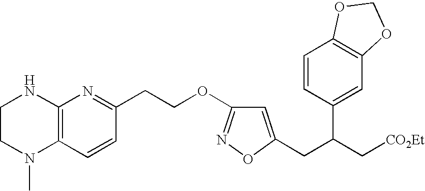

To 2-(1-methyl-1,2,3,4-tetrahydropyrido[2,3-b]pyrazin-6-yl)ethanol (110 mg, 0.57 mmol), ethyl 3-(1,3-benzodioxol-5-yl)-4-(3-hydroxyisoxazol-5-yl)butanoate.

(182 mg, 0.57 mmol), and triphenylphosphine (164 mg, 0.63 mmol) in anhydrous THF under N₂ gas at 0° C. added diisopropyl azodicarboxylate (124 μL, 0.63 mmol). The reaction mixture was stirred overnight at room temperature. The reaction mixture was concentrated under reduced pressure. The residual oil was purified by reversed phase HPLC using (H₂O/TFA)/CH₃CN as eluent (2.5 mL TFA in 4 L H₂O) to afford 100 mg of the title compound as yellow oil. H NMR (400 MHz, CD₃OD) δ 1.15 (t, 3H), 2.59-2.76 (m, 2H), 2.95-3.09 (m, 7H), 3.30 (t, 2H), 3.43 (m, 1H), 3.66 (t, 2H), 4.41 (q, 2H), 4.39 (t, 2H), 5.64 (s, 1H), 5.91 (s, 2H), 6.64-6.77 (m, 4H), 6.91 (d, 1H).
Step 11. Synthesis of 3-(1,3-benzodioxol-5-yl)-4-{3-[2-(1-methyl-1,2,3,4-tetrahydropyrido[2,3-b]pyrazin-6-yl)ethoxy]isoxazol-5-yl}butanoic acid

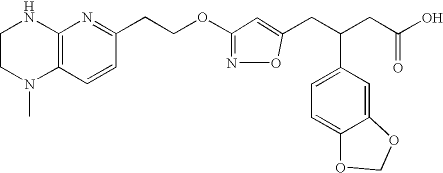

The product ethyl 3-(1,3-benzodioxol-5-yl)-4-{3-[2-(1-methyl-1,2,3,4-tetrahydropyrido[2,3-b]pyrazin-6-yl)ethoxy]isoxazol-5-yl}butanoate (100 mg, 0.20 mmol) was dissolved in 2 ml methanol and 2 ml 1N sodium hydroxide solution. The reaction was stirred at room temperature overnight, concentrated and acidified with 1 ml trifluoroacetic acid, then purified by reverse phase HPLC using (H₂O/TFA)/CH₃CN as eluent (2.5 mL TFA in 4 L H₂O) to yield 50 mg desired product as dark green oil. FAB-MS:(MH+)=467. H NMR (500 MHz, CD₃OD) δ 2.55-2.68 (m, 2H), 2.91-3.07 (m, 7H), 3.29 (t, 2H), 3.40 (m, 1H), 3.65 (t, 2H), 4.37 (t, 2H), 5.60(s, 1H), 5.88 (s, 2H), 6.61-6.74 (m, 4H), 6.87 (d, 1H). Anal Calcd. for C₂₄H₂₆N₄O₆ plus 1.4 CF₃COOH and 1H₂O: C, 49.97; H, 4.60; N, 8.70. Found: 49.94; H, 4.84; N, 8.56.

### EXAMPLE 126

3-(1,3-benzodioxol-5-yl)-4-(3-{2-[6-(methylamino)pyridin-2-yl]ethoxy}isoxazol-5-yl)butanoic acid 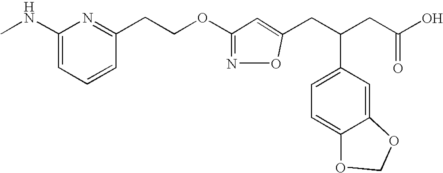
Step 1. Synthesis of 2-[6-(methylamino)pyridin-2-yl]ethanol.

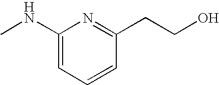

Synthesis was described in Patent No. WO 2002088118.
Step 2. Synthesis of ethyl 3-(1,3-benzodioxol-5-yl)-4-(3-{2-[6-(methylamino)pyridin-2-yl]ethoxy}isoxazol-5-yl)butanoate.

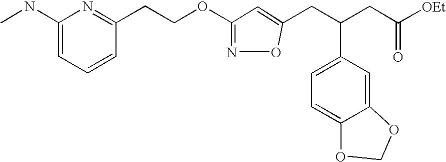

Same synthetic procedure as for Example 125, Step 10, using 2-[6-(methylamino)pyridin-2-yl]ethanol as the starting material.
Step 3. Synthesis of 3-(1,3-benzodioxol-5-yl)-4-(3-{2-[6-(methylamino)pyridin-2-yl]ethoxy}isoxazol-5-yl)butanoic acid

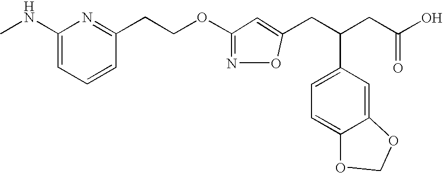

The product ethyl 3-(1,3-benzodioxol-5-yl)-4-(3-{2-[6-(methylamino)pyridin-2-yl]ethoxy}isoxazol-5-yl)butanoate (453 mg, 0.62 mmol) was dissolved in 3 ml methanol and 3 ml 1N sodium hydroxide solution. The reaction was stirred at room temperature overnight, concentrated and acidified with 1 ml trifluoroacetic acid, then purified by reverse phase HPLC using (H₂O/TFA)/CH₃CN as eluent (2.5 mL TFA in 4 L H₂O) to yield 135 mg desired product as yellow oil. FAB-MS:(MH+)=426. H NMR (500 MHz, CD₃OD) δ 2.55-2.68 (m, 2H), 2.93-3.05 (m, 5H), 3.23 (t, 2H), 3.40 (m, 1H), 4.47 (t, 2H), 5.61 (s, 1H), 5.88 (s, 2H), 6.64-6.74 (m, 3H), 6.89 (d, 1H), 7.81 (t, 1H). Anal Calcd. for C₂₂H₂₃N₃O₆ plus 1.2 CF₃COOH and 1H₂O: C, 50.50; H, 4.55; N, 7.24. Found: 50.23; H, 4.71; N, 7.27.

### EXAMPLE 127

3-(6-methoxypyridin-3-yl)-4-{3-[2-(5,6,7,8-tetrahydro-1,8-naphthyridin-2-yl)ethoxy]isoxazol-5-yl}butanoic acid 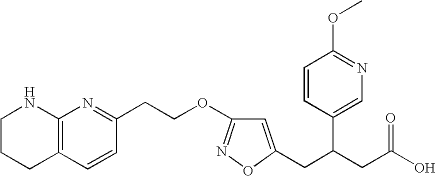
Step 1. Synthesis of 7-ethoxy-3-(6-methoxypyridin-3-yl)-5,7-dioxoheptanoic acid.

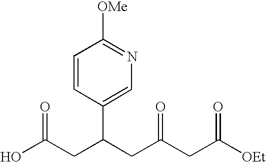

To a solution of anhydrous EtOAc (9.27 mL, 94.9 mmol) in anhydrous THF (37 mL) at −78° C. under Ar gas was slowly added lithium diisopropylamide (2M in heptane/THF/ethylbenzene, 47.5 mL, 94.9 mmol). The resulting solution was stirred at −78° C. for 25 min and added dropwise via cannula to a solution of the 4-(6-methoxypyridin-3-yl)dihydropyran-2,6(3H)-dione (synthesis described below)(10 g, 45.2 mmol) in anhydrous THF (250 mL) at −78° C. under Ar gas. The reaction mixture was stirred at −78° C. for 1.5 h. The reaction mixture was quenched with 2N HCl in ether (100 mL) and allowed to warm up to room temperature. To the reaction mixture was added water (200 mL) and extracted with EtOAc (2×100 mL). The aqueous layer was basified to PH=4 with 2N NaOH solution and extracted with EtOAc (3×150 mL). The organic layers were combined, washed with brine, dried over Na₂SO₄, and concentrated under reduced pressure. The resulting residue was purified by flash column chromatography using 80% EtOAc/hexane to give 2.28 g of the title compound as a brown oil. ¹H NMR (400 MHz, CDCl₃) δ 1.24 (t, 3H), 2.57-2.81 (m, 4H), 2.86-3.03 (m, 2H), 3.66 (m, 1H), 4.15 (m, 2H), 6.69 (d, 1H), 7.46 (dd, 1H), 8.05 (d, 1H).
Synthesis of 4-(6-methoxypyridin-3-yl)dihydropyran-2,6(3H)-dione: 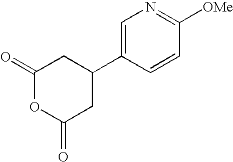
Step 1. Synthesis of dimethyl 3-(6-methoxypyridin-3-yl)pent-2-enedicarboxylate.

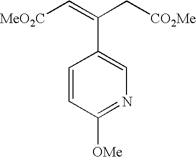

A mixture of dimethyl pent-2-enedicarboxylate (2.86 g, 18.09 mmol), Palladium (II) acetate (0.12 g, 0.53 mmol), tri-o-tolyphosphine (0.405 g, 1.33 mmol), and triethylamine (2.0 mL) in DMF (2.13 mL) was degassed and heated at 90 C. The 5-Bromo-2-methoxy pyridine (1) was added dropwise to the mixture and heated at 90 C overnight. The reaction mixture was cooled to rt and the solid was filtered. The filtrate was diluted with water and this mixture was extracted with ethyl acetate (3×100 mL). The organic layers were combined, washed with brine, dried over Na₂SO₄ and concentrated under reduced pressure. The resulting residue was purified by flash column chromatography using 5-25% EtOAc/Hexane to give dimethyl 3-(6-methoxypyridin-3-yl)pent-2-enedicarboxylate as light yellow oil (0.301 g, 21%). ¹H NMR (CD₃OD) δ 8.31 (d, 1H), 7.88-7.84 (m, 1H), 6.84 (d, 1H), 6.33 (s, 1H), 4.86 (s, 2H), 3.95 (s, 3H), 3.75 (s, 3H), 3.68 (s, 3H);
Step 2. Synthesis of dimethyl 3-(6-methoxypyridin-3-yl)pentanedicarboxylate.

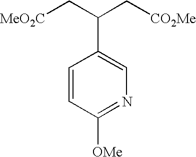

A standard par bottle was charged with dimethyl 3-(6-methoxypyridin-3-yl)pent-2-enedicarboxylate (0.301 g, 1.13 mmol) in MeOH and 4% Palladium on carbon. The hydrogenation was carried out at 5 psi at rt for two hours. MS (ESI+) for C₁₃H₁₇NO₅ m/z 268.40 (M+H)⁺.
Step 3. Synthesis of 3-(6-methoxypyridin-3-yl)pentanedioic acid.

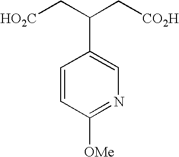

To dimethyl 3-(6-methoxypyridin-3-yl)pentanedicarboxylate (0.276 g, 1.034 mmol) in THF (17.20 mL) was added water (17.20 mL) and KOH (0.58 g). The reaction mixture was stirred at rt for overnight. Concentrated HCl was then added until the pH=2.0. During the addition, the temperature was kept below 50 C. The mixture was extracted with ethyl acetate (3×50 mL). The organic layers were combined, washed with brine, dried over Na₂SO₄ and concentrated to produce off white solid 3-(6-methoxypyridin-3-yl)pentanedioic acid (0.145 g, 59%). ¹H NMR (CD₃OD) δ 8.05 (d, 1H), 7.69-7.65 (m, 1H), 6.78 (d, 1H), 3.89 (s, 3H), 3.60-3.51 (m, 1H), 2.80-2.73 (m, 2H), 2.65-2.58 (m, 2H); MS (ESI+) for C₁₁H₁₃NO₅ m/z 240.30 (M+H)⁺.
Step 4. Synthesis of 4-(6-methoxypyridin-3-yl)dihydropyran-2,6(3H)-dione:

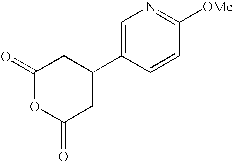

To 3-(6-methoxypyridin-3-yl)pentanedioic acid (0.276 g, 1.15 mmol) was added acetic anhydride (10.0 mL). The reaction mixture was stirred and heated at 100 C for 5 hours. The reaction mixture was cooled to rt. The solvent was removed under reduced pressure to give dark brown solid of 4-(6-methoxypyridin-3-yl)dihydropyran-2,6(3H)-dione (0.086 g, 34%). LCMS was done by diluting the sample with acetonitrile and adding 50 uL of Piperidine, LCMS indicated mass product 307.40 m/z (M+Piperidine).
Step 2. Synthesis of ethyl 4-(3-hydroxyisoxazol-5-yl)-3-(6-methoxypyridin-3-yl)butanoate

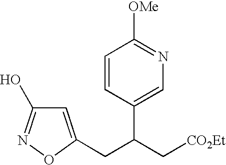

Same synthetic procedure as for Example 125, Step 9, using 7-ethoxy-3-(6-methoxypyridin-3-yl)-5,7-dioxoheptanoic acid as the starting material. H NMR (400 MHz, CDCl₃) δ 1.19 (t, 3H), 2.63-2.76 (m, 2H), 2.97-3.09 (m, 2H), 3.55 (m, 1H), 3.98 (s, 3H), 4.07 (m, 2H), 3.51 (s, 1H), 6.33 (d, 1H), 7.62 (dd, 1H), 8.14 (d, 1H).
Step 3. Synthesis of 7-(2-bromoethyl)-1,2,3,4-tetrahydro-1,8-naphthyridine.

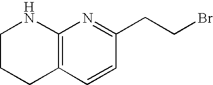

To a solution of (2-(5,6,7,8-tetrahydro-1,8-naphthyridin-2-yl)ethanol) (1 g, 5.62 mmol) in benzene (20 mL) at room temperature under argon was added thionyl bromide (0.65 mL, 8.42 mmol) and the reaction mixture was stirred at 75° C. overnight. After cooling to room temperature the solvent was removed in vacuo. The dark oil was purified by chromatography on silica gel (eluent: 40:60 CH₂Cl₂/ethyl acetate) to yield (7-(2-bromoethyl)-1,2,3,4-tetrahydro-1,8-naphthyridine).
Step 4. Synthesis of ethyl 3-(6-methoxypyridin-3-yl)-4-{3-[2-(5,6,7,8-tetrahydro-1,8-naphthyridin-2-yl)ethoxy]isoxazol-5-yl}butanoate.

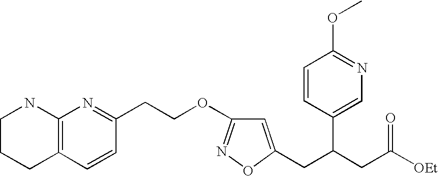

The mixture of 7-(2-bromoethyl)-1,2,3,4-tetrahydro-1,8-naphthyridine (156 mg, 0.65 mmol), DMF (6 mL), ethyl 4-(3-hydroxyisoxazol-5-yl)-3-(6-methoxypyridin-3-yl)butanoate (180 mg, 0.59 mmol) and K₂CO₃ (179 mg, 1.3 mmol) was heated to 60° C. overnight. The mixture was diluted with water, extracted with ethyl acetate. The ethyl acetate layer was washed with water, brine and then dried with Na₂SO₄. The solvent was removed and the residue was purified by reverse phase HPLC using (H₂O/TFA)/CH₃CN as eluent (2.5 mL TFA in 4 L H₂O) to afford 140 mg of the title compound as yellow oil. H NMR (400 MHz, CD₃OD) δ 0.98 (t, 3H), 1.71 (m, 2H), 2.50-2.70 (m, 4H), 2.78-3.00 (m, 4H), 3.34 (m, 3H), 3.76 (s, 3H), 3.86 (q, 2H), 4.27 (t, 2H), 5.54 (s, 1H), 6.51 (d, 1H), 6.73 (d, 1H), 7.43 (d, 1H), 7.60 (dd, 1H), 7.82 (d, 1H).
Step 5. Synthesis of 3-(6-methoxypyridin-3-yl)-4-{3-[2-(5,6,7,8-tetrahydro-1,8-naphthyridin-2-yl)ethoxy]isoxazol-5-yl}butanoic acid.

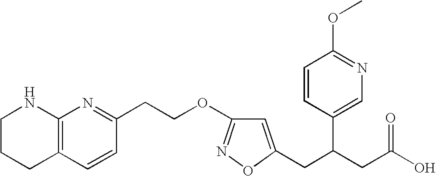

The product ethyl 3-(6-methoxypyridin-3-yl)-4-{3-[2-(5,6,7,8-tetrahydro-1,8-naphthyridin-2-yl)ethoxy]isoxazol-5-yl}butanoate (140 mg, 0.30 mmol) was dissolved in 2 ml methanol and 2 ml 1N sodium hydroxide solution. The reaction was stirred at room temperature overnight, concentrated and acidified with 1 ml trifluoroacetic acid, then purified by reverse phase HPLC using (H₂O/TFA)/CH₃CN as eluent (2.5 mL TFA in 4 L H₂O) to yield 85 mg desired product as yellow oil. FAB-MS:(MH+)=439. H NMR (500 MHz, CD₃OD) δ 1.92 (m, 2H), 2.62-2.79 (m, 2H), 2.82 (t, 2H), 3.00 (m, 1H), 3.08-3.14 (m, 3H), 3.49 (m, 3H), 3.90 (s, 1H), 4.42 (t, 2H), 5.68 (s, 1H), 6.65 (d, 1H), 6.83 (d, 1H), 7.57 (d, 1H), 7.71 (dd, 1H), 7.95 (d, 1H). Anal Calcd. for C₂₃H₂₆N₄O₅ plus 2.8CF₃COOH and 1H₂O: C, 44.28; H, 4.00; N, 7.22. Found: 44.25; H, 4.39; N, 7.20.

### EXAMPLE 128

3-(2,3-Dihydro-benzofuran-6-yl)-4-{5-[3-(5,6,7,8-tetrahydro-[1,8]naphthyridin-2-yl)-propyl]-4H-[1,2,4]triazol-3-yl}-butyric acid

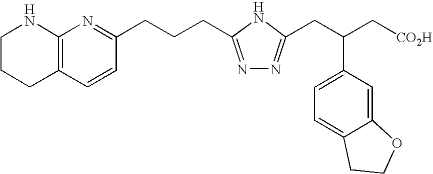

The activity of the compounds of the present invention was tested in the following assays. Compounds of the present invention antagonize the αᵥβ₃ integrin with an IC₅₀ of 0.1 nM to 100 μM in the 293-cell assay. Similarly these compounds also antagonized the αᵥβ₅ integrin with an IC₅₀ of <50 μM in the cell adhesion assay.

### Vitronectin Adhesion Assay

Materials

Human vitronectin receptors αᵥβ₃ and αᵥβ₅ were purified from human placenta as previously described [Pytela et al., Methods in Enzymology, 144:475489 (1987)]. Human vitronectin was purified from fresh frozen plasma as previously described [Yatohgo et al., Cell Structure and Function, 13:281-292 (1988)]. Biotinylated human vitronectin was prepared by coupling NHS-biotin from Pierce Chemical Company (Rockford, Ill.) to purified vitronectin as previously described [Charo et al., J. Biol. Chem., 266(3):1415-1421 (1991)]. Assay buffer, OPD substrate tablets, and RIA grade BSA were obtained from Sigma (St. Louis, Mo.). Anti-biotin antibody was obtained from Sigma (St. Louis, Mo.). Nalge Nunc-Immuno microtiter plates were obtained from Nalge Company (Rochester, N.Y.).

### Methods

### Solid Phase Receptor Assays

This assay was essentially the same as previously reported [Niiya et al., Blood, 70:475483 (1987)]. The purified human vitronectin receptors αᵥβ₃ and αᵥβ₅ were diluted from stock solutions to 1.0 μg/mL in Tris-buffered saline containing 1.0 mM Ca⁺⁺, Mg⁺⁺, and Mn⁺⁺, pH 7.4 (TBS⁺⁺⁺). The diluted receptors were immediately transferred to Nalge Nunc-Immuno microtiter plates at 100 μL/well (100 ng receptor/well). The plates were sealed and incubated overnight at 4° C. to allow the receptors to bind to the wells. All remaining steps were at room temperature. The assay plates were emptied and 200 μL of 1% RIA grade BSA in TBS⁺⁺⁺ (TBS⁺⁺⁺/BSA) were added to block exposed plastic surfaces. Following a 2 hour incubation, the assay plates were washed with TBS⁺⁺⁺ using a 96 well plate washer. Logarithmic serial dilution of the test compound and controls were made starting at a stock concentration of 2 mM and using 2 nM biotinylated vitronectin in TBS⁺⁺⁺/BSA as the diluent. This premixing of labeled ligand with test (or control) ligand, and subsequent transfer of 50 μL aliquots to the assay plate was carried out with a CETUS Propette robot; the final concentration of the labeled ligand was 1 nM and the highest concentration of test compound was 1.0×10⁻⁴ M. The competition occurred for two hours after which all wells were washed with a plate washer as before. Affinity purified horseradish peroxidase labeled goat anti-biotin antibody was diluted 1:2000 in TBS⁺⁺⁺/BSA and 125 μL was added to each well. After 45 minutes, the plates were washed and incubated with OPD/H₂O₂ substrate in 100 mM/L Citrate buffer, pH 5.0. The plate was read with a microtiter plate reader at a wavelength of 450 nm and when the maximum-binding control wells reached an absorbance of about 1.0, the final A₄₅₀ were recorded for analysis. The data were analyzed using a macro written for use with the EXCEL spreadsheet program. The mean, standard deviation, and % CV were determined for duplicate concentrations. The mean A₄₅₀ values were normalized to the mean of four maximum-binding controls (no competitor added)(B-MAX). The normalized values were subjected to a four parameter curve fit algorithm [Rodbard et al., Int. Atomic Energy Agency, Vienna, pp 469 (1977)], plotted on a semi-log scale, and the computed concentration corresponding to inhibition of 50% of the maximum binding of biotinylated vitronectin (IC₅₀) and corresponding R² was reported for those compounds exhibiting greater than 50% inhibition at the highest concentration tested; otherwise the IC₅₀ is reported as being greater than the highest concentration tested. α-[[2-[[5-[(aminoiminomethyl)amino]-1-oxopentyl]amino]-1-oxoethyl]amino]-3-pyridinepropanoic acid [U.S. Pat. No. 5,602,155 Example 1] which is a potent αᵥβ₃ antagonist (IC₅₀ in the range 3-10 nM) was included on each plate as a positive control.

### Purified IIb/IIIa Receptor Assay

Materials

Human fibrinogen receptor (IIb/IIIa) was purified from outdated platelets. (Pytela, R., Pierschbacher, M. D., Argraves, S., Suzuki, S., and Rouslahti, E. “Arginine-Glycine-Aspartic acid adhesion receptors”, Methods in Enzymology 144(1987):475-489.) Human vitronectin was purified from fresh frozen plasma as described in Yatohgo, T., Izumi, M., Kashiwagi, H., and Hayashi, M., “Novel purification of vitronectin from human plasma by heparin affinity chromatography,” Cell Structure and Function 13(1988):281-292. Biotinylated human vitronectin was prepared by coupling NHS-biotin from Pierce Chemical Company (Rockford, Ill.) to purified vitronectin as previously described. (Charo, I. F., Nannizzi, L., Phillips, D. R., Hsu, M. A., Scarborough, R. M., “Inhibition of fibrinogen binding to GP IIb/IIIa by a GP IIIa peptide”, J. Biol. Chem. 266(3)(1991): 1415-1421.) Assay buffer, OPD substrate tablets, and RIA grade BSA were obtained from Sigma (St. Louis, Mo.). Anti-biotin antibody was obtained from Sigma (St. Louis, Mo.). Nalge Nunc-Immuno microtiter plates were obtained from (Rochester, N.Y.). ADP reagent was obtained from Sigma (St. Louis, Mo.).

### Methods

### Solid Phase Receptor Assays

This assay is essentially the same reported in Niiya, K., Hodson, E., Bader, R., Byers-Ward, V. Koziol, J. A., Plow, E. F. and Ruggeri, Z. M., “Increased surface expression of the membrane glycoprotein IIb/IIIa complex induced by platelet activation: Relationships to the binding of fibrinogen and platelet aggregation”, Blood 70(1987):475-483. The purified human fibrinogen receptor (IIb/IIIa) was diluted from stock solutions to 1.0 μg/mL in Tris-buffered saline containing 1.0 mM Ca⁺⁺, Mg⁺⁺, and Mn⁺⁺, pH 7.4 (TBS⁺⁺⁺). The diluted receptor was immediately transferred to Nalge Nunc-Immuno microtiter plates at 100 μL/well (100 ng receptor/well). The plates were sealed and incubated overnight at 4° C. to allow the receptors to bind to the wells. All remaining steps were at room temperature. The assay plates were emptied and 200 μL of 1% RIA grade BSA in TBS⁺⁺⁺ (TBS⁺⁺⁺/BSA) were added to block exposed plastic surfaces. Following a 2 hour incubation, the assay plates were washed with TBS⁺⁺⁺ using a 96 well plate washer. Logarithmic serial dilution of the test compound and controls were made starting at a stock concentration of 2 mM and using 2 nM biotinylated vitronectin in TBS⁺⁺⁺/BSA as the diluent. This premixing of labeled ligand with test (or control) ligand, and subsequent transfer of 50 μL aliquots to the assay plate was carried out with a CETUS Propette robot; the final concentration of the labeled ligand was 1 nM and the highest concentration of test compound was 1.0×10⁻⁴ M. The competition occurred for two hours after which all wells were washed with a plate washer as before. Affinity purified horseradish peroxidase labeled goat anti-biotin antibody was diluted 1:2000 in TBS⁺⁺⁺/BSA and 125 μL were added to each well. After 45 minutes, the plates were washed and incubated with ODD/H₂O₂ substrate in 100 mM/L citrate buffer, pH 5.0. The plate was read with a microtiter plate reader at a wavelength of 450 nm and when the maximum-binding control wells reached an absorbance of about 1.0, the final A₄₅₀ were recorded for analysis. The data were analyzed using a macro written for use with the EXCELJ spreadsheet program. The mean, standard deviation, and % CV were determined for duplicate concentrations. The mean A₄₅₀ values were normalized to the mean of four maximum-binding controls (no competitor added)(B-MAX). The normalized values were subjected to a four parameter curve fit algorithm, [Robard et al., Int. Atomic Energy Agency, Vienna, pp 469 (1977)], plotted on a semi-log scale, and the computed concentration corresponding to inhibition of 50% of the maximum binding of biotinylated vitronectin (IC₅₀) and corresponding R² was reported for those compounds exhibiting greater than 50% inhibition at the highest concentration tested; otherwise the IC₅₀ is reported as being greater than the highest concentration tested. β-[[2-[[5-[(aminoiminomethyl)amino]-1-oxopentyl]amino]-1-oxoethyl]amino]-3-pyridinepropanoic acid [U.S. Pat. No. 5,602,155 Example 1] which is a potent αᵥβ₃ antagonist (IC₅₀ in the range 3-10 nM) was included on each plate as a positive control.

### Human Platelet Rich Plasma Assays

Healthy aspirin free donors were selected from a pool of volunteers. The harvesting of platelet rich plasma and subsequent ADP induced platelet aggregation assays were performed as described in Zucker, M. B., “Platelet Aggregation Measured by the Photometric Method”, Methods in Enzymology 169(1989):117-133. Standard venipuncture techniques using a butterfly allowed the withdrawal of 45 mL of whole blood into a 60 mL syringe containing 5 mL of 3.8% trisodium citrate. Following thorough mixing in the syringe, the anti-coagulated whole blood was transferred to a 50 mL conical polyethylene tube. The blood was centrifuged at room temperature for 12 minutes at 200×g to sediment non-platelet cells. Platelet rich plasma was removed to a polyethylene tube and stored at room temperature until used. Platelet poor plasma was obtained from a second centrifugation of the remaining blood at 2000×g for 15 minutes. Platelet counts are typically 300,000 to 500,000 per microliter. Platelet rich plasma (0.45 mL) was aliquoted into siliconized cuvettes and stirred (1100 rpm) at 37° C. for 1 minute prior to adding 50 uL of pre-diluted test compound. After 1 minute of mixing, aggregation was initiated by the addition of 50 uL of 200 uM ADP. Aggregation was recorded for 3 minutes in a Payton dual channel aggregometer (Payton Scientific, Buffalo, N.Y.). The percent inhibition of maximal response (saline control) for a series of test compound dilutions was used to determine a dose response curve. All compounds were tested in duplicate and the concentration of half-maximal inhibition (IC₅₀) was calculated graphically from the dose response curve for those compounds which exhibited 50% or greater inhibition at the highest concentration tested; otherwise, the IC₅₀ is reported as being greater than the highest concentration tested.

### Cell Assays for Potency and Selectivity

While the β₃ subunit of αᵥβ₃ is only known to complex with αᵥ or α_{IIb}, the αᵥ subunit complexes with multiple β subunits. The three αᵥ integrins most homologous with αᵥβ₃ are αᵥβ₁, αᵥβ₅ and αᵥβ₆, with 43%, 56% and 47% amino acid identity in the β subunits, respectively. To evaluate the selectivity of compounds between the integrins αᵥβ₃ and αᵥβ₆ cell-based assays were established using the 293 human embryonic kidney cell line. 293 cells express αᵥβ₁, but little to no detectable αᵥβ₃ or αᵥβ₆. cDNAs for 3 and β₆ were transfected separately into 293 cells to generate 293-β3 and 293-β6 cells, respectively. High surface expression of αᵥβ₃ and αᵥβ₆ was confirmed by flow cytometry. Conditions were established for each cell line in which cell adhesion to immobilized human vitronectin was mediated by the appropriate integrin, as determined by a panel of integrin-specific, neutralizing monoclonal antibodies. Briefly, cells were incubated with inhibitor in the presence of 200 uM Mn²⁺, allowed to adhere to immobilized vitronectin, washed, and adherent cells are detected endogenous alkaline phosphatase and para-nitrophenyl phosphate. An 8-point dose-response curve using either 10-fold or 3-fold dilutions of compound was evaluated by fitting a four-parameter logistic, nonlinear model (using SAS). To evaluate compound potency for membrane-bound αᵥβ₆ an additional cell-based adhesion assay was established using the HT-29 human colon carcinoma cell line. High surface expression of αᵥβ₆ on HT-29 cells was confirmed by flow cytometry. Conditions were established in which cell adhesion to immobilized human latency associated peptide (LAP) was mediated by the αᵥβ₆, as determined by a panel of integrin-specific, neutralizing monoclonal antibodies. Briefly, cells were incubated with inhibitor in the presence of 200 uM Mn²⁺, allowed to adhere to immobilized LAP, washed, and adherent cells are detected by quantifying endogenous alkaline phosphatase using para-nitrophenyl phosphate. An 8-point dose-response curve using either 10-fold or 3-fold dilutions of compound was evaluated by fitting a four-parameter logistic, nonlinear model (using SAS). The compounds evaluated were relatively ineffective at inhibition of αᵥβ₆-mediated cell adhesion. The selective antagonism of the αᵥβ₃ integrin is viewed as desirable in this class of compounds, as αᵥβ₆ may also play a role in normal physiological processes of tissue repair and cellular turnover that routinely occur in the skin and pulmonary tissues.

1. A pharmaceutical composition comprising a pharmaceutically acceptable carrier and a compound selected from the group consisting of:
3-(3,5-ditert-butylphenyl)-4-{3-[3-(5,6,7,8-tetrahydro-1,8-naphthyridin-2-yl)propyl]-1,2,4-oxadiazol-5-yl}butanoic acid (TFA salt);
3-(3-tert-butyl-5-iodophenyl)-4-{3-[3-(5,6,7,8-tetrahydro-1,8-naphthyridin-2-yl)propyl]-1,2,4-oxadiazol-5-yl}butanoic acid;
3-(3-tert-butyl-5-bromophenyl)-4-{3-[3-(5,6,7,8-tetrahydro-1,8-naphthyridin-2-yl)propyl]-1,2,4-oxadiazol-5-yl}butanoic acid;
3-(5-tert-Butyl-2-hydroxyphenyl)-4-{3-[3-(5,6,7,8-tetrahydro-1,8-naphthyridin-2-yl)propyl]-1,2,4-oxadiazol-5-yl}butanoic acid;
3-[3,5-Ditert-butyl-2-(carboxymethoxy)phenyl]-4-{3-[3-(5,6,7,8-tetrahydro-1,8-naphthyridin-2-yl)propyl]-1,2,4-oxadiazol-5-yl}butanoic acid;
3-(5-tert-Butyl-2-methoxyphenyl)-4-{3-[3-(5,6,7,8-tetrahydro-1,8-naphthyridin-2-yl)propyl]-1,2,4-oxadiazol-5-yl}butanoic acid;
3-(3,5-Ditert-butyl-4-methoxyphenyl)-4-{3-[3-(5,6,7,8-tetrahydro-1,8-naphthyridin-2-yl)propyl]-1,2,4-oxadiazol-5-yl}butanoic acid;
3-(3-tert-Butyl-5-[2,2,2-trifluoro-1-hydroxy-1-(trifluoromethyl)ethyl]-phenyl}-4-{3-[3-(5,6,7,8-tetrahydro-1,8-naphthyridin-2-yl)propyl]-1,2,4-oxadiazol-5-yl}butanoic acid;
3-(3,4-Dichlorophenyl)-4-{3-[3-(5,6,7,8-tetrahydro-1,8-naphthyridin-2-yl)propyl]-1,2,4-oxadiazol-5-yl}butanoic acid trifluoroacetate;
3-(3-Fluoro-4-methylphenyl)-4-{3-[3-(5,6,7,8-tetrahydro-1,8-naphthyridin-2-yl)propyl]-1,2,4-oxadiazol-5-yl}butanoic acid hydrochloride;
3-(4-Phenoxyphenyl)-4-{3-[3-(5,6,7,8-tetrahydro-1,8-naphthyridin-2-yl)propyl]-1,2,4-oxadiazol-5-yl}butanoic acid trifluoroacetate;
3-(1-Benzofuran-2-yl)-4-{3-[3-(5,6,7,8-tetrahydro-1,8-naphthyridin-2-yl)propyl]-1,2,4-oxadiazol-5-yl}butanoic acid trifluoroacetate;
3-[4-(Benzyloxy)phenyl]-4-{3-[3-(5,6,7,8-tetrahydro-1,8-naphthyridin-2-yl)propyl]-1,2,4-oxadiazol-5-yl}butanoic acid trifluoroacetate;
3-[4-(Methylsulfonyl)phenyl]-4-{3-[3-(5,6,7,8-tetrahydro-1,8-naphthyridin-2-yl)propyl]-1,2,4-oxadiazol-5-yl}butanoic acid trifluoroacetate;
4-{3-[3-(5,6,7,8-Tetrahydro-1,8-naphthyridin-2-yl)propyl]-1,2,4-oxadiazol-5-yl}-3-[4-(trifluoromethoxy)phenyl]butanoic acid trifluoroacetate;
3-(3-Furyl)-4-{3-[3-(5,6,7,8-tetrahydro-1,8-naphthyridin-2-yl)propyl]-1,2,4-oxadiazol-5-yl}butanoic acid trifluoroacetate;
4-{3-[3-(5,6,7,8-Tetrahydro-1,8-naphthyridin-2-yl)propyl]-1,2,4-oxadiazol-5-yl}-3-thien-3-ylbutanoic acid trifluoroacetate;
3-(2,3-Dihydro-1,4-benzodioxin-6-yl)-4-{3-[3-(5,6,7,8-tetrahydro-1,8-naphthyridin-2-yl)propyl]-1,2,4-oxadiazol-5-yl}butanoic acid hydrochloride;
4-{3-[3-(5,6,7,8-Tetrahydro-1,8-naphthyridin-2-yl)propyl]-1,2,4-oxadiazol-5-yl}-3-[3-(trifluoromethoxy)phenyl]butanoic acid hydrochloride;
4-{3-[3-(5,6,7,8-Tetrahydro-1,8-naphthyridin-2-yl)propyl]-1,2,4-oxadiazol-5-yl}-3-(3,4,5-trifluorophenyl)butanoic acid hydrochloride;
3-(2,2-Difluoro-1,3-benzodioxol-5-yl)-4-{3-[3-(5,6,7,8-tetrahydro-1,8-naphthyridin-2-yl)propyl]-1,2,4-oxadiazol-5-yl}butanoic acid hydrochloride;
3-[3-Fluoro-5-(trifluoromethyl)phenyl]-4-{3-[3-(5,6,7,8-tetrahydro-1,8-naphthyridin-2-yl)propyl]-1,2,4-oxadiazol-5-yl}butanoic acid hydrochloride;
3-(6-Methoxy-2-naphthyl)-4-{3-[3-(5,6,7,8-tetrahydro-1,8-naphthyridin-2-yl)propyl]-1,2,4-oxadiazol-5-yl}butanoic acid hydrochloride;
3-(6-Methoxypyridin-3-yl)-4-{3-[3-(5,6,7,8-tetrahydro-1,8-naphthyridin-2-yl)propyl]-1,2,4-oxadiazol-5-yl}butanoic acid;
3-(4-Cyanophenyl)-4-{3-[3-(5,6,7,8-tetrahydro-1,8-naphthyridin-2-yl)propyl]-1,2,4-oxadiazol-5-yl}butanoic acid;
3-(3-Cyanophenyl)-4-{3-[3-(5,6,7,8-tetrahydro-1,8-naphthyridin-2-yl)propyl]-1,2,4-oxadiazol-5-yl}butanoic acid;
3-benzyl-4-{3-[3-(5,6,7,8-tetrahydro-1,8-naphthyridin-2-yl)propyl]-1,2,4-oxadiazol-5-yl}butanoic acid trifluoroacetate;
3-(4-fluoro-3-methoxyphenyl)-4-{3-[3-(5,6,7,8-tetrahydro-1,8-naphthyridin-2-yl)propyl]-1,2,4-oxadiazol-5-yl}butanoic acid trifluoroacetate;
3-(3-Fluoro-5-methoxyphenyl)-4-{3-[3-(5,6,7,8-tetrahydro-1,8-naphthyridin-2-yl)propyl]-1,2,4-oxadiazol-5-yl}butanoic acid hydrochloride;
3-(2-Methyl-1,3-benzothiazol-5-yl)-4-{3-[3-(5,6,7,8-tetrahydro-1,8-naphthyridin-2-yl)propyl]-1,2,4-oxadiazol-5-yl}butanoic acid hydrochloride;
3-[2-(4-Chlorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride;
3-[2-(4-Methoxyphenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride;
3-(2-Methyl-1,3-benzothiazol-5-yl)-4-{3-[3-(5,6,7,8-tetrahydro-1,8-naphthyridin-2-yl)propyl]-1,2,4-oxadiazol-5-yl}butanoic acid hydrochloride;
3-[2-(4-Fluorophenyl)-1,3-thiazol-5-yl]4-{3-[3-(5,6,7,8-tetrahydro-1,8-naphthyridin-2-yl)propyl]-1,2,4-oxadiazol-5-yl}butanoic acid hydrochloride;
3-[2-(3,5-Difluorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride;
3-[2-(3,4-Difluorophenyl)-1,3-thiazol-5-yl]-4-{3-[3-(5,6,7,8-tetrahydro-1,8-naphthyridin-2-yl)propyl]-1,2,4-oxadiazol-5-yl}butanoic acid hydrochloride;
3-[2-(2-Fu ryl)-1,3-th iazol-5-yl]-4-{3-[3-(5,6,7,8-tetrahydro-1,8-naphthyridin-2-yl)propyl]-1,2,4-oxadiazol-5-yl}butanoic acid hydrochloride;
3-(3,4-Dimethoxyphenyl)-4-{3-[3-(5,6,7,8-tetrahydro-1,8-naphthyridin-2-yl)propyl] 1,2,4-oxadiazol-5-yl}butanoic acid trifluoroacetate;
3-(3,5-Dimethoxyphenyl)-4-{3-[3-(5,6,7,8-tetrahydro-1,8-naphthyridin-2-yl)propyl]1,2,4-oxadiazol-5-yl}butanoic acid trifluoroacetate;
3-(3,5-Dichlorophenyl)-4-{3-[3-(5,6,7,8-tetrahydro-1,8-naphthyridin-2-yl)propyl]-1,2,4-oxadiazol-5-yl}butanoic acid trifluoroacetate;
3-(3,5-Difluorophenyl)-4-{3-[3-(5,6,7,8-tetrahydro-1,8-naphthyridin-2-yl)propyl]-1,2,4-oxadiazol-5-yl}butanoic acid trifluoroacetate;
3-(3-Fluoro-4-methoxyphenyl)-4-{3-[3-(5,6,7,8-tetrahydro-1,8-naphthyridin-2-yl)propyl]-1,2,4-oxadiazol-5-yl}butanoic acid trifluoroacetate;
4-{3-[3-(5,6,7,8-Tetrahydro-1,8-naphthyridin-2-yl)propyl]-1,2,4-oxadiazol-5-yl}-3-[4-(trifluoromethyl)phenyl]butanoic acid trifluoroacetate;
3-(2-Methyl-1,3-thiazol-5-yl)-4-{3-[3-(5,6,7,8-tetrahydro-1,8-naphthyridin-2-yl)propyl]-1,2,4-oxadiazol-5-yl}butanoic acid trifluoroacetate;
3-(1-Phenyl-1H-pyrazol-4-yl)-4-{3-[3-(5,6,7,8-tetrahydro-1,8-naphthyridin-2-yl)propyl]-1,2,4-oxadiazol-5-yl}butanoic acid trifluoroacetate;
3-(1-Benzofuran-6-yl)-4-{3-[3-(5,6,7,8-tetrahydro-1,8-naphthyridin-2-yl)propyl]-1,2,4-oxadiazol-5-yl}butanoic acid hydrochloride;
3-(2,3-dihydro-1-benzofuran-6-yl)-4-{3-[3-(5,6,7,8-tetrahydro-1,8-naphthyridin-2-yl)propyl]-1,2,4-oxadiazol-5-yl}butanoic acid hydrochloride;
3-(1,3-Benzodioxol-5-yl)-4-(3-{3-[(pyridin-2-ylamino)methyl]phenyl}-1,2,4-oxadiazol-5-yl)butanoic acid hydrochloride;
3-(7-Fluoro-1,3-benzodioxol-5-yl)-4-{3-[3-(5,6,7,8-tetrahydro-1,8-naphthyridin-2-yl)propyl]-1,2,4-oxadiazol-5-yl}butanoic acid trifluoroacetate;
3-(1,3-Benzoxazol-6-yl)-4-{3-[3-(5,6,7,8-tetrahydro-1,8-naphthyridin-2-yl)propyl]-1,2,4-oxadiazol-5-yl}butanoic acid hydrochloride;
3-(3-Methyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate;
3-(3-Ethyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate;
3-(3-Phenyl-1,2,4-oxadiazol-5-yl)-4-{3-[3-(5,6,7,8-tetrahydro-1,8-naphthyridin-2-yl)propyl]-1,2,4-oxadiazol-5-yl}butanoic acid trifluoroacetate;
[1-Benzoyl-4-({3-[3-(5,6,7,8-tetrahydro-1,8-naphthyridin-2-yl)propyl]-1,2,4-oxadiazol-5-yl}methyl)piperidin-4-yl]acetic acid trifluoroacetate;
[1-Benzoyl-4-({3-[4-(pyridin-2-ylamino)butyl]-1,2,4-oxadiazol-5-yl}methyl)piperidin-4-yl]acetic acid trifluoroacetate;
[1-(tert-Butoxycarbonyl)-4-({3-[3-(5,6,7,8-tetrahydro-1,8-naphthyridin-2-yl)propyl]-1,2,4-oxadiazol-5-yl}methyl)piperidin-4-yl]acetic acid trifluoroacetate;
[1-(tert-Butoxycarbonyl)-4-({3-[4-(pyridin-2-ylamino)butyl]-1,2,4-oxadiazol-5-yl}methyl)piperidin-4-yl]acetic acid trifluoroacetate;
3-(4-Methyl phenyl)-4-{3-[3-(5,6,7,8-tetrahydro-1,8-naphthyridin-2-yl)propyl]-1,2,4-oxadiazol-5-yl}butanoic acid trifluoroacetate;
3-(3-Chlorophenyl)-4-{3-[3-(5,6,7,8-tetrahydro-1,8-naphthyridin-2-yl)propyl]-1,2,4-oxadiazol-5-yl}butanoic acid hydrochloride;
3-(4-Methoxy-3-methyl phenyl)-4-{3-[3-(5,6,7,8-tetrahydro-1,8-naphthyridin-2-yl)propyl]-1,2,4-oxadiazol-5-yl}butanoic acid hydrochloride;
3-[4-(Methylthio)phenyl]-4-{3-[3-(5,6,7,8-tetrahydro-1,8-naphthyridin-2-yl)propyl]-1,2,4-oxadiazol-5-yl}butanoic acid trifluoroacetate;
3-(1-Methyl-1H-indol-3-yl)-4-{3-[3-(5,6,7,8-tetrahydro-1,8-naphthyridin-2-yl)propyl]-1,2,4-oxadiazol-5-yl}butanoic acid trifluoroacetate;
3-(1,1′-Biphenyl-4-yl)-4-{3-[3-(5,6,7,8-tetrahydro-1,8-naphthyridin-2-yl)propyl]-1,2,4-oxadiazol-5-yl}butanoic acid hydrochloride;
3-(3-Bromophenyl)-4-{3-[3-(5,6,7,8-tetrahydro-1,8-naphthyridin-2-yl)propyl]-1,2,4-oxadiazol-5-yl}butanoic acid trifluoroacetate;
3-(4-Bromophenyl)-4-{3-[3-(5,6,7,8-tetrahydro-1,8-naphthyridin-2-yl)propyl]-1,2,4-oxadiazol-5-yl}butanoic acid hydrochloride;
3-(3-Phenoxyphenyl)-4-{3-[3-(5,6,7,8-tetrahydro-1,8-naphthyridin-2-yl)propyl]-1,2,4-oxadiazol-5-yl}butanoic acid trifluoroacetate;
3-[3-(Benzyloxy)phenyl]4-{3-[3-(5,6,7,8-tetrahydro-1,8-naphthyridin-2-yl)propyl]-1,2,4-oxadiazol-5-yl}butanoic acid hydrochloride;
3-(3-Bromo-4-methoxyphenyl)-4-{3-[3-(5,6,7,8-tetrahydro-1,8-naphthyridin-2-yl)propyl]-1,2,4-oxadiazol-5-yl}butanoic acid trifluoroacetate;
4-{3-[3-(5,6,7,8-Tetrahydro-1,8-naphthyridin-2-yl)propyl]-1,2,4-oxadiazol-5-yl}-3-(3,4,5-trimethoxyphenyl)butanoic acid trifluoroacetate;
3-(2-Naphthyl)-4-{3-[3-(5,6,7,8-tetrahydro-1,8-naphthyridin-2-yl)propyl]-1,2,4-oxadiazol-5-yl}butanoic acid hydrochloride;
3-(3-Nitrophenyl)-4-{3-[3-(5,6,7,8-tetrahydro-1,8-naphthyridin-2-yl)propyl]-1,2,4-oxadiazol-5-yl}butanoic acid hydrochloride;
3-(3-Methylphenyl)-4-{3-[3-(5,6,7,8-tetrahydro-1,8-naphthyridin-2-yl)propyl]-1,2,4-oxadiazol-5-yl}butanoic acid hydrochloride;
3-(2-Fu ryl)-4-{3-[3-(5,6,7,8-tetrahydro-1,8-naphthyridin-2-yl)propyl]-1,2,4-oxadiazol-5-yl}butanoic acid hydrochloride;
3-(2-Methyl phenyl)-4-{3-[3-(5,6,7,8-tetrahydro-1,8-naphthyridin-2-yl)propyl]-1,2,4-oxadiazol-5-yl}butanoic acid hydrochloride;
3-(1,3-benzodioxol-5-yl)-4-{3-[3-(3,4-dihydro-2H-pyrido[3,2-b][1,4]oxazin-6-yl)propyl]-1,2,4-oxadiazol-5-yl}butanoic acid, TFA;
4-{3-[3-(3,4-dihydro-2H-pyrido[3,2-b][1,4]oxazin-6-yl)propyl]-1,2,4-oxadiazol-5-yl}-3-(3,5-dimethoxyphenyl)butanoic acid, TFA;
3-Benzo[1,3]dioxol-5-yl-4-{3-[3-(6,7,8,9-tetrahydro-5-oxa-1,9-diaza-benzocyclohepten-2-yl)-propyl]-[1,2,4]oxadiazol-5-yl}-butyric acid;
3-(3-Fluoro-4-methoxyphenyl)-4-{3-[3-(6,7,8,9-tetrahydro-5-oxa-1,9-diaza-benzocyclohepten-2-yl)-propyl]-[1,2,4]oxadiazol-5-yl}-butyric acid;
3-(3,5-Difluorophenyl)-4-{3-[3-(6,7,8,9-tetrahydro-5-oxa-1,9-diaza-benzocyclohepten-2-yl)-propyl]-[1,2,4]oxadiazol-5-yl}-butyric acid;
3-(3,5-Dimethoxyphenyl)-4-{3-[3-(6,7,8,9-tetrahydro-5-oxa-1,9-diaza-benzocyclohepten-2-yl)-propyl]-[1,2,4]oxadiazol-5-yl}-butyric acid;
3-(2-Methylbenzothiazol-5-yl)-4-{3-[3-(6,7,8,9-tetrahydro-5-oxa-1,9-diaza-benzocyclohepten-2-yl)-propyl]-[1,2,4]oxadiazol-5-yl}-butyric acid;
3-(1,3-benzodioxol-5-yl)-4-{3-[3-(1,2,3,5-tetrahydropyrido[2,3-e][1,4]oxazepin-8-yl)propyl]-1,2,4-oxadiazol-5-yl}butanoic acid, TFA;
3-(3,5-dimethoxyphenyl)-4-{3-[3-(1,2,3,5-tetrahydropyrido[2,3-e][1,4]oxazepin-8-yl)propyl]-1,2,4-oxadiazol-5-yl}butanoic acid, TFA;
3-(1,3-Benzodioxol-5-yl)-4-(3-{3-[6-(methylamino)pyridin-2-yl]propyl}-1,2,4-oxadiazol-5-yl)butanoic acid hydrochloride;
3-(3-Fluorophenyl)-4-(3-{3-[6-(methylamino)pyridin-2-yl]propyl}-1,2,4-oxadiazol-5-yl)butanoic acid trifluoroacetate;
3-(1,3-benzodioxol-5-yl)-4-(3-{3-[6-(ethylamino)pyridin-2-yl]propyl}-1,2,4-oxadiazol-5-yl)butanoic acid trifluoroacetate;
3-(3-Fuorophenyl)-4-(3-{3-[6-(methylamino) pyridin-2-yl]propyl-1,2,4-oxadiazol-5-yl)butanoic acid trifluoroacetate;
3-(1,3-Benzodioxol-5-yl)-4-(3-{4-[(4-methylpyridin-2-yl)amino]butyl}-1,2,4-oxadiazol-5-yl)butanoic acid;
3-(1,3-benzodioxol-5-yl)-4-(3-{4-[(6-methylpyridin-2-yl)amino]butyl}-1,2,4-oxadiazol-5-yl)butanoic acid;
(2-{6-[2-(5,6,7,8-Tetrahydro-1,8-naphthyridin-2-yl)ethoxy]pyridin-3-yl}cyclopropyl)acetic acid;
3-Methyl-4-{6-[2-(5,6,7,8-tetrahydro-1,8-naphthyridin-2-yl)ethoxy]pyridin-3-yl}butanoic acid;
3-(1,3-benzodioxol-5-yl)-4-{5-[3-(5,6,7,8-tetra hydro-1,8-naphthyridin-2-yl)propyl]-1,3,4-oxadiazol-2-yl}butanoic acid trifluoroacetate;
3-(3-fluorophenyl)-4-{5-[3-(5,6,7,8-tetrahydro-1,8-naphthyridin-2-yl)propyl]-1,3,4-oxadiazol-2-yl}butanoic acid trifluoroacetate;
3-(3-Fluoro-4-methoxyphenyl)-4-{5-[3-(5,6,7,8-tetrahydro-1,8-naphthyridin-2-yl)propyl]-1,3,4-oxadiazol-2-yl}butanoic acid trifluoroacetate;
3-(3,5-Dimethoxyphenyl)-4-{5-[3-(5,6,7,8-tetrahydro-1,8-naphthyridin-2-yl)propyl]-1,3,4-oxadiazol-2-yl}butanoic acid trifluoroacetate;
3-(2-Methyl-1,3-thiazol-5-yl)-4-{5-[3-(5,6,7,8-tetrahydro-1,8-naphthyridin-2-yl)propyl]-1,3,4-oxadiazol-2-yl}butanoic acid trifluoroacetate;
3-(4-Fluorophenyl)-4-{5-[3-(5,6,7,8-tetrahydro-1,8-naphthyridin-2-yl)propyl]-1,3,4-oxadiazol-2-yl}butanoic acid trifluoroacetate;
3-(3,5-Difluorophenyl)-4-{5-[3-(5,6,7,8-tetrahydro-1,8-naphthyridin-2-yl)propyl]-1,3,4-oxadiazol-2-yl}butanoic acid trifluoroacetate;
3-(3,5-Difluorophenyl)-4-{5-[3-(5,6,7,8-tetrahydro-1,8-naphthyridin-2-yl)propyl]-1,3,4-thiadiazol-2-yl}butanoic acid trifluoroacetate;
3-(4-Fluorophenyl)-4-{5-[3-(5,6,7,8-tetrahydro-1,8-naphthyridin-2-yl)propyl]-1,3,4-thiadiazol-2-yl}butanoic acid trifluoroacetate;
3-(2-Methyl-1,3-thiazol-5-yl)-4-{5-[3-(5,6,7,8-tetrahydro-1,8-naphthyridin-2-yl)propyl]-1,3,4-thiadiazol-2-yl}butanoic acid trifluoroacetate;
3-(1,3-Benzodioxol-5-yl)-4-{5-[3-(5,6,7,8-tetrahydro-1,8-naphthyridin-2-yl)propyl]-1,3,4-thiadiazol-2-yl}butanoic acid trifluoroacetate;
3-(1,3-benzodioxol-5-yl)-4-{3-[2-(5,6,7,8-tetrahydro-1,8-naphthyridin-2-yl)ethoxy]isoxazol-5-yl}butanoic acid;
3-(1,3-benzodioxol-5-yl)-4-{5-[3-(5,6,7,8-tetrahydro-1,8-naphthyridin-2-yl)propyl]-2H-tetraazol-2-yl}butanoic acid;
3-(1,3-benzodioxol-5-yl)-4-{5-[3-(5,6,7,8-tetrahydro-1,8-naphthyridin-2-yl)propyl]-1H-tetraazol-1-yl}butanoic acid;
3-(1,3-benzodioxol-5-yl)-4-{3-[2-(5,6,7,8-tetrahydro-1,8-naphthyridin-2-yl)ethoxy]-1H-pyrazol-5-yl}butanoic acid;
3-(1,3-benzodioxol-5-yl)-4-{3-[3-(4,5-dihydro-1H-imidazol-2-ylamino)propoxy]isoxazol-5-yl}butanoic acid;
3-[2-(4-chlorophenyl)-1,3-thiazol-5-yl]-4-{3-[2-(5,6,7,8-tetrahydro-1,8-naphthyridin-2-yl)ethoxy]isoxazol-5-yl}butanoic acid;
3-Benzo[1,3]dioxol-5-yl-4-{3-[2-(6,7,8,9-tetrahydro-5-oxa-1,9-diaza-benzocyclohepten-2-yl)-ethoxy]-isoxazol-5-yl}-butyric acid;
3-Benzo[1,3]dioxol-5-yl-4-{3-oxo-2-[2-(6,7,8,9-tetrahydro-5-oxa-1,9-diaza-benzocyclohepten-2-yl)-ethyl]-2,3-dihydro-isoxazol-5-yl}-butyric acid;
3-(1,3-benzodioxol-5-yl)-4-{3-[2-(3,4-dihydro-2H-pyrido[3,2-b][1,4]oxazin-6-yl)ethoxy]isoxazol-5-yl}butanoic acid, TFA;
3-(1,3-benzodioxol-5-yl)-4-{2-[2-(3,4-dihydro-2H-pyrido[3,2-b][1,4]oxazin-6-yl)ethyl]-3-oxo-2,3-dihydroisoxazol-5-yl}butanoic acid, TFA;
3-(1,3-benzodioxol-5-yl)-4-{3-[2-(1,2,3,5-tetrahydropyrido[2,3-e][1,4]oxazepin-8-yl)ethoxy]isoxazol-5-yl}butanoic acid, TFA;
3-(1,3-benzodioxol-5-yl)-4-{3-oxo-2-[2-(1,2,3,5-tetrahydropyrido[2,3-e][1,4]oxazepin-8-yl)ethyl]-2,3-dihydroisoxazol-5-yl}butanoic acid, TFA;
3-(1,3-benzodioxol-5-yl)-4-(3-{2-[5-(methoxymethyl)-6-(methylamino)pyridin-2-yl]ethoxy}isoxazol-5-yl)butanoic acid, TFA;
3-(1,3-Benzodioxol-5-yl)-4-{3-[3-(5,5-dimethyl-5,6,7,8-tetrahydro-1,8-naphthyridin-2-yl)propyl]-1,2,4-oxadiazol-5-yl}butanoic acid;
3-(1,3-benzodioxol-5-yl)-4-{3-[3-(1-methyl-1,2,3,4-tetrahydropyrido[2,3-b]pyrazin-6-yl)propyl]-1,2,4-oxadiazol-5-yl}butanoic acid;
3-(2-methyl-1,3-benzothiazol-5-yl)-4-{3-[3-(1-methyl-1,2,3,4-tetrahydropyrido[2,3-b]pyrazin-6-yl)propyl]-1,2,4-oxadiazol-5-yl}butanoic acid;
3-(3-fluoro-4-methoxyphenyl)-4-{3-[3-(1-methyl-1,2,3,4-tetrahydropyrido[2,3-240 b]pyrazin-6-yl)propyl]-1,2,4-oxadiazol-5-yl}butanoic acid;
3-(6-methoxypyridin-3-yl)-4-{3-[3-(1-methyl-1,2,3,4-tetrahydropyrido[2,3-b]pyrazin-6-yl)propyl]-1,2,4-oxadiazol-5-yl}butanoic acid;
3-(1,3-benzodioxol-5-yl)-4-(3-{[2-(5,6,7,8-tetrahydro-1,8-naphthyridin-2-yl)ethyl]thio}-1H-1,2,4-triazol-5-yl)butanoic acid;
3-(1,3-benzodioxol-5-yl)-4-(1-methyl-5-{[2-(5,6,7,8-tetrahydro-1,8-naphthyridin-2-yl)ethyl]thio}-1H-1,2,4-triazol-3-yl)butanoic acid;
3-(1,3-benzodioxol-5-yl)-4-(4-methyl-5-{[2-(5,6,7,8-tetrahydro-1,8-naphthyridin-2-yl)ethyl]thio}-4H-1,2,4-triazol-3-yl)butanoic acid;
3-(1,3-benzodioxol-5-yl)-4-{3-[2-(1-methyl-1,2,3,4-tetrahydropyrido[2,3-b]pyrazin-6-250 yl)ethoxy]isoxazol-5-yl}butanoic acid;
3-(1,3-benzodioxol-5-yl)-4-(3-{2-[6-(methylamino)pyridin-2-yl]ethoxy}isoxazol-5-yl)butanoic acid; and
3-(6-methoxypyridin-3-yl)-4-{3-[2-(5,6,7,8-tetrahydro-1,8-naphthyridin-2-yl)ethoxy]isoxazol-5-yl}butanoic acid.

2. A method for the treatment or prevention of conditions mediated by the α_{V}β₃ integrin in a mammal in need of such treatment, the method comprising administering to the subject a therapeutically effective amount of a composition of claim 1.

3. The method according to claim 2 wherein the condition treated is selected from the group consisting of tumor metastasis, solid tumor growth, angiogenesis, osteoporosis, humoral hypercalcemia of malignancy, smooth muscle cell migration, restenosis, atheroscelorosis, macular degeneration, retinopathy, and arthritis.

4. A method for the treatment or prevention of conditions mediated by the α_{V}β₅ integrin in a mammal in need of such treatment, the method comprising administering to the subject a therapeutically effective amount of a composition of claim 1.

5. The method according to claim 4 wherein the condition treated is selected from the group consisting of tumor metastasis, solid tumor growth, angiogenesis, osteoporosis, humoral hypercalcemia of malignancy, smooth muscle cell migration, restenosis, atheroscelorosis, macular degeneration, retinopathy, and arthritis.
